(19) United States
(12) Patent Application Publication
(10) Pub. No.: US 2026/0256899 A1
(43) Pub. Date: Sep. 3, 2026

(54) PORPHYROMONAS GINGIVALIS ANTIGENIC CONSTRUCTS

(71) Applicant: SANOFI, Paris (FR)

(72) Inventors: Yves GIRERD-CHAMBAZ, Cambridge, MA (US); Andreas KARLSSON, Cambridge, MA (US); Fabienne PIRAS-DOUCE, Cambridge, MA (US); Khang ANH TRAN, Cambridge, MA (US)

(21) Appl. No.: 19/450,935

(22) Filed: Jan. 16, 2026

Related U.S. Application Data
(63) Continuation of application No. PCT/EP2024/070627, filed on Jul. 19, 2024.

(30) Foreign Application Priority Data
Jul. 19, 2023 (EP) 23306245.4
Dec. 18, 2023 (EP) 23307238.8

(51) Int. Cl. A61K 39/02; A61K 38/00; A61K 39/00; A61P 1/02; C07K 14/195; C12N 9/52

(52) U.S. Cl. CPC A61K 39/0216; A61P 1/02; C07K 14/195; C12N 9/52; A61K 38/00; A61K 2039/53; C07K 2319/42; C12Y 304/22037

(57) ABSTRACT

This invention relates to compositions (e.g. vaccine compositions) which can be used to immunise against P. gingivalis infections. The compositions comprise P. gingivalis antigens and antigen combinations which can be used to immunise against P. gingivalis, used in the form of nucleic acids (e.g. mRNAs) encoding antigenic proteins or in the form of recombinant protein antigens.

### RELATED APPLICATIONS

This application is a continuation of International Patent Application No. PCT/EP2024/070627, filed Jul. 19, 2024, which claims priority to European Patent Application Nos. 23307238.8, filed Dec. 18, 2023, and 23306245.4, filed Jul. 19, 2023, the entire disclosures of which are hereby incorporated by reference.

### SEQUENCE LISTING

The instant application contains a Sequence Listing which has been submitted electronically in XML format and is hereby incorporated by reference in its entirety. Said XML file, created on May 7, 2026, is named 773554_SA9-396PCCON_ST26.xml and is 605,716 bytes in size.

### FIELD OF THE INVENTION

The invention is in the field of treating and preventing Porphyromonas gingivalis (P. gingivalis) infections, such as periodontitis. In particular, the invention relates to antigens and antigen combinations which can be used to immunise against P. gingivalis, used in the form of nucleic acids (e.g. mRNAs) encoding antigenic proteins or in the form of recombinant protein antigens.

### BACKGROUND

Periodontitis is a chronic inflammatory disease of the tooth-supporting tissues (Bostanci and Belibasakis, 2012). It affects all age groups but has higher incidence in the elderly population. Its main symptoms are bleeding or swollen gums, pain and sometimes bad breath. It is characterized by the formation of periodontal pockets which support colonization by pathogenic bacteria and the formation of subgingival plaque. In its severe form, periodontitis can lead to the destruction of the periodontal ligament and the alveolar bone and eventual tooth loss (Kinane et al., 2017). Periodontitis is estimated to affect nearly 50% of the global population, making it one of the most prevalent inflammatory diseases and the major cause of tooth loss in adults (Mei et al., 2020). In 2022, WHO estimated that around 19% of the global adult population is affected by severe periodontal disease, representing more than 1 billion cases worldwide (WHO Global Oral Health Status Report, 2022).

Porphyromonas gingivalis (P. gingivalis) is a key etiological agent in periodontitis (or periodontal disease). A Gram-negative non-motile anaerobic pathogen, it requires vitamin K and iron in the form of heme or hemin for its growth and ferments amino acids to produce energy (Bostanci and Belibasakis, 2012). It is a secondary coloniser of the human oral cavity, adhering to primary colonisers in order to form communities and colonise the dental plaque. P. gingivalis resides mainly in the deep periodontal pockets characteristic of periodontitis and has been detected in 85% of subgingival plaque samples from chronic periodontitis patients (How et al., 2016). It is thought to induce periodontitis progression by remodelling the commensal bacterial community in the oral cavity to promote further colonisation by pathogenic bacteria which leads to an imbalance of the microbial biofilm state (or dysbiosis) (Xu et al., 2020). Apart from playing a key role in periodontitis, P. gingivalis is also considered to be a potential risk factor for the development of multiple systematic diseases, such atherosclerosis, cancer, Alzheimer's disease, diabetes and rheumatoid arthritis (Mei et al., 2020).

The major virulence factors of P. gingivalis include lipopolysaccharides, fimbriae, capsule proteins, gingipains and outer membrane vesicles (Xu et al., 2020). Gingipains belong to a family of cysteine proteinase enzymes. They account for 85% of the extracellular proteolytic activity and 99% of the “trypsin-like activity” of P. gingivalis. They are typically located on the cell surface or on the outer membrane vesicles of P. gingivalis strains, except for strain HG66 which also secretes soluble forms of gingipains into the extracellular environment (Li and Collyer, 2011).

Gingipains include arginine-specific gingipains (RgpA and RgpB) and lysine-specific gingipain (Kgp) which cleave polypeptides at the C-terminus after arginine residues or lysine residues, respectively. These three proteins are encoded by individual gene loci found in the genome of all P. gingivalis strains (Li and Collyer, 2011).

The primary function of gingipains is postulated to be the digestion of proteins for nutrition. For example, Kgp is proposed to cleave host heme proteins to provide P. gingivalis with heme for its growth. However, gingipains have recently been found to also participate in the pathogenesis of P. gingivalis. In particular, gingipains are thought to degrade collagen and fibrin/fibrinogen, thereby contributing to gingival tissue breakdown, inhibiting blood clotting and increasing bleeding of the periodontal tissues. Kgp and RgpA are also thought to mediate adhesion to host tissues and to promote co-aggregation of P. gingivalis with other oral pathogens and subsequent biofilm formation. Furthermore, gingipains have been suggested to modulate the host immune response, suppressing the ability of the innate and adaptive immune response to eliminate bacteria while increasing inflammation (Aleksijevic et al., 2022).

Current therapies for P. gingivalis-induced periodontitis include debridement (the removal of plaque and calculus) from teeth by scaling and, in more severe cases, surgery. Adjunctive therapies include the prescription of antibiotics and antimicrobials (Kinane et al., 2017), but these drugs are thought to have reduced efficacy against P. gingivalis because of its ability to form biofilms (Aleksijevic et al., 2022). Therefore, there is a need for an effective vaccine for treatment and/or prevention of P. gingivalis-associated disease. It is postulated that targeting the key virulence factors of P. gingivalis, such as gingipains, by preimmunization may reduce the ability of the bacteria to cause periodontitis or to migrate to distant tissues and instigate other inflammatory diseases (Mei et al., 2020).

One vaccine candidate for P. gingivalis was based on a modified Kgp protein containing portions of the proteinase catalytic domain and adhesin domains of Kgp (known as Kas2-A1-O'Brien-Simpson et al., 2011 and WO2011014947A1). However, there remains a need for improved P. gingivalis vaccines.

It is an object of the invention to provide antigens that are able to elicit functional antibody responses to inhibit the proteinase activity of the gingipain catalytic domain and the haemagglutination and adhesion functions mediated by the gingipain adhesion domains.

### DISCLOSURE OF THE INVENTION

The inventors have found that antigens derived from Kgp, RgpA and/or RgpB, as described herein, can be used to immunise against P. gingivalis. In particular, the inventors found that antigens derived from Kgp, RgpA or RgpB polypeptides of P. gingivalis domains that comprise certain portions of the Kgp, RgpA or RgpB polypeptide elicited robust B cell (i.e. antibody) responses when delivered by mRNAs encoding the relevant antigens.

Accordingly, the invention provides P. gingivalis polypeptides and nucleic acids comprising a nucleotide sequence encoding such polypeptides. Polypeptide antigens described herein may be delivered by, i.e. in the form of, a nucleic acid (e.g. mRNA) comprising a nucleotide sequence encoding said polypeptide.

The invention also provides compositions comprising a combination of (i) a Kgp-based polypeptide or nucleic acid, as described herein, and (ii) a RgpA-based polypeptide or nucleic acid, as described herein.

### Gingipains

The term “gingipain” as used herein refers to a P. gingivalis lysine-specific proteinase (Kgp), or one of the arginine-specific proteinases (RgpA and RgpB). The term “gingipains” is used to refer to Kgp, RgpA and RgpB. The terms “Kgp-based” and “RgpA-based” are used herein to refer to polypeptides and nucleic acids encoding polypeptides that contain Kgp or RgpA elements respectively. Polypeptides that contain Kgp and RgpA elements are referred to as “Kgp and RgpA-based”.

The domain structure of gingipains is highly conserved among P. gingivalis strains. Kgp and RgpA have the same basic modular structure from the N-terminus to C-terminus of the protein: a signal peptide, an N-terminal pro-peptide (which is cleaved in the mature proteins), a protease catalytic domain (Cat), and a C-terminal haemagglutinin/adhesin region composed of a domain of unknown function (DUF), specifically DUF2436, followed by three cleaved adhesion domains, specifically K1, K2 and K3 adhesin domains. These domains are interspersed with sequences containing adhesion binding motifs (ABMs), known as ABM1, ABM2 and ABM3. In the native Kgp and RgpA, a first ABM1 and a first ABM2 are located either side of the DUF2436 (i.e. a first ABM1 is located in N-terminally of the DUF2436, between the DUF2436 and Cat domain, and a first ABM2 is positioned C-terminally of the DUF2436). A second ABM1 is located C-terminally of the first ABM2, which in turn is followed by an ABM3. The second ABM1 and ABM3 are located N-terminally of the K1 adhesion domain. The K1 adhesion domain is followed by the K2 adhesion domain, and subsequently a second ABM2. Finally, a third ABM1 and a third ABM2 are located either side of the K3 adhesion domain, before the protein terminates with a C-terminal domain (Li and Collyer, 2011).

The arrangement of the different domains of Kgp and RgpA is illustrated in FIG. 1. As a particular example, FIGS. 2A and 2B show the wild-type sequence of Kgp and RgpA from P. gingivalis strain W50 in which each domain is highlighted and annotated according to residue position within the wild-type sequence.

The Cat, DUF2436 and K3 domains of Kgp and RgpA show high sequence divergence, whereas the adhesin domains K1, K2, ABM1, ABM2 and ABM3 are highly conserved between RgpA and Kgp. For example, the Cat domains of Kgp and RgpA share approximately 27% sequence identity, while the DUF2436 domains of Kgp and RgpA share approximately 53% sequence identity. In contrast each of the K1 and K2 adhesin domains of Kgp and RgpA share more than approximately 99% sequence identity respectively.

RgpB contains the signal peptide, the N-terminal pro-peptide, the protease catalytic domain and a short C-terminal domain. RgpB lacks the adhesin domain DUF2436, the adhesion binding motifs and the K1-K3 domains. The Cat domain of RgpB shares ~90% sequence identity with the Cat domain of RgpA but only 20-30% sequence identity with the Cat domain of Kgp (Li and Collyer, 2011).

In a first aspect the invention provides a nucleic acid comprising a nucleotide sequence encoding a polypeptide, wherein the polypeptide comprises:
i) at least a portion of a Porphyromonas gingivalis Lys-specific proteinase (Kgp) catalytic domain;
ii) at least a portion of a Porphyromonas gingivalis Kgp domain of unknown function 2436 (DUF2436);
iii) at least a portion of a Porphyromonas gingivalis Kgp K1 adhesin domain;
iv) a first Porphyromonas gingivalis Kgp portion that comprises an adhesin binding motif 1 (ABM1) and a first Porphyromonas gingivalis Kgp portion that comprises an adhesin binding motif 2 (ABM2);
v) a second Porphyromonas gingivalis Kgp portion that comprises an ABM1 and a second Porphyromonas gingivalis Kgp portion that comprises an ABM2; and
vi) a Porphyromonas gingivalis Kgp portion that comprises an ABM3.

In a second aspect, the invention provides a nucleic acid comprising a nucleotide sequence encoding a polypeptide, wherein the polypeptide comprises:
i) at least a portion of a Porphyromonas gingivalis Arg-specific proteinase A (RgpA) catalytic domain or Arg-specific proteinase B (RgpB) catalytic domain;
ii) at least a portion of a Porphyromonas gingivalis RgpA DUF2436;
iii) at least a portion of a Porphyromonas gingivalis RgpA K1 adhesin domain;
iv) a first Porphyromonas gingivalis RgpA portion that comprises an ABM1, and a first Porphyromonas gingivalis RgpA portion that comprises an ABM2;
v) a second Porphyromonas gingivalis RgpA portion that comprises an ABM1, and a second Porphyromonas gingivalis RgpA portion that comprises an ABM2; and
vi) a Porphyromonas gingivalis RgpA portion that comprises an ABM3.

In a third aspect, the invention provides a polypeptide comprising:
i) at least a portion of a Porphyromonas gingivalis Lys-specific proteinase (Kgp) catalytic domain;
ii) at least a portion of a Porphyromonas gingivalis Kgp domain of unknown function 2436 (DUF2436);
iii) at least a portion of a Porphyromonas gingivalis Kgp K1 adhesin domain;
iv) a first Porphyromonas gingivalis Kgp portion that comprises an adhesin binding motif 1 (ABM1) and a first Porphyromonas gingivalis Kgp portion that comprises an adhesin binding motif 2 (ABM2);
v) a second Porphyromonas gingivalis Kgp portion that comprises an ABM1 and a second Porphyromonas gingivalis Kgp portion that comprises an ABM2; and
vi) a Porphyromonas gingivalis Kgp portion that comprises an ABM3.

In a fourth aspect, the invention provides a polypeptide comprising:
i) at least a portion of a Porphyromonas gingivalis Arg-specific proteinase A (RgpA) catalytic domain or Arg-specific proteinase B (RgpB) catalytic domain;
ii) at least a portion of a Porphyromonas gingivalis RgpA DUF2436;
iii) at least a portion of a Porphyromonas gingivalis RgpA K1 adhesin domain;
iv) a first Porphyromonas gingivalis RgpA portion that comprises an ABM1, and a first Porphyromonas gingivalis RgpA portion that comprises an ABM2;
v) a second Porphyromonas gingivalis RgpA portion that comprises an ABM1, and a second Porphyromonas gingivalis RgpA portion that comprises an ABM2; and
vi) a Porphyromonas gingivalis RgpA portion that comprises an ABM3.

In another aspect, the invention provides a composition comprising any one of the nucleic acids of the invention, preferably wherein the composition is an immunogenic composition.

In another aspect, the invention provides a composition comprising a first nucleic acid and second nucleic acid of the invention, preferably wherein the composition is an immunogenic composition.

In another aspect, the invention provides a composition comprising any one of the polypeptides of the invention, preferably wherein the composition is an immunogenic composition.

In another aspect, the invention provides a composition comprising a first polypeptide and second polypeptide of the invention, preferably wherein the composition is an immunogenic composition.

In another aspect, the invention provides a vaccine comprising any one of the nucleic acids, any one of the polypeptides or any one of the compositions of the invention.

The modular nature of gingipains means that the nucleic acids and polypeptides of the invention may combine domains derived from different gingipains. Accordingly, the nucleic acids and polypeptides of the invention may comprise, for example, any of the catalytic domains described herein with any of the DUF2436 domains described herein. As another example, any of the portions of gingipains comprising an ABM1, as described herein may be combined with any of the DUF2436 domains described herein.

P. gingivalis Strains

The nucleic acids and polypeptides of the invention may be derived from any P. gingivalis strain. The modular structure of gingipains means that one domain of the nucleic acid or polypeptide may be derived from one strain of P. gingivalis and another domain derived from a different strain of P. gingivalis. In another embodiment, all domains of the nucleic acid or polypeptide are derived from the same strain of P. gingivalis.

Examples of P. gingivalis strains are shown in Table 1 along with their corresponding GenBank sequence. The skilled person is able to identify different domains of Kgp, RgpA or RgpB, or portions of Kgp, RgpA or RgpB comprising e.g. ABMs in a strain of P. gingivalis for example, by comparison with the sequences of the corresponding domains or portions of Kgp or RgpA in P. gingivalis strain W50, as disclosed herein and indicated in FIGS. 2A-2B. An example of a P. gingivalis strain W50 wild-type Kgp sequence is provided in SEQ ID NO:157, with the corresponding nucleic acid encoding this sequence in SEQ ID NO: 160. An example of a P. gingivalis strain W50 wild-type RgpA sequence is provided in SEQ ID NO:158, with the corresponding nucleic acid encoding this sequence in SEQ ID NO: 161. An example of a P. gingivalis strain W50 wild-type RgpB sequence is provided in SEQ ID NO: 159, with the corresponding nucleic acid encoding this sequence in SEQ ID NO: 162.

TABLE 1
Examples of P. gingivalis strains

| P. gingivalis strain | GenBank reference |
|---|---|
| W50 | GCA_023822425.1 |
| W83 | GCA_002892595.1 |
| ATCC33277 | GCA_000010505.1 |
| TDC60 | GCA_000270225.1 |
| ATCC49417 | GCA_028993465.1 |
| AFR5B1 | GCA_900157345.1 |
| 3A1 | GCA_900157325.1 |
| 11A | GCA_900157295.1 |
| 7BTORR | GCA_900157225.1 |
| 13_1 | GCA_900157285.1 |
| 15_9 | GCA_900157245.1 |
| 3_3 | GCA_900157215.1 |

The nucleic acids and polypeptides of the invention may be derived from P. gingivalis and comprise several domains that may correspond to domains in naturally occurring Kgp, RgpA or RgpB sequences. However, the nucleic acids do not encode polypeptides that are naturally occurring full-length Kgp, RgpA or RgpB polypeptides (or mature polypeptides). Similarly, the polypeptides of the invention are not naturally occurring full-length Kgp, RgpA or RgpB polypeptides (or mature polypeptides).

In other words, the nucleic acids of the invention may encode polypeptides that are modified relative to a naturally occurring full-length Kgp, RgpA or RgpB polypeptide. Similarly, the polypeptides of the invention are modified relative to a naturally occurring full-length Kgp, RgpA or RgpB polypeptides. A modified polypeptide may be a variant of a naturally occurring polypeptide with altered amino acid sequences due to, for example, amino acid substitutions, deletions, or insertions. A modified polypeptide may be a truncation or fragment of a naturally occurring polypeptide.

In any of the embodiments disclosed herein, the polypeptide may be a modified polypeptide according to the invention as described elsewhere herein. In any of the embodiments disclosed herein, the nucleic acid may encode a modified polypeptide according to the invention as described elsewhere herein.

The polypeptides of the invention may also be in the form of recombinant polypeptides. Thus, in any of the embodiments described herein, the polypeptide is a recombinant polypeptide.

### Adhesin Binding Motifs (ABMs)

Adhesin binding motifs (ABMs) are sequences found within native gingipains that are postulated to contribute to the adhesion function of gingipains. Three different gingipain ABMs have been described: ABM1, ABM2 and ABM3 (Li and Collyer, 2011). ABM1 was first described on the basis of the identification of conserved sequences (Slakeski et al., 1998). ABM2 and ABM3 were described according to sequences that were bound by certain antibodies (O'Brien-Simpson et al., 2005).

The nucleic acids and polypeptides of the invention comprise a first portion of a Kgp or RgpA comprising an ABM1, a first portion of a Kgp or RgpA comprising an ABM2, a second portion of a Kgp or RgpA comprising an ABM1 and a second portion of a Kgp or RgpA comprising an ABM2.

### Portions of Gingipains Comprising an ABM1

In some embodiments, the first Kgp portion comprising an ABM1 is derived from the P. gingivalis strain W50. In P. gingivalis strain W50, the first Kgp portion comprising an ABM1 has a sequence of SEQ ID NO: 106. Accordingly, in some embodiments, the first Kgp portion comprising an ABM1 comprises a sequence of SEQ ID NO: 106 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM1 is derived from the P. gingivalis strain W50. In P. gingivalis strain W50, the first RgpA portion comprising an ABM1 has a sequence of SEQ ID NO: 114. Accordingly, in some embodiments, the first RgpA portion comprising an ABM1 comprises a sequence of SEQ ID NO: 114 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM1 is derived from the P. gingivalis strain W50. In P. gingivalis strain W50, the second Kgp portion comprising an ABM1 has a sequence of SEQ ID NO: 110. Accordingly, in some embodiments, the second Kgp portion comprising an ABM1 comprises a sequence of SEQ ID NO: 110 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM1 is derived from the P. gingivalis strain W50. In P. gingivalis strain W50, the second RgpA portion comprising an ABM1 has a sequence of SEQ ID NO: 102. Accordingly, in some embodiments, the second RgpA portion comprising an ABM1 comprises a sequence of SEQ ID NO: 102 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Examples of portions of a gingipain (e.g. Kgp or RgpA) sequence comprising ABM1 sequences are provided in, along with a consensus sequence for ABM1. The consensus sequence for ABM1 is SEQ ID NO: 120.

TABLE 2
Portions of Kgp and RgpA sequences that comprise an ABM1. The consensus sequence
for ABM1 is underlined in each sequence.

| Sequence | SEQ ID NO | Gingipain |
|---|---|---|
| GEPSPYQPVSNLTATTQGQKVTLKWEAPSAKKAEG | SEQ ID NO: 106 | Kgp (first ABM1 |
| | | portion) |
| PVSNLTATTQGQKVTLKWEAPSAK | SEQ ID NO: 107 | Kgp (first ABM1 |
| | | portion) |
| PYQPVSQLTATTQGQKVTLKWEAPSAKKSEG | SEQ ID NO: 89 | Kgp (first ABM1 |
| | | portion) |
| GEPSPYPQPVSNLTATTQGQKVTLKWEAPS | SEQ ID NO: 108 | Kgp (first ABM1 |
| | | portion) |
| PVSNLTATTQGQKVTLKWEAPSA | SEQ ID NO: 109 | Kgp (first ABM1 |
| | | portion) |
| NEFAPVQNLTGSSVGQKVTLKWDAPN | SEQ ID NO: 110 | Kgp (second ABM1 |
| | | portion) |
| PVQNLTGSSVGQKVTLKWDAPN | SEQ ID NO: 111 | Kgp (second ABM1 |
| | | portion) |
| NEFAPVQQLTGSSVGQKVTLKWDA | SEQ ID NO: 92 | Kgp (second ABM1 |
| | | portion) |
| VGQKVTLKWDAPNGT | SEQ ID NO: 112 | Kgp (second ABM1 |
| | | portion) |
| PVQNLTGSSVGQKVTLKWDAPST | SEQ ID NO: 113 | Kgp (second ABM1 |
| | | portion) |
| NPYQPVSNLTATTQGQKVTLKWDAPSTKTNAT | SEQ ID NO: 114 | RgpA (first ABM1 |
| | | portion) |
| NPYQPVSNLAATTQGQKVTLKWDAPSTKTQAT | SEQ ID NO: 99 | RgpA (first ABM1 |
| | | portion) |
| GEPNPYPQPVSNLTATTQGQKVTLKWDAPS | SEQ ID NO: 115 | RgpA (first ABM1 |
| | | portion) |
| PVSNLTATTQGQKVTLKWDAPST | SEQ ID NO: 116 | RgpA (first ABM1 |
| | | portion) |
| NEFAPVQNLTGSAVGQKVTLKWDA | SEQ ID NO: 102 | RgpA (second |
| | | ABM1 portion) |
| VGQKVTLKWDAPNGT | SEQ ID NO: 117 | RgpA (second |
| | | ABM1 portion) |
| PVQNLTGSAVGQKVTLKWDAPNG | SEQ ID NO: 118 | RgpA (second |
| | | ABM1 portion) |
| NEFAPVQNLTGSAVGQKVTLKWDAPNG | SEQ ID NO: 119 | RgpA (second |
| | | ABM1 portion) |
| GQKVTLKWE/DA | SEQ ID NO: 120 | Consensus motif |

In some embodiments, the first Kgp portion comprising an ABM1 comprises a sequence of SEQ ID NO: 120, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM1 comprises a sequence of SEQ ID NO: 106, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM1 comprises a sequence of SEQ ID NO: 107, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM1 comprises a sequence of SEQ ID NO: 89, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM1 comprises a sequence of SEQ ID NO: 108, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM1 comprises a sequence of SEQ ID NO: 109, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM1 comprises a sequence from a Kgp that is bounded at the N-terminus by the Cat domain and at the C-terminus by the DUF2436, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a Kgp that is bounded at the N-terminus by the Cat domain and at the C-terminus by the DUF2436.

In some embodiments, the second Kgp portion comprising an ABM1 comprises a sequence of SEQ ID NO: 120, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM1 comprises a sequence of SEQ ID NO: 110, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM1 comprises a sequence of SEQ ID NO: 111, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM1 comprises a sequence of SEQ ID NO: 92, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM1 comprises a sequence of SEQ ID NO: 112, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM1 comprises a sequence of SEQ ID NO: 113, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM1 comprises a sequence from a Kgp that is bounded at the N-terminus by a ABM2 sequence and at the C-terminus by a ABM3 sequence, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a Kgp that is bounded at the N-terminus by a ABM2 sequence and at the C-terminus by a ABM3 sequence.

In some embodiments, the first RgpA portion comprising an ABM1 comprises a sequence of SEQ ID NO: 120, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM1 comprises a sequence of SEQ ID NO: 114, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM1 comprises a sequence of SEQ ID NO: 99, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM1 comprises a sequence of SEQ ID NO: 115, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM1 comprises a sequence of SEQ ID NO: 116, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM1 comprises a sequence from a RgpA that is bounded at the N-terminus by the Cat domain and at the C-terminus by the DUF2436, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a RgpA that is bounded at the N-terminus by the Cat domain and at the C-terminus by the DUF2436.

In some embodiments, the second RgpA portion comprising an ABM1 comprises a sequence of SEQ ID NO: 120, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM1 comprises a sequence of SEQ ID NO: 102, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM1 comprises a sequence of SEQ ID NO: 117, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM1 comprises a sequence of SEQ ID NO: 118, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM1 comprises a sequence of SEQ ID NO: 119, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM1 comprises a sequence from a RgpA that is bounded at the N-terminus by a ABM2 sequence and at the C-terminus by a ABM3 sequence, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a RgpA that is bounded at the N-terminus by a ABM2 sequence and at the C-terminus by a ABM3 sequence.

In the wild-type sequence of Kgp from P. gingivalis strain W50, the first Kgp portion that comprises an ABM1 is positioned between the Cat domain and the DUF2436 and is 35 amino acids in length and comprises the sequence of SEQ ID NO: 106. This sequence comprises an ABM1 of SEQ ID NO: 120. Accordingly, in some embodiments, the first Kgp portion that comprises an ABM1 is between 10 and 35 amino acids long and comprises the sequence of SEQ ID NO: 120.

In some embodiments, the first Kgp portion that comprises an ABM1 is between 15 and 30 amino acids long and comprises the sequence of SEQ ID NO: 120. In some embodiments, the first Kgp portion that comprises an ABM1 is between 20 and 25 amino acids long and comprises the sequence of SEQ ID NO: 120.

In some embodiments, the first Kgp portion that comprises an ABM1 is 10 amino acids long and comprises the sequence of SEQ ID NO: 120. In some embodiments, the first Kgp portion that comprises an ABM1 is 15 amino acids long and comprises the sequence of SEQ ID NO: 120. In some embodiments, the first Kgp portion that comprises an ABM1 is 20 amino acids long and comprises the sequence of SEQ ID NO: 120. In some embodiments, the first Kgp portion that comprises an ABM1 is 25 amino acids long and comprises the sequence of SEQ ID NO: 120. In some embodiments, the first Kgp portion that comprises an ABM1 is 30 amino acids long and comprises the sequence of SEQ ID NO: 120. In some embodiments, the first Kgp portion that comprises an ABM1 is 35 amino acids long and comprises the sequence of SEQ ID NO: 120.

In some embodiments, the first Kgp portion that comprises an ABM1 is 10, 11, 12, 13, 14, 15, 16, 17, 18, 19, 20, 21, 22, 23, 24, 25, 26, 27, 28, 29, 30, 31, 32, 33, 34 or 35 amino acids long and comprises the sequence of SEQ ID NO: 120.

In the wild-type sequence of Kgp from P. gingivalis strain W50, the second Kgp portion that comprises an ABM1 is positioned between the DUF and K1 adhesin domain. An ABM2 sequence is positioned N-terminally of the second portion that comprises an ABM1, and an ABM3 sequence is positioned C-terminally of the second portion that comprises an ABM1. In this context, the second Kgp portion that comprises an ABM1 is 26 amino acids long and comprises the sequence of SEQ ID NO: 110. This sequence comprises an ABM1 of SEQ ID NO: 120. Accordingly, in some embodiments, the second Kgp portion that comprises an ABM1 is between 10 and 26 amino acids long and comprises the sequence of SEQ ID NO: 120.

In some embodiments, the second Kgp portion that comprises an ABM1 is between 10 and 25 amino acids long and comprises the sequence of SEQ ID NO: 120. In some embodiments, the second Kgp portion that comprises an ABM1 is between 15 and 20 amino acids long and comprises the sequence of SEQ ID NO: 120.

In some embodiments, the second Kgp portion that comprises an ABM1 is 10 amino acids long and comprises the sequence of SEQ ID NO: 120. In some embodiments, the second Kgp portion that comprises an ABM1 is 15 amino acids long and comprises the sequence of SEQ ID NO: 120. In some embodiments, the second Kgp portion that comprises an ABM1 is 20 amino acids long and comprises the sequence of SEQ ID NO: 120. In some embodiments, the second Kgp portion that comprises an ABM1 is 25 amino acids long and comprises the sequence of SEQ ID NO: 120. In some embodiments, the second Kgp portion that comprises an ABM1 is 26 amino acids long and comprises the sequence of SEQ ID NO: 120.

In some embodiments, the second Kgp portion that comprises an ABM1 is 10, 11, 12, 13, 14, 15, 16, 17, 18, 19, 20, 21, 22, 23, 24, 25 or 26 amino acids long and comprises the sequence of SEQ ID NO: 120.

In the wild-type sequence of RgpA from P. gingivalis strain W50, the first RgpA portion that comprises an ABM1 is positioned between the Cat domain and the DUF domain and is 32 amino acids in length and comprises the sequence of SEQ ID NO: 114. This sequence comprises an ABM1 of SEQ ID NO: 120. Accordingly, in some embodiments, the first RgpA portion that comprises an ABM1 is between 10 and 32 amino acids long and comprises the sequence of SEQ ID NO: 120.

In some embodiments, the first RgpA portion that comprises an ABM1 is between 15 and 30 amino acids long and comprises the sequence of SEQ ID NO: 120. In some embodiments, the first RgpA portion that comprises an ABM1 is between 20 and 25 amino acids long and comprises the sequence of SEQ ID NO: 120.

In some embodiments, the first RgpA portion that comprises an ABM1 is 10 amino acids long and comprises the sequence of SEQ ID NO: 120. In some embodiments, the first RgpA portion that comprises an ABM1 is 15 amino acids long and comprises the sequence of SEQ ID NO: 120. In some embodiments, the first RgpA portion that comprises an ABM1 is 20 amino acids long and comprises the sequence of SEQ ID NO: 120. In some embodiments, the first RgpA portion that comprises an ABM1 is 25 amino acids long and comprises the sequence of SEQ ID NO: 120. In some embodiments, the first RgpA portion that comprises an ABM1 is 30 amino acids long and comprises the sequence of SEQ ID NO: 120. In some embodiments, the first RgpA portion that comprises an ABM1 is 32 amino acids long and comprises the sequence of SEQ ID NO: 120.

In some embodiments, the first RgpA portion that comprises an ABM1 is 10, 11, 12, 13, 14, 15, 16, 17, 18, 19, 20, 21, 22, 23, 24, 25, 26, 27, 28, 29, 30, 31 or 32 amino acids long and comprises the SEQ ID NO: 120.

In the wild-type sequence of RgpA from P. gingivalis strain W50, the second RgpA portion that comprises an ABM1 is positioned between the DUF and K1 adhesin domain. An ABM2 sequence is positioned N-terminally of the second portion that comprises an ABM1, and an ABM3 sequence is positioned C-terminally of the second portion that comprises an ABM1. In this context, the second RgpA portion that comprises an ABM1 is 27 amino acids long and comprises the sequence of SEQ ID NO: 119. This sequence comprises an ABM1 of SEQ ID NO: 120. Accordingly, in some embodiments, the second RgpA portion that comprises an ABM1 is between 10 and 27 amino acids long and comprises the sequence of SEQ ID NO: 120.

In some embodiments, the second RgpA portion that comprises an ABM1 is between 10 and 25 amino acids long and comprises the sequence of SEQ ID NO: 120. In some embodiments, the second RgpA portion that comprises an ABM1 is between 15 and 20 amino acids long and comprises the sequence of SEQ ID NO: 120.

In some embodiments, the second RgpA portion that comprises an ABM1 is 10 amino acids long and comprises the sequence of SEQ ID NO: 120. In some embodiments, the second RgpA portion that comprises an ABM1 is 15 amino acids long and comprises the sequence of SEQ ID NO: 120. In some embodiments, the second RgpA portion that comprises an ABM1 is 20 amino acids long and comprises the sequence of SEQ ID NO: 120. In some embodiments, the second RgpA portion that comprises an ABM1 is 25 amino acids long and comprises the sequence of SEQ ID NO: 120. In some embodiments, the second RgpA portion that comprises an ABM1 is 27 amino acids long and comprises the sequence of SEQ ID NO: 120.

In some embodiments, the second RgpA portion that comprises an ABM1 is 10, 11, 12, 13, 14, 15, 16, 17, 18, 19, 20, 21, 22, 23, 24, 25, 26 or 27 amino acids long and comprises the sequence of SEQ ID NO: 120.

### Portions of Gingipains Comprising an ABM2

In some embodiments, the first Kgp portion comprising an ABM2 is derived from the P. gingivalis strain W50. In P. gingivalis strain W50, first Kgp portion comprising an ABM2 has a sequence of SEQ ID NO: 121. Accordingly, in some embodiments, the first Kgp portion comprising an ABM2 comprises a sequence of SEQ ID NO: 121 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM2 is derived from the P. gingivalis strain W50. In P. gingivalis strain W50, the first RgpA portion comprising an ABM2 has a sequence of SEQ ID NO: 101. Accordingly, in some embodiments, the first RgpA portion comprising an ABM2 comprises a sequence of SEQ ID NO: 101 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM2 is derived from the P. gingivalis strain W50. In P. gingivalis strain W50, second Kgp portion comprising an ABM2 has a sequence of SEQ ID NO: 124. Accordingly, in some embodiments, the second Kgp portion comprising an ABM2 comprises a sequence of SEQ ID NO: 124 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM2 is derived from the P. gingivalis strain W50. In P. gingivalis strain W50 the, second RgpA portion comprising an ABM2 has a sequence of SEQ ID NO: 126. Accordingly, in some embodiments, the second RgpA portion comprising an ABM21 comprises a sequence of SEQ ID NO: 126 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Examples of portions of a gingipain (e.g. Kgp or RgpA) sequence comprising ABM2 sequences are provided in along with a consensus sequence for ABM2 in. The consensus sequence for ABM2 is SEQ ID NO: 130.

TABLE 3
Portions of Kgp and RgpA sequences that comprise an ABM2. The consensus sequence
for ABM2 is underlined in each sequence

| Sequence | SEQ ID NO | Gingipain |
|---|---|---|
| SYTYTVYRDGTKIKEGLTATTFEEDGVAAGNHEYCVEVKY | SEQ ID NO: | Kgp (first ABM2 |
| TAGVSPKVCKDVTVEGS | 121 | portion) |
| SYTYTVYRDGTKIKEGLTATTFEEDGVAA | SEQ ID NO: | Kgp (first ABM2 |
| | 122 | portion) |
| EAEDDSPASYTYTVYRDGTKIKEGLTATTFEEDGVAAGNHE | SEQ ID NO: 91 | Kgp (first ABM2 |
| YCVEVKYTAGVSPKVCKDVTVEGS | | portion) |
| SYTYTVYRDGTKIKEGLTATTFEEDGVAA | SEQ ID NO: | Kgp (first ABM2 |
| | 123 | portion) |
| TDYTYTVYRDGTKIKEGLTETTFEEDGVATGNHEYCVEVK | SEQ ID NO: | Kgp (second |
| YTAGVSPKKCVNVTVN | 124 | ABM2 portion) |
| TDYTYTVYRDGTKIKEGLTETTFEEDGVATGNHEYCVEVK | SEQ ID NO: 95 | Kgp (second |
| YTAGVSPKKCVDVTVN | | ABM2 portion) |
| EGLTATTFEEDGVAA | SEQ ID NO: | Kgp (second |
| | 125 | ABM2 portion) |
| TISEGGGSDYTYTVYRDGTKIKEGLTATTFEEDGVAAGNHE | SEQ ID NO: | RgpA (first |
| YCVEVKYTAGVSPKVCKDVTVEGS | 101 | ABM2 portion) |
| TDYTYTVYRDGTKIKEGLTETTFEEDGVATGNHEYCVEVK | SEQ ID NO: | RgpA (second |
| YTAGVSPKKCVNVTVN | 126 | ABM2 portion) |
| TDYTYTVYRDGTKIKEGLTETTFEEDGVATGNHEYCVEVK | SEQ ID NO: | RgpA (second |
| YTAGVSPKKCVQVTVN | 105 | ABM2 portion) |
| YTYTVYRDGTKIKEGLTETTFEEDGVA | SEQ ID NO: | RgpA (second |
| | 127 | ABM2 portion) |
| SYTYTVYRDGTKIKEGLTATTFEEDGVAA | SEQ ID NO: | RgpA (second |
| | 128 | ABM2 portion) |
| EGLTATTFEEDGVAA | SEQ ID NO: | RgpA (second |
| | 129 | ABM2 portion) |
| EGLTE/ATTFEEDGVA | SEQ ID NO: | Consensus motif |
| | 130 | |

In some embodiments, the first Kgp portion comprising an ABM2 comprises a sequence of SEQ ID NO: 130, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM2 comprises a sequence of SEQ ID NO: 121, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM2 comprises a sequence of SEQ ID NO: 122, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM2 comprises a sequence of SEQ ID NO: 91, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM2 comprises a sequence of SEQ ID NO: 123, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first Kgp portion comprising an ABM2 comprises a sequence from a Kgp that is bounded at the N-terminus by a DUF2436 and at the C-terminus by an ABM1 sequence, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a Kgp that is bounded at the N-terminus by a DUF2436 and at the C-terminus by an ABM1 sequence.

In some embodiments, the second Kgp portion comprising an ABM2 comprises a sequence of SEQ ID NO: 130, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM2 comprises a sequence of SEQ ID NO: 124, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM2 comprises a sequence of SEQ ID NO: 125, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM2 comprises a sequence of SEQ ID NO: 95, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second Kgp portion comprising an ABM2 comprises a sequence from a Kgp that is bounded at the N-terminus by a Kgp K2 adhesin domain and at the C-terminus by an ABM1 sequence, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a Kgp that is bounded at the N-terminus by a sequence from a Kgp that is bounded at the N-terminus by a Kgp K2 adhesin domain and at the C-terminus by an ABM1.

In some embodiments, the first RgpA portion comprising an ABM2 comprises a sequence of SEQ ID NO: 130, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM2 comprises a sequence of SEQ ID NO: 101, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM2 comprises a sequence from a RgpA that is bounded at the N-terminus by a DUF2436 and at the C-terminus by an ABM1 sequence, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a RgpA that is bounded at the N-terminus by a DUF2436 and at the C-terminus by an ABM1 sequence.

In some embodiments, the second RgpA portion comprising an ABM2 comprises a sequence of SEQ ID NO: 130, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM2 comprises a sequence of SEQ ID NO: 105, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM2 comprises a sequence of SEQ ID NO: 126, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM2 comprises a sequence of SEQ ID NO: 127, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM2 comprises a sequence of SEQ ID NO: 128, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM2 comprises a sequence of SEQ ID NO: 129, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the second RgpA portion comprising an ABM2 comprises a sequence from a RgpA that is bounded at the N-terminus by a RgpA K2 adhesin domain and at the C-terminus by an ABM1 sequence, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity to a sequence from a RgpA that is bounded at the N-terminus by a sequence from a RgpA that is bounded at the N-terminus by a RgpA K2 adhesin domain and at the C-terminus by an ABM1.

In the wild-type sequence of Kgp from P. gingivalis strain W50, the first Kgp portion that comprises an ABM2 is positioned between the DUF2436 and K1 adhesin domain. The DUF2436 is positioned N-terminally of the first portion that comprises an ABM2. An ABM1 sequence and an ABM3 sequence are positioned C-terminally of the first portion that comprises an ABM2. In this context, the first Kgp portion that comprises an ABM2 is 65 amino acids in length and comprises the sequence of SEQ ID NO: 91. This sequence comprises an ABM2 of SEQ ID NO: 130. Accordingly, in some embodiments, the first Kgp portion that comprises an ABM2 is between 14 and 65 amino acids long and comprises the sequence of SEQ ID NO: 130.

In some embodiments, the first Kgp portion that comprises an ABM2 is between 20 and 60 amino acids long and comprises the sequence of SEQ ID NO: 130. In some embodiments, the first Kgp portion that comprises an ABM2 is between 25 and 55 amino acids long and comprises the sequence of SEQ ID NO:

130. In some embodiments, the first Kgp portion that comprises an ABM2 is between 30 and 50 amino acids long and comprises the sequence of SEQ ID NO: 130. In some embodiments, the first Kgp portion that comprises an ABM2 is between 35 and 45 amino acids long and comprises the sequence of SEQ ID NO: 130. In some embodiments, the first Kgp portion that comprises an ABM2 is between 35 and 40 amino acids long and comprises the sequence of SEQ ID NO: 130.

In some embodiments, the first Kgp portion that comprises an ABM2 is 14 amino acids long and comprises the sequence of SEQ ID NO: 130. In some embodiments, the first Kgp portion that comprises an ABM2 is 20 amino acids long and comprises the sequence of SEQ ID NO: 130. In some embodiments, the first Kgp portion that comprises an ABM2 is 25 amino acids long and comprises the sequence of SEQ ID NO: 130. In some embodiments, the first Kgp portion that comprises an ABM2 is 30 amino acids long and comprises the sequence of SEQ ID NO: 130. In some embodiments, the first Kgp portion that comprises an ABM2 is 35 amino acids long and comprises the sequence of SEQ ID NO: 130. In some embodiments, the first Kgp portion that comprises an ABM2 is 40 amino acids long and comprises the sequence of SEQ ID NO: 130. In some embodiments, the first Kgp portion that comprises an ABM2 is 45 amino acids long and comprises the sequence of SEQ ID NO: 130. In some embodiments, the first Kgp portion that comprises an ABM2 is 50 amino acids long and comprises the sequence of SEQ ID NO: 130. In some embodiments, the first Kgp portion that comprises an ABM2 is 55 amino acids long and comprises the sequence of SEQ ID NO: 130. In some embodiments, the first Kgp portion that comprises an ABM2 is 60 amino acids long and comprises the sequence of SEQ ID NO: 130. In some embodiments, the first Kgp portion that comprises an ABM2 is 65 amino acids long and comprises the sequence of SEQ ID NO: 130.

In some embodiments, the first Kgp portion that comprises an ABM2 is 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 amino acids long and comprises the sequence of SEQ ID NO: 130.

In the wild-type sequence of Kgp from P. gingivalis strain W50, the second Kgp portion that comprises an ABM2 is positioned between the K2 and K3 adhesin domains. The second Kgp portion that comprises ABM2 is 56 amino acids long in length and comprises the sequence of SEQ ID NO: 124. This sequence comprises an ABM2 of SEQ ID NO: 130. Accordingly, in some embodiments, the second Kgp portion that comprises an ABM2 is between 14 and 56 amino acids long and comprises the sequence of SEQ ID NO: 130.

In some embodiments, the second Kgp portion that comprises an ABM2 is between 20 and 50 amino acids long and comprises the sequence of SEQ ID NO: 130. In some embodiments, the second Kgp portion that comprises an ABM2 is between 25 and 45 amino acids long and comprises the sequence of SEQ ID NO: 130. In some embodiments, the second Kgp portion that comprises an ABM2 is between 30 and 40 amino acids long and comprises the sequence of SEQ ID NO: 130. In some embodiments, the second Kgp portion that comprises an ABM2 is between 35 and 40 amino acids long and comprises the sequence of SEQ ID NO: 130.

In some embodiments, the second Kgp portion that comprises an ABM2 is 14 amino acids long and comprises the sequence of SEQ ID NO: 130. In some embodiments, the second Kgp portion that comprises an ABM2 is 20 amino acids long and comprises the sequence of SEQ ID NO: 130. In some embodiments, the second Kgp portion that comprises an ABM2 is 25 amino acids long and comprises the sequence of SEQ ID NO: 130. In some embodiments, the second Kgp portion that comprises an ABM2 is 30 amino acids long and comprises the sequence of SEQ ID NO: 130. In some embodiments, the second Kgp portion that comprises an ABM2 is 35 amino acids long and comprises the sequence of SEQ ID NO: 130. In some embodiments, the second Kgp portion that comprises an ABM2 is 40 amino acids long and comprises the sequence of SEQ ID NO: 130. In some embodiments, the second Kgp portion that comprises an ABM2 is 45 amino acids long and comprises the sequence of SEQ ID NO: 130. In some embodiments, the second Kgp portion that comprises an ABM2 is 50 amino acids long and comprises the sequence of SEQ ID NO: 130. In some embodiments, the second Kgp portion that comprises an ABM2 is 56 amino acids long and comprises the sequence of SEQ ID NO: 130.

In some embodiments, the second Kgp portion that comprises an ABM2 is 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 or 56 amino acids long and comprises the sequence of SEQ ID NO: 130.

In the wild-type sequence of RgpA from P. gingivalis strain W50, the first RgpA portion that comprises an ABM2 is positioned between the DUF2436 and K1 adhesin domain. The DUF2436 is positioned N-terminally of the first portion that comprises an ABM2. An ABM1 sequence and an ABM3 sequence are positioned C-terminally of the first portion that comprises an ABM2. In this context, the first RgpA portion that comprises an ABM2 is 65 amino acids in length and comprises the sequence of SEQ ID NO: 101. This sequence comprises an ABM2 of SEQ ID NO: 130 Accordingly, in some embodiments, the first RgpA portion that comprises an ABM2 is between 14 and 65 amino acids long and comprises the sequence of SEQ ID NO: 130.

In some embodiments, the first RgpA portion that comprises an ABM2 is between 20 and 60 amino acids long and comprises the sequence of SEQ ID NO: 130. In some embodiments, the first RgpA portion that comprises an ABM2 is between 25 and 55 amino acids long and comprises the sequence of SEQ ID NO: 130. In some embodiments, the first RgpA portion that comprises an ABM2 is between 30 and 50 amino acids long and comprises the sequence of SEQ ID NO: 130. In some embodiments, the first RgpA portion that comprises an ABM2 is between 35 and 45 amino acids long and comprises the sequence of SEQ ID NO: 130. In some embodiments, the first RgpA portion that comprises an ABM2 is between 35 and 40 amino acids long and comprises the sequence of SEQ ID NO: 130.

In some embodiments, the first RgpA portion that comprises an ABM2 is 14 amino acids long and comprises the sequence of SEQ ID NO: 130. In some embodiments, the first RgpA portion that comprises an ABM2 is 20 amino acids long and comprises the sequence of SEQ ID NO: 130. In some embodiments, the first RgpA portion that comprises an ABM2 is 25 amino acids long and comprises the sequence of SEQ ID NO: 130. In some embodiments, the first RgpA portion that comprises an ABM2 is 30 amino acids long and comprises the sequence of SEQ ID NO: 130. In some embodiments, the first RgpA portion that comprises an ABM2 is 35 amino acids long and comprises the sequence of SEQ ID NO: 130. In some embodiments, the first RgpA portion that comprises an ABM2 is 40 amino acids long and comprises the sequence of SEQ ID NO: 130. In some embodiments, the first RgpA portion that comprises an ABM2 is 45 amino acids long and comprises the sequence of SEQ ID NO: 130. In some embodiments, the first RgpA portion that comprises an ABM2 is 50 amino acids long and comprises the sequence of SEQ ID NO: 130. In some embodiments, the first RgpA portion that comprises an ABM2 is 55 amino acids long and comprises the sequence of SEQ ID NO: 130. In some embodiments, the first RgpA portion that comprises an ABM2 is 60 amino acids long and comprises the sequence of SEQ ID NO: 130. In some embodiments, the first RgpA portion that comprises an ABM2 is 65 amino acids long and comprises the sequence of SEQ ID NO: 130.

In some embodiments, the first RgpA portion that comprises an ABM2 is 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or 65 amino acids long and comprises the sequence of SEQ ID NO: 130.

In the wild-type sequence of RgpA from P. gingivalis strain W50, the second RgpA portion that comprises an ABM2 is positioned between the K2 and K3 adhesin domains. The second RgpA portion that comprises ABM2 is 56 amino acids long in length and comprises the sequence of SEQ ID NO: 126. This sequence comprises an ABM2 of SEQ ID NO: 130. Accordingly, in some embodiments, the second RgpA portion that comprises an ABM2 is between 14 and 56 amino acids long and comprises the sequence of SEQ ID NO: 130.

In some embodiments, the second RgpA portion that comprises an ABM2 is between 20 and 50 amino acids long and comprises the sequence of SEQ ID NO: 130. In some embodiments, the second RgpA portion that comprises an ABM2 is between 25 and 45 amino acids long and comprises the sequence of SEQ ID NO: 130. In some embodiments, the second RgpA portion that comprises an ABM2 is between 30 and 40 amino acids long and comprises the sequence of SEQ ID NO: 130. In some embodiments, the second RgpA portion that comprises an ABM2 is between 35 and 40 amino acids long and comprises the sequence of SEQ ID NO: 130.

In some embodiments, the second RgpA portion that comprises an ABM2 is 14 amino acids long and comprises the sequence of SEQ ID NO: 130. In some embodiments, the second RgpA portion that comprises an ABM2 is 20 amino acids long and comprises the sequence of SEQ ID NO: 130. In some embodiments, the second RgpA portion that comprises an ABM2 is 25 amino acids long and comprises the sequence of SEQ ID NO: 130. In some embodiments, the second RgpA portion that comprises an ABM2 is 30 amino acids long and comprises the sequence of SEQ ID NO: 130. In some embodiments, the second RgpA portion that comprises an ABM2 is 35 amino acids long and comprises the sequence of SEQ ID NO: 130. In some embodiments, the second RgpA portion that comprises an ABM2 is 40 amino acids long and comprises the sequence of SEQ ID NO: 130. In some embodiments, the second RgpA portion that comprises an ABM2 is 45 amino acids long and comprises the sequence of SEQ ID NO: 130. In some embodiments, the second RgpA portion that comprises an ABM2 is 50 amino acids long and comprises the sequence of SEQ ID NO: 130. In some embodiments, the second RgpA portion that comprises an ABM2 is 56 amino acids long and comprises the sequence of SEQ ID NO: 130.

In some embodiments, the second RgpA portion that comprises an ABM2 is 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 or 56 amino acids long and comprises the sequence of SEQ ID NO: 130.

### Pairs of ABM1 and ABM2 Containing Sequences

The first Kgp portion comprising an ABM1 is positioned N-terminally of the DUF2436 and the first Kgp portion comprising an ABM2 is positioned C-terminally of the DUF2436. A modelled three-dimensional structure of amino acids 229-1732 of native Kgp from P. gingivalis strain W50 using Alphafold2 (shown in FIG. 3) suggests that the first portion of Kgp comprising an ABM1 and the first portion of Kgp comprising an ABM2 may associate to form a first fibronectin type III-like domain. The fibronectin type III-like domain is beta-sandwich structure comprising a first beta sheet of three strands and a second beta sheet of four strands. The first Kgp portion comprising an ABM1 forms the N-terminal portion of a first fibronectin type III-like domain, and contributes two strands to the beta sheet comprising three strands. The first Kgp portion comprising ABM2 forms the C-terminal portion of a first fibronectin type III-like domain, and contributes one strand of the beta sheet comprising three strands and four strands of the second beta sheet. This fibronectin type III-like domain contains an ABM1 and ABM2 motif, and may play a role in mediating adhesion function of the gingipain. Indeed, gingipains are known to interact with fibronectin (Li and Collyer, 2011) and fibronectin type III domains are known to mediate interaction with fibronectin. Moreover, fibronectin type III-like domains have been found in other bacteria species and are postulated to perform a similar function (Konkel et al., 2010). It may therefore be advantageous for the polypeptide to present the ABM1 and ABM2 motifs in a three-dimensional structure that resembles the wild-type protein.

Accordingly, in some embodiments, the first Kgp portion comprising an ABM1 is capable of forming an N-terminal portion of a first fibronectin type III-like domain, for example a beta sheet comprising two strands. In some embodiments the first Kgp portion comprising an ABM2 is capable of forming a C-terminal portion of a first fibronectin type III-like domain, for example a beta sheet with four strands and a further beta sheet strand. In some embodiments, the first Kgp portion comprising an ABM1 is capable of forming an N-terminal portion of a first fibronectin type III-like domain, for example a beta sheet comprising two strands and the first Kgp portion comprising an ABM2 is capable of forming a C-terminal portion of a first fibronectin type III-like domain, for example a beta sheet with four strands and a further beta sheet strand. In certain such embodiments, the first Kgp portion comprising an ABM1 and the first Kgp portion comprising an ABM2 is capable of forming a first fibronectin type III-like domain having a beta-sandwich structure.

In some embodiments, the first RgpA portion comprising an ABM1 is capable of forming an N-terminal portion of a first fibronectin type III-like domain, for example a beta sheet comprising two strands. In some embodiments the first RgpA portion comprising an ABM2 is capable of forming a C-terminal portion of a first fibronectin type III-like domain, for example a beta sheet with four strands and a further beta sheet strand. In some embodiments, the first RgpA portion comprising an ABM1 is capable of forming an N-terminal portion of a first fibronectin type III-like domain, for example a beta sheet comprising two strands and the first RgpA portion comprising an ABM2 is capable of forming a C-terminal portion of a first fibronectin type III-like domain, for example a beta sheet with four strands and a further beta sheet strand. In certain such embodiments, the first RgpA portion comprising an ABM1 and the first RgpA portion comprising an ABM2 is capable of forming a first fibronectin type III-like domain having a beta-sandwich structure.

In some embodiments, the first Kgp portion comprising an ABM1 and the first Kgp portion comprising an ABM2 each comprise a sequence as shown in, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In certain such embodiments, the first Kgp portion comprising an ABM1 and the first Kgp portion comprising an ABM2 are capable of forming a first fibronectin type III-like domain, as described in the preceding paragraphs.

TABLE 4
Combinations of first Kgp portions comprising an
ABM1 and first Kgp portions comprising an ABM2

| First Kgp portion | First Kgp portion |
|---|---|
| comprising an ABM1 | comprising an ABM2 |
| SEQ ID NO: 106 | SEQ ID NO: 121 |
| SEQ ID NO: 89 | SEQ ID NO: 91 |
| SEQ ID NO: 120 | SEQ ID NO: 130 |

In some embodiments, the first RgpA portion comprising an ABM1 and the first RgpA portion comprising an ABM2 each comprise a sequence as shown in, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In certain such embodiments, the first RgpA portion comprising an ABM1 and the first RgpA portion comprising an ABM2 are capable of forming a first fibronectin type III-like domain, as described in the preceding paragraphs.

TABLE 5
Combinations of first RgpA portions comprising an
ABM1 and first RgpA portions comprising an ABM2

| First RgpA portion | First RgpA portion |
|---|---|
| comprising an ABM1 | comprising an ABM2 |
| SEQ ID NO: 114 | SEQ ID NO: 101 |
| SEQ ID NO: 99 | SEQ ID NO: 101 |
| SEQ ID NO: 120 | SEQ ID NO: 130 |

It is also apparent from FIG. 3, that the second Kgp portion comprising an ABM1 and the second Kgp portion comprising an ABM2 associate to form a second fibronectin type III-like domain. Specifically, the second Kgp portion comprising an ABM1 forms the N-terminal portion of a second fibronectin type III-like domain, and contributes two strands to the beta sheet comprising three strands. The second Kgp portion comprising an ABM2 forms the C-terminal portion of a second fibronectin type III-like domain, and contributes one strand of the beta sheet comprising three strands and four strands of the second beta sheet. The N-terminal portion of the second fibronectin type III-like domain associates with the C-terminal portion of the second fibronectin type III-like domain to form a beta-sandwich structure.

Accordingly, in some embodiments, the second Kgp portion comprising an ABM1 is capable of forming an N-terminal portion of a second fibronectin type III-like domain, for example a beta sheet comprising two strands. In some embodiments the second Kgp portion comprising an ABM2 is capable of forming a C-terminal portion of a second fibronectin type III-like domain, for example a beta sheet with four strands and a further beta sheet strand. In some embodiments, the second Kgp portion comprising an ABM1 is capable of forming an N-terminal portion of a second fibronectin type III-like domain, for example a beta sheet comprising two strands and the second Kgp portion comprising an ABM2 is capable of forming a C-terminal portion of a second fibronectin type III-like domain, for example a beta sheet with four strands and a further beta sheet strand. In certain such embodiments, the second Kgp portion comprising an ABM1 and the second Kgp portion comprising an ABM2 is capable of forming a second fibronectin type III-like domain having a beta-sandwich structure.

In some embodiments, the second RgpA portion comprising an ABM1 is capable of forming an N-terminal portion of a second fibronectin type III-like domain, for example a beta sheet comprising two strands. In some embodiments the second RgpA portion comprising an ABM2 is capable of forming a C-terminal portion of a second fibronectin type III-like domain, for example a beta sheet with four strands and a further beta sheet strand. In some embodiments, the second RgpA portion comprising an ABM1 is capable of forming an N-terminal portion of a second fibronectin type III-like domain, for example a beta sheet comprising two strands and the second RgpA portion comprising an ABM2 is capable of forming a C-terminal portion of a second fibronectin type III-like domain, for example a beta sheet with four strands and a further beta sheet strand. In certain such embodiments, the second RgpA portion comprising an ABM1 and the second RgpA portion comprising an ABM2 is capable of forming a second fibronectin type III-like domain having a beta-sandwich structure.

In some embodiments, the second Kgp portion comprising an ABM1 and the second Kgp portion comprising an ABM2 each comprise a sequence as shown in, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In certain such embodiments, the second Kgp portion comprising an ABM1 and the second Kgp portion comprising an ABM2 are capable of forming a second fibronectin type III-like domain, as described in the preceding paragraphs.

TABLE 6
Combinations of second Kgp portions comprising an
ABM1 and second Kgp portions comprising an ABM2

| Second Kgp | Second Kgp |
|---|---|
| portion | portion |
| comprising | comprising |
| an ABM1 | an ABM2 |
| SEQ ID NO: 110 | SEQ ID NO: 124 |
| SEQ ID NO: 92 | SEQ ID NO: 95 |
| SEQ ID NO: 120 | SEQ ID NO: 130 |

In some embodiments, the second RgpA portion comprising an ABM1 and the second RgpA portion comprising an ABM2 each comprise a sequence as shown in, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In certain such embodiments, the second RgpA portion comprising an ABM1 and the second RgpA portion comprising an ABM2 are capable of forming a second fibronectin type III-like domain, as described in the preceding paragraphs.

TABLE 7
Combinations of second RgpA portions comprising an
ABM1 and second RgpA portions comprising an ABM2

| Second RgpA | Second RgpA |
|---|---|
| portion | portion |
| comprising | comprising |
| an ABM1 | an ABM2 |
| SEQ ID NO: 102 | SEQ ID NO: 126 |
| SEQ ID NO: 102 | SEQ ID NO: 105 |
| SEQ ID NO: 120 | SEQ ID NO: 130 |

The nucleic acids and polypeptides of the invention comprising combinations of a first Kgp portion comprising an ABM1 and first Kgp portion comprising an ABM2 that are described in may comprise a second Kgp portion comprising an ABM1 and a second Kgp portion comprising an ABM2 that are described in Error! Reference source not found, for example, as shown in Error! Reference source not found. below. Thus, in some embodiments, the first Kgp portion comprising an ABM1 and the first Kgp portion comprising an ABM2 each comprise a sequence as shown in Error! Reference source not found, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto, and the second Kgp portion comprising an ABM1 and the second Kgp portion comprising an ABM2 each comprise a sequence as shown in Error! Reference source not found, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the first Kgp portion comprising an ABM1, the first Kgp portion comprising an ABM2, the second Kgp portion comprising an ABM1 and the second Kgp portion comprising an ABM2 each comprise a sequence as shown in Error! Reference source not found, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In certain such embodiments, the first Kgp portion comprising an ABM1 and the first Kgp portion comprising an ABM2 are capable of forming a first fibronectin type III-like domain, and the second Kgp portion comprising an ABM1 and the second Kgp portion comprising an ABM2 are capable of forming a second fibronectin type III-like domain, as described in the preceding paragraphs.

TABLE 8
Combinations of first Kgp portions comprising an ABM1 and an
ABM2 and second Kgp portions comprising an ABM1 and an ABM2

| First Kgp | First Kgp | Second Kgp | Second Kgp |
|---|---|---|---|
| portion | portion | portion | portion |
| comprising | comprising | comprising | comprising |
| an ABM1 | an ABM2 | an ABM1 | an ABM2 |
| SEQ ID NO: 106 | SEQ ID NO: 121 | SEQ ID NO: 110 | SEQ ID NO: 124 |
| SEQ ID NO: 89 | SEQ ID NO: 91 | SEQ ID NO: 92 | SEQ ID NO: 95 |
| SEQ ID NO: 120 | SEQ ID NO: 130 | SEQ ID NO: 120 | SEQ ID NO: 130 |

The nucleic acids and polypeptides of the invention comprising combinations of a first RgpA portion comprising an ABM1 and first RgpA portion comprising an ABM2 that are described in may comprise a second RgpA portion comprising an ABM1 and a second RgpA portion comprising an ABM2 that are described in Error! Reference source not found, for example, as shown in Error! Reference source not found. below. Thus, in some embodiments, the first RgpA portion comprising an ABM1 and the second RgpA portion comprising an ABM2 each comprise a sequence as shown in Error! Reference source not found, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto, and the second RgpA portion comprising an ABM1 and the second RgpA portion comprising an ABM2 each comprise a sequence as shown in Error! Reference source not found, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the first RgpA portion comprising an ABM1, the first RgpA portion comprising an ABM2, the second RgpA portion comprising an ABM1 and the second RgpA portion comprising an ABM2 each comprise a sequence as shown in Error! Reference source not found, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In certain such embodiments, the first RgpA portion comprising an ABM1 and the first RgpA portion comprising an ABM2 are capable of forming a first fibronectin type III-like domain, and the second RgpA portion comprising an ABM1 and the second RgpA portion comprising an ABM2 are capable of forming a second fibronectin type III-like domain, as described in the preceding paragraphs.

TABLE 9
Combinations of first RgpA portions comprising an ABM1 and an
ABM2 and second RgpA portions comprising an ABM1 and an ABM2

| First RgpA | First RgpA | Second RgpA | Second RgpA |
|---|---|---|---|
| portion | portion | portion | portion |
| comprising | comprising | comprising | comprising |
| an ABM1 | an ABM2 | an ABM1 | an ABM2 |
| SEQ ID NO: 114 | SEQ ID NO: 101 | SEQ ID NO: 102 | SEQ ID NO: 126 |
| SEQ ID NO: 99 | SEQ ID NO: 101 | SEQ ID NO: 102 | SEQ ID NO: 105 |
| SEQ ID NO: 120 | SEQ ID NO: 130 | SEQ ID NO: 120 | SEQ ID NO: 130 |

In some embodiments, the nucleic acids or polypeptides of the invention comprise a first Kgp portion comprising an ABM1, a first Kgp portion comprising an ABM2, a second Kgp portion comprising an ABM1, a second Kgp portion comprising an ABM2, a first RgpA portion comprising an ABM1 and a first RgpA portion comprising an ABM2. In certain such embodiments, the first Kgp portion comprising an ABM1 and the first Kgp portion comprising an ABM2 each comprise a sequence as shown in (or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99% identity thereto) and the second Kgp portion comprising an ABM1 and the second Kgp portion comprising an ABM2 each comprise a sequence as shown in Error! Reference source not found. (or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99% identity thereto), for example sequences as described in Error! Reference source not found. (or a sequence that has at least 70% (e.g. at least 75, 80, 85, 86, 87, 88, 89, 90, 91, 92, 93, 94, 95, 96, 97, 98 or 99%) identity thereto), and the first RgpA portion comprising an ABM1 and a first RgpA portion comprising an ABM2 each comprise a sequence as shown in Error! Reference source not found. (or a sequence that has at least 70% (e.g. at least 75, 80, 85, 86, 87, 88, 89, 90, 91, 92, 93, 94, 95, 96, 97, 98 or 99%) identity thereto).

For example, the first Kgp portion comprising an ABM1, the second Kgp portion comprising an ABM2, the second Kgp portion comprising an ABM1 and the second Kgp portion comprising an ABM2, the first RgpA portion comprising an ABM1 and a first RgpA portion comprising an ABM2 each comprise a sequence as shown in each comprise a sequence as shown in, or a sequence that has at least 70% (e.g. at least 75, 80, 85, 86, 87, 88, 89, 90, 91, 92, 93, 94, 95, 96, 97, 98 or 99%) identity thereto. In certain such embodiments, the first Kgp portion comprising an ABM1 and the first Kgp portion comprising an ABM2 are capable of forming a first fibronectin type III-like domain, and the second Kgp portion comprising an ABM1 and the second Kgp portion comprising an ABM2 are capable of forming a second fibronectin type III-like domain, and the first RgpA portion comprising an ABM1 and a first RgpA portion comprising an ABM2 are capable of forming a third fibronectin type III-like domain, as described in the preceding paragraphs.

TABLE 10
Combinations of first Kgp portions comprising an ABM1 and an ABM2 and second Kgp portions
comprising an ABM1 and an ABM2 and first RgpA portions comprising an ABM1 and an ABM2.

| First Kgp | First Kgp | Second Kgp | Second Kgp | First RgpA | First RgpA |
|---|---|---|---|---|---|
| portion | portion | portion | portion | portion | portion |
| comprising | comprising | comprising | comprising | comprising | comprising |
| an ABM1 | an ABM2 | an ABM1 | an ABM2 | an ABM1 | an ABM2 |
| SEQ ID NO: 106 | SEQ ID NO: 121 | SEQ ID NO: 110 | SEQ ID NO: 124 | SEQ ID NO: 114 | SEQ ID NO: 101 |
| SEQ ID NO: 89 | SEQ ID NO: 91 | SEQ ID NO: 92 | SEQ ID NO: 95 | SEQ ID NO: 99 | SEQ ID NO: 101 |
| SEQ ID NO: 120 | SEQ ID NO: 130 | SEQ ID NO: 120 | SEQ ID NO: 130 | SEQ ID NO: 120 | SEQ ID NO: 130 |

The skilled person can determine whether particular sequences of interest form a fibronectin type III-like domain through structural modelling in the same way that the Kgp structure was modelled by the inventors. For instance, the skilled person can substitute the first ABM1 sequence and/or the first ABM2 sequence in the first portion of wild type Kgp with said sequences of interest. The skilled person can then model the structure of the Kgp protein comprising said sequences of interest using AlphaFold2 and assess whether said sequences form a fold that is structurally homologous to a fibronectin type III-like domain. The structural homology to a fibronectin type III-like domain can be assessed visually as fibronectin type III-like domains are known have a conserved beta sandwich fold comprising one beta sheet containing three beta strands and one beta sheet containing four strands. Alternatively, the structural homology can be assessed by protein structure comparison servers, such as DALI (ekhidna2.biocenter.helsinki.fi/dali/lsinki.fi).

The skilled person can also determine whether particular sequences of interest form a fibronectin type III-like domain using a functional assay. Fibronectin type III-like domains are also known to mediate interactions with fibronectin. Thus, the skilled person can perform an enzyme-linked immunosorbent assay (ELISA) to determine whether a Kgp construct comprising particular sequences of interest supports fibronectin binding activity. In the ELISA, fibronectin is immobilised on the surface of polystyrene microplate wells and a Kgp construct comprising said sequences of interest is added to the wells in serial dilutions. The wells are washed with buffer and bound Kgp proteins are detected with a high-affinity antibody. A similar method was used to test whether the fibronectin type III-like domains of FlpA in C. jejuni mediate binding to fibronectin (Konkel et al., 2010).

### Portions of Gingipains Comprising an ABM3

Wild-type Kgp and RgpA contain an ABM3 motif which, as illustrated in FIGS. 1, 2A and 2B, is positioned C-terminally of the DUF2436 and N-terminally of the K1 adhesin domain. The modular nature of Kgp means that a portion of Kgp comprising an ABM3 may be included in any of the nucleic acids or polypeptides described herein that comprise other portions of Kgp. The same modular structure of RgpA means that a portion of RgpA comprising an ABM3 may be included in any of the nucleic acids of polypeptides described herein that comprise other portions of RgpA.

In some embodiments, the nucleic acids and polypeptides of the invention comprise a portion of a Kgp comprising ABM3. In some embodiments, the nucleic acids and polypeptides of the invention comprise a portion of a RgpA comprising ABM3.

In some embodiments, the first Kgp portion comprising an ABM3 is derived from the P. gingivalis strain W50. In P. gingivalis strain W50, the Kgp portion comprising an ABM3 has a sequence of SEQ ID NO: 131. Accordingly, in some embodiments, the Kgp portion comprising an ABM3 comprises a sequence of SEQ ID NO: 131 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the first RgpA portion comprising an ABM3 is derived from the P. gingivalis strain W50. In P. gingivalis strain W50, the RgpA portion comprising an ABM3 has a sequence of SEQ ID NO: 135. Accordingly, in some embodiments, the RgpA portion comprising an ABM3 comprises a sequence of SEQ ID NO: 135 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Examples of portions of a Kgp or RgpA sequence comprising ABM3 sequences are provided in along with a consensus sequence for ABM3. The consensus sequence for ABM3 is SEQ ID NO: 139.

TABLE 11
Portions of Kgp and RgpA that comprise ABM3 sequences. The consensus ABM3
sequence is underlined in each sequence.

| Sequence | SEQ ID NO | Gingipain |
|---|---|---|
| GTPNPNPNPNPNPGTTLSESF | SEQ ID NO: 131 | Kgp |
| PQGTPNPNPNPNPNPGTTLSESF | SEQ ID NO: 94 | Kgp |
| VTLKWDAPNGTPNPNPNPNPNPGTTLSESF | SEQ ID NO: 132 | Kgp |
| GTPNPNPNPNPNPNPGT | SEQ ID NO: 133 | Kgp |
| PNGTP(NP)₁₋₆GTT(T)LSESF | SEQ ID NO: 134 | Kgp |
| PNGTPNPNPNPNPNPNPGTTTLSESF | SEQ ID NO: 135 | RgpA |
| PQGTPNPNPNPNPNPNPGTTTLSESF | SEQ ID NO: 103 | RgpA |
| VTLKWDAPNGTPNPNPNPNPNPGTTLSESF | SEQ ID NO: 136 | RgpA |
| GTPNPNPNPNPNPNPGT | SEQ ID NO: 137 | RgpA |
| PNGTP(NP)₁₋₆GTT(T)LSESF | SEQ ID NO: 138 | RgpA |
| GTPNPNPNPNPNP(NP)GT | SEQ ID NO: 139 | Consensus motif |

In some embodiments, the portion of Kgp comprising an ABM3 comprises a sequence of SEQ ID NO: 139, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of Kgp comprising an ABM3 comprises a sequence of SEQ ID NO: 131, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of Kgp comprising an ABM3 comprises a sequence of SEQ ID NO: 132, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of Kgp comprising an ABM3 comprises a sequence of SEQ ID NO: 94, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of Kgp comprising an ABM3 comprises a sequence of SEQ ID NO: 133, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of Kgp comprising an ABM3 comprises a sequence of SEQ ID NO: 134, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of RgpA comprising an ABM3 comprises a sequence of SEQ ID NO: 139, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of RgpA comprising an ABM3 comprises a sequence of SEQ ID NO: 135, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of RgpA comprising an ABM3 comprises a sequence of SEQ ID NO: 103, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of RgpA comprising an ABM3 comprises a sequence of SEQ ID NO: 136, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of RgpA comprising an ABM3 comprises a sequence of SEQ ID NO: 137, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the portion of RgpA comprising an ABM3 comprises a sequence of SEQ ID NO: 138, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In the wild-type sequence of Kgp from P. gingivalis strain W50, the portion of Kgp that comprises an ABM3 is positioned C-terminally of the DUF2436 and N-terminally of the K1 adhesin domain. An ABM1 sequence is positioned N-terminally and adjacent to the portion that comprises ABM3. In this context, the Kgp portion that comprises an ABM3 is 30 amino acids in length and comprises the sequence of SEQ ID NO: 132. This sequence comprises an ABM3 of SEQ ID NO: 139. Accordingly, in some embodiments, the Kgp portion that comprises an ABM3 is between 15 and 30 amino acids long and comprises the sequence of SEQ ID NO: 139.

In some embodiments, the Kgp portion that comprises an ABM3 is between 17 and 26 amino acids long and comprises the sequence of SEQ ID NO: 139. In some embodiments, the Kgp portion that comprises an ABM3 is between 20 and 25 amino acids long and comprises the sequence of SEQ ID NO: 139.

In some embodiments, the Kgp portion that comprises an ABM3 is 15 amino acids long and comprises the sequence of SEQ ID NO: 139. In some embodiments, the Kgp portion that comprises an ABM3 is 17 amino acids long and comprises the sequence of SEQ ID NO: 139. In some embodiments, the Kgp portion that comprises an ABM3 is 20 amino acids long and comprises the sequence of SEQ ID NO: 139. In some embodiments, the Kgp portion that comprises an ABM3 is 25 amino acids long and comprises the sequence of SEQ ID NO: 139. In some embodiments, the Kgp portion that comprises an ABM3 is 26 amino acids long and comprises the sequence of SEQ ID NO: 139. In some embodiments, the first Kgp portion that comprises an ABM3 is 30 amino acids long and comprises the sequence of SEQ ID NO: 139.

In the wild-type sequence of RgpA from P. gingivalis strain W50, the portion of RgpA that comprises an ABM3 is positioned C-terminally of the DUF2436 and N-terminally of the K1 adhesin domain. An ABM1 sequence is positioned N-terminally of the portion that comprises an ABM3 and the K1 adhesin domain is positioned C-terminally of and adjacent to the portion that comprises an ABM3. In this context, the RgpA portion that comprises an ABM3 is 30 amino acids in length and comprises the sequence of SEQ ID NO: 136. This sequence comprises an ABM3 of SEQ ID NO: 139. Accordingly, in some embodiments, the RgpA portion that comprises an ABM3 is between 15 and 30 amino acids long and comprises the sequence of SEQ ID NO: 139.

In some embodiments, the RgpA portion that comprises an ABM3 is between 17 and 26 amino acids long and comprises the sequence of SEQ ID NO: 139. In some embodiments, the RgpA portion that comprises an ABM3 is between 20 and 25 amino acids long and comprises the sequence of SEQ ID NO: 139.

In some embodiments, the RgpA portion that comprises an ABM3 is 15 amino acids long and comprises the sequence of SEQ ID NO: 139. In some embodiments, the RgpA portion that comprises an ABM3 is 17 amino acids long and comprises the sequence of SEQ ID NO: 139. In some embodiments, the RgpA portion that comprises an ABM3 is 20 amino acids long and comprises the sequence of SEQ ID NO: 139. In some embodiments, the RgpA portion that comprises an ABM3 is 25 amino acids long and comprises the sequence of SEQ ID NO: 139. In some embodiments, the RgpA portion that comprises an ABM3 is 26 amino acids long and comprises the sequence of SEQ ID NO: 139. In some embodiments, the first RgpA portion that comprises an ABM3 is 30 amino acids long and comprises the sequence of SEQ ID NO: 139.

Position of ABM Comprising Sequences with Respect to Other Gingipain Domains

The first Kgp or RgpA portion that comprises an ABM1 may be distinct from its neighbouring domains (i.e. the Cat domain and the DUF2436) or it may overlap with one or both of its neighbouring domains. In certain embodiments, the first Kgp or RgpA portion that comprises an ABM1 is distinct from the Cat domain and the DUF2436. In certain embodiments, a peptide linker may be positioned between the Cat domain and the first Kgp or RgpA portion that comprises an ABM1. In certain embodiments, a peptide linker may be positioned between the first Kgp or RgpA portion that comprises an ABM1 and the DUF2436. In certain embodiments, a peptide linker may be positioned between the Cat domain and the first Kgp or RgpA portion that comprises an ABM1 and a peptide linker may positioned between the first Kgp or RgpA portion that comprises an ABM1 and the DUF2436.

In some embodiments, the first Kgp or RgpA portion that comprises an ABM1 overlaps with the Cat domain. In certain such embodiments, the first Kgp or RgpA portion that comprises an ABM1 overlaps with the Cat domain and is distinct from the DUF2436.

The first Kgp or RgpA portion that comprises an ABM2 may be distinct from its neighbouring domains (i.e. the DUF2436 and Kgp or RgpA portion comprising ABM1) or it may overlap with one or both of its neighbouring domains. In certain embodiments, the first Kgp or RgpA portion that comprises an ABM2 is distinct from the DUF2436 and Kgp or RgpA portion comprising ABM1. In certain embodiments, a peptide linker may be positioned between the Cat domain and the first Kgp or RgpA portion that comprises an ABM1. In certain embodiments, a peptide linker may be positioned between the first Kgp or RgpA portion that comprises an ABM1 and the DUF2436. In certain embodiments, a peptide linker may be positioned between the Cat domain and the first Kgp or RgpA portion that comprises an ABM1 and a peptide linker may positioned between the first Kgp or RgpA portion that comprises an ABM1 and the DUF2436.

The second Kgp or RgpA portion that comprises an ABM1 may be distinct from its neighbouring domains (i.e. the first Kgp or RgpA portion comprising ABM2 and the Kgp or RgpA portion comprising ABM3) or it may overlap with one or both of its neighbouring domains. In certain embodiments, the second Kgp or RgpA portion that comprises an ABM1 is distinct from first the Kgp or RgpA portion comprising ABM2 and the Kgp or RgpA portion comprising ABM3. In certain embodiments, a peptide linker may be positioned between first Kgp or RgpA portion comprising ABM2 and the second Kgp or RgpA portion that comprises an ABM1. In certain embodiments, a peptide linker may be positioned between the second Kgp or RgpA portion that comprises an ABM1 and the Kgp or RgpA portion comprising ABM3. In certain embodiments, a peptide linker may be positioned between first Kgp or RgpA portion comprising ABM2 and the second Kgp or RgpA portion that comprises an ABM1, and a peptide linker may be positioned between the second Kgp or RgpA portion that comprises an ABM1 and the Kgp or RgpA portion comprising ABM3.

In some embodiments, the second Kgp or RgpA portion that comprises an ABM1 overlaps with the Kgp or RgpA portion comprising ABM3. In certain such embodiments, the Kgp or RgpA portion that comprises an ABM1 is distinct from first the Kgp or RgpA portion comprising ABM2 and overlaps with the Kgp or RgpA portion comprising ABM3.

The second Kgp or RgpA portion that comprises an ABM2 may be distinct from its neighbouring domain (i.e. the K2 adhesin domain) or it may overlap with one or both of its neighbouring domain. In certain embodiments, the second Kgp or RgpA portion that comprises an ABM2 is distinct from the K2 adhesin domain. In certain embodiments, a peptide linker may be positioned between second Kgp or RgpA portion comprising ABM2 and the K2 adhesin domain.

The Kgp or RgpA portion that comprises an ABM3 may be distinct from its neighbouring domain (i.e. the second Kgp or RgpA portion comprising ABM1 and the K1 adhesin domain) or it may overlap with its neighbouring domains. In certain embodiments, the Kgp or RgpA portion that comprises an ABM3 overlaps with the second Kgp or RgpA portion comprising ABM1. In certain embodiments, the Kgp or RgpA portion that comprises an ABM3 overlaps with the K1 adhesin domain. In certain embodiments, the Kgp or RgpA portion that comprises an ABM3 overlaps with the second Kgp or RgpA portion comprising ABM1 and the K1 adhesin domain.

### Domain of Unknown Function (DUF)

The nucleic acids and polypeptides of the invention comprise at least a portion of a domain of unknown function (DUF), as disclosed herein. The modular nature of gingipains is such that any of the DUFs disclosed herein may be combined with any of the other domains disclosed herein. For instance, any of the above disclosed sequences comprising ABMs may be combined with any of the DUFs described in the subsequent paragraphs.

A domain of unknown function (DUF) is a protein domain for which a function has not been characterised. As such, a domain that is initially designated as a DUF may later be renamed once a function is established, or alternatively grouped with an existing family of domains that has already been characterized. DUFs have been catalogued in the Pfam database (pfam.xfam.org), with each conserved DUF being assigned a number (DUF1, DUF2, etc.). This means that a DUF found in a first protein may be assigned the same number as a DUF found in a second protein when the two DUFs show a sufficient degree of homology. The Pfam database is currently part of the InterPro database (www.ebi.ac.uk/interpro/), a database which classifies proteins beyond merely DUFs (Paysan-Lafosse et al., 2022).

A DUF has been identified in P. gingivalis Kgp and RgpA and has been classified as DUF2436 (Dashper et al., 2017). In the InterPro database, DUF2436 is assigned the entry number IPR018832. The nucleic acids and polypeptides of the invention comprise at least a portion of a DUF2436.

The skilled person can determine whether a particular sequence is at least a portion of a DUF2436 through comparison with known DUF2436 sequences. For example, the InterPro database allows a particular sequence to be searched, thereby allowing identification of sequences that comprises at least a portion of a DUF2436. DUF2436 is found in many different organisms and proteins and any DUF2436 may be used in the invention, regardless of whether its particular sequence is found in P. gingivalis. For instance, using a DUF2436 from a non-gingipain protein, or a non-P. gingivalis species may allow the remaining P. gingivalis domains of the polypeptide to fold into a structure that is sufficiently similar to the three-dimensional structure of a wild-type gingipain.

Typically, the at least a portion of DUF2436 according to the invention is derived from a P. gingivalis DUF2436. In some embodiments, the DUF2436 is derived from a P. gingivalis Kgp. In some embodiments, the DUF2436 is derived from a P. gingivalis RgpA.

In some embodiments, the at least a portion of Kgp DUF2436 is derived from the P. gingivalis strain W50. In P. gingivalis strain W50, Kgp DUF2436 has a sequence of SEQ ID NO: 168. Accordingly, in some embodiments, the at least a portion of the Kgp DUF2436 comprises at least a portion of SEQ ID NO: 168 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp DUF2436 comprises a sequence of SEQ ID NO: 168 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of RgpA DUF2436 is derived from the P. gingivalis strain W50. In P. gingivalis strain W50, RgpA DUF2436 has a sequence of SEQ ID NO: 171. Accordingly, in some embodiments, the at least a portion of the RgpA DUF2436 comprises at least a portion of SEQ ID NO: 171 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA DUF2436 comprises a sequence of SEQ ID NO: 171 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Examples of DUF2436 sequences that may be used according to the invention are provided in below.

TABLE 12
Examples of DUF2436 sequences

| Sequence | SEQ ID NO: | Gingpipain |
|---|---|---|
| SREVKRIGDGLFVTIEPANDVRANEAKVVLAADNVWGDNTGYQ | SEQ ID NO: | Kgp (162 aa) |
| FLLDADHNTFGSVIPATGPLFTGTASSNLYSANFEYLIPANADPVV | 168 | |
| TTQNIIVTGQGEVVIPGGVYDYCITNPEPASGKMWIAGDGGNQPA | | |
| RYDDFTFEAGKKYTFTMRRAGMGDGTDM | | |
| SREVKRIGDGLFVTIEPANDVRANEAKVVLAADNVWGDNTGYQ | SEQ ID NO: 90 | Kgp (162 aa) |
| FLLDADHNTFGSVIPATGPLFTGTASSNLYSANFEYLIPANADPVV | | |
| TTQNIIVTGQGEVVIPGGVYDYCITNPEPASGKMWIAGDGGNQPA | | |
| RYDDFTFEAGKKYTFTMRRAGMGDGTDM | | |
| SREVKRIGDGLFVTIEPANDVRANEAKVVLAADNVWGDNTGYQ | SEQ ID NO: | Kgp W83 |
| FLLDADHNTFGSVIPATGPLFTGTASSNLYSANFEYLVPANADPV | 169 | (162 aa) |
| VTTQNIIVTGQGEVVIPGGVYDYCITNPEPASGKMWIAGDGGNQP | | |
| ARYDDFTFEAGKKYTFTMRRAGMGDGTDM | | |
| SREVKRIGDGLFVTIEPANDVRANEAKVVLAADNVWGDNTGYQ | SEQ ID NO: | Kgp ATCC |
| FLLDADHNTFGSVIPATGPLFTGTASSNLYSANFEYLIPANADPVV | 170 | 33277 (162 aa) |
| TTQNIIVTGQGEVVIPGGVYDYCITNPEPASGKMWIAGDGGNQPA | | |
| RYDDFTFEAGKKYTFTMRRAGMGDGTDM | | |
| TNTARSVDGIRELVLLSVSDAPELLRSGQAEIVLEAHDVWNDGSG | SEQ ID NO: | RgpA (163 aa) |
| YQILLDADHDQYGQVIPSDTHTLWPNCSVPANLFAPFEYTVPENA | 171 | |
| DPSCSPTNMIMDGTASVNIPAGTYDFAIAAPQANAKIWIAGQGPT | | |
| KEDDYVFEAGKKYHFLMKKMGSGDGTEL | | |
| TNTARSVDGIRELVLLSVSDAPELLRSGQAEIVLEAHDVWNDGSG | SEQ ID NO: | RgpA (163 aa) |
| YQILLDADHDQYGQVIPSDTHTLWPNCAVPANLFAPFEYTVPEN | 100 | |
| ADPSCSPTNMIMDGTASVNIPAGTYDFAIAAPQANAKIWIAGQGP | | |
| TKEDDYVFEAGKKYHFLMKKMGSGDGTEL | | |
| ARSVDGIRELVLLSVSDAPELLRSGQAEIVLEAHDVWNDGSGYQI | SEQ ID NO: | RgpA (160 aa) |
| LLDADHDQYGQVIPSDTHTLWPNCSVPANLFAPFEYTVPENADPS | 172 | |
| CSPTNMIMDGTASVNIPAGTYDFAIAAPQANAKIWIAGQGPTKED | | |
| DYVFEAGKKYHFLMKKMGSGDGTEL | | |
| WGDNTGYQFLLDADHNTFGSVIPATGPLFTGTASSNLYSANFEYL | SEQ ID NO: | KAS2-A1 |
| IPANADPVVTTQNIIVTGQGEVVIPGGVYDYCITNPEPASGKMWI | 173 | (127 aa) |
| AGDGGNQPARYDDFTFEAGKKYTFTMRRAGMGDGTDM | | |

In some embodiments, the at least a portion of the Kgp DUF2436 comprises at least a portion of SEQ ID NO: 90 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp DUF2436 comprises a sequence of SEQ ID NO: 90 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp DUF2436 comprises at least a portion of SEQ ID NO: 169 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp DUF2436 comprises a sequence of SEQ ID NO: 169 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp DUF2436 comprises at least a portion of SEQ ID NO: 170 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp DUF2436 comprises a sequence of SEQ ID NO: 170 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA DUF2436 comprises at least a portion of SEQ ID NO: 100 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA DUF2436 comprises a sequence of SEQ ID NO: 100 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA DUF2436 comprises at least a portion of SEQ ID NO: 172 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA DUF2436 comprises a sequence of SEQ ID NO: 172 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA DUF2436 comprises at least a portion of SEQ ID NO: 173 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA DUF2436 comprises a sequence of SEQ ID NO: 173 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

The inventors have found that nucleic acids and polypeptides comprising a full-length DUF2436 may be particularly advantageous in eliciting an immune response. Accordingly, in certain embodiments, the nucleic acid or polypeptide of the invention comprises a full-length DUF2436.

A full-length DUF2436 domain refers to a DUF2436 that has not been truncated relative to a corresponding wild-type sequence. Accordingly, in some embodiments, the at least a portion of the DUF2436 is a full-length DUF2436 that is the same length as a corresponding wild-type DUF2436 sequence.

By way of example, DUF2436 present in Kgp from P. gingivalis strain W50 is 162 amino acids long. Thus, in some embodiments, the full-length Kgp DUF2436 is at least 162 amino acids long (for example, 162 amino acids long). DUF2436 present in RgpA from P. gingivalis strain W50 is 163 amino acids long. Thus, in some embodiments, the full-length RgpA DUF2436 is at least 163 amino acids long (for example, 163 amino acids long).

In some embodiments, the full-length Kgp DUF2436 is at least 160 amino acids long (for example 160 amino acids long). In some embodiments, the full-length Kgp DUF2436 is at least 161 amino acids long for example 161 amino acids long). In some embodiments, the full-length Kgp DUF2436 is at least 162 amino acids long (for example 162 amino acids long). In some embodiments, the full-length Kgp DUF2436 is at least 163 amino acids long (for example 166 amino acids long). In some embodiments, the full-length RgpA DUF2436 is 160 amino acids long. In some embodiments, the full-length RgpA DUF2436 is at least 161 amino acids long (for example 161 amino acids long). In some embodiments, the full-length RgpA DUF2436 is at least 162 amino acids long (for example 162 amino acids long). In some embodiments, the full-length RgpA DUF2436 is at least 163 amino acids long (for example 163 amino acids long).

In some embodiments, the at least a portion of the Kgp DUF2436 is a full-length DUF2436 and comprises the sequence of SEQ ID NO: 168 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp DUF2436 is a full-length DUF2436 and comprises the sequence of SEQ ID NO: 90 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp DUF2436 is a full-length DUF2436 and comprises the sequence of SEQ ID NO: 169 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp DUF2436 is a full-length DUF2436 and comprises the sequence of SEQ ID NO: 170 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA DUF2436 is a full-length DUF2436 and comprises the sequence of SEQ ID NO: 171 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA DUF2436 is a full-length DUF2436 and comprises the sequence of SEQ ID NO: 100 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA DUF2436 is a full-length DUF2436 and comprises the sequence of SEQ ID NO: 172 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Truncations of the DUF2436 may also be made without significantly altering the properties of the resulting polypeptide. Thus, in some embodiments, the at least a portion of the DUF2436 is a truncated DUF2436 wherein, the truncated DUF2436 is truncated by between 1 and 35 amino acids. In certain such embodiments, the DUF2436 is truncated by between 1 and 30 amino acids, 1 and 25 amino acids, 1 and 20 amino acids, 1 and 15 amino acids, 1 and 10 amino acids, 1 and 5 amino acids. In some embodiments the DUF2436 is truncated by 30 amino acids. In some embodiments the DUF2436 is truncated by 25 amino acids. In some embodiments the DUF2436 is truncated by 20 amino acids. In some embodiments the DUF2436 is truncated by 15 amino acids. In some embodiments the DUF2436 is truncated by 10 amino acids. In some embodiments the DUF2436 is truncated by 5 amino acids.

The truncation may be at the N-terminus or C-terminus of the DUF. Accordingly, in some embodiments, the DUF2436 is truncated by between 1 and 35 amino acids at the N-terminus. Thus, in some embodiments, the at least a portion of the DUF2436 is a truncated DUF2436 wherein, the truncated DUF2436 is truncated by between 1 and 35 amino acids at the N-terminus. In certain such embodiments, the DUF2436 is truncated by between 1 and 30 amino acids at the N-terminus, 1 and 25 amino acids at the N-terminus, 1 and 20 amino acids at the N-terminus, 1 and 15 amino acids at the N-terminus, 1 and 10 amino acids at the N-terminus, 1 and 5 amino acids at the N-terminus. In some embodiments the DUF2436 is truncated by 30 amino acids at the N-terminus. In some embodiments the DUF2436 is truncated by 25 amino acids at the N-terminus. In some embodiments the DUF2436 is truncated by 20 amino acids at the N-terminus. In some embodiments the DUF2436 is truncated by 15 amino acids at the N-terminus. In some embodiments the DUF2436 is truncated by 10 amino acids at the N-terminus. In some embodiments the DUF2436 is truncated by 5 amino acids at the N-terminus.

In some embodiments, the DUF2436 is truncated by between 1 and 35 amino acids at the C-terminus. Thus, in some embodiments, the at least a portion of the DUF2436 is a truncated DUF2436 wherein, the truncated DUF2436 is truncated by between 1 and 35 amino acids at the C-terminus. In certain such embodiments, the DUF2436 is truncated by between 1 and 30 amino acids at the C-terminus, 1 and 25 amino acids at the C-terminus, 1 and 20 amino acids at the C-terminus, 1 and 15 amino acids at the C-terminus, 1 and 10 amino acids at the C-terminus, 1 and 5 amino acids at the C-terminus. In some embodiments the DUF2436 is truncated by 30 amino acids at the C-terminus. In some embodiments the DUF2436 is truncated by 25 amino acids at the C-terminus. In some embodiments the DUF2436 is truncated by 20 amino acids at the C-terminus. In some embodiments the DUF2436 is truncated by 15 amino acids at the C-terminus. In some embodiments the DUF2436 is truncated by 10 amino acids at the C-terminus. In some embodiments the DUF2436 is truncated by 5 amino acids at the C-terminus.

In some embodiments, the truncated DUF2436 comprises at least a portion of SEQ ID NO: 173 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Variants of a DUF2436 may also be employed in the invention. Such variants may be to remove glycosylation sites, as described elsewhere herein.

### Catalytic Domain (Cat Domain)

The nucleic acids and polypeptides of the invention comprise at least a portion of a catalytic domain from Kgp and/or at least a portion of a catalytic domain from RgpA or RgpB, as disclosed herein. The modular nature of gingipains is such that any of the catalytic domains disclosed herein may be combined with any of the other domains disclosed herein. For instance, any of the above disclosed sequences comprising ABMs and or DUFs may be combined with any of the catalytic domains described in the subsequent paragraphs.

Kgp, RgpA and RgpB are lysine-specific and arginine-specific cysteine proteinases belonging to the C25 peptidase family in which the proteinase activity is mediated by a catalytic domain that is positioned at the N-terminus of the active wild-type protein. The catalytic domain as defined herein comprises the C25 peptidase domain and the immunoglobulin fold at its C-terminus (C25C) (Dashper et al., 2017). The nucleic acids and polypeptides of the invention include at least a portion of Kgp, RgpA and/or RgpB catalytic domain with a view to eliciting an antibody response that inhibits the proteinase function of Kgp, RgpA and/or RgpB. The InterPro database entry for the C25 peptidase domain is IPR001769. The InterPro database entry for the C25C domain is IPR005536.

In some embodiments, the at least a portion of Kgp catalytic domain according to the invention is derived from a P. gingivalis Kgp.

In some embodiments, the at least a portion of RgpA catalytic domain according to the invention is derived from a P. gingivalis RgpA.

In some embodiments, the at least a portion of RgpB catalytic domain according to the invention is derived from a P. gingivalis RgpB.

In some embodiments, the at least a portion of Kgp catalytic domain is derived from the P. gingivalis strain W50. In P. gingivalis strain W50, the Kgp catalytic domain has a sequence of SEQ ID NO: 174. Accordingly, in some embodiments, the at least a portion of the Kgp catalytic domain comprises at least a portion of SEQ ID NO: 174 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 174 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of RgpA catalytic domain is derived from the P. gingivalis strain W50. In P. gingivalis strain W50, RgpA catalytic domain has a sequence of SEQ ID NO: 178. Accordingly, in some embodiments, the at least a portion of the RgpA catalytic domain comprises at least a portion of SEQ ID NO: 178 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA catalytic domain comprises a sequence of SEQ ID NO: 178 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of RgpB catalytic domain is derived from the P. gingivalis strain W50. In P. gingivalis strain W50, RgpB catalytic domain has a sequence of SEQ ID NO: 251. Accordingly, in some embodiments, the at least a portion of the RgpB catalytic domain comprises at least a portion of SEQ ID NO: 251 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpB catalytic domain comprises a sequence of SEQ ID NO: 251 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Examples of Kgp, RgpA and RgpB catalytic domain sequences that may be used according to the invention are provided in below.

TABLE 13
Examples of Kgp, RgpA and RgpB catalytic domain sequences

| Sequence | SEQ ID NO: | Gingpipain |
|---|---|---|
| DVYTDHGDLYNTPVRMLVVAGAKFKEALKPWLTWKAQKGFYL | SEQ ID NO: | Kgp |
| DVHYTDEAEVGTTNASIKAFIHKKYNDGLAASAAPVFLAL VGDT | 174 | |
| DVISGEKGKKTKKVTDLYYSAVDGDYFPEMYTFRMSASSPEELT | | |
| NIIDKVLMYEKATMPDKSYLEKVLLIAGADYSWNSQVGQPTIKY | | |
| GMQYYYNQEHGYTDVYNYLKAPYTGCYSHLNTGVSFANYTAH | | |
| GSETAWADPLLTTSQLKALTNKDKYFLAIGNCCITAQFDYVQPCF | | |
| GEVITRVKEKGAYAYIGSSPNSYWGEDYYWSVGANAVFGVQPTF | | |
| EGTSMGSYDATFLEDSYNTVNSIMWAGNLAATHAGNIGNITHIG | | |
| AHYYWEAYHVLGDGSVMPYRAMPKTNTYTLPASLPQNQASYSI | | |
| QASAGSYVAISKDGVLYGTGVANASGVATVSMTKQITENGNYD | | |
| VVITRSNYLPVIKQIQV | | |
| DVYTDHGDLYNTPVRMLVVAGAKFKEALKPWLTWKAQKGFYL | SEQ ID NO: 64 | Kgp |
| DVHYTDEAEVGTTNASIKAFIHKKYNDGLAASAAPVFLALVGDT | | |
| DVISGEKGKKSKKVTDLYYSAVDGDYFPEMYTFRMSASSPEELT | | |
| NIIDKVLMYEKATMPDKSYLEKVLLIAGADYSWNSQVGQPTIKY | | |
| GMQYYYNQEHGYTDVYNYLKAPYTGCYSHLNTGVSFANYTAH | | |
| GSETAWADPLLTTSQLKALTNKDKYFLAIGNCSITAQFDYVQPCF | | |
| GEVITRVKEKGAYAYIGSSPNSYWGEDYYWSVGANAVFGVQPTF | | |
| EGTSMGSYDATFLEDSYNTVNSIMWAGNLAATHAGNIGNITHIG | | |
| AHYYWEAYHVLGDGSVMPYRAMPKTNTYTLPASLPQNQASYSI | | |
| QASAGSYVAISKDGVLYGTGVANASGVATVSMTKQITENGNYD | | |
| VVITRSNYLPVIKQIQVGEPS | | |
| KAPYTGCYSHLNTGVSFANYTAHGSETAWADPLLTTSQLKALTN | SEQ ID NO: | Kgp |
| KDK | 175 | |
| KAPYTGCYSHLNTGVSFANYVAHGSETAWADPLLTTSQLKALTN | SEQ ID NO: 88 | Kgp |
| KDK | | |
| NTGVSFANYTAHGSETAWADPLLTTSQLKALTNKDK | SEQ ID NO: 61 | Kgp |
| NTGVSFANYVAHGSETAWADPLLTTSQLKALTNKDK | SEQ ID NO: | Kgp |
| | 163 | |
| DVYTDHGDLYNTPVRMLVVAGAKFKEALKPWLTWKAQKGFYL | SEQ ID NO: | Kgp |
| DVHYTDEAEVGTTNASIKAFIHKKYNDGLAASAAPVFLALVGDT | 176 | |
| DVISGEKGKKTKKVTDLYYSAVDGDYFPEMYTFRMSASSPEELT | | |
| NIIDKVLMYEKATMPDKSYLEKVLLIAGADYSWNSQVGQPTIKY | | |
| GMQYYYNQEHGYTDVYNYLKAPYTGCYSHLNTGVSFANYTAH | | |
| GSETAWADPLLTTSQLKALTNKDKYFLAIGNCCITAQFDYVQPCF | | |
| GEVITRVKEKGAYAYIGSSPNSYWGEDYYWSVGANAVFGVQPTF | | |
| EGTSMGSYDATFLEDSYNTVNSIMWAGNLAATHAGNIGNITHIG | | |
| AHYYWEAYHVLGDGSVMPYRAMPKTNTYTLPASLPQNQASYSI | | |
| QASAGSYVAISKDGVLYGTGVANASGVATVSMTKQITENGNYD | | |
| VVITRSNYLPVIKQIQV | | |
| DVYTDHGDLYNTPVRMLVVAGAKFKEALKPWLTWKAQKGFYL | SEQ ID NO: | Kgp |
| DVHYTDEAEVGTTNASIKAFIHKKYNDGLAASAAPVFLALVGDT | 177 | |
| DVISGEKGKKTKKVTDLYYSAVDGDYFPEMYTFRMSASSPEELT | | |
| NIIDKVLMYEKATMPDKSYLEKALLIAGADSYWNPKIGQQTIKY | | |
| AVQYYYNQDHGYTDVYSYPKAPYTGCYSHLNTGVGFANYTAH | | |
| GSETSWADPSLTATQVKALTNKDKYFLAIGNCCVTAQFDYPQPC | | |
| FGEVMTRVKEKGAYAYIGSSPNSYWGEDYYWSVGANAVFGVQP | | |
| TFEGTSMGSYDATFLEDSYNTVNSIMWAGNLAATHAGNIGNITHI | | |
| GAHYYWEAYHVLGDGSVMPYRAMPKTNTYTLPASLPQNQASYS | | |
| IQASAGSYVAISKDGVLYGTGVANASGVATVNMTKQITENGNYD | | |
| VVITRSNYLPVIKQIQ | | |
| EPGRYTPVEEKQNGRMIVIVAKKYEGDIKDFVDWKNQRGLRTEV | SEQ ID NO: | RgpA |
| KVAEDIASPVTANAIQQFVKQEYEKEGNDLTYVLLIGDHKDIPAK | 178 | |
| ITPGIKSDQVYGQIVGNDHYNEVFIGRFSCESKEDLKTQIDRTIHY | | |
| ERNITTEDKWLGQALCIASAEGGPSADNGESDIQHENVIANLLTQ | | |
| YGYTKIIKCYDPGVTPKNIIDAFNGGISLANYTGHGSETAWGTSHF | | |
| GTTHVKQLTNSNQLPFIFDVACVNGDFLFSMPCFAEALMRAQKD | | |
| GKPTGTVAIIASTINQSWASPMRGQDEMNEILCEKHPNNIKRTFG | | |
| GVTMNGMFAMVEKYKKDGEKMLDTWTVFGDPSLLVRTLVPTK | | |
| MQVTAPAQINLTDASVNVSCDYNGAIATISANGKMFGSAVVENG | | |
| TATINLTGLTNESTLTLTVVGYNKETVIKTINTNGEP | | |
| EPGRYTPVEEKQNGRMIVIVAKKYEGDIKDFVDWKNQRGLRTEV | SEQ ID NO: 98 | RgpA |
| KVAEDIASPVTANAIQQFVKQEYEKEGNDLTYVLLIGDHKDIPAK | | |
| ITPGIKSDQVYGQIVGNDHYNEVFIGRFSCESKEDLKTQIDRTIHY | | |
| ERNIATEDKWLGQALCIASAEGGPSADNGESDIQHENVIANLLTQ | | |
| YGYTKIIKCYDPGVTPKNIIDAFNGGISLANYAGHGSETAWGTSH | | |
| FGTTHVKQLTNSNQLPFIFDVASVNGDFLFSMPCFAEALMRAQK | | |
| DGKPTGTVAIIASTIQQSWASPMRGQDEMNEILCEKHPNNIKRTF | | |
| GGVTMNGMFAMVEKYKKDGEKMLDTWTVFGDPSLLVRTLVPT | | |
| KMQVTAPAQINLADASVQVSCDYNGAIATISANGKMFGSAVVEQ | | |
| GTATIQLTGLTQESTLTLTVVGYNKETVIKTINTNGEP | | |
| GVTPKNIIDAFNGGISLANYTGHGSETAWGTSHFGTTHVKQLTNS | SEQ ID NO: | RgpA |
| NQ | 179 | |
| GVTPKNIIDAFNGGISLAQYTGHGSETAWGTSHFGTTHVKQLTNS | SEQ ID NO: 97 | RgpA |
| NQ | | |
| NGGISLANYTGHGSETAWGTSHFGTTHVKQLTNSNQ | SEQ ID NO: | RgpA |
| | 180 | |
| NGGISLAQYTGHGSETAWGTSHFGTTHVKQLTNSNQ | SEQ ID NO: | RgpA |
| | 166 | |
| EPGRYTPVEEKQNGRMIVIVAKKYEGDIKDFVDWKNQRGLRTEV | SEQ ID NO: | RgpA |
| KVAEDIASPVTANAIQQFVKQEYEKEGNDLTYVLLVGDHKDIPA | 181 | |
| KITPGIKSDQVYGQIVGNDHYNEVFIGRFSCESKEDLKTQIDRTIH | | |
| YERNITTEDKWLGQALCIASAEGGPSADNGESDIQHENVIANLLT | | |
| QYGYTKIIKCYDPGVTPKNIIDAFNGGISLVNYTGHGSETAWGTS | | |
| HFGTTHVKQLTNSNQLPFIFDVACVNGDFLFSMPCFAEALMRAQ | | |
| KDGKPTGTVAIIASTINQSWASPMRGQDEMNEILCEKHPNNIKRT | | |
| FGGVTMNGMFAMVEKYKKDGEKMLDTWTVFGDPSLLVRTLVP | | |
| TKMQVTAPAQINLTDASVNVSCDYNGAIATISANGKMFGSAVVE | | |
| NGTATINLTGLTNESTLTLTVVGYNKETVIKTINTNGEPN | | |
| EATRYTPVEEKENGRMIVIVAKKYEGDIKDFVDWKNQRGLRTEV | SEQ ID NO: | RgpB |
| KVAEDIASPVTANAIQQFVKQEYEKEGNDLTYVLLVGDHKDIPA | 251 | |
| KITPGIKSDQVYGQIVGNDHYNEVFIGRFSCESKEDLKTQIDRTIH | | |
| YERNITTEDKWLGQALCIASAEGGPSADNGESDIQHENVIANLLT | | |
| QYGYTKIIKCYDPGVTPKNIIDAFNGGISLVNYTGHGSETAWGTS | | |
| HFGTTHVKQLTNSNQLPFIFDVACVNGDFLFSMPCFAEALMRAQ | | |
| KDGKPTGTVAIIASTINQSWASPMRGQDEMNEILCEKHPNNIKRT | | |
| FGGVTMNGMFAMVEKYKKDGEKMLDTWTVFGDPSLLVRTLVP | | |
| TEMQVTAPANISASAQTFEVACDYNGAIATLSDDGDMVGTAIVK | | |
| DGKAIIKLNESIADETNLTLTVVGYNKVTVIKDVKVEG | | |
| GVTPKNIIDAFNGGISLVNYTGHGSETAWGTSHFGTTHVKQLTNS | SEQ ID NO: | RgpB |
| NQ | 252 | |
| NGGISLVNYTGHGSETAWGTSHFGTTHVKQLTNSNQ | SEQ ID NO: | RgpB |
| | 253 | |

In some embodiments, the at least a portion of the Kgp catalytic domain comprises at least a portion of SEQ ID NO: 64 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 64 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp catalytic domain comprises at least a portion of SEQ ID NO: 174 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 174 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp catalytic domain comprises at least a portion of SEQ ID NO: 175 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 175 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp catalytic domain comprises at least a portion of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp catalytic domain comprises at least a portion of SEQ ID NO: 61 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 61 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp catalytic domain comprises at least a portion of SEQ ID NO: 163 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 163 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp catalytic domain comprises at least a portion of SEQ ID NO: 176 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 176 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp catalytic domain comprises at least a portion of SEQ ID NO: 177 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp catalytic domain comprises a sequence of SEQ ID NO: 177 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA catalytic domain comprises at least a portion of SEQ ID NO: 98 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA catalytic domain comprises a sequence of SEQ ID NO: 98 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA catalytic domain comprises at least a portion of SEQ ID NO: 179 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA catalytic domain comprises a sequence of SEQ ID NO: 179 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA catalytic domain comprises at least a portion of SEQ ID NO: 97 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA catalytic domain comprises a sequence of SEQ ID NO: 97 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA catalytic domain comprises at least a portion of SEQ ID NO: 180 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA catalytic domain comprises a sequence of SEQ ID NO: 180 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA catalytic domain comprises at least a portion of SEQ ID NO: 66 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA catalytic domain comprises a sequence of SEQ ID NO: 66 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA catalytic domain comprises at least a portion of SEQ ID NO: 181 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA catalytic domain comprises a sequence of SEQ ID NO: 181 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpB catalytic domain comprises at least a portion of SEQ ID NO: 251 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpB catalytic domain comprises a sequence of SEQ ID NO: 251 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Preferably, the Kgp, RgpA or RgpB catalytic domain is modified in order to inactivate its proteinase function. This ensures that the polypeptide does not mediate the negative effects associated with Kgp, RgpA or RgpB proteinase function. It is possible to inactive the proteinase function of the catalytic domain in different ways. For instance, one or more residues within the active site of the catalytic domain may be mutated. Alternatively, or in addition, the catalytic domain may be truncated in order to form an inactivated catalytic domain.

Some of the catalytic domain sequences in are inactivated by mutation and/or truncation. In particular, the Kgp catalytic domain of SEQ ID NO: 64 and the RgpA catalytic domain of SEQ ID NO: 98 are inactivated by mutation. The Kgp catalytic domains of SEQ ID NOs: 88 and 163, the RgpA catalytic domains of SEQ ID NOs: 97 and 166, and the RgpB catalytic domains of SEQ ID NOs: 252 and 253 are inactivated by truncation.

In some embodiments, the at least a portion of a Kgp catalytic domain comprises a mutation that inactivates proteinase activity. In certain such embodiments, the mutation that inactivates proteinase activity is a cysteine to serine mutation at position 477 (C477S), wherein the mutation position corresponds to position 477 of the wild-type Kgp sequence of SEQ ID NO: 157.

For example, in the catalytic domains of SEQ ID NOs: 64, 174, 176 and 178 the position that corresponds to position 477 of the wild-type Kgp sequence of SEQ ID NO: 157 is position 249. Thus, the mutation that inactivates proteinase activity is a cysteine to serine mutation at position 249 (C249S) in the context of these catalytic domains.

In some embodiments, the at least a portion of a RgpA catalytic domain comprises a mutation that inactivates proteinase activity. In certain such embodiments, the mutation that inactivates proteinase activity is a cysteine to serine mutation at position 471 (C471S), wherein the mutation position corresponds to position 471 of the wild-type RgpA sequence of SEQ ID NO: 158.

For example, in the catalytic domains of SEQ ID NOs: 98, 179 and 181, the position that corresponds to position 471 of the wild-type RgpA sequence of SEQ ID NO: 158 is position C248. Thus, the mutation that inactivates proteinase activity is a cysteine to seine mutation at position 248 (C248S) in the context of these catalytic domains.

In some embodiments, the at least a portion of a RgpB catalytic domain comprises a mutation that inactivates proteinase activity. In certain such embodiments, the mutation that inactivates proteinase activity is a cysteine to serine mutation at position 473 (C473S), wherein the mutation position corresponds to position 473 of the wild-type RgpB sequence of SEQ ID NO: 159.

The inventors have found that nucleic acids and polypeptides comprising a full-length catalytic domain may be particularly advantageous in eliciting an immune response. Accordingly, in some embodiments, the at least a portion of a Kgp catalytic domain comprises a full-length Kgp catalytic domain. In some embodiments, the at least a portion of a RgpA catalytic domain comprises a full-length RgpA catalytic domain. In some embodiments, the at least a portion of a RgpB catalytic domain comprises a full-length RgpB catalytic domain.

A full-length Kgp, RgpA or RgpB catalytic domain refers to a Kgp, RgpA or RgpB catalytic domain that has not been truncated relative to a corresponding wild-type sequence. Accordingly, in some embodiments, the at least a portion of the Kgp catalytic domain is a full-length Kgp catalytic domain that is the same length as a corresponding wild-type Kgp catalytic domain sequence. In some embodiments, the at least a portion of the RgpA catalytic domain is a full-length RgpA catalytic domain that is the same length as a corresponding wild-type RgpA catalytic domain sequence. In some embodiments, the at least a portion of the RgpB catalytic domain is a full-length RgpB catalytic domain that is the same length as a corresponding wild-type RgpB catalytic domain sequence.

By way of example, Kgp catalytic domain present in Kgp from P. gingivalis strain W50 is 452 amino acids long. Thus, in some embodiments, the full-length Kgp catalytic domain is at least 452 amino acids in length (for example 452 amino acids long). The catalytic domain present in RgpA from P. gingivalis strain W50 is 438 amino acids long. Thus, in some embodiments, the full-length RgpA catalytic domain is at least 438 amino acids long (for example 438 amino acids long). The catalytic domain present in RgpB from P. gingivalis strain W50 is 437 amino acids long. Thus, in some embodiments, the full-length RgpB catalytic domain is at least 437 amino acids long (for example 437 amino acids long). The Kgp catalytic domain present in Kgp from other P. gingivalis strains may be of different length to the Kgp catalytic domain present in Kgp from P. gingivalis strain W50. Similarly, the RgpA catalytic domain present in RgpA from other P. gingivalis strains may be of different length to the RgpA catalytic domain present in RgpA from P. gingivalis strain W50. The RgpB catalytic domain present in RgpB from other P. gingivalis strains may also be of different length to the RgpB catalytic domain present in RgpB from P. gingivalis strain W50.

Accordingly, in some embodiments, the full-length Kgp catalytic domain is at least 448 amino acids long (for example 448 amino acids). In some embodiments, the full-length Kgp catalytic domain is at least 449 amino acids long (for example 449 amino acids). In some embodiments, the full-length Kgp catalytic domain is at least 450 amino acids long (for example 450 amino acids). In some embodiments, the full-length Kgp catalytic domain is at least 451 amino acids long (for example 451 amino acids). In some embodiments, the full-length Kgp catalytic domain is at least 456 amino acids long (for example 456 amino acids long).

In some embodiments, the full-length RgpA catalytic domain is at least 434 amino acids long (for example 434 amino acids). In some embodiments, the full-length RgpA catalytic domain is at least 435 amino acids long (for example 435 amino acids). In some embodiments, the full-length RgpA catalytic domain is at least 436 amino acids long (for example 436 amino acids). In some embodiments, the full-length RgpA catalytic domain is at least 437 amino acids long (for example 437 amino acids).

In some embodiments, the full-length RgpB catalytic domain is at least 433 amino acids long (for example 433 amino acids). In some embodiments, the full-length RgpB catalytic domain is at least 434 amino acids long (for example 434 amino acids). In some embodiments, the full-length RgpB catalytic domain is at least 435 amino acids long (for example 435 amino acids). In some embodiments, the full-length RgpB catalytic domain is at least 436 amino acids long (for example 436 amino acids).

Truncations of the Kgp, RgpA or RgpB catalytic domain may also be made without significantly altering the properties of the resulting polypeptide, e.g. the polypeptide is capable of eliciting antibodies that are able to block the catalytic function of Kgp, RgpA and/or RgpB. Accordingly, in some embodiments, the at least a portion of a Kgp catalytic domain is a truncated Kgp catalytic domain. In some embodiments, the at least a portion of a RgpA catalytic domain is a truncated RgpA catalytic domain. In some embodiments, the at least a portion of a RgpB catalytic domain is a truncated RgpB catalytic domain.

Thus, in some embodiments, the at least a portion of the Kgp, RgpA or RgpB catalytic domain is a truncated catalytic domain, wherein the truncated Kgp, RgpA or RgpB catalytic domain is truncated by between 1 and 35 amino acids. In certain such embodiments, the Kgp catalytic domain is truncated by between 1 and 30 amino acids, 1 and 25 amino acids, 1 and 20 amino acids, 1 and 15 amino acids, 1 and 10 amino acids, 1 and 5 amino acids. In certain such embodiments, the RgpA catalytic domain is truncated by between 1 and 30 amino acids, 1 and 25 amino acids, 1 and 20 amino acids, 1 and 15 amino acids, 1 and 10 amino acids, 1 and 5 amino acids. In certain such embodiments, the RgpB catalytic domain is truncated by between 1 and 30 amino acids, 1 and 25 amino acids, 1 and 20 amino acids, 1 and 15 amino acids, 1 and 10 amino acids, 1 and 5 amino acids. In some embodiments, the Kgp, RgpA or RgpB catalytic domain is truncated by 30 amino acids. In some embodiments, the Kgp, RgpA or RgpB catalytic domain is truncated by 25 amino acids. In some embodiments, the Kgp, RgpA or RgpB catalytic domain is truncated by 20 amino acids. In some embodiments, the Kgp, RgpA or RgpB catalytic domain is truncated by 15 amino acids. In some embodiments, the Kgp, RgpA or RgpB catalytic domain is truncated by 10 amino acids. In some embodiments, the Kgp, RgpA or RgpB catalytic domain is truncated by 5 amino acids.

A truncated Kgp, RgpA or RgpB catalytic domain may be advantageous as the amino acid sequence of the active site may be maintained as in the wild-type, and may thus elicit antibodies that are specific for the native Kgp, RgpA or RgpB active site. Accordingly, in some embodiments, the at least a portion of a Kgp catalytic domain is a truncated Kgp catalytic domain, wherein the truncation inactivates proteinase activity. In some embodiments, the at least a portion of a RgpA catalytic domain is a truncated RgpA catalytic domain, wherein the truncation inactivates proteinase activity. In some embodiments, the at least a portion of a RgpB catalytic domain is a truncated RgpB catalytic domain, wherein the truncation inactivates proteinase activity.

An example of a truncated Kgp catalytic domain in which proteinase activity is inactivated is a Lys-gingipain active site peptide (KAS peptide). A KAS peptide is a portion of the Kgp catalytic domain that comprises a portion of the Kgp catalytic domain active site. Examples of different KAS peptides are listed in, in particular, Kas2 peptides and extended Kas2 peptides.

In some embodiments, the truncated Kgp catalytic domain (e.g. KAS peptide) is at least 36 amino acids long (for example 36 amino acids) and comprises a portion of the Kgp catalytic domain active site. In other embodiments, the truncated Kgp catalytic domain (e.g. extended KAS2 peptide) is at least 47 amino acids (for example 47 amino acids) and comprises a portion of the Kgp catalytic domain active site. In some embodiments, the truncated Kgp catalytic domain comprises a KAS peptide, wherein the KAS peptide comprises a Kas2 peptide according to SEQ ID NO: 61 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In other embodiments, the KAS peptide comprises a Kas2 peptide according to SEQ ID NO: 163 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the truncated Kgp catalytic domain comprises a KAS peptide, wherein the KAS peptide comprises an extended Kas2 peptide according to SEQ ID NO: 175 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In other embodiments, the KAS peptide comprises an extended Kas2 peptide according to SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

An example of a truncated RgpA or RgpB catalytic domain in which proteinase activity is inactivated is a Arg-gingipain active site peptide (RAS peptide). A RAS peptide is a portion of the RgpA or RgpB catalytic domain that comprises a portion of the RgpA or RgpB catalytic domain active site. Examples of different RAS peptides are listed in, in particular, Ras2 peptides and extended Ras2 peptides.

In some embodiments, the truncated RgpA or RgpB catalytic domain (e.g. RAS peptide) is at least 36 amino acids long (for example 36 amino acids) and comprises a portion of the RgpA or RgpB catalytic domain active site. In other embodiments, the truncated RgpA or RgpB catalytic domain (e.g. extended RAS2 peptide) is at least 47 amino acids (for example 47 amino acids) and comprises a portion of the RgpA or RgpB catalytic domain active site.

In some embodiments, the truncated RgpA catalytic domain comprises a RAS peptide, wherein the RAS peptide comprises a Ras2 peptide according to SEQ ID NO: 180 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In other embodiments, the RAS peptide comprises a Ras2 peptide according to SEQ ID NO: 166 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the truncated RgpA catalytic domain comprises a RAS peptide, wherein the RAS peptide comprises an extended Ras2 peptide according to SEQ ID NO: 179 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In other embodiments the RAS peptide comprises an extended Ras2 peptide according to SEQ ID NO: 166 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the truncated RgpB catalytic domain comprises a RAS peptide, wherein the RAS peptide comprises a Ras2 peptide according to SEQ ID NO: 253 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the truncated RgpB catalytic domain comprises a RAS peptide, wherein the RAS peptide comprises an extended Ras2 peptide according to SEQ ID NO: 252 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Variants of a Kgp, RgpA or RgpB catalytic domains may also be employed the invention. Such variants may be to remove glycosylation sites, as described elsewhere herein.

As discussed elsewhere herein, the catalytic domain of Kgp has low sequence conservation with the catalytic domain of RgpA and RgpB. It may therefore be advantageous for the nucleic acids and polypeptides to comprise at least a portion of a catalytic domain of Kgp and at least a portion of a catalytic domain of RgpA or RgpB as this may elicit an immune response that is able to inactivate the catalytic activities of both Kgp and RgpA or RgpB. Alternatively, a composition in which a first nucleic acid or polypeptide comprises at least a portion of a catalytic domain of Kgp and a second nucleic acid or polypeptide comprises at least a portion of a catalytic domain of RgpA or RgpB may be advantageous.

Accordingly, any of the at least a portion of Kgp catalytic domains defined in the preceding paragraphs may be combined with any of the at least a portion of RgpA catalytic domains defined in the preceding paragraphs. In other embodiments, the at least a portion of Kgp catalytic domains defined in the preceding paragraphs may be combined with any of the at least a portion of RgpB catalytic domains defined in the preceding paragraphs.

In some embodiments, the nucleic acid or polypeptide comprises (i) at least a portion of a Kgp catalytic domain wherein the at least a portion of the Kgp catalytic domain is a truncated Kgp catalytic domain which comprises a KAS peptide, wherein the KAS peptide comprises a Kas2 peptide according to SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto and (ii) at least a portion of a RgpA catalytic domain wherein the at least a portion of the RgpA catalytic domain is a truncated RgpA catalytic domain which comprises a RAS peptide, wherein the RAS peptide comprises a Ras2 peptide according to SEQ ID NO: 97 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the nucleic acid or polypeptide comprises at least a portion of a Kgp catalytic domain and at least a portion of a RgpA or RgpB catalytic domain. The full-length Kgp and full-length RgpA or RgpB catalytic domains may be too long to be combined in a single nucleic acid or polypeptide alongside the other Kgp and RgpA or RgpB domains that are also present in the nucleic acid or polypeptide (e.g. DUF2436, the portions of Kgp or RgpA comprising ABMs and the K1 adhesin domain). Using a truncated Kgp catalytic domain and/or truncated RgpA or RgpB catalytic domain can be used to circumvent any issue with the nucleic acid or polypeptide becoming too long to e.g. express correctly.

Accordingly, in some embodiments, the at least a portion of a Kgp catalytic domain is a truncated Kgp catalytic domain and the at least a portion of a RgpA catalytic domain is a truncated RgpA catalytic domain. Any of the truncated Kgp catalytic domains disclosed herein may be used in combination with any of the truncated RgpA catalytic domains disclosed herein. For instance, in some embodiments, the least a portion of a Kgp catalytic domain is a KAS peptide (for example, an extended Kas2 peptide of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto) and the at least a portion of a RgpA catalytic domain is a RAS peptide (for example, an extended Ras2 peptide of SEQ ID NO: 97 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto). In other embodiments, the least a portion of a Kgp catalytic domain is a KAS peptide (for example, an extended Kas2 peptide of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto) and the at least a portion of a RgpA catalytic domain is a truncated RgpA catalytic domain. In other embodiments, at least a portion of a Kgp catalytic domain is a truncated Kgp catalytic domain and the at least a portion of a RgpA catalytic domain is a RAS peptide (for example, an extended Ras2 peptide of SEQ ID NO: 97 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto).

In other embodiments, the at least a portion of a Kgp catalytic domain is a full-length catalytic domain and the at least a portion of a RgpA catalytic domain is a truncated RgpA catalytic domain. For instance, in certain such embodiments, the at least a portion of a Kgp catalytic domain is a full-length catalytic domain and the at least a portion of a RgpA catalytic domain is a RAS peptide (for example, an extended Ras2 peptide of SEQ ID NO: 97 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto).

In other embodiments, the at least a portion of a Kgp catalytic domain is a truncated Kgp catalytic domain and the at least a portion of a RgpA catalytic domain is a full-length RgpA catalytic domain. For instance, in certain such embodiments, the at least a portion of a Kgp catalytic domain is a KAS peptide (for example, an extended Kas2 peptide of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto) and the at least a portion of a RgpA catalytic domain is a full-length RgpA catalytic domain.

In some embodiments, the at least a portion of a Kgp catalytic domain is a truncated Kgp catalytic domain and the at least a portion of a RgpB catalytic domain is a truncated RgpB catalytic domain. Any of the truncated Kgp catalytic domains disclosed herein may be used in combination with any of the truncated RgpB catalytic domains disclosed herein. For instance, in some embodiments, the least a portion of a Kgp catalytic domain is a KAS peptide (for example, an extended Kas2 peptide of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto) and the at least a portion of a RgpB catalytic domain is a RAS peptide (for example, an extended Ras2 peptide of SEQ ID NO: 252 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto). In other embodiments, the least a portion of a Kgp catalytic domain is a KAS peptide (for example, an extended Kas2 peptide of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto) and the at least a portion of a RgpB catalytic domain is a truncated RgpB catalytic domain. In other embodiments, at least a portion of a Kgp catalytic domain is a truncated Kgp catalytic domain and the at least a portion of a RgpB catalytic domain is a RAS peptide (for example, an extended Ras2 peptide of SEQ ID NO: 252 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto).

In other embodiments, the at least a portion of a Kgp catalytic domain is a full-length catalytic domain and the at least a portion of a RgpB catalytic domain is a truncated RgpB catalytic domain. For instance, in certain such embodiments, the at least a portion of a Kgp catalytic domain is a full-length catalytic domain and the at least a portion of a RgpB catalytic domain is a RAS peptide (for example, an extended Ras2 peptide of SEQ ID NO: 252 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto).

In other embodiments, the at least a portion of a Kgp catalytic domain is a truncated Kgp catalytic domain and the at least a portion of a RgpB catalytic domain is a full-length RgpB catalytic domain. For instance, in certain such embodiments, the at least a portion of a Kgp catalytic domain is a KAS peptide (for example, an extended Kas2 peptide of SEQ ID NO: 88 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto) and the at least a portion of a RgpB catalytic domain is a full-length RgpB catalytic domain.

### K1 and K2 Adhesin Domains

The nucleic acids and polypeptides of the invention comprise at least a portion of a Kgp K1 adhesin domain and/or at least a portion of RgpA K1 adhesin domain, as disclosed herein. In some embodiments, the nucleic acids and polypeptides further comprise at least a portion of Kgp K2 adhesin domain and/or at least a portion of a RgpA K1 adhesin domain.

The modular nature of gingipains is such that any of the K1 adhesin domains disclosed herein may be combined with any of the other domains disclosed herein. For instance, any of the above disclosed sequences comprising ABMs, DUFs or Cat domains may be combined with any of the K1 adhesin domains described in the subsequent paragraphs. Similarly, any of the K2 adhesin domains disclosed herein may be combined with any of the other domains disclosed herein. For instance, any of the above disclosed sequences comprising ABMs, DUFs or Cat domains may be combined with any of the K2 adhesin domains described in the subsequent paragraphs. Likewise, any of the K1 adhesin domains disclosed herein may be combined with any of the other K2 adhesin domains disclosed herein.

Wild-type Kgp and RgpA contain three adhesin domains, known as K1, K2 and K3. The three domains are structurally homologous to one another with conserved sequence motifs present in K1, K2 and K3, although the percentage sequence identity between each adhesin is relatively low (e.g. there is approximately 40% sequence identity between K1 and K2). However, there is very high sequence identity between Kgp K1 adhesin domain and RgpA K1 adhesin domain, Kgp K2 adhesin domain and RgpA K2 adhesin domain, respectively.

The three domains are members of the cleaved adhesin domain family, designated IPR011628 in the InterPro database. The cleaved adhesin domains of Kgp and RgpA are thought to have several functions in P. gingivalis, including adhesion to and colonisation of host tissues, and to promote co-aggregation of P. gingivalis with other oral pathogens and subsequent biofilm formation (Li and Collyer, 2011; Dashper et al., 2017). For example, the cleaved adhesin domains have been reported to bind to haemoglobin, human serum albumin and fibrinogen (Li et al., 2011; Ganuelas et al., 2013). These proteins are abundant in the blood and are most likely targeted by P. gingivalis during early colonisation. In addition, the cleaved adhesin domains have been shown to induce in vitro haemolysis of erythrocytes, thereby enabling P. gingivalis to acquire essential haem form erythrocytes (Li et al., 2011; Ganuelas et al., 2013).

The nucleic acids and polypeptides of the invention comprise at least a portion of a Kgp K1 adhesin domain and/or at least a portion of a RgpA K1 adhesin domain with a view to eliciting an antibody response that inhibits the adhesion functions of Kgp and RgpA.

The at least a portion of Kgp or RgpA K1 adhesin domain according to the invention is derived from a P. gingivalis Kgp or RgpA.

In some embodiments, the at least a portion of Kgp K1 adhesin domain is derived from the P. gingivalis strain W50. In P. gingivalis strain W50, the Kgp K1 adhesin domain has a sequence of SEQ ID NO: 182. Accordingly, in some embodiments, the at least a portion of the Kgp K1 adhesin domain comprises at least a portion of SEQ ID NO: 182 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp K1 adhesin domain comprises a sequence of SEQ ID NO: 182 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of RgpA K1 adhesin domain is derived from the P. gingivalis strain W50. In P. gingivalis strain W50, RgpA K1 adhesin domain has a sequence of SEQ ID NO: 185. Accordingly, in some embodiments, the at least a portion of the RgpA K1 adhesin domain comprises at least a portion of SEQ ID NO: 185 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA KI adhesin domain comprises a sequence of SEQ ID NO: 185 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Examples of K1 adhesin domain sequences that may be used according to the invention are provided in.

TABLE 14
Examples of K1 adhesin domain sequences

| Sequence | SEQ ID NO: | Gingpipain |
|---|---|---|
| GTTLSESFENGIPASWKTIDADGDGHGWKPGNAPGIAGYNSNGC | SEQ ID NO: | Kgp |
| VYSESFGLGGIGVLTPDNYLITPALDLPNGGKLTFWVCAQDANY | 182 | |
| ASEHYAVYASSTGNDASNFTNALLEETITAKGVRSPKAIRGRIQG | | |
| TWRQKTVDLPAGTKYVAFRHFQSTDMFYIDLDEVEI | | |
| GTTLSESFENGIPASWKTIDADGDGHGWKPGNAPGIAGYNSNGC | SEQ ID NO: | Kgp |
| VYSESFGLGGIGVLTPDNYLITPALDLPNGGKLTFWVCAQDANY | 183 | |
| ASEHYAVYASSTGNDASNFTNALLEETITAKGVRSPKAIRGRIQG | | |
| TWRQKTVDLPAGTKYVAFRHFQSTDMFYIDLDEVEI | | |
| GTTTLSESFENGIPASWKTIDADGDGHGWKPGNAPGIAGYNSNG | SEQ ID NO: | Kgp |
| CVYSESFGLGGIGVLTPDNYLITPALDLPNGGKLTFWVCAQDAN | 184 | |
| YASEHYAVYASSTGNDASNFTNALLEETITAKGVRSPEAIRGRIQ | | |
| GTWRQKTVDLPAGTKYVAFRHFQSTDMFYIDLDEVEI | | |
| GTTTLSESFENGIPASWKTIDADGDGHGWKPGNAPGIAGYNSNG | SEQ ID NO: | RgpA |
| CVYSESFGLGGIGVLTPDNYLITPALDLPNGGKLTFWVCAQDAN | 185 | |
| YASEHYAVYASSTGNDASNFTNALLEETITAKGVRSPKAIRGRIQ | | |
| GTWRQKTVDLPAGTKYVAFRHFQSTDMFYIDLDEVEI | | |
| GTTTLSESFENGIPASWKTIDADGDGHGWKPGNAPGIAGYNSNG | SEQ ID NO: | RgpA |
| CVYSESFGLGGIGVLTPDNYLITPALDLPNGGKLTFWVCAQDAN | 186 | |
| YASEHYAVYASSTGNDASNFTNALLEETITAKGVRSPEAIRGRIQ | | |
| GTWRQKTVDLPAGTKYVAFRHFQSTDMFYIDLDEVEI | | |

In some embodiments, the at least a portion of the Kgp K1 adhesin domain comprises at least a portion of SEQ ID NO: 183 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp K1 adhesin domain comprises a sequence of SEQ ID NO: 183 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp K1 adhesin domain comprises at least a portion of SEQ ID NO: 184 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp K1 adhesin domain comprises a sequence of SEQ ID NO: 184 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA K1 adhesin domain comprises at least a portion of SEQ ID NO: 186 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp K1 adhesin domain comprises a sequence of SEQ ID NO: 186 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

The inventors have found that nucleic acids and polypeptides comprising a full-length K1 adhesin domain may be particularly advantageous in eliciting an immune response. This may be because a polypeptide comprising a full-length K1 adhesin domain is capable of folding into a three-dimensional structure that resembles the wild-type three-dimensional structure of K1 adhesin domain, thereby enabling the construct to elicit production of antibodies that recognise conformational epitopes within the K1 adhesin domain. Accordingly, in some embodiments, the at least a portion of a K1 adhesin domain comprises a full-length K1 adhesin domain. In some embodiments, the at least a portion of a RgpA K1 adhesin domain comprises a full-length RgpA K1 adhesin domain.

A full-length Kgp or RgpA K1 adhesin domain refers to a Kgp or RgpA K1 adhesin domain that has not been truncated relative to a corresponding wild-type K1 adhesin domain. Accordingly, in some embodiments, the at least a portion of the Kgp K1 adhesin domain is a full-length Kgp K1 adhesin domain that is the same length as a corresponding wild-type Kgp K1 adhesin domain. In some embodiments, the at least a portion of the RgpA K1 adhesin domain is a full-length RgpA K1 adhesin domain that is the same length as a corresponding wild-type RgpA K1 adhesin domain.

By way of example, Kgp K1 adhesin domain present in Kgp from P. gingivalis strain W50 is 169 amino acids long. Thus, in some embodiments, the full-length Kgp K1 adhesin domain is at least 169 amino acids in length (for example 169 amino acids long). The K1 adhesin domain present in RgpA from P. gingivalis strain W50 is 170 amino acids long. Thus, in some embodiments, the full-length RgpA K1 adhesin domain is at least 170 amino acids long (for example 170 amino acids long).

The Kgp K1 domain present in Kgp from other P. gingivalis strains may be of different length to the Kgp K1 domain present in Kgp from P. gingivalis strain W50. Similarly, the RgpA K1 domain present in Kgp from other P. gingivalis strains may be of different length to the RgpA K1 domain present in RgpA from P. gingivalis strain W50.

Accordingly, in some embodiments, the full-length Kgp K1 adhesin domain is at least 165 amino acids long (for example 165 amino acids long). In some embodiments, the full-length Kgp K1 adhesin domain is at least 166 amino acids long (for example 166 amino acids long). In some embodiments, the full-length Kgp K1 adhesin domain is at least 167 amino acids long (for example 167 amino acids long). In some embodiments, the full-length Kgp K1 adhesin domain is at least 168 amino acids long (for example 168 amino acids long). In some embodiments, the full-length Kgp K1 adhesin is at least 170 amino acids long (for example 170 amino acids long).

In some embodiments, the full-length RgpA K1 adhesin domain is at least 166 amino acids long (for example 166 amino acids long). In some embodiments, the full-length RgpA K1 adhesin domain is at least 167 amino acids long (for example 167 amino acids long). In some embodiments, the full-length RgpA K1 adhesin domain is at least 168 amino acids long (for example 168 amino acids long). In some embodiments, the full-length RgpA K1 adhesin domain is at least 169 amino acids long (for example 169 amino acids long). Truncations of the Kgp or RgpA K1 adhesin domain may also be made without significantly altering the properties of the resulting polypeptide, e.g. the resulting polypeptide is still capable of eliciting antibodies that block Kgp and/or RgpA adhesion function. Accordingly, in some embodiments, the at least a portion of a Kgp K1 adhesin domain is a truncated Kgp K1 adhesin domain. In some embodiments, the at least a portion of a RgpA K1 adhesin domain is a truncated RgpA K1 adhesin domain.

Thus, in some embodiments, the at least a portion of the Kgp or RgpA K1 adhesin domain is a truncated Kgp or RgpA K1 adhesin domain, wherein the truncated Kgp or RgpA K1 adhesin domain is truncated by between 1 and 35 amino acids. In certain such embodiments, the Kgp or RgpA K1 adhesin domain is truncated by between 1 and 30 amino acids, 1 and 25 amino acids, 1 and 20 amino acids, 1 and 15 amino acids, 1 and 10 amino acids, 1 and 5 amino acids. In certain such embodiments, the Kgp K1 adhesin domain is truncated by between 1 and 30 amino acids, 1 and 25 amino acids, 1 and 20 amino acids, 1 and 15 amino acids, 1 and 10 amino acids, 1 and 5 amino acids. In certain such embodiments, the RgpA K1 adhesin domain is truncated by between 1 and 30 amino acids, 1 and 25 amino acids, 1 and 20 amino acids, 1 and 15 amino acids, 1 and 10 amino acids, 1 and 5 amino acids. In some embodiments, the Kgp or RgpA K1 adhesin domain is truncated by 30 amino acids. In some embodiments, the Kgp or RgpA K1 adhesin domain is truncated by 25 amino acids. In some embodiments, the Kgp or RgpA K1 adhesin domain is truncated by 20 amino acids. In some embodiments, the Kgp or RgpA K1 adhesin domain is truncated by 15 amino acids. In some embodiments, the Kgp or RgpA K1 adhesin domain is truncated by 10 amino acids. In some embodiments the Kgp or RgpA K1 adhesin domain is truncated by 5 amino acids.

In some embodiments, the truncated Kgp K1 adhesin domain comprises the sequence GTTTLSESF (SEQ ID NO: 191).

In some embodiments, the truncated RgpA K1 adhesin domain comprises the sequence of GTTTLSESF (SEQ ID NO: 192).

As can be seen in FIGS. 2A-2B, in the wild-type Kgp and RgpA sequences, the C-terminal portion of ABM3 overlaps with the N-terminal portion of the K1 adhesin domain. Accordingly, in some embodiments, the portion of Kgp comprising ABM3 and the at least a portion of the Kgp K1 adhesin domain overlap (for example, eight C-terminal residues of a portion of Kgp comprising ABM3 are also the eight N-terminal residues of the Kgp K1 adhesin domain). In some embodiments, the portion of RgpA comprising ABM3 and the at least a portion of the RgpA K1 adhesin domain overlap (for example, nine C-terminal residues of a portion of Kgp comprising ABM3 are also the nine N-terminal residues of RgpA K1 adhesin domain).

Examples of sequences that may be used according to the invention in which a Kgp or RgpA portion comprising ABM3 and a Kgp or RgpA K1 adhesin domain overlap are provided in.

TABLE 15
Examples K1 adhesin domain sequences and ABM3 sequences

| Sequence | SEQ ID NO: | Gingpipain |
|---|---|---|
| PQGTPNPNPNPNPNPGTTLSESFENGIPASWKTIDADGDGHGWKP | SEQ ID NO: 93 | Kgp |
| GNAPGIAGYNSNGCVYSESFGLGGIGVLTPDNYLITPALDLPNGG | | |
| KLTFWVCAQDANYASEHYAVYASSTGNDASNFANALLEETITAK | | |
| GVRSPKAIRGRIQGTWRQKTVDLPAGTKYVAFRHFQSTDMFYID | | |
| LDEVEIKAN | | |
| PQGTPNPNPNPNPNPGTTLSESF | SEQ ID NO: 94 | Kgp |
| PQGTPNPNPNPNPNPNPGTTTLSESF | SEQ ID NO: | RgpA |
| | 103 | |

Accordingly, in some embodiments, the Kgp portion comprising ABM3 and the Kgp K1 adhesin domain together comprise a sequence of SEQ ID NO: 93 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the Kgp portion comprising ABM3 and the Kgp K1 adhesin domain together comprise a sequence of SEQ ID NO: 94 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the Kgp portion comprising ABM3 and the Kgp K1 adhesin domain together comprise a sequence of SEQ ID NO: 103 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Variants of a Kgp or RgpA K1 adhesin domains may also be employed the invention. Such variants may be to remove glycosylation sites, as described elsewhere herein.

The nucleic acids and polypeptides of the invention may contain at least a portion of an additional cleaved adhesin domain, in addition to the at least portion of a Kgp and/or RgpA K1 adhesin domain. The inclusion of at least a portion of the K2 adhesin domain may allow the resulting polypeptide to form a structure that more closely resembles the wild-type gingipain structure, thereby providing additional three-dimensional epitopes that may be useful in raising an immune response. Alternatively, or in addition, the inclusion of at least a portion of the K2 adhesin domain may mean the polypeptide is able to elicit antibodies that are specific to the K2 domain that are capable of inhibiting K2-specific functions of Kgp and/or RgpA. Accordingly, in some embodiments, the nucleic acids and polypeptides of the invention may contain at least a portion of a Kgp K2 adhesin domain. In some embodiments, the nucleic acids and polypeptides of the invention may contain at least a portion of a RgpA K2 adhesin domain.

In some embodiments, the Kgp or RgpA K2 adhesin domain is full-length. By way of example, Kgp K2 adhesin domain present in Kgp from P. gingivalis strain W50 is 172 amino acids long. Thus, in some embodiments, the full-length Kgp K2 adhesin domain is at least 172 amino acids in length (for example 172 amino acids long). By way of example, RgpA K2 adhesin domain present in RgpA from P. gingivalis strain W50 is 172 amino acids long. Thus, in some embodiments, the full-length RgpA K2 adhesin domain is at least 172 amino acids in length (for example 172 amino acids long).

The Kgp K2 domain present in Kgp from other P. gingivalis strains may be of different length to the Kgp K2 domain present in Kgp from P. gingivalis strain W50. Similarly, the RgpA K2 domain present in Kgp from other P. gingivalis strains may be of different length to the RgpA K2 domain present in RgpA from P. gingivalis strain W50.

Accordingly, in some embodiments, the full-length Kgp or RgpA K2 adhesin domain is at least 168 amino acids long (for example 168 amino acids). In some embodiments, the full-length Kgp or RgpA K2 adhesin domain is at least 169 amino acids long (for example 169 amino acids). In some embodiments, the full-length Kgp or RgpA K2 adhesin domain is at least 170 amino acids long (for example 170 amino acids). In some embodiments, the full-length Kgp or RgpA K2 adhesin domain is at least 171 amino acids long (for example 171 amino acids). In some embodiments, the full-length Kgp or RgpA K2 adhesin domain is at least 171 amino acids long (for example 171 amino acids). In some embodiments, the full-length Kgp or RgpA K2 adhesin domain is at least 176 amino acids long (for example 176 amino acids). In some embodiments, the full-length Kgp or RgpA K2 adhesin domain is at least 178 amino acids long (for example 178 amino acids).

Truncations of the Kgp or RgpA K2 adhesin domain may also be made without significantly altering the properties of the resulting polypeptide, e.g. the resulting polypeptide is still capable of eliciting antibodies that block Kgp and/or RgpA adhesion function. Accordingly, in some embodiments, the at least a portion of a Kgp K2 adhesin domain is a truncated Kgp K2 adhesin domain. In some embodiments, the at least a portion of a RgpA K2 adhesin domain is a truncated RgpA K2 adhesin domain.

Thus, in some embodiments, the at least a portion of the Kgp or RgpA K2 adhesin domain is a truncated Kgp or RgpA K2 adhesin domain, wherein the truncated Kgp or RgpA K2 adhesin domain is truncated by between 1 and 35 amino acids. In certain such embodiments, the Kgp K2 adhesin domain is truncated by between 1 and 30 amino acids, 1 and 25 amino acids, 1 and 20 amino acids, 1 and 15 amino acids, 1 and 10 amino acids, 1 and 5 amino acids. In certain such embodiments, the RgpA K2 adhesin domain is truncated by between 1 and 30 amino acids, 1 and 25 amino acids, 1 and 20 amino acids, 1 and 15 amino acids, 1 and 10 amino acids, 1 and 5 amino acids. In some embodiments, the Kgp or RgpA K2 adhesin domain is truncated by 30 amino acids. In some embodiments, the Kgp or RgpA K2 adhesin domain is truncated by 25 amino acids. In some embodiments, the Kgp or RgpA K2 adhesin domain is truncated by 20 amino acids. In some embodiments, the Kgp or RgpA K2 adhesin domain is truncated by 15 amino acids. In some embodiments, the Kgp or RgpA K2 adhesin domain is truncated by 10 amino acids. In some embodiments the Kgp or RgpA K2 adhesin domain is truncated by 5 amino acids.

In some embodiments, the at least a portion of Kgp or RgpA K2 adhesin domain is derived from the P. gingivalis strain W50. In P. gingivalis strain W50, the Kgp K2 adhesin domain has a sequence of SEQ ID NO: 187. Accordingly, in some embodiments, the at least a portion of the Kgp K2 adhesin domain comprises at least a portion of SEQ ID NO: 187 or a sequence that has at least 70% (e.g. at least 90 or 95%) identity thereto. For example, the at least a portion of the Kgp K2 adhesin domain comprises a sequence of SEQ ID NO: 187 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In P. gingivalis strain W50, RgpA K2 adhesin domain has a sequence of SEQ ID NO: 189. Accordingly, in some embodiments, the at least a portion of the RgpA K2 adhesin domain comprises at least a portion of SEQ ID NO: 189 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA K2 adhesin domain comprises a sequence of SEQ ID NO: 189 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

Examples of K2 adhesin domain sequences that may be used according to the invention are provided in

TABLE 16
Examples of K2 adhesin domain sequences.

| Sequence | SEQ ID NO: | Gingpipain |
|---|---|---|
| FTETFESSTHGEAPAEWTTIDADGDGQGWLCLSSGQLDWLTAHG | SEQ ID NO: | Kgp |
| GSNVVSSFSWNGMALNPDNYLISKDVTGATKVKYYYAVNDGFP | 187 | |
| GDHYAVMISKTGTNAGDFTVVFEETPNGINKGGARFGLSTEANG | | |
| AKPQSVWIERTVDLPAGTKYVAFRHYNCSDLNYILLDDIQF | | |
| KRADFTETFESSTHGEAPAEWTTIDADGDGQGWLCLSSGQLDWL | SEQ ID NO: 96 | Kgp |
| TAHGGSNVVSSFSWNGMALNPDNYLISKDVTGATKVKYYYAVN | | |
| DGFPGDHYAVMISKTGTNAGDFTVVFEETPNGINKGGARFGLSTE | | |
| ANGAKPQSVWIERTVDLPAGTKYVAFRHYQCSDLNYILLDDIQFT | | |
| M | | |
| FTETFESSTHGEAPAEWTTIDADGDGQGWLCLSSGQLDWLTAHG | SEQ ID NO: | Kgp |
| GSNVVSSFSWNGMALNPDNYLISKDVTGATKVKYYYAVNDGFP | 188 | |
| GDHYAVMISKTGTNAGDFTVVFEETPNGINKGGARFGLSTEANG | | |
| AKPQSVWIERTVDLPAGTKYVAFRHYNCSDLNYILLDDIQF | | |
| FTETFESSTHGEAPAEWTTIDADGDGQGWLCLSSGQLDWLTAHG | SEQ ID NO: | RgpA |
| GTNVVASFSWNGMALNPDNYLISKDVTGATKVKYYYAVNDGFP | 189 | |
| GDHYAVMISKTGTNAGDFTVVFEETPNGINKGGARFGLSTEANG | | |
| AKPQSVWIERTVDLPAGTKYVAFRHYNCSDLNYILLDDIQF | | |
| DFTETFESSTHGEAPAEWTTIDADGDGQGWLCLSSGQLDWLTAH | SEQ ID NO: | RgpA |
| GGTNVVASFSWNGMALNPDNYLISKDVTGATKVKYYYAVNDGF | 104 | |
| PGDHYAVMISKTGTNAGDFTVVFEETPNGINKGGARFGLSTEAN | | |
| GAKPQSVWIERTVDLPAGTKYVAFRHYQCSDLNYILLDDIQFTM | | |
| G | | |
| FTETFESSTHGEAPAEWTTIDADGDGQGWLCLSSGQLDWLTAHG | SEQ ID NO: | RgpA |
| GTNVVASFSWNGMALNPDNYLISKDVTGATKVKYYYAVNDGFP | 190 | |
| GDHYAVMISKTGTNAGDFTVVFEETPNGINKGGARFGLSTEANG | | |
| AKPQSVWIERTVDLPAGTKYVAFRHYNCSDLNYILLDDIQF | | |

In some embodiments, the at least a portion of the Kgp K2 adhesin domain comprises at least a portion of SEQ ID NO: 96 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp K2 adhesin domain comprises a sequence of SEQ ID NO: 96 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the Kgp K2 adhesin domain comprises at least a portion of SEQ ID NO: 188 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the Kgp K2 adhesin domain comprises a sequence of SEQ ID NO: 188 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA K2 adhesin domain comprises at least a portion of SEQ ID NO: 104 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA K2 adhesin domain comprises a sequence of SEQ ID NO: 104 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the at least a portion of the RgpA K2 adhesin domain comprises at least a portion of SEQ ID NO: 190 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. For example, the at least a portion of the RgpA K2 adhesin domain comprises a sequence of SEQ ID NO: 190 or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

The inventors have found that nucleic acids and polypeptides comprising a full-length K2 adhesin domain may be particularly advantageous in eliciting an immune response. This may be because a polypeptide comprising a full-length K2 adhesin domain is capable of folding into a three-dimensional structure that resembles the wild-type three-dimensional structure of K2 adhesin domain, thereby enabling the construct to elicit production of antibodies that recognise conformational epitopes within the K2 adhesin domain. Accordingly, in some embodiments, the at least a portion of a K2 adhesin domain comprises a full-length K2 adhesin domain. In some embodiments, the at least a portion of a RgpA K2 adhesin domain comprises a full-length RgpA K2 adhesin domain.

A full-length Kgp or RgpA K2 adhesin domain refers to a Kgp or RgpA K2 adhesin domain that has not been truncated relative to a corresponding wild-type K2 adhesin domain. Accordingly, in some embodiments, the at least a portion of the Kgp K2 adhesin domain is a full-length Kgp K2 adhesin domain that is the same length as a corresponding wild-type Kgp K2 adhesin domain. In some embodiments, the at least a portion of the RgpA K2 adhesin domain is a full-length RgpA K2 adhesin domain that is the same length as a corresponding wild-type RgpA K2 adhesin domain.

By way of example, Kgp K2 adhesin domain present in Kgp from P. gingivalis strain W50 is 172 amino acids long. Thus, in some embodiments, the full-length Kgp K2 adhesin domain is at least 172 amino acids in length (for example, 172 amino acids long). The K2 adhesin domain present in RgpA from P. gingivalis strain W50 is 172 amino acids long. Thus, in some embodiments, the full-length RgpA K2 adhesin domain is at least 172 amino acids long (for example, 172 amino acids long).

Variants of a Kgp or RgpA K1 adhesin domains may also be employed the invention. Such variants may be to remove glycosylation sites, as described elsewhere herein.

In some embodiments, the nucleic acid or the polypeptide comprises at least a portion of a Kgp K1 adhesin domain and at least a portion of a Kgp K2 adhesin domain. In certain such embodiments, the at least a portion of a Kgp K1 adhesin domain and at least a portion of a Kgp K2 adhesin domain each comprise a sequence as shown in, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

TABLE 17
Combinations of K1 adhesin domain sequences
and K2 adhesin domain sequences

| Kgp K1 | Kgp K2 |
|---|---|
| adhesin domain | adhesin domain |
| SEQ ID NO: 182 | SEQ ID NO: 185 |
| SEQ ID NO: 93 | SEQ ID NO: 96 |
| SEQ ID NO: 94 | SEQ ID NO: 96 |

In some embodiments, the nucleic acid or the polypeptide comprises at least a portion of a Kgp K1 adhesin domain, at least a portion of a Kgp K2 adhesin domain and at least a portion of RgpA K1 adhesin domain. In certain such embodiments, the at least a portion of a Kgp K1 adhesin domain and at least a portion of a Kgp K2 adhesin domain each comprise a sequence as shown in, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto, and the RgpA K1 adhesin domain comprise a sequence of SEQ ID NO: 103.

In some embodiments, the nucleic acid or the polypeptide comprises at least a portion of a RgpA K1 adhesin domain and at least a portion of a RgpA K2 adhesin domain. In certain embodiments, the at least a portion of a RgpA K1 adhesin domain and at least a portion of a RgpA K2 adhesin domain each comprise a sequence as shown in, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

TABLE 18
Combinations of RgpA K1 adhesin domain sequences
and RgpA K2 adhesin domain sequences

| RgpA K1 | RgpA K2 |
|---|---|
| adhesin domain | adhesin domain |
| SEQ ID NO: 185 | SEQ ID NO: 189 |
| SEQ ID NO: 103 | SEQ ID NO: 104 |

Positioning of gingipain domains and sequence motifs

The domains described in any of the preceding sections (i.e. a first Kgp portion comprising ABM1, a first portion comprising ABM2, a second portion comprising ABM1, a second portion comprising ABM2, at least a portion of a DUF2436, at least a portion of a Kgp catalytic domain and at least a portion of a Kgp K1 adhesin domains) can be combined to produce or nucleic acids encoding polypeptides or polypeptide of the invention.

The modular nature of the Kgp structure means that the different gingipain domains of the nucleic acids and polypeptides may be arranged in any order. However, typically, the gingipain domains are arranged in same order as the domains are arranged in the wild-type Kgp and RgpA proteins. Arranging the domains in the same order as the wild-type Kgp and RgpA proteins may enhance folding of the polypeptide in a way that more closely resembles the wild-type Kgp and RgpA, thereby allowing for e.g. conformational epitopes to be retained.

As can be seen from FIG. 1, the order of the domains in wild-type Kgp and RgpA is from the N-terminus to the C-terminus: propeptide; catalytic domain; first portion comprising ABM1; DUF2436; first portion comprising ABM2; second portion comprising ABM1; portion comprising ABM3; K1; K2; second portion comprising ABM2; K3; C-terminal domain. For embodiments that comprise a subset of these domains the domains that are present are positioned in the same N-terminal to C-terminal order with the omission of any domains from the wild-type sequence.

For instance, in some embodiments, the polypeptides or nucleic acids encoding polypeptides of the invention comprise; i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; and vi) a Kgp portion that comprises ABM3; wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2.

In some embodiments, the polypeptides or nucleic acids encoding polypeptides of the invention comprise; i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

In some embodiments, the polypeptides or nucleic acids encoding polypeptides of the invention comprise; i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2; RgpA catalytic domain.

In some embodiments, the polypeptides or nucleic acids encoding polypeptides of the invention comprise; i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; vi) a Kgp portion that comprises ABM3; and vii) at least a portion of a RgpA catalytic domain, wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2; RgpA catalytic domain.

In some embodiments, the polypeptides or nucleic acids encoding polypeptides of the invention comprise; i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; vi) a Kgp portion that comprises ABM3; vii) at least a portion of a Kgp K2 adhesin domain; and viii) at least a portion of a RgpA catalytic domain, wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; Kgp K2; second Kgp portion comprising ABM2; RgpA catalytic domain.

In some embodiments, the polypeptides or nucleic acids encoding polypeptides of the invention comprise; i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; vi) a Kgp portion that comprises ABM3; vii) at least a portion of a RgpA K2 adhesin domain; and viii) at least a portion of a RgpA catalytic domain, wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; RgpA K2; second Kgp portion comprising ABM2; RgpA catalytic domain.

In some embodiments, the polypeptides or nucleic acids encoding polypeptides of the invention comprise; i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; vi) a Kgp portion that comprises ABM3; vii) at least a portion of a Kgp K2 adhesin domain; and viii) at least a portion of a RgpA catalytic domain; ix) a first RgpA portion that comprises ABM1 and a first RgpA portion that comprises ABM2; x) at least a portion of a RgpA DUF2436, wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; Kgp K2; second Kgp portion comprising ABM2; first RgpA portion comprising ABM1; RgpA DUF2436; first RgpA portion comprising ABM2; RgpA catalytic domain.

In some embodiments, the polypeptides or nucleic acids encoding polypeptides of the invention comprise; i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; vi) a Kgp portion that comprises ABM3; vii) at least a portion of a RgpA K2 adhesin domain; and viii) at least a portion of a RgpA catalytic domain; ix) a first RgpA portion that comprises ABM1 and a first RgpA portion that comprises ABM2; x) at least a portion of a RgpA DUF2436, wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; RgpA K2; second Kgp portion comprising ABM2; first RgpA portion comprising ABM1; RgpA DUF2436; first RgpA portion comprising ABM2; RgpA catalytic domain.

Examples of nucleic acids encoding polypeptides or polypeptides of the invention are provided in Table 19. Other examples of nucleic acids encoding polypeptides or polypeptides of the invention are the sequences provided in Table 19, wherein the N-terminal methionine is absent. This table also provides nucleic acid sequences that encode the polypeptide, which also form part of the invention.

TABLE 19
Examples of polypeptides of the invention, or polypeptides encoded by nucleic acids
of the invention

| | | Exemplary | |
|---|---|---|---|
| | Amino acid | Nucleic acid | |
| Amino acid sequence | SEQ ID NO: | SEQ ID NOs: | Designation |
| MDVYTDHGDLYNTPVRMLVVAGAKFKEAL | SEQ ID NO: 1 | SEQ ID NO: 21 | Kgp_1A_mCat |
| KPWLTWKAQKGFYLDVHYTDEAEVGTTNAS | | SEQ ID NO: 22 | |
| IKAFIHKKYNDGLAASAAPVFLALVGDTDVIS | | | |
| GEKGKKSKKVTDLYYSAVDGDYFPEMYTFR | | | |
| MSASSPEELTNIIDKVLMYEKATMPDKSYLEK | | | |
| VLLIAGADYSWNSQVGQPTIKYGMQYYYNQ | | | |
| EHGYTDVYNYLKAPYTGCYSHLNTGVSFAN | | | |
| YTAHGSETAWADPLLTTSQLKALTNKDKYFL | | | |
| AIGNCSITAQFDYVQPCFGEVITRVKEKGAYA | | | |
| YIGSSPNSYWGEDYYWSVGANAVFGVQPTFE | | | |
| GTSMGSYDATFLEDSYNTVNSIMWAGNLAA | | | |
| THAGNIGNITHIGAHYYWEAYHVLGDGSVMP | | | |
| YRAMPKTNTYTLPASLPQNQASYSIQASAGS | | | |
| YVAISKDGVLYGTGVANASGVATVSMTKQIT | | | |
| ENGNYDVVITRSNYLPVIKQIQVGEPSPYQPV | | | |
| SNLTATTQGQKVTLKWEAPSAKKSEGSREVK | | | |
| RIGDGLFVTIEPANDVRANEAKVVLAADNVW | | | |
| GDNTGYQFLLDADHNTFGSVIPATGPLFTGTA | | | |
| SSNLYSANFEYLIPANADPVVTTQNIIVTGQG | | | |
| EVVIPGGVYDYCITNPEPASGKMWIAGDGGN | | | |
| QPARYDDFTFEAGKKYTFTMRRAGMGDGTD | | | |
| MEAEDDSPASYTYTVYRDGTKIKEGLTATTF | | | |
| EEDGVAAGNHEYCVEVKYTAGVSPKVCKDV | | | |
| TVEGSNEFAPVQNLTGSSVGQKVTLKWDAPN | | | |
| GTPNPNPNPNPNPGTTLSESFENGIPASWKTID | | | |
| ADGDGHGWKPGNAPGIAGYNSNGCVYSESF | | | |
| GLGGIGVLTPDNYLITPALDLPNGGKLTFWVC | | | |
| AQDANYASEHYAVYASSTGNDASNFTNALL | | | |
| EETITAKGVRSPKAIRGRIQGTWRQKTVDLPA | | | |
| GTKYVAFRHFQSTDMFYIDLDEVEIKANGGG | | | |
| SPTPTDYTYTVYRDGTKIKEGLTETTFEEDGV | | | |
| ATGNHEYCVEVKYTAGVSPKKCVNVTVN | | | |
| MDVYTDHGDLYNTPVRMLVVAGAKFKEAL | SEQ ID NO: 6 | SEQ ID NO: 31 | Kgp_1B_mCat |
| KPWLTWKAQKGFYLDVHYTDEAEVGTTNAS | | SEQ ID NO: 32 | |
| IKAFIHKKYNDGLAASAAPVFLALVGDTDVIS | | | |
| GEKGKKSKKVTDLYYSAVDGDYFPEMYTFR | | | |
| MSASSPEELTNIIDKVLMYEKATMPDKSYLEK | | | |
| VLLIAGADYSWNSQVGQPTIKYGMQYYYNQ | | | |
| EHGYTDVYNYLKAPYTGCYSHLNTGVSFAN | | | |
| YTAHGSETAWADPLLTTSQLKALTNKDKYFL | | | |
| AIGNCSITAQFDYVQPCFGEVITRVKEKGAYA | | | |
| YIGSSPNSYWGEDYYWSVGANAVFGVQPTFE | | | |
| GTSMGSYDATFLEDSYNTVNSIMWAGNLAA | | | |
| THAGNIGNITHIGAHYYWEAYHVLGDGSVMP | | | |
| YRAMPKTNTYTLPASLPQNQASYSIQASAGS | | | |
| YVAISKDGVLYGTGVANASGVATVSMTKQIT | | | |
| ENGNYDVVITRSNYLPVIKQIQVGEPSPYQPV | | | |
| SNLTATTQGQKVTLKWEAPSAKKSEGSREVK | | | |
| RIGDGLFVTIEPANDVRANEAKVVLAADNVW | | | |
| GDNTGYQFLLDADHNTFGSVIPATGPLFTGTA | | | |
| SSNLYSANFEYLIPANADPVVTTQNIIVTGQG | | | |
| EVVIPGGVYDYCITNPEPASGKMWIAGDGGN | | | |
| QPARYDDFTFEAGKKYTFTMRRAGMGDGTD | | | |
| MEAEDDSPASYTYTVYRDGTKIKEGLTATTF | | | |
| EEDGVAAGNHEYCVEVKYTAGVSPKVCKDV | | | |
| TVEGSNEFAPVQNLTGSSVGQKVTLKWDAPN | | | |
| GTPNPNPNPNPNPGTTLSESFPTDYTYTVYRD | | | |
| GTKIKEGLTETTFEEDGVATGNHEYCVEVKY | | | |
| TAGVSPKKCVNVTVN | | | |
| MNTGVSFANYTAHGSETAWADPLLTTSQLK | SEQ ID NO: 11 | SEQ ID NO: 41 | Kgp_2A |
| ALTNKDKPYQPVSNLTATTQGQKVTLKWEA | | SEQ ID NO: 42 | |
| PSAKKSEGSREVKRIGDGLFVTIEPANDVRAN | | | |
| EAKVVLAADNVWGDNTGYQFLLDADHNTF | | | |
| GSVIPATGPLFTGTASSNLYSANFEYLIPANAD | | | |
| PVVTTQNIIVTGQGEVVIPGGVYDYCITNPEP | | | |
| ASGKMWIAGDGGNQPARYDDFTFEAGKKYT | | | |
| FTMRRAGMGDGTDMEAEDDSPASYTYTVYR | | | |
| DGTKIKEGLTATTFEEDGVAAGNHEYCVEVK | | | |
| YTAGVSPKVCKDVTVEGSNEFAPVQNLTGSS | | | |
| VGQKVTLKWDAPNGTPNPNPNPNPNPGTTLS | | | |
| ESFENGIPASWKTIDADGDGHGWKPGNAPGI | | | |
| AGYNSNGCVYSESFGLGGIGVLTPDNYLITPA | | | |
| LDLPNGGKLTFWVCAQDANYASEHYAVYAS | | | |
| STGNDASNFTNALLEETITAKGVRSPKAIRGRI | | | |
| QGTWRQKTVDLPAGTKYVAFRHFQSTDMFY | | | |
| IDLDEVEIKANGGGSPTPTDYTYTVYRDGTKI | | | |
| KEGLTETTFEEDGVATGNHEYCVEVKYTAGV | | | |
| SPKKCVNVTVN | | | |
| MNTGVSFANYTAHGSETAWADPLLTTSQLK | SEQ ID NO: 16 | SEQ ID NO: 51 | Kgp_2B |
| ALTNKDKPYQPVSNLTATTQGQKVTLKWEA | | SEQ ID NO: 52 | |
| PSAKKSEGSREVKRIGDGLFVTIEPANDVRAN | | | |
| EAKVVLAADNVWGDNTGYQFLLDADHNTF | | | |
| GSVIPATGPLFTGTASSNLYSANFEYLIPANAD | | | |
| PVVTTQNIIVTGQGEVVIPGGVYDYCITNPEP | | | |
| ASGKMWIAGDGGNQPARYDDFTFEAGKKYT | | | |
| FTMRRAGMGDGTDMEAEDDSPASYTYTVYR | | | |
| DGTKIKEGLTATTFEEDGVAAGNHEYCVEVK | | | |
| YTAGVSPKVCKDVTVEGSNEFAPVQNLTGSS | | | |
| VGQKVTLKWDAPNGTPNPNPNPNPNPGTTLS | | | |
| ESFPTDYTYTVYRDGTKIKEGLTETTFEEDGV | | | |
| ATGNHEYCVEVKYTAGVSPKKCVNVTVN | | | |
| MEPGRYTPVEEKQNGRMIVIVAKKYEGDIKD | SEQ ID NO: 367 | | 1B_RgpA |
| FVDWKNQRGLRTEVKVAEDIASPVTANAIQQ | | | |
| FVKQEYEKEGNDLTYVLLIGDHKDIPAKITPG | | | |
| IKSDQVYGQIVGNDHYNEVFIGRFSCESKEDL | | | |
| KTQIDRTIHYERNITTEDKWLGQALCIASAEG | | | |
| GPSADNGESDIQHENVIANLLTQYGYTKIIKC | | | |
| YDPGVTPKNIIDAFNGGISLANYTGHGSETAW | | | |
| GTSHFGTTHVKQLTNSNQLPFIFDVASVNGDF | | | |
| LFSMPCFAEALMRAQKDGKPTGTVAIIASTIN | | | |
| QSWASPMRGQDEMNEILCEKHPNNIKRTFGG | | | |
| VTMNGMFAMVEKYKKDGEKMLDTWTVFGD | | | |
| PSLLVRTLVPTKMQVTAPAQINLTDASVNVS | | | |
| CDYNGAIATISANGKMFGSAVVENGTATINL | | | |
| TGLTNESTLTLTVVGYNKETVIKTINTNGEPN | | | |
| PYQPVSNLTATTQGQKVTLKWDAPSTKTNAT | | | |
| TNTARSVDGIRELVLLSVSDAPELLRSGQAEI | | | |
| VLEAHDVWNDGSGYQILLDADHDQYGQVIP | | | |
| SDTHTLWPNCSVPANLFAPFEYTVPENADPSC | | | |
| SPTNMIMDGTASVNIPAGTYDFAIAAPQANA | | | |
| KIWIAGQGPTKEDDYVFEAGKKYHFLMKKM | | | |
| GSGDGTELTISEGGGSDYTYTVYRDGTKIKEG | | | |
| LTATTFEEDGVAAGNHEYCVEVKYTAGVSPK | | | |
| VCKDVTVEGSNEFAPVQNLTGSAVGQKVTL | | | |
| KWDAPNGTPNPNPNPNPNPNPGTTTLSESFGK | | | |
| RADFTETFESSTHGEAPAEWTTIDADGDGQG | | | |
| WLCLSSGQLDWLTAHGGTNVVASFSWNGM | | | |
| ALNPDNYLISKDVTGATKVKYYYAVNDGFP | | | |
| GDHYAVMISKTGTNAGDFTVVFEETPNGINK | | | |
| GGARFGLSTEANGAKPQSVWIERTVDLPAGT | | | |
| KYVAFRHYNCSDLNYILLDDIQFTMGGSPTPT | | | |
| DYTYTVYRDGTKIKEGLTETTFEEDGVATGN | | | |
| HEYCVEVKYTAGVSPKKCVNVTVN | | | |
| MNPYQPVSNLTATTQGQKVTLKWDAPSTKT | SEQ ID NO: 371 | | 2B_RgpA |
| NATTNTARSVDGIRELVLLSVSDAPELLRSGQ | | | |
| AEIVLEAHDVWNDGSGYQILLDADHDQYGQ | | | |
| VIPSDTHTLWPNCSVPANLFAPFEYTVPENAD | | | |
| PSCSPTNMIMDGTASVNIPAGTYDFAIAAPQA | | | |
| NAKIWIAGQGPTKEDDYVFEAGKKYHFLMK | | | |
| KMGSGDGTELTISEGGGSDYTYTVYRDGTKI | | | |
| KEGLTATTFEEDGVAAGNHEYCVEVKYTAG | | | |
| VSPKVCKDVTVEGSNEFAPVQNLTGSAVGQK | | | |
| VTLKWDAPNGTPNPNPNPNPNPNPGTTTLSES | | | |
| FGKRADFTETFESSTHGEAPAEWTTIDADGD | | | |
| GQGWLCLSSGQLDWLTAHGGTNVVASFSWN | | | |
| GMALNPDNYLISKDVTGATKVKYYYAVNDG | | | |
| FPGDHYAVMISKTGTNAGDFTVVFEETPNGIN | | | |
| KGGARFGLSTEANGAKPQSVWIERTVDLPAG | | | |
| TKYVAFRHYNCSDLNYILLDDIQFTMGGSPTP | | | |
| TDYTYTVYRDGTKIKEGLTETTFEEDGVATG | | | |
| NHEYCVEVKYTAGVSPKKCVNVTVNGVTPK | | | |
| NIIDAFNGGISLANYTGHGSETAWGTSHFGTT | | | |
| HVKQLTNSNQ | | | |
| MKAPYTGCYSHLNTGVSFANYTAHGSETAW | SEQ ID NO: 375 | | 2A-KR |
| ADPLLTTSQLKALTNKDKPYQPVSNLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLTGSSVGQKVTLKWDAPNGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFTNALLEETITA | | | |
| KGVRSPKAIRGRIQGTWRQKTVDLPAGTKYV | | | |
| AFRHFQSTDMFYIDLDEVEIKANGGGSPTPTD | | | |
| YTYTVYRDGTKIKEGLTETTFEEDGVATGNH | | | |
| EYCVEVKYTAGVSPKKCVNVTVNGVTPKNII | | | |
| DAFNGGISLANYTGHGSETAWGTSHFGTTHV | | | |
| KQLTNSNQ | | | |
| MKAPYTGCYSHLNTGVSFANYTAHGSETAW | SEQ ID NO: 379 | | 2A-K2-KR |
| ADPLLTTSQLKALTNKDKPYQPVSNLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLTGSSVGQKVTLKWDAPNGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFTNALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGSNVVSSFSWNGMALNPDNYL | | | |
| ISKDVTGATKVKYYYAVNDGFPGDHYAVMI | | | |
| SKTGTNAGDFTVVFEETPNGINKGGARFGLST | | | |
| EANGAKPQSVWIERTVDLPAGTKYVAFRHY | | | |
| NCSDLNYILLDDIQFTMGGSPTPTDYTYTVYR | | | |
| DGTKIKEGLTETTFEEDGVATGNHEYCVEVK | | | |
| YTAGVSPKKCVNVTVNGVTPKNIIDAFNGGIS | | | |
| LANYTGHGSETAWGTSHFGTTHVKQLTNSN | | | |
| Q | | | |
| MKAPYTGCYSHLNTGVSFANYTAHGSETAW | SEQ ID NO: 403 | | 2A-K2-KR-G3 |
| ADPLLTTSQLKALTNKDKPYQPVSNLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLTGSSVGQKVTLKWDAPNGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFTNALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGTNVVASFSWNGMALNPDNY | | | |
| LISKDVTGATKVKYYYAVNDGFPGDHYAVM | | | |
| ISKTGTNAGDFTVVFEETPNGINKGGARFGLS | | | |
| TEANGAKPQSVWIERTVDLPAGTKYVAFRHY | | | |
| NCSDLNYILLDDIQFTMGGSPTPTDYTYTVYR | | | |
| DGTKIKEGLTETTFEEDGVATGNHEYCVEVK | | | |
| YTAGVSPKKCVNVTVNGVTPKNIIDAFNGGIS | | | |
| LANYTGHGSETAWGTSHFGTTHVKQLTNSN | | | |
| Q | | | |
| MKAPYTGCYSHLNTGVSFANYTAHGSETAW | SEQ ID NO: 415 | | 2A-K2-KR-G3 |
| ADPLLTTSQLKALTNKDKPYQPVSNLTATTQ | | | (dup) |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLTGSSVGQKVTLKWDAPNGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFTNALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGTNVVASFSWNGMALNPDNY | | | |
| LISKDVTGATKVKYYYAVNDGFPGDHYAVM | | | |
| ISKTGTNAGDFTVVFEETPNGINKGGARFGLS | | | |
| TEANGAKPQSVWIERTVDLPAGTKYVAFRHY | | | |
| NCSDLNYILLDDIQFTMGGSPTPGGSPTPTDY | | | |
| TYTVYRDGTKIKEGLTETTFEEDGVATGNHE | | | |
| YCVEVKYTAGVSPKKCVNVTVNGVTPKNIID | | | |
| AFNGGISLANYTGHGSETAWGTSHFGTTHVK | | | |
| QLTNSNQ | | | |
| MKAPYTGCYSHLNTGVSFANYTAHGSETAW | SEQ ID NO: 383 | | 2A-K2DUF-KR |
| ADPLLTTSQLKALTNKDKPYQPVSNLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLTGSSVGQKVTLKWDAPNGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFTNALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGSNVVSSFSWNGMALNPDNYL | | | |
| ISKDVTGATKVKYYYAVNDGFPGDHYAVMI | | | |
| SKTGTNAGDFTVVFEETPNGINKGGARFGLST | | | |
| EANGAKPQSVWIERTVDLPAGTKYVAFRHY | | | |
| NCSDLNYILLDDIQFTMGGSPTPTDYTYTVYR | | | |
| DGTKIKEGLTETTFEEDGVATGNHEYCVEVK | | | |
| YTAGVSPKKCVNVTVNNPYQPVSNLTATTQG | | | |
| QKVTLKWDAPSTKTNATTNTARSVDGIRELV | | | |
| LLSVSDAPELLRSGQAEIVLEAHDVWNDGSG | | | |
| YQILLDADHDQYGQVIPSDTHTLWPNCSVPA | | | |
| NLFAPFEYTVPENADPSCSPTNMIMDGTASVN | | | |
| IPAGTYDFAIAAPQANAKIWIAGQGPTKEDDY | | | |
| VFEAGKKYHFLMKKMGSGDGTELTISEGGGS | | | |
| DYTYTVYRDGTKIKEGLTATTFEEDGVAAGN | | | |
| HEYCVEVKYTAGVSPKVCKDVTVEGSGVTP | | | |
| KNIIDAFNGGISLANYTGHGSETAWGTSHFGT | | | |
| THVKQLTNSNQ | | | |
| MKAPYTGCYSHLNTGVSFANYTAHGSETAW | SEQ ID NO: 409 | | 2A-K2DUF-KR-G3 |
| ADPLLTTSQLKALTNKDKPYQPVSNLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLTGSSVGQKVTLKWDAPNGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFTNALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGTNVVASFSWNGMALNPDNY | | | |
| LISKDVTGATKVKYYYAVNDGFPGDHYAVM | | | |
| ISKTGTNAGDFTVVFEETPNGINKGGARFGLS | | | |
| TEANGAKPQSVWIERTVDLPAGTKYVAFRHY | | | |
| NCSDLNYILLDDIQFTMGGSPTPTDYTYTVYR | | | |
| DGTKIKEGLTETTFEEDGVATGNHEYCVEVK | | | |
| YTAGVSPKKCVNVTVNNPYQPVSNLTATTQG | | | |
| QKVTLKWDAPSTKTNATTNTARSVDGIRELV | | | |
| LLSVSDAPELLRSGQAEIVLEAHDVWNDGSG | | | |
| YQILLDADHDQYGQVIPSDTHTLWPNCSVPA | | | |
| NLFAPFEYTVPENADPSCSPTNMIMDGTASVN | | | |
| IPAGTYDFAIAAPQANAKIWIAGQGPTKEDDY | | | |
| VFEAGKKYHFLMKKMGSGDGTELTISEGGGS | | | |
| DYTYTVYRDGTKIKEGLTATTFEEDGVAAGN | | | |
| HEYCVEVKYTAGVSPKVCKDVTVEGSGVTP | | | |
| KNIIDAFNGGISLANYTGHGSETAWGTSHFGT | | | |
| THVKQLTNSNQ | | | |
| MKAPYTGCYSHLNTGVSFANYTAHGSETAW | SEQ ID NO: 397 | | Kgp_2A_ext |
| ADPLLTTSQLKALTNKDKPYQPVSNLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLTGSSVGQKVTLKWDAPNGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFTNALLEETITA | | | |
| KGVRSPKAIRGRIQGTWRQKTVDLPAGTKYV | | | |
| AFRHFQSTDMFYIDLDEVEIKANGGGSPTPTD | | | |
| YTYTVYRDGTKIKEGLTETTFEEDGVATGNH | | | |
| EYCVEVKYTAGVSPKKCVNVTVN | | | |

In some embodiments, the polypeptide comprises a sequence according to SEQ ID NO: 1, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 6, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 11, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 16, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 367, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto. In some embodiments, the polypeptide comprises a sequence according to SEQ ID NO: 371, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 375, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 379, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 383, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 397, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 403, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 415, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 409, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

The N-terminal methionine in any of the above embodiments may be omitted from the polypeptide. Accordingly, in some embodiments, the polypeptide comprises a sequence according to SEQ ID NO: 254 (i.e. SEQ ID NO: 1 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 259 (i.e. SEQ ID NO: 6 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 264 (i.e. SEQ ID NO: 11 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 269 (i.e. SEQ ID NO: 16 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 368 (i.e. SEQ ID NO: 367 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 372 (i.e. SEQ ID NO: 371 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 376 (i.e. SEQ ID NO: 375 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 380 (i.e. SEQ ID NO: 379 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 384 (i.e. SEQ ID NO: 383 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 398 (i.e. SEQ ID NO: 397 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 404 (i.e. SEQ ID NO: 403 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 416 (i.e. SEQ ID NO: 415 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 410 (i.e. SEQ ID NO: 409 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the nucleic acid encoding the polypeptide comprises a sequence according to SEQ ID NO: 21.

In some embodiments, the nucleic acid encoding the polypeptide comprises a sequence according to SEQ ID NO: 22.

In some embodiments, the nucleic acid encoding the polypeptide comprises a sequence according to SEQ ID NO: 31.

In some embodiments, the nucleic acid encoding the polypeptide comprises a sequence according to SEQ ID NO: 32.

In some embodiments, the nucleic acid encoding the polypeptide comprises a sequence according to SEQ ID NO: 41.

In some embodiments, the nucleic acid encoding the polypeptide comprises a sequence according to SEQ ID NO: 42.

In some embodiments, the nucleic acid encoding the polypeptide comprises a sequence according to SEQ ID NO: 51.

In some embodiments, the nucleic acid encoding the polypeptide comprises a sequence according to SEQ ID NO: 52.

In certain embodiments, the amino acid sequence of the polypeptides of the invention is encoded by a codon-optimized polynucleotide sequence.

### Variants of Polypeptides

The sequences of the polypeptides as described herein may comprise one or more mutations or modifications.

In some embodiments, the polypeptides as described herein may comprise one or more conservative amino acid substitutions.

### Mutation of Glycosylation Sites

Glycosylation may occur in eukaryotic cells but not in prokaryotic cells. “Glycosylation” as used herein refers to the addition of a saccharide unit to a protein. In particular, N-linked glycosylation is the attachment of glycan to an amide nitrogen of an asparagine (Asn; N) residue of a protein. The process of attachment results in a glycosylated protein. This glycan may be a polysaccharide. Glycosylation can occur at any asparagine residue in a protein that is accessible to and recognised by glycosylating enzymes following translation of the protein, and is most common at accessible asparagines that are part of an NXS/T motif, wherein the second amino acid residue following the asparagine is a serine or threonine. A non-human glycosylation pattern can render a polypeptide undesirably reactogenic when used to elicit antibodies. Additionally, glycosylation of a polypeptide that is not normally glycosylated (such as polypeptides described herein) may alter its immunogenicity. For example, glycosylation can mask important immunogenic epitopes within a protein. Thus, to reduce or eliminate glycosylation, either asparagine residues or serine/threonine residues can be modified, for example, by substitution to another amino acid.

In certain embodiments, a polypeptide as described herein comprises at least one mutated glycosylation site, for example at least one mutated N-linked glycosylation site and/or at least one O-linked glycosylation site. In some embodiments, one or more (e.g. all)N-glycosylation sites in a polypeptide as described herein are removed. The removal of an N-glycosylation site may decrease glycosylation of the polypeptide. In some embodiments, a polypeptide as described herein has decreased glycosylation relative to the corresponding wild-type polypeptide. The decreased glycosylation relative to the corresponding wild-type polypeptide may be observed in one or all of the domains of the polypeptide. For example, the at least a portion of the Kgp catalytic domain may have decreased glycosylation relative to the corresponding wild-type portion of the Kgp catalytic domain. In certain embodiments, all domains of the polypeptide have decreased glycosylation relative to the corresponding wild-type domains. The removal of N-glycosylation sites may eliminate N-glycosylation of the polypeptide.

In certain embodiments, the modification comprises a substitution of one or more (e.g. all) of an N, S, and T amino acid in an NXS/T sequence motif, wherein X corresponds to any amino acid. In some embodiments, an N, S, or T amino acid is substituted with a conservative amino acid substitution.

Exemplary mutated glycosylation sites within Kgp or RgpA that may be mutated are shown below in and Error! Reference source not found. Accordingly, in any of the nucleic acid or polypeptides of the invention disclosed herein the one or more (e.g. all) mutation positions within a Kgp corresponds to a position of the wild-type sequence of SEQ ID NO: 157 that is specified in Error! Reference source not found. In any of the nucleic acid or polypeptides of the invention disclosed herein the one or more (e.g. all) mutation positions within a RgpA corresponds to one or more (e.g. all) positions of the wild-type sequence of SEQ ID NO: 158 that is specified in Error! Reference source not found . . .

TABLE 20
Exemplary mutated glycosylation sites in
Kgp from P. gingivalis strain W50.

| Glycosylation | | |
|---|---|---|
| site in WT Kgp | | |
| sequence | Sequence | Exemplary |
| (SEQ ID NO: 157) | motif | substitution |
| N284-S286 | NaS | N284Q |
| N440-T442 | NyT | T442V |
| N572-T574 | NiT | T574V |
| N643-S645 | NaS | S645A |
| N691-T693 | NIT | N691Q |
| N691-T693 | NIT | T693A |
| N950-T952 | NIT | N950Q |
| N950-T952 | NIT | T952A |
| N968-T970 | NgT | N968Q |
| T1089-T1091 | NfT | T1089A |
| N1316-S1318 | NcS | N1316Q |
| N1390-T1392 | NIT | N1390D |

TABLE 21
Exemplary mutated glycosylation sites in
RgpA from P. gingivalis strain W50.

| Glycosylation | | |
|---|---|---|
| site in WT RgpA | | |
| sequence | Sequence | Exemplary |
| (SEQ ID NO: 158) | motif | substitution |
| N361-T363 | NiT | T363A |
| N434-T436 | NyT | N434Q |
| N434-T436 | NyT | T436A |
| N508-S510 | NqS | N508Q |
| N590-T592 | NIT | T592A |
| N597-S599 | NvS | N597Q |
| N623-T625 | NgT | N623Q |
| N629-T631 | NIT | N629Q |
| N635-S637 | NeS | N635Q |
| N669-T671 | NIT | T671A |
| N691-T693 | NaT | N691Q |
| N764-S766 | NcS | S766A |
| N929-T931 | NIT | T931A |
| N947-T949 | NgT | N947Q |
| N1298-S1300 | NcS | N1298Q |
| N1372-Q1374 | NvT | N1372Q |

In some embodiments, the polypeptides described herein comprise one or more (e.g. all) mutations shown in. In some embodiments, the polypeptides described herein comprise one or more (e.g. all) mutations shown in Error! Reference source not found. In some embodiments, the polypeptides described herein comprise one or more (e.g. all) mutations shown in Error! Reference source not found, and one or more mutations (e.g. all) shown in Error! Reference source not found . . .

In some embodiments, the Kgp-based polypeptides described herein comprise a single amino acid substitution at one or more (e.g. all) positions corresponding to an N-glycosylation site in a native P. gingivalis Kgp polypeptide (e.g. SEQ ID NO: 157). In some embodiments, a Kgp-based polypeptide described herein comprises one or more (e.g. all) amino acid substitutions at positions corresponding to 284, 442, 574 645, 691, 950, 968, 1089, 1316 and 1390 of SEQ ID NO: 157. In some embodiments, a Kgp-based polypeptide described herein comprises one or more (e.g. all) amino acid substitutions at positions corresponding to 284, 442, 574, 645, 691, 950, 968, 1089 of SEQ ID NO: 157. In some embodiments, a Kgp-based polypeptide described herein comprises one or more (e.g. all) amino acid substitutions at positions corresponding to 284, 442, 574, 645, 691, 950, 968 of SEQ ID NO: 157. In some embodiments, a Kgp-based polypeptide described herein comprises one or more (e.g. all) amino acid substitutions at positions corresponding to 442, 691, 950, 968, 1089, of SEQ ID NO: 157. In some embodiments, a Kgp-based polypeptide described herein comprises one or more (e.g. all) amino acid substitutions at positions corresponding to 442, 691, 950, 968, of SEQ ID NO: 157.

In some embodiments, the RgpA-based polypeptides described herein comprise a single amino acid substitution at one or more (e.g. all) positions corresponding to an N-glycosylation site in a native P. gingivalis RgpA polypeptide (e.g. SEQ ID NO: 158). In some embodiments, a RgpA-based polypeptide described herein comprises one or more (e.g. all) amino acid substitutions at positions corresponding to 363, 434 and 436, 508, 592, 597, 623, 629, 635, 671, 691, 766, 931, 947, 1298, 1372 of SEQ ID NO: 158. In some embodiments, a RgpA-based polypeptide described herein comprises one or more (e.g. all) amino acid substitutions at positions corresponding to 363, 436, 508, 592, 597, 623, 629, 635, 671, 691, 766, 931, 947, 1298, 1372 of SEQ ID NO: 158. In some embodiments, a RgpA-based polypeptide described herein comprises one or more (e.g. all) amino acid substitution at positions 434, 671, 69, 766, 931, 947, 1298, 1372 of SEQ ID NO: 158.

In some embodiments, the Kgp and RgpA-based polypeptides described herein comprise a single amino acid substitution at one or more (e.g. all) positions corresponding to an N-glycosylation site in a native P. gingivalis Kgp polypeptide (e.g. SEQ ID NO: 157) and in a native P. gingivalis RgpA polypeptide (e.g. SEQ ID NO: 158). In some embodiments, a Kgp and RgpA-based polypeptide described herein comprises a single amino acid substitution at one or more (e.g. all) positions corresponding to 442, 691, 950, 968, 1089, 1390 of SEQ ID NO: 157 and 434 of SEQ ID NO: 158. In some embodiments, a Kgp and RgpA-based polypeptide described herein comprises a single amino acid substitution at one or more (e.g. all) positions corresponding to 442, 691, 950, 968, 1089, 1390 of SEQ ID NO: 157 and 434 of SEQ ID NO: 158. In some embodiments, a Kgp and RgpA-based polypeptide described herein comprises a single amino acid substitution at one or more (e.g. all) positions corresponding to 442, 691, 950, 968, 1089, 1316 and 1390 of SEQ ID NO: 157 and 434 of SEQ ID NO: 158. In some embodiments, a Kgp and RgpA-based polypeptide described herein comprises a single amino acid substitution at one or more (e.g. all) positions corresponding to 442, 691, 950, 968, 1089, 1316, 1390 of SEQ ID NO: 157 and 434, 671, 691, 766 of SEQ ID NO: 158.

Examples of polypeptides of the invention are provided in Table 11.

TABLE 22
Examples of polypeptides of the invention, or polypeptides encoded by nucleic acids
of the invention

| | | Exemplary | |
|---|---|---|---|
| | Amino acid | Nucleic acid | |
| Amino acid sequence | SEQ ID NO: | SEQ ID NOS: | Designation |
| MDVYTDHGDLYNTPVRMLVVAGAKFKEAL | SEQ ID NO: 359 | | Kgp_1A |
| KPWLTWKAQKGFYLDVHYTDEAEVGTTQAS | | | |
| IKAFIHKKYNDGLAASAAPVFLALVGDTDVIS | | | |
| GEKGKKSKKVTDLYYSAVDGDYFPEMYTFR | | | |
| MSASSPEELTNIIDKVLMYEKATMPDKSYLEK | | | |
| VLLIAGADYSWNSQVGQPTIKYGMQYYYNQ | | | |
| EHGYTDVYNYLKAPYTGCYSHLNTGVSFAN | | | |
| YVAHGSETAWADPLLTTSQLKALTNKDKYFL | | | |
| AIGNCSITAQFDYVQPCFGEVITRVKEKGAYA | | | |
| YIGSSPNSYWGEDYYWSVGANAVFGVQPTFE | | | |
| GTSMGSYDATFLEDSYNTVNSIMWAGNLAA | | | |
| THAGNIGNIVHIGAHYYWEAYHVLGDGSVM | | | |
| PYRAMPKTNTYTLPASLPQNQASYSIQASAGS | | | |
| YVAISKDGVLYGTGVANAAGVATVSMTKQI | | | |
| TENGNYDVVITRSNYLPVIKQIQVGEPSPYQP | | | |
| VSQLTATTQGQKVTLKWEAPSAKKSEGSREV | | | |
| KRIGDGLFVTIEPANDVRANEAKVVLAADNV | | | |
| WGDNTGYQFLLDADHNTFGSVIPATGPLFTG | | | |
| TASSNLYSANFEYLIPANADPVVTTQNIIVTG | | | |
| QGEVVIPGGVYDYCITNPEPASGKMWIAGDG | | | |
| GNQPARYDDFTFEAGKKYTFTMRRAGMGDG | | | |
| TDMEAEDDSPASYTYTVYRDGTKIKEGLTAT | | | |
| TFEEDGVAAGNHEYCVEVKYTAGVSPKVCK | | | |
| DVTVEGSNEFAPVQQLTGSSVGQKVTLKWD | | | |
| APQGTPNPNPNPNPNPGTTLSESFENGIPASW | | | |
| KTIDADGDGHGWKPGNAPGIAGYNSNGCVY | | | |
| SESFGLGGIGVLTPDNYLITPALDLPNGGKLTF | | | |
| WVCAQDANYASEHYAVYASSTGNDASNFAN | | | |
| ALLEETITAKGVRSPKAIRGRIQGTWRQKTVD | | | |
| LPAGTKYVAFRHFQSTDMFYIDLDEVEIKAN | | | |
| GGGSPTPTDYTYTVYRDGTKIKEGLTETTFEE | | | |
| DGVATGNHEYCVEVKYTAGVSPKKCVDVTV | | | |
| N | | | |
| MDVYTDHGDLYNTPVRMLVVAGAKFKEAL | SEQ ID NO: 361 | | Kgp_1B |
| KPWLTWKAQKGFYLDVHYTDEAEVGTTQAS | | | |
| IKAFIHKKYNDGLAASAAPVFLALVGDTDVIS | | | |
| GEKGKKSKKVTDLYYSAVDGDYFPEMYTFR | | | |
| MSASSPEELTNIIDKVLMYEKATMPDKSYLEK | | | |
| VLLIAGADYSWNSQVGQPTIKYGMQYYYNQ | | | |
| EHGYTDVYNYLKAPYTGCYSHLNTGVSFAN | | | |
| YVAHGSETAWADPLLTTSQLKALTNKDKYFL | | | |
| AIGNCSITAQFDYVQPCFGEVITRVKEKGAYA | | | |
| YIGSSPNSYWGEDYYWSVGANAVFGVQPTFE | | | |
| GTSMGSYDATFLEDSYNTVNSIMWAGNLAA | | | |
| THAGNIGNIVHIGAHYYWEAYHVLGDGSVM | | | |
| PYRAMPKTNTYTLPASLPQNQASYSIQASAGS | | | |
| YVAISKDGVLYGTGVANAAGVATVSMTKQI | | | |
| TENGNYDVVITRSNYLPVIKQIQVGEPSPYQP | | | |
| VSQLTATTQGQKVTLKWEAPSAKKSEGSREV | | | |
| KRIGDGLFVTIEPANDVRANEAKVVLAADNV | | | |
| WGDNTGYQFLLDADHNTFGSVIPATGPLFTG | | | |
| TASSNLYSANFEYLIPANADPVVTTQNIIVTG | | | |
| QGEVVIPGGVYDYCITNPEPASGKMWIAGDG | | | |
| GNQPARYDDFTFEAGKKYTFTMRRAGMGDG | | | |
| TDMEAEDDSPASYTYTVYRDGTKIKEGLTAT | | | |
| TFEEDGVAAGNHEYCVEVKYTAGVSPKVCK | | | |
| DVTVEGSNEFAPVQQLTGSSVGQKVTLKWD | | | |
| APQGTPNPNPNPNPNPGTTLSESFPTDYTYTV | | | |
| YRDGTKIKEGLTETTFEEDGVATGNHEYCVE | | | |
| VKYTAGVSPKKCVDVTVN | | | |
| MNTGVSFANYVAHGSETAWADPLLTTSQLK | SEQ ID NO: 363 | | Kgp_2A |
| ALTNKDKPYQPVSQLTATTQGQKVTLKWEA | | | |
| PSAKKSEGSREVKRIGDGLFVTIEPANDVRAN | | | |
| EAKVVLAADNVWGDNTGYQFLLDADHNTF | | | |
| GSVIPATGPLFTGTASSNLYSANFEYLIPANAD | | | |
| PVVTTQNIIVTGQGEVVIPGGVYDYCITNPEP | | | |
| ASGKMWIAGDGGNQPARYDDFTFEAGKKYT | | | |
| FTMRRAGMGDGTDMEAEDDSPASYTYTVYR | | | |
| DGTKIKEGLTATTFEEDGVAAGNHEYCVEVK | | | |
| YTAGVSPKVCKDVTVEGSNEFAPVQQLTGSS | | | |
| VGQKVTLKWDAPQGTPNPNPNPNPNPGTTLS | | | |
| ESFENGIPASWKTIDADGDGHGWKPGNAPGI | | | |
| AGYNSNGCVYSESFGLGGIGVLTPDNYLITPA | | | |
| LDLPNGGKLTFWVCAQDANYASEHYAVYAS | | | |
| STGNDASNFANALLEETITAKGVRSPKAIRGR | | | |
| IQGTWRQKTVDLPAGTKYVAFRHFQSTDMF | | | |
| YIDLDEVEIKANGGGSPTPTDYTYTVYRDGT | | | |
| KIKEGLTETTFEEDGVATGNHEYCVEVKYTA | | | |
| GVSPKKCVDVTVN | | | |
| MNTGVSFANYVAHGSETAWADPLLTTSQLK | SEQ ID NO: 365 | | Kgp_2B |
| ALTNKDKPYQPVSQLTATTQGQKVTLKWEA | | | |
| PSAKKSEGSREVKRIGDGLFVTIEPANDVRAN | | | |
| EAKVVLAADNVWGDNTGYQFLLDADHNTF | | | |
| GSVIPATGPLFTGTASSNLYSANFEYLIPANAD | | | |
| PVVTTQNIIVTGQGEVVIPGGVYDYCITNPEP | | | |
| ASGKMWIAGDGGNQPARYDDFTFEAGKKYT | | | |
| FTMRRAGMGDGTDMEAEDDSPASYTYTVYR | | | |
| DGTKIKEGLTATTFEEDGVAAGNHEYCVEVK | | | |
| YTAGVSPKVCKDVTVEGSNEFAPVQQLTGSS | | | |
| VGQKVTLKWDAPQGTPNPNPNPNPNPGTTLS | | | |
| ESFPTDYTYTVYRDGTKIKEGLTETTFEEDGV | | | |
| ATGNHEYCVEVKYTAGVSPKKCVDVTVN | | | |
| MEPGRYTPVEEKQNGRMIVIVAKKYEGDIKD | SEQ ID NO: 369 | | 1B_RgpA |
| FVDWKNQRGLRTEVKVAEDIASPVTANAIQQ | | | |
| FVKQEYEKEGNDLTYVLLIGDHKDIPAKITPG | | | |
| IKSDQVYGQIVGNDHYNEVFIGRFSCESKEDL | | | |
| KTQIDRTIHYERNIATEDKWLGQALCIASAEG | | | |
| GPSADNGESDIQHENVIANLLTQYGYTKIIKC | | | |
| YDPGVTPKNIIDAFNGGISLANYAGHGSETA | | | |
| WGTSHFGTTHVKQLTNSNQLPFIFDVASVNG | | | |
| DFLFSMPCFAEALMRAQKDGKPTGTVAIIAST | | | |
| IQQSWASPMRGQDEMNEILCEKHPNNIKRTF | | | |
| GGVTMNGMFAMVEKYKKDGEKMLDTWTVF | | | |
| GDPSLLVRTLVPTKMQVTAPAQINLADASVQ | | | |
| VSCDYNGAIATISANGKMFGSAVVEQGTATI | | | |
| QLTGLTQESTLTLTVVGYNKETVIKTINTNGE | | | |
| PNPYQPVSNLAATTQGQKVTLKWDAPSTKTQ | | | |
| ATTNTARSVDGIRELVLLSVSDAPELLRSGQA | | | |
| EIVLEAHDVWNDGSGYQILLDADHDQYGQVI | | | |
| PSDTHTLWPNCAVPANLFAPFEYTVPENADPS | | | |
| CSPTNMIMDGTASVNIPAGTYDFALAAPQAN | | | |
| AKIWIAGQGPTKEDDYVFEAGKKYHFLMKK | | | |
| MGSGDGTELTISEGGGSDYTYTVYRDGTKIK | | | |
| EGLTATTFEEDGVAAGNHEYCVEVKYTAGV | | | |
| SPKVCKDVTVEGSNEFAPVQNLAGSAVGQK | | | |
| VTLKWDAPQGTPNPNPNPNPNPNPGTTTLSES | | | |
| FGKRADFTETFESSTHGEAPAEWTTIDADGD | | | |
| GQGWLCLSSGQLDWLTAHGGTNVVASFSWN | | | |
| GMALNPDNYLISKDVTGATKVKYYYAVNDG | | | |
| FPGDHYAVMISKTGTNAGDFTVVFEETPNGIN | | | |
| KGGARFGLSTEANGAKPQSVWIERTVDLPAG | | | |
| TKYVAFRHYQCSDLNYILLDDIQFTMGGSPTP | | | |
| TDYTYTVYRDGTKIKEGLTETTFEEDGVATG | | | |
| NHEYCVEVKYTAGVSPKKCVQVTVN | | | |
| MNPYQPVSNLAATTQGQKVTLKWDAPSTKT | SEQ ID NO: 373 | | 2B_RgpA |
| QATTNTARSVDGIRELVLLSVSDAPELLRSGQ | | | |
| AEIVLEAHDVWNDGSGYQILLDADHDQYGQ | | | |
| VIPSDTHTLWPNCAVPANLFAPFEYTVPENAD | | | |
| PSCSPTNMIMDGTASVNIPAGTYDFAIAAPQA | | | |
| NAKIWIAGQGPTKEDDYVFEAGKKYHFLMK | | | |
| KMGSGDGTELTISEGGGSDYTYTVYRDGTKI | | | |
| KEGLTATTFEEDGVAAGNHEYCVEVKYTAG | | | |
| VSPKVCKDVTVEGSNEFAPVQNLAGSAVGQ | | | |
| KVTLKWDAPQGTPNPNPNPNPNPNPGTTTLS | | | |
| ESFGKRADFTETFESSTHGEAPAEWTTIDADG | | | |
| DGQGWLCLSSGQLDWLTAHGGTNVVASFSW | | | |
| NGMALNPDNYLISKDVTGATKVKYYYAVND | | | |
| GFPGDHYAVMISKTGTNAGDFTVVFEETPNGI | | | |
| NKGGARFGLSTEANGAKPQSVWIERTVDLPA | | | |
| GTKYVAFRHYQCSDLNYILLDDIQFTMGGSP | | | |
| TPTDYTYTVYRDGTKIKEGLTETTFEEDGVAT | | | |
| GNHEYCVEVKYTAGVSPKKCVQVTVNGVTP | | | |
| KNIIDAFNGGISLAQYTGHGSETAWGTSHFGT | | | |
| THVKQLTNSNQ | | | |
| MKAPYTGCYSHLNTGVSFANYVAHGSETAW | SEQ ID NO: 377 | | 2A-KR |
| ADPLLTTSQLKALTNKDKPYQPVSQLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQQLTGSSVGQKVTLKWDAPQGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFANALLEETITA | | | |
| KGVRSPKAIRGRIQGTWRQKTVDLPAGTKYV | | | |
| AFRHFQSTDMFYIDLDEVEIKANGGGSPTPTD | | | |
| YTYTVYRDGTKIKEGLTETTFEEDGVATGNH | | | |
| EYCVEVKYTAGVSPKKCVDVTVNGVTPKNII | | | |
| DAFNGGISLAQYTGHGSETAWGTSHFGTTHV | | | |
| KQLTNSNQ | | | |
| MKAPYTGCYSHLNTGVSFANYVAHGSETAW | SEQ ID NO: 381 | | 2A-K2-KR |
| ADPLLTTSQLKALTNKDKPYQPVSQLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQQLTGSSVGQKVTLKWDAPQGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFANALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGSNVVSSFSWNGMALNPDNYL | | | |
| ISKDVTGATKVKYYYAVNDGFPGDHYAVMI | | | |
| SKTGTNAGDFTVVFEETPNGINKGGARFGLST | | | |
| EANGAKPQSVWIERTVDLPAGTKYVAFRHY | | | |
| QCSDLNYILLDDIQFTMGGSPTPTDYTYTVYR | | | |
| DGTKIKEGLTETTFEEDGVATGNHEYCVEVK | | | |
| YTAGVSPKKCVDVTVNGVTPKNIIDAFNGGIS | | | |
| LAQYTGHGSETAWGTSHFGTTHVKQLTNSN | | | |
| Q | | | |
| MKAPYTGCYSHLNTGVSFANYVAHGSETAW | SEQ ID NO: 385 | | 2A_K2DUF_KR |
| ADPLLTTSQLKALTNKDKPYQPVSQLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQQLTGSSVGQKVTLKWDAPQGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFANALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGSNVVSSFSWNGMALNPDNYL | | | |
| ISKDVTGATKVKYYYAVNDGFPGDHYAVMI | | | |
| SKTGTNAGDFTVVFEETPNGINKGGARFGLST | | | |
| EANGAKPQSVWIERTVDLPAGTKYVAFRHY | | | |
| QCSDLNYILLDDIQFTMGGSPTPTDYTYTVYR | | | |
| DGTKIKEGLTETTFEEDGVATGNHEYCVEVK | | | |
| YTAGVSPKKCVDVTVNNPYQPVSNLAATTQ | | | |
| GQKVTLKWDAPSTKTQATTNTARSVDGIREL | | | |
| VLLSVSDAPELLRSGQAEIVLEAHDVWNDGS | | | |
| GYQILLDADHDQYGQVIPSDTHTLWPNCAVP | | | |
| ANLFAPFEYTVPENADPSCSPTNMIMDGTASV | | | |
| NIPAGTYDFAIAAPQANAKIWIAGQGPTKEDD | | | |
| YVFEAGKKYHFLMKKMGSGDGTELTISEGG | | | |
| GSDYTYTVYRDGTKIKEGLTATTFEEDGVAA | | | |
| GNHEYCVEVKYTAGVSPKVCKDVTVEGSGV | | | |
| TPKNIIDAFNGGISLAQYTGHGSETAWGTSHF | | | |
| GTTHVKQLTNSNQ | | | |
| MDVYTDHGDLYNTPVRMLVVAGAKFKEAL | SEQ ID NO: 387 | | Kgp_1A_G2 |
| KPWLTWKAQKGFYLDVHYTDEAEVGTTQAS | | | |
| IKAFIHKKYNDGLAASAAPVFLALVGDTDVIS | | | |
| GEKGKKSKKVTDLYYSAVDGDYFPEMYTFR | | | |
| MSASSPEELTNIIDKVLMYEKATMPDKSYLEK | | | |
| VLLIAGADYSWNSQVGQPTIKYGMQYYYNQ | | | |
| EHGYTDVYNYLKAPYTGCYSHLNTGVSFAN | | | |
| YVAHGSETAWADPLLTTSQLKALTNKDKYFL | | | |
| AIGNCSITAQFDYVQPCFGEVITRVKEKGAYA | | | |
| YIGSSPNSYWGEDYYWSVGANAVFGVQPTFE | | | |
| GTSMGSYDATFLEDSYNTVNSIMWAGNLAA | | | |
| THAGNIGNIVHIGAHYYWEAYHVLGDGSVM | | | |
| PYRAMPKTNTYTLPASLPQNQASYSIQASAGS | | | |
| YVAISKDGVLYGTGVANAAGVATVSMTKQI | | | |
| TENGNYDVVITRSNYLPVIKQIQVGEPSPYQP | | | |
| VSNLAATTQGQKVTLKWEAPSAKKSEGSREV | | | |
| KRIGDGLFVTIEPANDVRANEAKVVLAADNV | | | |
| WGDNTGYQFLLDADHNTFGSVIPATGPLFTG | | | |
| TASSNLYSANFEYLIPANADPVVTTQNIIVTG | | | |
| QGEVVIPGGVYDYCITNPEPASGKMWIAGDG | | | |
| GNQPARYDDFTFEAGKKYTFTMRRAGMGDG | | | |
| TDMEAEDDSPASYTYTVYRDGTKIKEGLTAT | | | |
| TFEEDGVAAGNHEYCVEVKYTAGVSPKVCK | | | |
| DVTVEGSNEFAPVQNLAGSSVGQKVTLKWD | | | |
| APQGTPNPNPNPNPNPGTTLSESFENGIPASW | | | |
| KTIDADGDGHGWKPGNAPGIAGYNSNGCVY | | | |
| SESFGLGGIGVLTPDNYLITPALDLPNGGKLTF | | | |
| WVCAQDANYASEHYAVYASSTGNDASNFAN | | | |
| ALLEETITAKGVRSPKAIRGRIQGTWRQKTVD | | | |
| LPAGTKYVAFRHFQSTDMFYIDLDEVEIKAN | | | |
| GGGSPTPTDYTYTVYRDGTKIKEGLTETTFEE | | | |
| DGVATGNHEYCVEVKYTAGVSPKKCVDVTV | | | |
| N | | | |
| MNTGVSFANYVAHGSETAWADPLLTTSQLK | SEQ ID NO: 389 | | Kgp_2A_G2 |
| ALTNKDKPYQPVSNLAATTQGQKVTLKWEA | | | |
| PSAKKSEGSREVKRIGDGLFVTIEPANDVRAN | | | |
| EAKVVLAADNVWGDNTGYQFLLDADHNTF | | | |
| GSVIPATGPLFTGTASSNLYSANFEYLIPANAD | | | |
| PVVTTQNIIVTGQGEVVIPGGVYDYCITNPEP | | | |
| ASGKMWIAGDGGNQPARYDDFTFEAGKKYT | | | |
| FTMRRAGMGDGTDMEAEDDSPASYTYTVYR | | | |
| DGTKIKEGLTATTFEEDGVAAGNHEYCVEVK | | | |
| YTAGVSPKVCKDVTVEGSNEFAPVQNLAGSS | | | |
| VGQKVTLKWDAPQGTPNPNPNPNPNPGTTLS | | | |
| ESFENGIPASWKTIDADGDGHGWKPGNAPGI | | | |
| AGYNSNGCVYSESFGLGGIGVLTPDNYLITPA | | | |
| LDLPNGGKLTFWVCAQDANYASEHYAVYAS | | | |
| STGNDASNFANALLEETITAKGVRSPKAIRGR | | | |
| IQGTWRQKTVDLPAGTKYVAFRHFQSTDMF | | | |
| YIDLDEVEIKANGGGSPTPTDYTYTVYRDGT | | | |
| KIKEGLTETTFEEDGVATGNHEYCVEVKYTA | | | |
| GVSPKKCVDVTVN | | | |
| MKAPYTGCYSHLNTGVSFANYVAHGSETAW | SEQ ID NO: 391 | | 2A-KR_G2 |
| ADPLLTTSQLKALTNKDKPYQPVSNLAATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLAGSSVGQKVTLKWDAPQGTPNP | | | |
| NPNPNPNPGTTLSESFENGIPASWKTIDADGD | | | |
| GHGWKPGNAPGIAGYNSNGCVYSESFGLGGI | | | |
| GVLTPDNYLITPALDLPNGGKLTFWVCAQDA | | | |
| NYASEHYAVYASSTGNDASNFANALLEETIT | | | |
| AKGVRSPKAIRGRIQGTWRQKTVDLPAGTKY | | | |
| VAFRHFQSTDMFYIDLDEVEIKANGGGSPTPT | | | |
| DYTYTVYRDGTKIKEGLTETTFEEDGVATGN | | | |
| HEYCVEVKYTAGVSPKKCVDVTVNGVTPKNI | | | |
| IDAFNGGISLAQYTGHGSETAWGTSHFGTTH | | | |
| VKQLTNSNQ | | | |
| MKAPYTGCYSHLNTGVSFANYVAHGSETAW | SEQ ID NO: 393 | | 2A-K2-KR_G2 |
| ADPLLTTSQLKALTNKDKPYQPVSNLAATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLAGSSVGQKVTLKWDAPQGTPNP | | | |
| NPNPNPNPGTTLSESFENGIPASWKTIDADGD | | | |
| GHGWKPGNAPGIAGYNSNGCVYSESFGLGGI | | | |
| GVLTPDNYLITPALDLPNGGKLTFWVCAQDA | | | |
| NYASEHYAVYASSTGNDASNFANALLEETIT | | | |
| AKGVRSPKAIRGRIQGTWRQKTVDLPAGTKY | | | |
| VAFRHFQSTDMFYIDLDEVEIKANGKRADFT | | | |
| ETFESSTHGEAPAEWTTIDADGDGQGWLCLS | | | |
| SGQLDWLTAHGGSNVVSSFSWNGMALNPDN | | | |
| YLISKDVTGATKVKYYYAVNDGFPGDHYAV | | | |
| MISKTGTNAGDFTVVFEETPNGINKGGARFGL | | | |
| STEANGAKPQSVWIERTVDLPAGTKYVAFRH | | | |
| YQCSDLNYILLDDIQFTMGGSPTPTDYTYTVY | | | |
| RDGTKIKEGLTETTFEEDGVATGNHEYCVEV | | | |
| KYTAGVSPKKCVDVTVNGVTPKNIIDAFNGG | | | |
| ISLAQYTGHGSETAWGTSHFGTTHVKQLTNS | | | |
| NQ | | | |
| MKAPYTGCYSHLNTGVSFANYVAHGSETAW | SEQ ID NO: 395 | | 2A_K2DUF_ |
| ADPLLTTSQLKALTNKDKPYQPVSNLAATTQ | | | KR_G2 |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLAGSSVGQKVTLKWDAPQGTPNP | | | |
| NPNPNPNPGTTLSESFENGIPASWKTIDADGD | | | |
| GHGWKPGNAPGIAGYNSNGCVYSESFGLGGI | | | |
| GVLTPDNYLITPALDLPNGGKLTFWVCAQDA | | | |
| NYASEHYAVYASSTGNDASNFANALLEETIT | | | |
| AKGVRSPKAIRGRIQGTWRQKTVDLPAGTKY | | | |
| VAFRHFQSTDMFYIDLDEVEIKANGKRADFT | | | |
| ETFESSTHGEAPAEWTTIDADGDGQGWLCLS | | | |
| SGQLDWLTAHGGSNVVSSFSWNGMALNPDN | | | |
| YLISKDVTGATKVKYYYAVNDGFPGDHYAV | | | |
| MISKTGTNAGDFTVVFEETPNGINKGGARFGL | | | |
| STEANGAKPQSVWIERTVDLPAGTKYVAFRH | | | |
| YQCSDLNYILLDDIQFTMGGSPTPTDYTYTVY | | | |
| RDGTKIKEGLTETTFEEDGVATGNHEYCVEV | | | |
| KYTAGVSPKKCVDVTVNNPYQPVSNLAATT | | | |
| QGQKVTLKWDAPSTKTQATTNTARSVDGIRE | | | |
| LVLLSVSDAPELLRSGQAEIVLEAHDVWNDG | | | |
| SGYQILLDADHDQYGQVIPSDTHTLWPNCAV | | | |
| PANLFAPFEYTVPENADPSCSPTNMIMDGTAS | | | |
| VNIPAGTYDFAIAAPQANAKIWIAGQGPTKED | | | |
| DYVFEAGKKYHFLMKKMGSGDGTELTISEG | | | |
| GGSDYTYTVYRDGTKIKEGLTATTFEEDGVA | | | |
| AGNHEYCVEVKYTAGVSPKVCKDVTVEGSG | | | |
| VTPKNIIDAFNGGISLAQYTGHGSETAWGTSH | | | |
| FGTTHVKQLTNSNQ | | | |
| MKAPYTGCYSHLNTGVSFANYVAHGSETAW | SEQ ID NO: 399 | | Kgp_2A |
| ADPLLTTSQLKALTNKDKPYQPVSNLAATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQNLAGSSVGQKVTLKWDAPQGTPNP | | | |
| NPNPNPNPGTTLSESFENGIPASWKTIDADGD | | | |
| GHGWKPGNAPGIAGYNSNGCVYSESFGLGGI | | | |
| GVLTPDNYLITPALDLPNGGKLTFWVCAQDA | | | |
| NYASEHYAVYASSTGNDASNFANALLEETIT | | | |
| AKGVRSPKAIRGRIQGTWRQKTVDLPAGTKY | | | |
| VAFRHFQSTDMFYIDLDEVEIKANGGGSPTPT | | | |
| DYTYTVYRDGTKIKEGLTETTFEEDGVATGN | | | |
| HEYCVEVKYTAGVSPKKCVDVTVN | | | |
| MKAPYTGCYSHLNTGVSFANYVAHGSETAW | SEQ ID NO: 405 | | 2A-K2-KR_G3 |
| ADPLLTTSQLKALTNKDKPYQPVSQLTATTQ | | | |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQQLTGSSVGQKVTLKWDAPQGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFANALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGTNVVASFSWNGMALNPDNY | | | |
| LISKDVTGATKVKYYYAVNDGFPGDHYAVM | | | |
| ISKTGTNAGDFTVVFEETPNGINKGGARFGLS | | | |
| TEANGAKPQSVWIERTVDLPAGTKYVAFRHY | | | |
| QCSDLNYILLDDIQFTMGGSPTPTDYTYTVYR | | | |
| DGTKIKEGLTETTFEEDGVATGNHEYCVEVK | | | |
| YTAGVSPKKCVDVTVNGVTPKNIIDAFNGGIS | | | |
| LAQYTGHGSETAWGTSHFGTTHVKQLTNSN | | | |
| Q | | | |
| MKAPYTGCYSHLNTGVSFANYVAHGSETAW | SEQ ID NO: 417 | | 2A-K2-KR_G3 |
| ADPLLTTSQLKALTNKDKPYQPVSQLTATTQ | | | (dup) |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQQLTGSSVGQKVTLKWDAPQGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFANALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGTNVVASFSWNGMALNPDNY | | | |
| LISKDVTGATKVKYYYAVNDGFPGDHYAVM | | | |
| ISKTGTNAGDFTVVFEETPNGINKGGARFGLS | | | |
| TEANGAKPQSVWIERTVDLPAGTKYVAFRHY | | | |
| QCSDLNYILLDDIQFTMGGSPTPGGSPTPTDY | | | |
| TYTVYRDGTKIKEGLTETTFEEDGVATGNHE | | | |
| YCVEVKYTAGVSPKKCVDVTVNGVTPKNIID | | | |
| AFNGGISLAQYTGHGSETAWGTSHFGTTHVK | | | |
| QLTNSNQ | | | |
| MKAPYTGCYSHLNTGVSFANYVAHGSETAW | SEQ ID NO: 411 | | 2A_K2DUF_ |
| ADPLLTTSQLKALTNKDKPYQPVSQLTATTQ | | | KR_G3 |
| GQKVTLKWEAPSAKKSEGSREVKRIGDGLFV | | | |
| TIEPANDVRANEAKVVLAADNVWGDNTGYQ | | | |
| FLLDADHNTFGSVIPATGPLFTGTASSNLYSA | | | |
| NFEYLIPANADPVVTTQNIIVTGQGEVVIPGG | | | |
| VYDYCITNPEPASGKMWIAGDGGNQPARYD | | | |
| DFTFEAGKKYTFTMRRAGMGDGTDMEAEDD | | | |
| SPASYTYTVYRDGTKIKEGLTATTFEEDGVA | | | |
| AGNHEYCVEVKYTAGVSPKVCKDVTVEGSN | | | |
| EFAPVQQLTGSSVGQKVTLKWDAPQGTPNPN | | | |
| PNPNPNPGTTLSESFENGIPASWKTIDADGDG | | | |
| HGWKPGNAPGIAGYNSNGCVYSESFGLGGIG | | | |
| VLTPDNYLITPALDLPNGGKLTFWVCAQDAN | | | |
| YASEHYAVYASSTGNDASNFANALLEETITA | | | |
| KGVRSPKAIRGRIQGTWRQKTVDLPAGTKYV | | | |
| AFRHFQSTDMFYIDLDEVEIKANGKRADFTET | | | |
| FESSTHGEAPAEWTTIDADGDGQGWLCLSSG | | | |
| QLDWLTAHGGTNVVASFSWNGMALNPDNY | | | |
| LISKDVTGATKVKYYYAVNDGFPGDHYAVM | | | |
| ISKTGTNAGDFTVVFEETPNGINKGGARFGLS | | | |
| TEANGAKPQSVWIERTVDLPAGTKYVAFRHY | | | |
| QCSDLNYILLDDIQFTMGGSPTPTDYTYTVYR | | | |
| DGTKIKEGLTETTFEEDGVATGNHEYCVEVK | | | |
| YTAGVSPKKCVDVTVNNPYQPVSNLAATTQ | | | |
| GQKVTLKWDAPSTKTQATTNTARSVDGIREL | | | |
| VLLSVSDAPELLRSGQAEIVLEAHDVWNDGS | | | |
| GYQILLDADHDQYGQVIPSDTHTLWPNCAVP | | | |
| ANLFAPFEYTVPENADPSCSPTNMIMDGTASV | | | |
| NIPAGTYDFAIAAPQANAKIWIAGQGPTKEDD | | | |
| YVFEAGKKYHFLMKKMGSGDGTELTISEGG | | | |
| GSDYTYTVYRDGTKIKEGLTATTFEEDGVAA | | | |
| GNHEYCVEVKYTAGVSPKVCKDVTVEGSGV | | | |
| TPKNIIDAFNGGISLAQYTGHGSETAWGTSHF | | | |
| GTTHVKQLTNSNQ | | | |

In some embodiments, the polypeptide comprises a sequence according to SEQ ID NO: 359, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 361, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 363, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 365, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 369, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 373, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 377, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 381, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 385, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 387, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 389, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 391, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 393, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 395, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 399, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 405, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 417, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 411, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 360 (i.e. SEQ ID NO: 359 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 362 (i.e. SEQ ID NO: 361 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 364 (i.e. SEQ ID NO: 363 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 366 (i.e. SEQ ID NO: 365 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 370 (i.e. SEQ ID NO: 369 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 374 (i.e. SEQ ID NO: 373 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 378 (i.e. SEQ ID NO: 377 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 382 (i.e. SEQ ID NO: 381 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 386 (i.e. SEQ ID NO: 385 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 388 (i.e. SEQ ID NO: 387 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 390 (i.e. SEQ ID NO: 389 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 392 (i.e. SEQ ID NO: 391 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 394 (i.e. SEQ ID NO: 393 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 396 (i.e. SEQ ID NO: 395 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 400 (i.e. SEQ ID NO: 399 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 406 (i.e. SEQ ID NO: 405 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 418 (i.e. SEQ ID NO: 417 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 412 (i.e. SEQ ID NO: 411 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

### Secretion Signal Peptide Sequences

A polypeptide of the invention as described herein may comprise a secretion signal peptide sequence. The secretion signal peptide may be cleaved in post-translation processing of the polypeptides described herein. The mature form of the polypeptide may therefore not comprise the secretion signal peptide sequence. However, a nucleotide sequence encoding a secretion signal peptide sequence may be present in nucleic acids described herein encoding the polypeptides described herein.

In some embodiments, the polypeptide of the invention as described herein may comprise viral or eukaryotic (e.g. human) secretion signal peptide (SS) sequences. The use of viral or eukaryotic secretion signal peptide sequences attached to a polypeptide described herein may offer numerous advantages for immunogenic compositions. When expressed from an mRNA, especially in a eukaryotic cell, a polypeptide of the invention comprising a SS sequence may have increased extracellular expression relative to the polypeptide without the SS sequence. The increased extracellular expression may promote higher immunogenicity and by extension, better vaccine efficacy.

Viral SS sequences may be found in publicly accessible databases (e.g., the NCBI or UniProt databases) which include an annotated viral polypeptide sequence and identify the start and end position of an experimentally validated SS.

In certain embodiments, the SS sequence as well as the location of the SS sequence cleavage site for a given known input polypeptide sequence may be predicted by using the SignalP algorithm. The SignalP algorithm (and more particularly SignalP v6.0) is described in further detail in Armenteros et al. (Nature Biotechnology. 37:420-423. 2019), Teufel et al. (Nature Biotechnology. 40:1023-1025. 2022), and services.healthtech.dtu.dk/services/SignalP-6.0/, each of which is incorporated herein by reference in their entirety. The strength of the prediction is assessed based on a cumulative rank score that considers the likelihood of detecting canonical features of the signal sequence (SS likelihood score) and the probability of cleavage at the cleavage site (cleavage probability score).

In certain embodiments, the SS sequence is a viral SS sequence. In certain embodiments, the viral secretion signal peptide sequence is derived from a viral sequence in a virus able to infect humans. The phrase “influenza”, “SARS CoV-2”, “varicella-zoster virus (VZV)”, “measles”, “rubella”, “rabies,” “Ebola,” and “smallpox” preceding the phrase “secretion signal peptide sequence” indicates that the secretion signal peptide was derived from the virus corresponding to that name.

In certain embodiments, the viral secretion signal peptide is derived from a viral sequence selected from the group consisting of: an influenza secretion signal peptide sequence, a SARS CoV-2 secretion signal peptide sequence, a varicella-zoster virus (VZV) secretion signal peptide sequence, a measles secretion signal peptide sequence, a rubella secretion signal peptide sequence, a mumps secretion signal peptide sequence, an Ebola secretion signal peptide sequence, a rabies secretion signal peptide sequence, and a smallpox secretion signal peptide sequence. These particular signal peptides are derived from viral sequences in viruses which have been administered to humans as vaccines (live-attenuated, inactivated or mRNA), with demonstrated strong safety profiles.

In certain embodiments, the viral secretion signal peptide is selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, a rabies virus glycoprotein (Rabies G) secretion signal peptide sequence, and a smallpox 6 kDa IC protein secretion signal peptide sequence.

In certain embodiments, the viral secretion signal peptide comprises an HA secretion signal peptide sequence from influenza A or influenza B, preferably from influenza A.

In certain embodiments, the viral secretion signal peptide comprises a signal peptide described in PCT/EP2023/062066, which is incorporated by reference herein in its entirety.

Exemplary viral secretion signal peptide amino acid sequences of the disclosure are shown below in. Exemplary viral secretion signal peptide amino acid sequences derived from Influenza A or B of the disclosure are shown below in Table 23.1.

TABLE 23
Viral Secretion Signal Peptide (SS) Amino Acid Sequences

| NAME OF THE | | | |
|---|---|---|---|
| PROTEIN | ORGANISM | STRAIN | SEQUENCE OF THE SS |
| HA (H1N1) | Influenza virus | A/New Caledonia/20/1999 | MKAKLLVLLCTFTATYA (SEQ ID |
| | | | NO: 193) |
| HA (H1N1pdm) | Influenza virus | A/California/7/2009 | MKAILVVLLYTFATANA (SEQ ID |
| | | | NO: 67) |
| HA (H3N2) | Influenza virus | A/Moscow/10/1999 | MKTIIALSYILCLVFA |
| | | | (SEQ ID NO: 194) |
| HAB | Influenza virus | B/Phuket/3073/2013 | MKAIIVLLMVVTSNA |
| | | | (SEQ ID NO: 195) |
| Spike | SARS COV-2 | Wuhan-1 | MFVFLVLLPLVS |
| | | | (SEQ ID NO: 196) |
| Spike | SARS COV-2 | Wuhan-1 (long version) | MFLLTTKRTMFVFLVLLPLVS |
| | | | (SEQ ID NO: 197) |
| gB | VZV | Oka strain | MSPCGYYSKWRNRDRPEYRRNLR |
| | | | FRRFFSSIHPNAAAGSGFNGPGVFIT |
| | | | SVTGVWLCFLCIFSMFVTAVVS |
| | | | (SEQ ID NO: 198) |
| gE | VZV | Oka strain | MGTVNKPVVGVLMGFGIITGTLRIT |
| | | | NPVRA |
| | | | (SEQ ID NO: 199) |
| gI | VZV | Oka strain | MFLIQCLISA VIFYIQVTNA |
| | | | (SEQ ID NO: 200) |
| gK | VZV | Oka strain | MQALGIKTEHFIIMCLLSGHA |
| | | | (SEQ ID NO: 201) |
| F | Measles | Edmonston-Zagreb strain | MGLKVNVSAIFMAVLLTLQTPTG |
| | | | (SEQ ID NO: 202) |
| E1 | Rubella | RA27/3 strain | MGAAAALTAVVLQGYNPPAYG |
| | | | (SEQ ID NO: 203) |
| E2 | Rubella | RA27/3 strain | MGAPQAFLAGLLLAAVAVGTARA |
| | | | (SEQ ID NO: 204) |
| F | Mumps | Miyahara strain | MKVFLVTCLGFAVFSSSVC |
| | | | (SEQ ID NO: 205) |
| GP | Ebola | Mayinga-76 strain | MGVTGILQLPRDRFKRTSFFLWVII |
| | | | LFQRTFS |
| | | | (SEQ ID NO: 206) |
| 6 kDa IC | Smallpox | Germany 91-3 strain | MRSLIIFLLFPSIIYS |
| | | | (SEQ ID NO: 207) |
| Rabies G | Rabies | Rabies Pasteur strain | MVPQALLFVPLLVFPLCFG |
| | | | (SEQ ID NO: 208) |

TABLE 23
Influenza virus A and B Specific Viral Secretion Signal Peptide (SS) Amino Acid Sequences

| STRAIN NAME OR ID | SEQUENCE OF THE SS |
|---|---|
| A/Beijing/262/95 (H1N1)-like virt [symbol] | MKAKLLVLLCTFTATYA |
| | (SEQ ID NO: 193) |
| A/Brisbane/02/2018 | MKAILVVLLYTFTTANA |
| (H1N1)pdm09-like virus | (SEQ ID NO: 209) |
| A/Brisbane/59/2007 (H1N1)-lik [symbol] | MKVKLLVLLCTFTATYA |
| virus | (SEQ ID NO: 210) |
| A/California/7/2009 (H1N1)-lik [symbol] | MKAILVVLLYTFATANA |
| virus | (SEQ ID NO: 67) |
| A/Guangdong- | MKAILVVLLYTFTTANA |
| Maonan/SWL1536/2019 | (SEQ ID NO: 209) |
| (H1N1)pdm09-like virus | |
| A/Hawaii/70/2019 (H1N1)pdm0 [symbol] | MKAILVVLLYTFTTANA |
| like virus | (SEQ ID NO: 209) |
| A/Michigan/45/2015 | MKAILVVLLYTFTTANA |
| (H1N1)pdm09-like virus | (SEQ ID NO: 209) |
| A/New Caledonia/20/99 (H1N1 [symbol] | MKAKLLVLLCTFTATYA |
| like virus | (SEQ ID NO: 193) |
| A/Solomon Islands/3/2006 (H1N1 [symbol] | MKVKLLVLLCTFTATYA |
| like virus | (SEQ ID NO: 210) |
| A/Sydney/5/2021 (H1N1)pdm09 [symbol] | MKAILVVMLYTFTTANA |
| like virus | (SEQ ID NO: 211) |
| A/Victoria/2570/2019 | MKAILVVMLYTFTTANA |
| (H1N1)pdm09-like virus | (SEQ ID NO: 211) |
| A/Victoria/4897/2022 | MKAILVVMLYTFTTANA |
| (H1N1)pdm09-like virus | (SEQ ID NO: 211) |
| A/Wisconsin/588/2019 | MKAILVVMLYTFTTANA |
| (H1N1)pdm09-like virus | (SEQ ID NO: 211) |
| A/Wisconsin/67/2022 | MKAILVVMLYTFTTANA |
| (H1N1)pdm09-like virus | (SEQ ID NO: 211) |
| H1N1 CONSENSUS SEQ # | MKAILVVLLYTFTTANA |
| (without substitutions) | (SEQ ID NO: 209) |
| A/Brisbane/10/2007 (H3N2)-lik [symbol] | MKTIIALSYILCLVFT |
| virus | (SEQ ID NO: 212) |
| A/California/7/2004 (H3N2)-lik [symbol] | MKTIIALSYILCLVFA |
| virus | (SEQ ID NO: 194) |
| A/Cambodia/e0826360/2020 | MKTIIALSYILCLVFA |
| (H3N2)-like virus | (SEQ ID NO: 194) |
| A/Darwin/6/2021 (H3N2)-lik [symbol] | MKTIIALSNILCLVFA |
| virus | (SEQ ID NO: 213) |
| A/Darwin/9/2021 (H3N2)-lik [symbol] | MKTIIALSNILCLVFA |
| virus | (SEQ ID NO: 213) |
| A/Fujian/411/2002 (H3N2)-lik [symbol] | MKTIIALSYILCLVFA |
| virus | (SEQ ID NO: 194) |
| A/Hong Kong/2671/2019 (H3N2 [symbol] | MKAIIALSNILCLVFA |
| like virus | (SEQ ID NO: 214) |
| A/Hong Kong/45/2019 (H3N2)-lik [symbol] | MKAIIALSNILCLVFA |
| virus | (SEQ ID NO: 214) |
| A/Hong Kong/4801/2014 (H3N2 [symbol] | MKTIIALSYILCLVFA |
| like virus | (SEQ ID NO: 194) |
| A/Kansas/14/2017 (H3N2)-lik [symbol] | MKTIIALSCILCLVFA |
| virus | (SEQ ID NO: 215) |
| A/Moscow/10/99 (H3N2)-like virt [symbol] | MKTIIALSYILCLVFA |
| | (SEQ ID NO: 194) |
| A/Perth/16/2009 (H3N2)-like viru [symbol] | MKTIIALSYILCLVFA |
| | (SEQ ID NO: 194) |
| A/Singapore/INFIMH-16- | MKTIIALSYILCLVFA |
| 0019/2016 (H3N2)-like virus | (SEQ ID NO: 194) |
| A/South Australia/34/201 [symbol] | MKTIIALSYILCLVFA |
| (H3N2)-like virus | (SEQ ID NO: 194) |
| A/Switzerland/8060/2017 (H3N2 [symbol] | MKTIALSYILCLVFA |
| like virus | (SEQ ID NO: 194) |
| A/Switzerland/9715293/2013 | MKTIIALSYILCLVFA |
| (H3N2)-like virus | (SEQ ID NO: 194) |
| A/Sydney/5/97 (H3N2)-like virus | MKTIIALSYILCLVFA |
| | (SEQ ID NO: 194) |
| A/Texas/50/2012 (H3N2)-like virt [symbol] | MKTIIALSYILCLVFA |
| | (SEQ ID NO: 194) |
| A/Victoria/361/2011 (H3N2)-lil [symbol] | MKTIIALSHILCLVFA |
| virus | (SEQ ID NO: 216) |
| A/Wellington/1/2004 (H3N2)-lik [symbol] | MKTIIALSYILCLVFA |
| virus | (SEQ ID NO: 194) |
| A/Wisconsin/67/2005 (H3N2)-lik [symbol] | MKTIIALSYILCLVFA |
| virus | (SEQ ID NO: 194) |
| H3N2 CONSENSUS SEQ # | MKTIIALSYILCLVFA |
| (without substitutions) | (SEQ ID NO: 194) |
| B/Austria/1359417/2021 | MKAIIVLLMVVTSNA |
| (B/Victoria lineage)-like | (SEQ ID NO: 195) |
| B/Brisbane/60/2008-like virus | MKAIIVLLMVVTSNA |
| | (SEQ ID NO: 195) |
| B/Colorado/06/2017-like vin [symbol] | MKAIIVLLMVVTSSA |
| (B/Victoria/2/87 lineage) | (SEQ ID NO: 217) |
| B/Hong Kong/330/2001-like virus | MKAIIVLLMVVTSNA |
| | (SEQ ID NO: 195) |
| B/Malaysia/2506/2004-like virus | MKAIIVLLMVVTSNA |
| | (SEQ ID NO: 195) |
| B/Washington/02/2019 (B/Victor [symbol] | MKAIIVLLMVVTSNA |
| lineage)-like virus | (SEQ ID NO: 195) |
| B/Beijing/184/93-like virus | MKAIIVLLMVVTSNA |
| | (SEQ ID NO: 195) |
| B/Florida/4/2006-like virus | MKAIIVLLMVVTSNA |
| | (SEQ ID NO: 195) |
| B/Massachusetts/2/2012-like virus | MKAIIVLLMVVTSNA |
| | (SEQ ID NO: 195) |
| B/Phuket/3073/2013-like virus | MKAIIVLLMVVTSNA |
| | SEQ ID NO: 195) |
| B/Sichuan/379/99-like virus | MEAIIVLLMVVTSNA |
| | (SEQ ID NO: 218) |
| INFLUENZA | MKAIIVLLMVVTSNA |
| VICTORIA/YAMAGATA | (SEQ ID NO: 195) |
| CONSENSUS SEQUENC [symbol] | |
| (without substitutions) | |
| H1N1 CONSENSUS SEQ #2 (wit [symbol] | MKX₁X₂LX₃VX₄LX₅TFX₆X₇X₈X₉A |
| substitutions) | X₁ is selected from A and V; X₂ is selected from I and K; |
| | X₃ is selected from V and L; X₄ is selected from Land M; |
| | X₅ is selected from Y and C; X₆ is selected from T and A |
| | X₇ is selected from T and A; X₈ is selected from A and T; |
| | and X₉ is selected from N and Y |
| | (SEQ ID NO: 219) |
| H3N2 CONSENSUS SEQ #2 (wit [symbol] | MKX₁IIALSX₂ILCLVFX₃ |
| substitutions) | X₁ is selected from T and A; X₂ is selected from Y, N, C, |
| | and H; and X₃ is selected from T and A |
| | (SEQ ID NO: 220) |
| INFLUENZA B VICTORL | MKAIIVLLMVVTSX₁A |
| CONSENSUS SEQ (wit [symbol] | X₁ is selected from S and N |
| substitutions) | (SEQ ID NO: 221) |
| INFLUENZA B YAMAGAT | MX₁AIIVLLMVVTSNA |
| CONSENSUS SEQ (wit [symbol] | X₁ is selected from K and E |
| substitutions) | (SEQ ID NO: 222) |

[symbol]

indicates data missing or illegible when filed

In certain embodiments, the secretion signal peptide has a sequence of SEQ ID NO: 67.

The secretion signal peptide sequence may be positioned at the N terminus or the C terminus (e.g. at the N terminus) of a polypeptide described herein.

In certain embodiments, the SS amino acid sequence is encoded by a codon-optimized polynucleotide sequence.

In certain embodiments, the viral secretion signal peptide is attached to the antigenic prokaryotic polypeptide with a linker.

Examples of polypeptides of the invention that comprise a secretion signal peptide are provided in. This table also provides nucleic acid sequences that encode the polypeptide, which also form part of the invention. Corresponding polypeptides in which glycosylation sites have been mutated are also included in this table. The mutations in these polypeptides are examples of the above discussed glycosylation mutants.

TABLE 24
Examples of polypeptides of the invention, or polypeptides
encoded by nucleic acids of the invention

| | Amino acid SEQ | Nucleic acid SEQ | |
|---|---|---|---|
| Amino acid sequence | ID NO: | ID NO: | Designation |
| MKAILVVLLYTFATANADVYTDHGDLYNTP | SEQ ID NO: 2 | SEQ ID NO: 23 | Kgp_1A_mCat_SP |
| VRMLVVAGAKFKEALKPWLTWKAQKGFYL | | SEQ ID NO: 24 | |
| DVHYTDEAEVGTTNASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYTAHGSETAWADPLL | | | |
| TTSQLKALTNKDKYFLAIGNCSITAQFDYVQP | | | |
| CFGEVITRVKEKGAYAYIGSSPNSYWGEDYY | | | |
| WSVGANAVFGVQPTFEGTSMGSYDATFLEDS | | | |
| YNTVNSIMWAGNLAATHAGNIGNITHIGAHY | | | |
| YWEAYHVLGDGSVMPYRAMPKTNTYTLPAS | | | |
| LPQNQASYSIQASAGSYVAISKDGVLYGTGV | | | |
| ANASGVATVSMTKQITENGNYDVVITRSNYL | | | |
| PVIKQIQVGEPSPYQPVSNLTATTQGQKVTLK | | | |
| WEAPSAKKSEGSREVKRIGDGLFVTIEPANDV | | | |
| RANEAKVVLAADNVWGDNTGYQFLLDADH | | | |
| NTFGSVIPATGPLFTGTASSNLYSANFEYLIPA | | | |
| NADPVVTTQNIIVTGQGEVVIPGGVYDYCITN | | | |
| PEPASGKMWIAGDGGNQPARYDDFTFEAGK | | | |
| KYTFTMRRAGMGDGTDMEAEDDSPASYTYT | | | |
| VYRDGTKIKEGLTATTFEEDGVAAGNHEYCV | | | |
| EVKYTAGVSPKVCKDVTVEGSNEFAPVQNLT | | | |
| GSSVGQKVTLKWDAPNGTPNPNPNPNPNPGT | | | |
| TLSESFENGIPASWKTIDADGDGHGWKPGNA | | | |
| PGIAGYNSNGCVYSESFGLGGIGVLTPDNYLI | | | |
| TPALDLPNGGKLTFWVCAQDANYASEHYAV | | | |
| YASSTGNDASNFTNALLEETITAKGVRSPKAI | | | |
| RGRIQGTWRQKTVDLPAGTKYVAFRHFQSTD | | | |
| MFYIDLDEVEIKANGGGSPTPTDYTYTVYRD | | | |
| GTKIKEGLTETTFEEDGVATGNHEYCVEVKY | | | |
| TAGVSPKKCVNVTVN | | | |
| MKAILVVLLYTFATANADVYTDHGDLYNTP | SEQ ID NO: 7 | SEQ ID NO: 33 | Kgp_1B_mCat_SP |
| VRMLVVAGAKFKEALKPWLTWKAQKGFYL | | SEQ ID NO: 34 | |
| DVHYTDEAEVGTTNASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYTAHGSETAWADPLL | | | |
| TTSQLKALTNKDKYFLAIGNCSITAQFDYVQP | | | |
| CFGEVITRVKEKGAYAYIGSSPNSYWGEDYY | | | |
| WSVGANAVFGVQPTFEGTSMGSYDATFLEDS | | | |
| YNTVNSIMWAGNLAATHAGNIGNITHIGAHY | | | |
| YWEAYHVLGDGSVMPYRAMPKTNTYTLPAS | | | |
| LPQNQASYSIQASAGSYVAISKDGVLYGTGV | | | |
| ANASGVATVSMTKQITENGNYDVVITRSNYL | | | |
| PVIKQIQVGEPSPYQPVSNLTATTQGQKVTLK | | | |
| WEAPSAKKSEGSREVKRIGDGLFVTIEPANDV | | | |
| RANEAKVVLAADNVWGDNTGYQFLLDADH | | | |
| NTFGSVIPATGPLFTGTASSNLYSANFEYLIPA | | | |
| NADPVVTTQNIIVTGQGEVVIPGGVYDYCITN | | | |
| PEPASGKMWIAGDGGNQPARYDDFTFEAGK | | | |
| KYTFTMRRAGMGDGTDMEAEDDSPASYTYT | | | |
| VYRDGTKIKEGLTATTFEEDGVAAGNHEYCV | | | |
| EVKYTAGVSPKVCKDVTVEGSNEFAPVQNLT | | | |
| GSSVGQKVTLKWDAPNGTPNPNPNPNPNPGT | | | |
| TLSESFPTDYTYTVYRDGTKIKEGLTETTFEE | | | |
| DGVATGNHEYCVEVKYTAGVSPKKCVNVTV | | | |
| N | | | |
| MKAILVVLLYTFATANANTGVSFANYTAHGS | SEQ ID NO: 12 | SEQ ID NO: 43 | Kgp_2A_SP |
| ETAWADPLLTTSQLKALTNKDKPYQPVSNLT | | SEQ ID NO: 44 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQNLTGSSVGQKVTLKWDAPNGTPN | | | |
| PNPNPNPNPGTTLSESFENGIPASWKTIDADG | | | |
| DGHGWKPGNAPGIAGYNSNGCVYSESFGLG | | | |
| GIGVLTPDNYLITPALDLPNGGKLTFWVCAQ | | | |
| DANYASEHYAVYASSTGNDASNFTNALLEET | | | |
| ITAKGVRSPKAIRGRIQGTWRQKTVDLPAGT | | | |
| KYVAFRHFQSTDMFYIDLDEVEIKANGGGSP | | | |
| TPTDYTYTVYRDGTKIKEGLTETTFEEDGVAT | | | |
| GNHEYCVEVKYTAGVSPKKCVNVTVN | | | |
| MKAILVVLLYTFATANANTGVSFANYTAHGS | SEQ ID NO: 17 | SEQ ID NO: 53 | Kgp_2B_SP |
| ETAWADPLLTTSQLKALTNKDKPYQPVSNLT | | SEQ ID NO: 54 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQNLTGSSVGQKVTLKWDAPNGTPN | | | |
| PNPNPNPNPGTTLSESFPTDYTYTVYRDGTKI | | | |
| KEGLTETTFEEDGVATGNHEYCVEVKYTAGV | | | |
| SPKKCVNVTVN | | | |
| MKAILVVLLYTFATANADVYTDHGDLYNTP | SEQ ID NO: 3 | SEQ ID NO: 25 | Kgp_1A_mCat_mGly_ |
| VRMLVVAGAKFKEALKPWLTWKAQKGFYL | | SEQ ID NO: 26 | SP |
| DVHYTDEAEVGTTQASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYVAHGSETAWADPL | | | |
| LTTSQLKALTNKDKYFLAIGNCSITAQFDYVQ | | | |
| PCFGEVITRVKEKGAYAYIGSSPNSYWGEDY | | | |
| YWSVGANAVFGVQPTFEGTSMGSYDATFLE | | | |
| DSYNTVNSIMWAGNLAATHAGNIGNIVHIGA | | | |
| HYYWEAYHVLGDGSVMPYRAMPKTNTYTLP | | | |
| ASLPQNQASYSIQASAGSYVAISKDGVLYGTG | | | |
| VANAAGVATVSMTKQITENGNYDVVITRSNY | | | |
| LPVIKQIQVGEPSPYQPVSQLTATTQGQKVTL | | | |
| KWEAPSAKKSEGSREVKRIGDGLFVTIEPAND | | | |
| VRANEAKVVLAADNVWGDNTGYQFLLDAD | | | |
| HNTFGSVIPATGPLFTGTASSNLYSANFEYLIP | | | |
| ANADPVVTTQNIIVTGQGEVVIPGGVYDYCIT | | | |
| NPEPASGKMWIAGDGGNQPARYDDFTFEAG | | | |
| KKYTFTMRRAGMGDGTDMEAEDDSPASYTY | | | |
| TVYRDGTKIKEGLTATTFEEDGVAAGNHEYC | | | |
| VEVKYTAGVSPKVCKDVTVEGSNEFAPVQQL | | | |
| TGSSVGQKVTLKWDAPQGTPNPNPNPNPNPG | | | |
| TTLSESFENGIPASWKTIDADGDGHGWKPGN | | | |
| APGIAGYNSNGCVYSESFGLGGIGVLTPDNYL | | | |
| ITPALDLPNGGKLTFWVCAQDANYASEHYAV | | | |
| YASSTGNDASNFANALLEETITAKGVRSPKAI | | | |
| RGRIQGTWRQKTVDLPAGTKYVAFRHFQSTD | | | |
| MFYIDLDEVEIKANGGGSPTPTDYTYTVYRD | | | |
| GTKIKEGLTETTFEEDGVATGNHEYCVEVKY | | | |
| TAGVSPKKCVDVTVN | | | |
| MKAILVVLLYTFATANADVYTDHGDLYNTP | SEQ ID NO: 279 | SEQ ID NO: 289 | Kgp_1A_mCat_mGly_ |
| VRMLVVAGAKFKEALKPWLTWKAQKGFYL | | | SP(G2) |
| DVHYTDEAEVGTTQASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYVAHGSETAWADPL | | | |
| LTTSQLKALTNKDKYFLAIGNCSITAQFDYVQ | | | |
| PCFGEVITRVKEKGAYAYIGSSPNSYWGEDY | | | |
| YWSVGANAVFGVQPTFEGTSMGSYDATFLE | | | |
| DSYNTVNSIMWAGNLAATHAGNIGNIVHIGA | | | |
| HYYWEAYHVLGDGSVMPYRAMPKTNTYTLP | | | |
| ASLPQNQASYSIQASAGSYVAISKDGVLYGTG | | | |
| VANAAGVATVSMTKQITENGNYDVVITRSNY | | | |
| LPVIKQIQVGEPSPYQPVSNLAATTQGQKVTL | | | |
| KWEAPSAKKSEGSREVKRIGDGLFVTIEPAND | | | |
| VRANEAKVVLAADNVWGDNTGYQFLLDAD | | | |
| HNTFGSVIPATGPLFTGTASSNLYSANFEYLIP | | | |
| ANADPVVTTQNIIVTGQGEVVIPGGVYDYCIT | | | |
| NPEPASGKMWIAGDGGNQPARYDDFTFEAG | | | |
| KKYTFTMRRAGMGDGTDMEAEDDSPASYTY | | | |
| TVYRDGTKIKEGLTATTFEEDGVAAGNHEYC | | | |
| VEVKYTAGVSPKVCKDVTVEGSNEFAPVQNL | | | |
| AGSSVGQKVTLKWDAPQGTPNPNPNPNPNPG | | | |
| TTLSESFENGIPASWKTIDADGDGHGWKPGN | | | |
| APGIAGYNSNGCVYSESFGLGGIGVLTPDNYL | | | |
| ITPALDLPNGGKLTFWVCAQDANYASEHYAV | | | |
| YASSTGNDASNFANALLEETITAKGVRSPKAI | | | |
| RGRIQGTWRQKTVDLPAGTKYVAFRHFQSTD | | | |
| MFYIDLDEVEIKANGGGSPTPTDYTYTVYRD | | | |
| GTKIKEGLTETTFEEDGVATGNHEYCVEVKY | | | |
| TAGVSPKKCVDVTVN | | | |
| MKAILVVLLYTFATANADVYTDHGDLYNTP | SEQ ID NO: 8 | SEQ ID NO: 35 | Kgp_1B_mCat_mGly_ |
| VRMLVVAGAKFKEALKPWLTWKAQKGFYL | | SEQ ID NO: 36 | SP |
| DVHYTDEAEVGTTQASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYVAHGSETAWADPL | | | |
| LTTSQLKALTNKDKYFLAIGNCSITAQFDYVQ | | | |
| PCFGEVITRVKEKGAYAYIGSSPNSYWGEDY | | | |
| YWSVGANAVFGVQPTFEGTSMGSYDATFLE | | | |
| DSYNTVNSIMWAGNLAATHAGNIGNIVHIGA | | | |
| HYYWEAYHVLGDGSVMPYRAMPKTNTYTLP | | | |
| ASLPQNQASYSIQASAGSYVAISKDGVLYGTG | | | |
| VANAAGVATVSMTKQITENGNYDVVITRSNY | | | |
| LPVIKQIQVGEPSPYQPVSQLTATTQGQKVTL | | | |
| KWEAPSAKKSEGSREVKRIGDGLFVTIEPAND | | | |
| VRANEAKVVLAADNVWGDNTGYQFLLDAD | | | |
| HNTFGSVIPATGPLFTGTASSNLYSANFEYLIP | | | |
| ANADPVVTTQNIIVTGQGEVVIPGGVYDYCIT | | | |
| NPEPASGKMWIAGDGGNQPARYDDFTFEAG | | | |
| KKYTFTMRRAGMGDGTDMEAEDDSPASYTY | | | |
| TVYRDGTKIKEGLTATTFEEDGVAAGNHEYC | | | |
| VEVKYTAGVSPKVCKDVTVEGSNEFAPVQQL | | | |
| TGSSVGQKVTLKWDAPQGTPNPNPNPNPNPG | | | |
| TTLSESFPTDYTYTVYRDGTKIKEGLTETTFEE | | | |
| DGVATGNHEYCVEVKYTAGVSPKKCVDVTV | | | |
| N | | | |
| MKAILVVLLYTFATANANTGVSFANYVAHGS | SEQ ID NO: 13 | SEQ ID NO: 45 | Kgp_2A_mGly_SP |
| ETAWADPLLTTSQLKALTNKDKPYQPVSQLT | | SEQ ID NO: 46 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQQLTGSSVGQKVTLKWDAPQGTPN | | | |
| PNPNPNPNPGTTLSESFENGIPASWKTIDADG | | | |
| DGHGWKPGNAPGIAGYNSNGCVYSESFGLG | | | |
| GIGVLTPDNYLITPALDLPNGGKLTFWVCAQ | | | |
| DANYASEHYAVYASSTGNDASNFANALLEET | | | |
| ITAKGVRSPKAIRGRIQGTWRQKTVDLPAGT | | | |
| KYVAFRHFQSTDMFYIDLDEVEIKANGGGSP | | | |
| TPTDYTYTVYRDGTKIKEGLTETTFEEDGVAT | | | |
| GNHEYCVEVKYTAGVSPKKCVDVTVN | | | |
| MKAILVVLLYTFATANANTGVSFANYVAHGS | SEQ ID NO: 280 | SEQ ID NO: 290 | Kgp_2A_mGly_SP |
| ETAWADPLLTTSQLKALTNKDKPYQPVSNLA | | | (G2) |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQNLAGSSVGQKVTLKWDAPQGTP | | | |
| NPNPNPNPNPGTTLSESFENGIPASWKTIDAD | | | |
| GDGHGWKPGNAPGIAGYNSNGCVYSESFGL | | | |
| GGIGVLTPDNYLITPALDLPNGGKLTFWVCA | | | |
| QDANYASEHYAVYASSTGNDASNFANALLE | | | |
| ETITAKGVRSPKAIRGRIQGTWRQKTVDLPAG | | | |
| TKYVAFRHFQSTDMFYIDLDEVEIKANGGGS | | | |
| PTPTDYTYTVYRDGTKIKEGLTETTFEEDGVA | | | |
| TGNHEYCVEVKYTAGVSPKKCVDVTVN | | | |
| MKAILVVLLYTFATANAKAPYTGCYSHLNTG | SEQ ID NO: 297 | SEQ ID NO: 298 | Kgp_ext_2A_mGly_SP |
| VSFANYVAHGSETAWADPLLTTSQLKALTNK | | | (G2) |
| DKPYQPVSNLAATTQGQKVTLKWEAPSAKK | | | |
| SEGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQNLAGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGGGSPTPTDYTYTVYRDGTKIKEGLT | | | |
| ETTFEEDGVATGNHEYCVEVKYTAGVSPKKC | | | |
| VDVTVN | | | |
| MKAILVVLLYTFATANANTGVSFANYVAHGS | SEQ ID NO: 18 | SEQ ID NO: 55 | Kgp_2B_mGly_SP |
| ETAWADPLLTTSQLKALTNKDKPYQPVSQLT | | SEQ ID NO: 56 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQQLTGSSVGQKVTLKWDAPQGTPN | | | |
| PNPNPNPNPGTTLSESFPTDYTYTVYRDGTKI | | | |
| KEGLTETTFEEDGVATGNHEYCVEVKYTAGV | | | |
| SPKKCVDVTVN | | | |
| MKAILVVLLYTFATANAEPGRYTPVEEKQNG | SEQ ID NO: 73 | SEQ ID NO: 78 | RgpA_1B_mCat_mGly |
| RMIVIVAKKYEGDIKDFVDWKNQRGLRTEVK | | SEQ ID NO: 79 | SP |
| VAEDIASPVTANAIQQFVKQEYEKEGNDLTY | | | |
| VLLIGDHKDIPAKITPGIKSDQVYGQIVGNDH | | | |
| YNEVFIGRFSCESKEDLKTQIDRTIHYERNIAT | | | |
| EDKWLGQALCIASAEGGPSADNGESDIQHEN | | | |
| VIANLLTQYGYTKIIKCYDPGVTPKNIIDAFNG | | | |
| GISLANYAGHGSETAWGTSHFGTTHVKQLTN | | | |
| SNQLPFIFDVASVNGDFLFSMPCFAEALMRAQ | | | |
| KDGKPTGTVAIIASTIQQSWASPMRGQDEMN | | | |
| EILCEKHPNNIKRTFGGVTMNGMFAMVEKYK | | | |
| KDGEKMLDTWTVFGDPSLLVRTLVPTKMQV | | | |
| TAPAQINLADASVQVSCDYNGAIATISANGK | | | |
| MFGSAVVEQGTATIQLTGLTQESTLTLTVVG | | | |
| YNKETVIKTINTNGEPNPYQPVSNLAATTQGQ | | | |
| KVTLKWDAPSTKTQATTNTARSVDGIRELVL | | | |
| LSVSDAPELLRSGQAEIVLEAHDVWNDGSGY | | | |
| QILLDADHDQYGQVIPSDTHTLWPNCAVPAN | | | |
| LFAPFEYTVPENADPSCSPTNMIMDGTASVNI | | | |
| PAGTYDFAIAAPQANAKIWIAGQGPTKEDDY | | | |
| VFEAGKKYHFLMKKMGSGDGTELTISEGGGS | | | |
| DYTYTVYRDGTKIKEGLTATTFEEDGVAAGN | | | |
| HEYCVEVKYTAGVSPKVCKDVTVEGSNEFAP | | | |
| VQNLAGSAVGQKVTLKWDAPQGTPNPNPNP | | | |
| NPNPNPGTTTLSESFGKRADFTETFESSTHGE | | | |
| APAEWTTIDADGDGQGWLCLSSGQLDWLTA | | | |
| HGGTNVVASFSWNGMALNPDNYLISKDVTG | | | |
| ATKVKYYYAVNDGFPGDHYAVMISKTGTNA | | | |
| GDFTVVFEETPNGINKGGARFGLSTEANGAK | | | |
| PQSVWIERTVDLPAGTKYVAFRHYQCSDLNY | | | |
| ILLDDIQFTMGGSPTPTDYTYTVYRDGTKIKE | | | |
| GLTETTFEEDGVATGNHEYCVEVKYTAGVSP | | | |
| KKCVQVTVN | | | |
| MKAILVVLLYTFATANANPYQPVSNLAATTQ | SEQ ID NO: 74 | SEQ ID NO: 80 | RgpA_2B_mGly_SP |
| GQKVTLKWDAPSTKTQATTNTARSVDGIREL | | SEQ ID NO: 81 | |
| VLLSVSDAPELLRSGQAEIVLEAHDVWNDGS | | | |
| GYQILLDADHDQYGQVIPSDTHTLWPNCAVP | | | |
| ANLFAPFEYTVPENADPSCSPTNMIMDGTASV | | | |
| NIPAGTYDFAIAAPQANAKIWIAGQGPTKEDD | | | |
| YVFEAGKKYHFLMKKMGSGDGTELTISEGG | | | |
| GSDYTYTVYRDGTKIKEGLTATTFEEDGVAA | | | |
| GNHEYCVEVKYTAGVSPKVCKDVTVEGSNE | | | |
| FAPVQNLAGSAVGQKVTLKWDAPQGTPNPN | | | |
| PNPNPNPNPGTTTLSESFGKRADFTETFESSTH | | | |
| GEAPAEWTTIDADGDGQGWLCLSSGQLDWL | | | |
| TAHGGTNVVASFSWNGMALNPDNYLISKDV | | | |
| TGATKVKYYYAVNDGFPGDHYAVMISKTGT | | | |
| NAGDFTVVFEETPNGINKGGARFGLSTEANG | | | |
| AKPQSVWIERTVDLPAGTKYVAFRHYQCSDL | | | |
| NYILLDDIQFTMGGSPTPTDYTYTVYRDGTKI | | | |
| KEGLTETTFEEDGVATGNHEYCVEVKYTAGV | | | |
| SPKKCVQVTVNGVTPKNIIDAFNGGISLAQYT | | | |
| GHGSETAWGTSHFGTTHVKQLTNSNQ | | | |
| MKAILVVLLYTFATANAKAPYTGCYSHLNTG | SEQ ID NO: 75 | SEQ ID NO: 82 | Kgp_2A_Ras2_mGly_ |
| VSFANYVAHGSETAWADPLLTTSQLKALTNK | | SEQ ID NO: 83 | SP |
| DKPYQPVSQLTATTQGQKVTLKWEAPSAKKS | | | |
| EGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQQLTGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGGGSPTPTDYTYTVYRDGTKIKEGLT | | | |
| ETTFEEDGVATGNHEYCVEVKYTAGVSPKKC | | | |
| VDVTVNGVTPKNIIDAFNGGISLAQYTGHGSE | | | |
| TAWGTSHFGTTHVKQLTNSNQ | | | |
| MKAILVVLLYTFATANAKAPYTGCYSHLNTG | SEQ ID NO: 283 | SEQ ID NO: 291 | Kgp_2A_Ras2_mGly_ |
| VSFANYVAHGSETAWADPLLTTSQLKALTNK | | SEQ ID NO: 292 | SP_G2 |
| DKPYQPVSNLAATTQGQKVTLKWEAPSAKK | | | |
| SEGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQNLAGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGGGSPTPTDYTYTVYRDGTKIKEGLT | | | |
| ETTFEEDGVATGNHEYCVEVKYTAGVSPKKC | | | |
| VDVTVNGVTPKNIIDAFNGGISLAQYTGHGSE | | | |
| TAWGTSHFGTTHVKQLTNSNQ | | | |
| MKAILVVLLYTFATANAKAPYTGCYSHLNTG | SEQ ID NO: 76 | SEQ ID NO: 84 | Kgp_2A_K2_Ras2_ |
| VSFANYVAHGSETAWADPLLTTSQLKALTNK | | SEQ ID NO: 85 | mGly_SP |
| DKPYQPVSQLTATTQGQKVTLKWEAPSAKKS | | | |
| EGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQQLTGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGKRADFTETFESSTHGEAPAEWTTID | | | |
| ADGDGQGWLCLSSGQLDWLTAHGGSNVVSS | | | |
| FSWNGMALNPDNYLISKDVTGATKVKYYYA | | | |
| VNDGFPGDHYAVMISKTGTNAGDFTVVFEET | | | |
| PNGINKGGARFGLSTEANGAKPQSVWIERTV | | | |
| DLPAGTKYVAFRHYQCSDLNYILLDDIQFTM | | | |
| GGSPTPTDYTYTVYRDGTKIKEGLTETTFEED | | | |
| GVATGNHEYCVEVKYTAGVSPKKCVDVTVN | | | |
| GVTPKNIIDAFNGGISLAQYTGHGSETAWGTS | | | |
| HFGTTHVKQLTNSNQ | | | |
| MKAILVVLLYTFATANAKAPYTGCYSHLNTG | SEQ ID NO: 284 | SEQ ID NO: 293 | Kgp_2A_K2_Ras2_ |
| VSFANYVAHGSETAWADPLLTTSQLKALTNK | | SEQ ID NO: 294 | mGly_SP_G2 |
| DKPYQPVSNLAATTQGQKVTLKWEAPSAKK | | | |
| SEGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQNLAGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGKRADFTETFESSTHGEAPAEWTTID | | | |
| ADGDGQGWLCLSSGQLDWLTAHGGSNVVSS | | | |
| FSWNGMALNPDNYLISKDVTGATKVKYYYA | | | |
| VNDGFPGDHYAVMISKTGTNAGDFTVVFEET | | | |
| PNGINKGGARFGLSTEANGAKPQSVWIERTV | | | |
| DLPAGTKYVAFRHYQCSDLNYILLDDIQFTM | | | |
| GGSPTPTDYTYTVYRDGTKIKEGLTETTFEED | | | |
| GVATGNHEYCVEVKYTAGVSPKKCVDVTVN | | | |
| GVTPKNIIDAFNGGISLAQYTGHGSETAWGTS | | | |
| HFGTTHVKQLTNSNQ | | | |
| MKAILVVLLYTFATANAKAPYTGCYSHLNTG | SEQ ID NO: 401 | | Kgp_2A_K2_Ras2_ |
| VSFANYVAHGSETAWADPLLTTSQLKALTNK | | | mGly_SP_G3 |
| DKPYQPVSQLTATTQGQKVTLKWEAPSAKKS | | | |
| EGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQQLTGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGKRADFTETFESSTHGEAPAEWTTID | | | |
| ADGDGQGWLCLSSGQLDWLTAHGGTNVVA | | | |
| SFSWNGMALNPDNYLISKDVTGATKVKYYY | | | |
| AVNDGFPGDHYAVMISKTGTNAGDFTVVFEE | | | |
| TPNGINKGGARFGLSTEANGAKPQSVWIERT | | | |
| VDLPAGTKYVAFRHYQCSDLNYILLDDIQFT | | | |
| MGGSPTPTDYTYTVYRDGTKIKEGLTETTFEE | | | |
| DGVATGNHEYCVEVKYTAGVSPKKCVDVTV | | | |
| NGVTPKNIIDAFNGGISLAQYTGHGSETAWGT | | | |
| SHFGTTHVKQLTNSNQ | | | |
| MKAILVVLLYTFATANAKAPYTGCYSHLNTG | SEQ ID NO: 413 | | Kgp_2A_K2_Ras2_ |
| VSFANYVAHGSETAWADPLLTTSQLKALTNK | | | mGly_SP_G3 (dup) |
| DKPYQPVSQLTATTQGQKVTLKWEAPSAKKS | | | |
| EGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQQLTGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGKRADFTETFESSTHGEAPAEWTTID | | | |
| ADGDGQGWLCLSSGQLDWLTAHGGTNVVA | | | |
| SFSWNGMALNPDNYLISKDVTGATKVKYYY | | | |
| AVNDGFPGDHYAVMISKTGTNAGDFTVVFEE | | | |
| TPNGINKGGARFGLSTEANGAKPQSVWIERT | | | |
| VDLPAGTKYVAFRHYQCSDLNYILLDDIQFT | | | |
| MGGSPTPGGSPTPTDYTYTVYRDGTKIKEGL | | | |
| TETTFEEDGVATGNHEYCVEVKYTAGVSPKK | | | |
| CVDVTVNGVTPKNIIDAFNGGISLAQYTGHGS | | | |
| ETAWGTSHFGTTHVKQLTNSNQ | | | |
| MKAILVVLLYTFATANAKAPYTGCYSHLNTG | SEQ ID NO: 77 | SEQ ID NO: 86 | Kgp_2A_K2_RgpA_ |
| VSFANYVAHGSETAWADPLLTTSQLKALTNK | | SEQ ID NO: 87 | DUF_Ras2_mGly_SP |
| DKPYQPVSQLTATTQGQKVTLKWEAPSAKKS | | | |
| EGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQQLTGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGKRADFTETFESSTHGEAPAEWTTID | | | |
| ADGDGQGWLCLSSGQLDWLTAHGGSNVVSS | | | |
| FSWNGMALNPDNYLISKDVTGATKVKYYYA | | | |
| VNDGFPGDHYAVMISKTGTNAGDFTVVFEET | | | |
| PNGINKGGARFGLSTEANGAKPQSVWIERTV | | | |
| DLPAGTKYVAFRHYQCSDLNYILLDDIQFTM | | | |
| GGSPTPTDYTYTVYRDGTKIKEGLTETTFEED | | | |
| GVATGNHEYCVEVKYTAGVSPKKCVDVTVN | | | |
| NPYQPVSNLAATTQGQKVTLKWDAPSTKTQ | | | |
| ATTNTARSVDGIRELVLLSVSDAPELLRSGQA | | | |
| EIVLEAHDVWNDGSGYQILLDADHDQYGQVI | | | |
| PSDTHTLWPNCAVPANLFAPFEYTVPENADPS | | | |
| CSPTNMIMDGTASVNIPAGTYDFAIAAPQAN | | | |
| AKIWIAGQGPTKEDDYVFEAGKKYHFLMKK | | | |
| MGSGDGTELTISEGGGSDYTYTVYRDGTKIK | | | |
| EGLTATTFEEDGVAAGNHEYCVEVKYTAGV | | | |
| SPKVCKDVTVEGSGVTPKNIIDAFNGGISLAQ | | | |
| YTGHGSETAWGTSHFGTTHVKQLTNSNQ | | | |
| MKAILVVLLYTFATANAKAPYTGCYSHLNTG | SEQ ID NO: 285 | SEQ ID NO: 295 | Kgp_2A_K2_RgpA_ |
| VSFANYVAHGSETAWADPLLTTSQLKALTNK | | SEQ ID NO: 296 | DUF_Ras2_mGly_SP_ |
| DKPYQPVSNLAATTQGQKVTLKWEAPSAKK | | | G2 |
| SEGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQNLAGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGKRADFTETFESSTHGEAPAEWTTID | | | |
| ADGDGQGWLCLSSGQLDWLTAHGGSNVVSS | | | |
| FSWNGMALNPDNYLISKDVTGATKVKYYYA | | | |
| VNDGFPGDHYAVMISKTGTNAGDFTVVFEET | | | |
| PNGINKGGARFGLSTEANGAKPQSVWIERTV | | | |
| DLPAGTKYVAFRHYQCSDLNYILLDDIQFTM | | | |
| GGSPTPTDYTYTVYRDGTKIKEGLTETTFEED | | | |
| GVATGNHEYCVEVKYTAGVSPKKCVDVTVN | | | |
| NPYQPVSNLAATTQGQKVTLKWDAPSTKTQ | | | |
| ATTNTARSVDGIRELVLLSVSDAPELLRSGQA | | | |
| EIVLEAHDVWNDGSGYQILLDADHDQYGQVI | | | |
| PSDTHTLWPNCAVPANLFAPFEYTVPENADPS | | | |
| CSPTNMIMDGTASVNIPAGTYDFAIAAPQAN | | | |
| AKIWIAGQGPTKEDDYVFEAGKKYHFLMKK | | | |
| MGSGDGTELTISEGGGSDYTYTVYRDGTKIK | | | |
| EGLTATTFEEDGVAAGNHEYCVEVKYTAGV | | | |
| SPKVCKDVTVEGSGVTPKNIIDAFNGGISLAQ | | | |
| YTGHGSETAWGTSHFGTTHVKQLTNSNQ | | | |
| MKAILVVLLYTFATANAKAPYTGCYSHLNTG | SEQ ID NO: 407 | | Kgp_2A_K2_RgpA_ |
| VSFANYVAHGSETAWADPLLTTSQLKALTNK | | | EUF_Ras2_mGly_SP_ |
| DKPYQPVSQLTATTQGQKVTLKWEAPSAKKS | | | G3 |
| EGSREVKRIGDGLFVTIEPANDVRANEAKVV | | | |
| LAADNVWGDNTGYQFLLDADHNTFGSVIPA | | | |
| TGPLFTGTASSNLYSANFEYLIPANADPVVTT | | | |
| QNIIVTGQGEVVIPGGVYDYCITNPEPASGKM | | | |
| WIAGDGGNQPARYDDFTFEAGKKYTFTMRR | | | |
| AGMGDGTDMEAEDDSPASYTYTVYRDGTKI | | | |
| KEGLTATTFEEDGVAAGNHEYCVEVKYTAG | | | |
| VSPKVCKDVTVEGSNEFAPVQQLTGSSVGQK | | | |
| VTLKWDAPQGTPNPNPNPNPNPGTTLSESFEN | | | |
| GIPASWKTIDADGDGHGWKPGNAPGIAGYNS | | | |
| NGCVYSESFGLGGIGVLTPDNYLITPALDLPN | | | |
| GGKLTFWVCAQDANYASEHYAVYASSTGND | | | |
| ASNFANALLEETITAKGVRSPKAIRGRIQGTW | | | |
| RQKTVDLPAGTKYVAFRHFQSTDMFYIDLDE | | | |
| VEIKANGKRADFTETFESSTHGEAPAEWTTID | | | |
| ADGDGQGWLCLSSGQLDWLTAHGGTNVVA | | | |
| SFSWNGMALNPDNYLISKDVTGATKVKYYY | | | |
| AVNDGFPGDHYAVMISKTGTNAGDFTVVFEE | | | |
| TPNGINKGGARFGLSTEANGAKPQSVWIERT | | | |
| VDLPAGTKYVAFRHYQCSDLNYILLDDIQFT | | | |
| MGGSPTPTDYTYTVYRDGTKIKEGLTETTFEE | | | |
| DGVATGNHEYCVEVKYTAGVSPKKCVDVTV | | | |
| NNPYQPVSNLAATTQGQKVTLKWDAPSTKT | | | |
| QATTNTARSVDGIRELVLLSVSDAPELLRSGQ | | | |
| AEIVLEAHDVWNDGSGYQILLDADHDQYGQ | | | |
| VIPSDTHTLWPNCAVPANLFAPFEYTVPENAD | | | |
| PSCSPTNMIMDGTASVNIPAGTYDFAIAAPQA | | | |
| NAKIWIAGQGPTKEDDYVFEAGKKYHFLMK | | | |
| KMGSGDGTELTISEGGGSDYTYTVYRDGTKI | | | |
| KEGLTATTFEEDGVAAGNHEYCVEVKYTAG | | | |
| VSPKVCKDVTVEGSGVTPKNIIDAFNGGISLA | | | |
| QYTGHGSETAWGTSHFGTTHVKQLTNSNQ | | | |

In some embodiments, the polypeptide comprises a sequence according to SEQ ID NO: 2, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 7, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 12, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 17, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 3, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 279, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 8, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 13, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 280, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 297, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 18, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 73, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 74, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 75, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 283, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 76, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 284, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 401, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 413, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 77, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 285, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 407, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

The N-terminal methionine in any of the above embodiments may be omitted from the polypeptide. Accordingly, in some embodiments, the polypeptide comprises a sequence according to SEQ ID NO: 255 (i.e. SEQ ID NO: 2 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 260 (i.e. SEQ ID NO: 7 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 265 (i.e. SEQ ID NO: 12 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 270 (i.e. SEQ ID NO: 17 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 256 (i.e. SEQ ID NO: 3 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 281 (i.e. SEQ ID NO: 279) without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 261 (i.e. SEQ ID NO: 8 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 266 (i.e. SEQ ID NO: 13 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 299 (i.e., SEQ ID NO: 297 without the N-terminal methionine), or a sequence that has at least 70% (e.g., at least 75, 80, 85, 90 or 95%; or e.g. at least 75, 80, 85, 86, 87, 88, 89, 90, 91, 92, 93, 94, 95, 96, 97, 98 or 99%) identity thereto. In some embodiments, the polypeptide comprises a sequence according to SEQ ID NO: 282 (i.e. SEQ ID NO: 280 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 271 (i.e. SEQ ID NO: 18 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 274 (i.e. SEQ ID NO: 73 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 275 (i.e. SEQ ID NO: 74 without the N-terminal methionine), or sequence that has at least 70% (e.g. at least 75, 80, 85, 90 or 95%) (e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 276 (i.e. SEQ ID NO: 75 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 286 (i.e. SEQ ID NO: 283 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 277 (i.e. SEQ ID NO: 76 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 287 (i.e. SEQ ID NO: 284 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 402 (i.e. SEQ ID NO: 401 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 414 (i.e. SEQ ID NO: 413 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 278 (i.e. SEQ ID NO: 77 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 288 (i.e. SEQ ID NO: 285 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 408 (i.e. SEQ ID NO: 407 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the nucleic acid encoding the polypeptide comprises a sequence according to SEQ ID NO: 23. In some embodiments, the nucleic acid encoding the polypeptide comprises a sequence according to SEQ ID NO: 24.

In some embodiments, the nucleic acid encoding the polypeptide comprises a sequence according to SEQ ID NO: 33. In some embodiments, the nucleic acid encoding the polypeptide comprises a sequence according to SEQ ID NO: 34.

In some embodiments, the nucleic acid encoding the polypeptide comprises a sequence according to SEQ ID NO: 43. In some embodiments, the nucleic acid encoding the polypeptide comprises a sequence according to SEQ ID NO: 44.

In some embodiments, the nucleic acid encoding the polypeptide comprises a sequence according to SEQ ID NO: 53. In some embodiments, the nucleic acid encoding the polypeptide comprises a sequence according to SEQ ID NO: 54.

In some embodiments, the nucleic acid encoding the polypeptide comprises a sequence according to SEQ ID NO: 25. In some embodiments, the nucleic acid encoding the polypeptide comprises a sequence according to SEQ ID NO: 26.

In some embodiments, the nucleic acid encoding the polypeptide comprises a sequence according to SEQ ID NO: 289.

In some embodiments, the nucleic acid encoding the polypeptide comprises a sequence according to SEQ ID NO: 35. In some embodiments, the nucleic acid encoding the polypeptide comprises a sequence according to SEQ ID NO: 36.

In some embodiments, the nucleic acid encoding the polypeptide comprises a sequence according to SEQ ID NO: 45. In some embodiments, the nucleic acid encoding the polypeptide comprises a sequence according to SEQ ID NO: 46.

In some embodiments, the nucleic acid encoding the polypeptide comprises a sequence according to SEQ ID NO: 290.

In some embodiments, the nucleic acid encoding the polypeptide comprises a sequence according to SEQ ID NO: 298.

In some embodiments, the nucleic acid encoding the polypeptide comprises a sequence according to SEQ ID NO: 55. In some embodiments, the nucleic acid encoding the polypeptide comprises a sequence according to SEQ ID NO: 56.

In some embodiments, the nucleic acid encoding the polypeptide comprises a sequence according to SEQ ID NO: 78. In some embodiments, the nucleic acid encoding the polypeptide comprises a sequence according to SEQ ID NO: 79.

In some embodiments, the nucleic acid encoding the polypeptide comprises a sequence according to SEQ ID NO: 80. In some embodiments, the nucleic acid encoding the polypeptide comprises a sequence according to SEQ ID NO: 81.

In some embodiments, the nucleic acid encoding the polypeptide comprises a sequence according to SEQ ID NO: 82. In some embodiments, the nucleic acid encoding the polypeptide comprises a sequence according to SEQ ID NO: 83.

In some embodiments, the nucleic acid encoding the polypeptide comprises a sequence according to SEQ ID NO: 291. In some embodiments, the nucleic acid encoding the polypeptide comprises a sequence according to SEQ ID NO: 292.

In some embodiments, the nucleic acid encoding the polypeptide comprises a sequence according to SEQ ID NO: 84. In some embodiments, the nucleic acid encoding the polypeptide comprises a sequence according to SEQ ID NO: 85.

In some embodiments, the nucleic acid encoding the polypeptide comprises a sequence according to SEQ ID NO: 293. In some embodiments, the nucleic acid encoding the polypeptide comprises a sequence according to SEQ ID NO: 294.

In some embodiments, the nucleic acid encoding the polypeptide comprises a sequence according to SEQ ID NO: 86. In some embodiments, the nucleic acid encoding the polypeptide comprises a sequence according to SEQ ID NO: 87.

In some embodiments, the nucleic acid encoding the polypeptide comprises a sequence according to SEQ ID NO: 295. In some embodiments, the nucleic acid encoding the polypeptide comprises a sequence according to SEQ ID NO: 296.

In certain embodiments, the amino acid sequence of the polypeptides of the invention is encoded by a codon-optimized polynucleotide sequence.

Heterologous transmembrane domains (TMBs)

The polypeptides of the invention as described herein may comprise a heterologous transmembrane domain. The inclusion of a TMB may be advantageous as this will localise the antigen to the cell membrane. This may reduce antigen intracellular localisation and further promote higher immunogenicity relative to the antigen without the TMB sequence. The polypeptides comprising a heterologous transmembrane domain may also comprise a secretion signal peptide sequence. In some embodiments, a nucleic acid described herein encoding the polypeptide that comprises a heterologous transmembrane domain also comprises a nucleotide sequence encoding a secretion signal peptide sequence.

The TMB may be from any known TMB in the art, including but not limited to, TMBs from eukaryotic transmembrane proteins (e.g., mammalian transmembrane proteins, such as human transmembrane proteins), TMBs from prokaryotic transmembrane proteins, and TMBs from viral transmembrane proteins. TMBs may further be identified through in silico prediction algorithms, for example, in the TMHMM prediction method described in Krogh et al. (J Mol Biol. 305 (3): 567-580. 2001) and services.healthtech.dtu.dk/services/TMHMM-2.0/, each of which is incorporated herein by reference in their entirety. Some features of TMBs are described in further detail in Albers et al. (Chapter 2-cell membrane structures and functions. Basic Neurochemistry eighth edition. Pages 26-39. 2012), incorporated herein by reference. TMBs are typically, but not exclusively, comprised predominantly of nonpolar (hydrophobic) amino acid residues and may traverse a lipid bilayer once or several times. The skilled person knows well methods to determine the hydrophobicity of an amino acid. See Simm et al. (2016), Biol Res., 49 (1): 31; Wimlet and White (1996), Nat Struct Biol., 3 (10): 842-848;
blanco.biomol.uci.edu/hydrophobicity_scales.html; and www.cgl.ucsf.edu/chimera/docs/UsersGuide/midas/hydrophob.html.

In certain embodiments the TMB: (a) comprises or consists of 15 to 50 amino acid residues, preferably 15 to 30 amino acid residues, more preferably 18 to 25 amino acid residues; and/or (b) comprises at least 50% of hydrophobic amino acid residues, preferably selected in the group consisting of: alanine, isoleucine, leucine, valine, phenylalanine, tryptophane and tyrosine; and/or (c) comprises at least one alpha helix.

The TMBs usually comprise alpha helices, each helix containing 18-21 amino acids, which is sufficient to span the lipid bilayer. Accordingly, in certain embodiments, the transmembrane domain comprises one or more alpha helices.

In certain embodiments, the transmembrane domain is derived from an integral membrane protein, as further defined hereafter and in Albers et al., An “integral membrane protein” (also known as an intrinsic membrane protein) is a membrane protein that is permanently attached to the lipid membrane. In certain embodiments, the transmembrane domain is derived from an integral polytopic protein. An integral polytopic protein is one that spans the entire membrane. In certain embodiments, the transmembrane domain is derived from a single pass (trans) membrane protein, more particularly a bitopic membrane protein, e.g., of Type I or Type II. Single-pass membrane proteins cross the membrane only once (i.e., a bitopic membrane protein), while multi-pass membrane proteins weave in and out, crossing several times. Single pass transmembrane proteins can be categorized as Type I, which are positioned such that their carboxyl-terminus is towards the cytosol, or Type II, which have their amino-terminus towards the cytosol. In certain embodiments, the transmembrane domain is derived from an integral monotopic protein. An integral monotopic protein is one that is associated with the membrane from only one side and does not span the lipid bilayer completely.

In certain embodiments, the heterologous transmembrane domain is derived from a non-human sequence.

In certain embodiments, the heterologous transmembrane domain is derived from a viral sequence. The phrase “influenza”, “SARS CoV-2”, “varicella-zoster virus (VZV)”, “measles”, “rubella”, “rabies,” “Ebola,” and “smallpox” preceding the phrase “transmembrane domain sequence” indicates that the transmembrane domain sequence was derived from the virus corresponding to that name.

In certain embodiments, the heterologous transmembrane domain is derived from a viral transmembrane domain sequence selected from the group consisting of: an influenza transmembrane domain sequence, a SARS CoV-2 transmembrane domain sequence, a varicella-zoster virus (VZV) transmembrane domain sequence, a measles transmembrane domain sequence, a rubella transmembrane domain sequence, a mumps transmembrane domain sequence, a rabies transmembrane domain sequence, and an Ebola transmembrane domain sequence. These particular transmembrane domains are derived from viral sequences in viruses which have been administered to humans as vaccines (live-attenuated, inactivated or mRNA), with demonstrated strong safety profiles.

In certain embodiments, the heterologous transmembrane domain is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence, a rabies virus glycoprotein (Rabies G) transmembrane domain sequence, and an Ebola GP protein transmembrane domain sequence.

In certain embodiments, the heterologous transmembrane domain comprises an HA transmembrane domain sequence from influenza A or influenza B, preferably from influenza A.

Exemplary viral transmembrane domain amino acid sequences of the disclosure are shown below in.

TABLE 25
Viral Transmembrane Domain (TMB) Signal Amino Acid Sequences

| NAME OF THE | | | |
|---|---|---|---|
| PROTEIN | ORGANISM | STRAIN | SEQUENCE OF THE TMB |
| HA (H1N1) | Influenza virus | A/New Caledonia/20/1999 | ILAIYSTVASSLVLLVSLGAISF |
| | | | (SEQ ID NO: 223) |
| HA (H1N1pdm) | Influenza virus | A/California/7/2009 | ILAIYSTVASSLVLVVSLGAISF |
| | | | (SEQ ID NO: 224) |
| HA (H3N2) | Influenza virus | A/Moscow/10/1999 | ILWISFAISCFLLCVVLLGFI |
| | | | (SEQ ID NO: 225) |
| HA B | Influenza virus | B/Phuket/3073/2013 | STAASSLAVTLMLAIFIVYMV |
| | | | (SEQ ID NO: 226) |
| Spike | SARS COV-2 | Wuhan-1 | WYIWLGFIAGLIAIVMVTIML |
| | | | (SEQ ID NO: 227) |
| gB | VZV | Oka strain | FGALAVGLLVLAGLVAAFFAY |
| | | | (SEQ ID NO: 228) |
| gE | VZV | Oka strain | AAWTGGLAAVVLLCLVIFLIC |
| | | | (SEQ ID NO: 229) |
| gI | VZV | Oka strain | IIIPIVASVMILTAMVIVIVI |
| | | | (SEQ ID NO: 230) |
| gK | VZV | Oka strain | YFWCVQLKMIFFAWFVYGMYL |
| | | | (SEQ ID NO: 231) |
| F | Measles | Edmonston-Zagreb strain | IVYILIA VCLGGLIGIPALIC |
| | | | (SEQ ID NO: 232) |
| E1 | Rubella | RA27/3 strain | LDHAFAAFVLLVPWVLIFMVC |
| | | | (SEQ ID NO: 233) |
| E2 | Rubella | RA27/3 strain | WWQLTLGAICALLLAGLLACC |
| | | | (SEQ ID NO: 234) |
| F | Mumps | Miyahara strain | IVAALVLSILSIIISLLFCCW |
| | | | (SEQ ID NO: 235) |
| GP | Ebola | Mayinga-76 strain | WIPAGIGVTGVIIA VIALFCI |
| | | | (SEQ ID NO: 236) |
| Rabies G | Rabies | Rabies Pasteur strain | VLLSAGALTALMLIIFLMTCW |
| | | | (SEQ ID NO: 237) |

In certain embodiments, the TMB sequence has a sequence of GGSILAIYSTVASSLVLVVSLGAISFGG (SEQ ID NO: 70).

In certain embodiments, the heterologous TMB sequence is positioned at the N-terminus or the C-terminus (e.g. C-terminus) of a polypeptide described herein.

In certain embodiments, the TMB amino acid sequence is encoded by a codon-optimized polynucleotide sequence.

In certain embodiments, the TMB is attached to a polypeptide described herein with a linker.

Examples of polypeptides of the invention that comprise a TMB are provided in. This table also provides nucleic acid sequences that encode the polypeptide, which also form part of the invention. Corresponding polypeptides in which glycosylation sites have been mutated are also included in this table. The mutations in these polypeptides are examples of the above discussed glycosylation mutants.

TABLE 26
Examples of polypeptides of the invention,
or polypeptides encoded by nucleic acids of the invention

| | Amino acid SEQ | Nucleic acid SEQ | |
|---|---|---|---|
| Amino acid sequence | ID NO: | ID NO: | Designation |
| MKAILVVLLYTFATANADVYTDHGDLYNTP | SEQ ID NO: 4 | SEQ ID NO: 27 | Kgp_1A_mCat_SP_TM |
| VRMLVVAGAKFKEALKPWLTWKAQKGFYL | | SEQ ID NO: 28 | |
| DVHYTDEAEVGTTNASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYTAHGSETAWADPLL | | | |
| TTSQLKALTNKDKYFLAIGNCSITAQFDYVQP | | | |
| CFGEVITRVKEKGAYAYIGSSPNSYWGEDYY | | | |
| WSVGANAVFGVQPTFEGTSMGSYDATFLEDS | | | |
| YNTVNSIMWAGNLAATHAGNIGNITHIGAHY | | | |
| YWEAYHVLGDGSVMPYRAMPKTNTYTLPAS | | | |
| LPQNQASYSIQASAGSYVAISKDGVLYGTGV | | | |
| ANASGVATVSMTKQITENGNYDVVITRSNYL | | | |
| PVIKQIQVGEPSPYQPVSNLTATTQGQKVTLK | | | |
| WEAPSAKKSEGSREVKRIGDGLFVTIEPANDV | | | |
| RANEAKVVLAADNVWGDNTGYQFLLDADH | | | |
| NTFGSVIPATGPLFTGTASSNLYSANFEYLIPA | | | |
| NADPVVTTQNIIVTGQGEVVIPGGVYDYCITN | | | |
| PEPASGKMWIAGDGGNQPARYDDFTFEAGK | | | |
| KYTFTMRRAGMGDGTDMEAEDDSPASYTYT | | | |
| VYRDGTKIKEGLTATTFEEDGVAAGNHEYCV | | | |
| EVKYTAGVSPKVCKDVTVEGSNEFAPVQNLT | | | |
| GSSVGQKVTLKWDAPNGTPNPNPNPNPNPGT | | | |
| TLSESFENGIPASWKTIDADGDGHGWKPGNA | | | |
| PGIAGYNSNGCVYSESFGLGGIGVLTPDNYLI | | | |
| TPALDLPNGGKLTFWVCAQDANYASEHYAV | | | |
| YASSTGNDASNFTNALLEETITAKGVRSPKAI | | | |
| RGRIQGTWRQKTVDLPAGTKYVAFRHFQSTD | | | |
| MFYIDLDEVEIKANGGGSPTPTDYTYTVYRD | | | |
| GTKIKEGLTETTFEEDGVATGNHEYCVEVKY | | | |
| TAGVSPKKCVNVTVNggsILAIYSTVASSLVLV | | | |
| VSLGAISFgg | | | |
| MKAILVVLLYTFATANADVYTDHGDLYNTP | SEQ ID NO: 9 | SEQ ID NO: 37 | Kgp_1B_mCat_SP_TM |
| VRMLVVAGAKFKEALKPWLTWKAQKGFYL | | SEQ ID NO: 38 | |
| DVHYTDEAEVGTTNASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYTAHGSETAWADPLL | | | |
| TTSQLKALTNKDKYFLAIGNCSITAQFDYVQP | | | |
| CFGEVITRVKEKGAYAYIGSSPNSYWGEDYY | | | |
| WSVGANAVFGVQPTFEGTSMGSYDATFLEDS | | | |
| YNTVNSIMWAGNLAATHAGNIGNITHIGAHY | | | |
| YWEAYHVLGDGSVMPYRAMPKTNTYTLPAS | | | |
| LPQNQASYSIQASAGSYVAISKDGVLYGTGV | | | |
| ANASGVATVSMTKQITENGNYDVVITRSNYL | | | |
| PVIKQIQVGEPSPYQPVSNLTATTQGQKVTLK | | | |
| WEAPSAKKSEGSREVKRIGDGLFVTIEPANDV | | | |
| RANEAKVVLAADNVWGDNTGYQFLLDADH | | | |
| NTFGSVIPATGPLFTGTASSNLYSANFEYLIPA | | | |
| NADPVVTTQNIIVTGQGEVVIPGGVYDYCITN | | | |
| PEPASGKMWIAGDGGNQPARYDDFTFEAGK | | | |
| KYTFTMRRAGMGDGTDMEAEDDSPASYTYT | | | |
| VYRDGTKIKEGLTATTFEEDGVAAGNHEYCV | | | |
| EVKYTAGVSPKVCKDVTVEGSNEFAPVQNLT | | | |
| GSSVGQKVTLKWDAPNGTPNPNPNPNPNPGT | | | |
| TLSESFPTDYTYTVYRDGTKIKEGLTETTFEE | | | |
| DGVATGNHEYCVEVKYTAGVSPKKCVNVTV | | | |
| NggsILAIYSTVASSLVLVVSLGAISFgg | | | |
| MKAILVVLLYTFATANANTGVSFANYTAHGS | SEQ ID NO: 14 | SEQ ID NO: 47 | Kgp_2A_SP_TM |
| ETAWADPLLTTSQLKALTNKDKPYQPVSNLT | | SEQ ID NO: 48 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQNLTGSSVGQKVTLKWDAPNGTPN | | | |
| PNPNPNPNPGTTLSESFENGIPASWKTIDADG | | | |
| DGHGWKPGNAPGIAGYNSNGCVYSESFGLG | | | |
| GIGVLTPDNYLITPALDLPNGGKLTFWVCAQ | | | |
| DANYASEHYAVYASSTGNDASNFTNALLEET | | | |
| ITAKGVRSPKAIRGRIQGTWRQKTVDLPAGT | | | |
| KYVAFRHFQSTDMFYIDLDEVEIKANGGGSP | | | |
| TPTDYTYTVYRDGTKIKEGLTETTFEEDGVAT | | | |
| GNHEYCVEVKYTAGVSPKKCVNVTVNggsIL | | | |
| AIYSTVASSLVLVVSLGAISFgg | | | |
| MKAILVVLLYTFATANANTGVSFANYTAHGS | SEQ ID NO: 19 | SEQ ID NO: 57 | Kgp_2B_SP_TM |
| ETAWADPLLTTSQLKALTNKDKPYQPVSNLT | | SEQ ID NO: 58 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQNLTGSSVGQKVTLKWDAPNGTPN | | | |
| PNPNPNPNPGTTLSESFPTDYTYTVYRDGTKI | | | |
| KEGLTETTFEEDGVATGNHEYCVEVKYTAGV | | | |
| SPKKCVNVTVNggsILAIYSTVASSLVLVVSLG | | | |
| AISFgg | | | |
| MKAILVVLLYTFATANADVYTDHGDLYNTP | SEQ ID NO: 5 | SEQ ID NO: 29 | Kgp_1A_mCat_mGly_ |
| VRMLVVAGAKFKEALKPWLTWKAQKGFYL | | SEQ ID NO: 30 | SP_TM |
| DVHYTDEAEVGTTQASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYVAHGSETAWADPL | | | |
| LTTSQLKALTNKDKYFLAIGNCSITAQFDYVQ | | | |
| PCFGEVITRVKEKGAYAYIGSSPNSYWGEDY | | | |
| YWSVGANAVFGVQPTFEGTSMGSYDATFLE | | | |
| DSYNTVNSIMWAGNLAATHAGNIGNIVHIGA | | | |
| HYYWEAYHVLGDGSVMPYRAMPKTNTYTLP | | | |
| ASLPQNQASYSIQASAGSYVAISKDGVLYGTG | | | |
| VANAAGVATVSMTKQITENGNYDVVITRSNY | | | |
| LPVIKQIQVGEPSPYQPVSQLTATTQGQKVTL | | | |
| KWEAPSAKKSEGSREVKRIGDGLFVTIEPAND | | | |
| VRANEAKVVLAADNVWGDNTGYQFLLDAD | | | |
| HNTFGSVIPATGPLFTGTASSNLYSANFEYLIP | | | |
| ANADPVVTTQNIIVTGQGEVVIPGGVYDYCIT | | | |
| NPEPASGKMWIAGDGGNQPARYDDFTFEAG | | | |
| KKYTFTMRRAGMGDGTDMEAEDDSPASYTY | | | |
| TVYRDGTKIKEGLTATTFEEDGVAAGNHEYC | | | |
| VEVKYTAGVSPKVCKDVTVEGSNEFAPVQQL | | | |
| TGSSVGQKVTLKWDAPQGTPNPNPNPNPNPG | | | |
| TTLSESFENGIPASWKTIDADGDGHGWKPGN | | | |
| APGIAGYNSNGCVYSESFGLGGIGVLTPDNYL | | | |
| ITPALDLPNGGKLTFWVCAQDANYASEHYAV | | | |
| YASSTGNDASNFANALLEETITAKGVRSPKAI | | | |
| RGRIQGTWRQKTVDLPAGTKYVAFRHFQSTD | | | |
| MFYIDLDEVEIKANGGGSPTPTDYTYTVYRD | | | |
| GTKIKEGLTETTFEEDGVATGNHEYCVEVKY | | | |
| TAGVSPKKCVDVTVNggsILAIYSTVASSLVLV | | | |
| VSLGAISFgg | | | |
| MKAILVVLLYTFATANADVYTDHGDLYNTP | SEQ ID NO: 10 | SEQ ID NO: 39 | Kgp_1B_mCat_mGly_ |
| VRMLVVAGAKFKEALKPWLTWKAQKGFYL | | SEQ ID NO: 40 | SP_TM |
| DVHYTDEAEVGTTQASIKAFIHKKYNDGLAA | | | |
| SAAPVFLALVGDTDVISGEKGKKSKKVTDLY | | | |
| YSAVDGDYFPEMYTFRMSASSPEELTNIIDKV | | | |
| LMYEKATMPDKSYLEKVLLIAGADYSWNSQ | | | |
| VGQPTIKYGMQYYYNQEHGYTDVYNYLKAP | | | |
| YTGCYSHLNTGVSFANYVAHGSETAWADPL | | | |
| LTTSQLKALTNKDKYFLAIGNCSITAQFDYVQ | | | |
| PCFGEVITRVKEKGAYAYIGSSPNSYWGEDY | | | |
| YWSVGANAVFGVQPTFEGTSMGSYDATFLE | | | |
| DSYNTVNSIMWAGNLAATHAGNIGNIVHIGA | | | |
| HYYWEAYHVLGDGSVMPYRAMPKTNTYTLP | | | |
| ASLPQNQASYSIQASAGSYVAISKDGVLYGTG | | | |
| VANAAGVATVSMTKQITENGNYDVVITRSNY | | | |
| LPVIKQIQVGEPSPYQPVSQLTATTQGQKVTL | | | |
| KWEAPSAKKSEGSREVKRIGDGLFVTIEPAND | | | |
| VRANEAKVVLAADNVWGDNTGYQFLLDAD | | | |
| HNTFGSVIPATGPLFTGTASSNLYSANFEYLIP | | | |
| ANADPVVTTQNIIVTGQGEVVIPGGVYDYCIT | | | |
| NPEPASGKMWIAGDGGNQPARYDDFTFEAG | | | |
| KKYTFTMRRAGMGDGTDMEAEDDSPASYTY | | | |
| TVYRDGTKIKEGLTATTFEEDGVAAGNHEYC | | | |
| VEVKYTAGVSPKVCKDVTVEGSNEFAPVQQL | | | |
| TGSSVGQKVTLKWDAPQGTPNPNPNPNPNPG | | | |
| TTLSESFPTDYTYTVYRDGTKIKEGLTETTFEE | | | |
| DGVATGNHEYCVEVKYTAGVSPKKCVDVTV | | | |
| NggsILAIYSTVASSLVLVVSLGAISFgg | | | |
| MKAILVVLLYTFATANANTGVSFANYVAHGS | SEQ ID NO: 15 | SEQ ID NO: 49 | Kgp_2A_mGly_SP_TM |
| ETAWADPLLTTSQLKALTNKDKPYQPVSQLT | | SEQ ID NO: 50 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQQLTGSSVGQKVTLKWDAPQGTPN | | | |
| PNPNPNPNPGTTLSESFENGIPASWKTIDADG | | | |
| DGHGWKPGNAPGIAGYNSNGCVYSESFGLG | | | |
| GIGVLTPDNYLITPALDLPNGGKLTFWVCAQ | | | |
| DANYASEHYAVYASSTGNDASNFANALLEET | | | |
| ITAKGVRSPKAIRGRIQGTWRQKTVDLPAGT | | | |
| KYVAFRHFQSTDMFYIDLDEVEIKANGGGSP | | | |
| TPTDYTYTVYRDGTKIKEGLTETTFEEDGVAT | | | |
| GNHEYCVEVKYTAGVSPKKCVDVTVNggsIL | | | |
| AIYSTVASSLVLVVSLGAISFgg | | | |
| MKAILVVLLYTFATANANTGVSFANYVAHGS | SEQ ID NO: 20 | SEQ ID NO: 59 | Kgp_2B_mGly_SP_TM |
| ETAWADPLLTTSQLKALTNKDKPYQPVSQLT | | SEQ ID NO: 60 | |
| ATTQGQKVTLKWEAPSAKKSEGSREVKRIGD | | | |
| GLFVTIEPANDVRANEAKVVLAADNVWGDN | | | |
| TGYQFLLDADHNTFGSVIPATGPLFTGTASSN | | | |
| LYSANFEYLIPANADPVVTTQNIIVTGQGEVVI | | | |
| PGGVYDYCITNPEPASGKMWIAGDGGNQPAR | | | |
| YDDFTFEAGKKYTFTMRRAGMGDGTDMEAE | | | |
| DDSPASYTYTVYRDGTKIKEGLTATTFEEDG | | | |
| VAAGNHEYCVEVKYTAGVSPKVCKDVTVEG | | | |
| SNEFAPVQQLTGSSVGQKVTLKWDAPQGTPN | | | |
| PNPNPNPNPGTTLSESFPTDYTYTVYRDGTKI | | | |
| KEGLTETTFEEDGVATGNHEYCVEVKYTAGV | | | |
| SPKKCVDVTVNggsILAIYSTVASSLVLVVSLG | | | |
| AISFgg | | | |

In some embodiments, the polypeptide comprises a sequence according to SEQ ID NO: 4, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 9, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 14, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 19, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 5, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 10, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 15, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 20, or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

The N-terminal methionine in any of the above embodiments may be omitted from the polypeptide. Accordingly, in some embodiments, the polypeptide comprises a sequence according to SEQ ID NO: 257 (i.e. SEQ ID NO: 4 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 262 (i.e. SEQ ID NO: 9 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 267 (i.e. SEQ ID NO: 14 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 272 (i.e. SEQ ID NO: 19 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 258 (i.e. SEQ ID NO: 5 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 263 (i.e. SEQ ID NO: 10 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 268 (i.e. SEQ ID NO: 15 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the polypeptide comprises a sequence according to SEQ ID NO: 273 (i.e. SEQ ID NO: 20 without the N-terminal methionine), or sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

In some embodiments, the nucleic acid encoding the polypeptide comprises a sequence according to SEQ ID NO: 27. In some embodiments, the nucleic acid encoding the polypeptide comprises a sequence according to SEQ ID NO: 28.

In some embodiments, the nucleic acid encoding the polypeptide comprises a sequence according to SEQ ID NO: 37. In some embodiments, the nucleic acid encoding the polypeptide comprises a sequence according to SEQ ID NO: 38.

In some embodiments, the nucleic acid encoding the polypeptide comprises a sequence according to SEQ ID NO: 47. In some embodiments, the nucleic acid encoding the polypeptide comprises a sequence according to SEQ ID NO: 48.

In some embodiments, the nucleic acid encoding the polypeptide comprises a sequence according to SEQ ID NO: 57. In some embodiments, the nucleic acid encoding the polypeptide comprises a sequence according to SEQ ID NO: 58.

In some embodiments, the nucleic acid encoding the polypeptide comprises a sequence according to SEQ ID NO: 29. In some embodiments, the nucleic acid encoding the polypeptide comprises a sequence according to SEQ ID NO: 30.

In some embodiments, the nucleic acid encoding the polypeptide comprises a sequence according to SEQ ID NO: 39. In some embodiments, the nucleic acid encoding the polypeptide comprises a sequence according to SEQ ID NO: 40.

In some embodiments, the nucleic acid encoding the polypeptide comprises a sequence according to SEQ ID NO: 49. In some embodiments, the nucleic acid encoding the polypeptide comprises a sequence according to SEQ ID NO: 50.

In some embodiments, the nucleic acid encoding the polypeptide comprises a sequence according to SEQ ID NO: 59. In some embodiments, the nucleic acid encoding the polypeptide comprises a sequence according to SEQ ID NO: 60.

In certain embodiments, the amino acid sequence of the polypeptides of the invention is encoded by a codon-optimized polynucleotide sequence.

### Linkers

In certain embodiments of the disclosure, the secretion signal peptide (SS) sequence or transmembrane domain (TMB) are directly fused to a polypeptide described herein (i.e., there is no linker, such as an amino acid linker, connecting the SS sequence or TMB to the polypeptide described herein). In certain embodiments, the Kgp or RgpA domains of the polypeptides described herein are fused directly to one another (i.e., there is no linker, such as an amino acid linker, connecting the SS sequence or TMB to the polypeptide described herein)

In other embodiments, the SS sequences and TMBs of the disclosure are optionally attached to a polypeptide described herein with a linker. In certain embodiments, the linker is an amino acid linker. In the certain embodiments, the amino acid linker is 1-10 amino acids in length (e.g., the amino acid linker has a length of 1 amino acid, 2 amino acids, 3 amino acids, 4 amino acids, 5 amino acids, 6 amino acids, 7 amino acids, 8 amino acids, 9 amino acids, or 10 amino acids). In other embodiments, the Kgp or RgpA domains of the polypeptides described herein are attached to one another with a linker. In certain embodiments, the linker is an amino acid linker. In the certain embodiments, the amino acid linker is 1-10 amino acids in length (e.g., the amino acid linker has a length of 1 amino acid, 2 amino acids, 3 amino acids, 4 amino acids, 5 amino acids, 6 amino acids, 7 amino acids, 8 amino acids, 9 amino acids, or 10 amino acids).

Illustrative examples of linkers include glycine polymers (Gly) n, where n is an integer of at least one, two, three, four, five, six, seven, or eight; glycine-serine polymers (GlySer) n, where n is an integer of at least one, two, three, four, five, six, seven, or eight; glycine-alanine polymers; alanine-serine polymers; and other flexible linkers known in the art.

Glycine and glycine-serine polymers are relatively unstructured and flexible, and therefore may be able to serve as a neutral tether between the SS sequence and/or TMB and the polypeptides described herein. In certain embodiments, the linker is SGS or GSG. In some embodiments, the linker is GGS and/or GG.

Other exemplary linkers include, but are not limited to, the following amino acid sequences: GGG; DGGGS (SEQ ID NO: 241); TGEKP (SEQ ID NO: 242) (Liu et al. Proc. Natl. Acad. Sci. 94:5525-5530. 1997); GGRR (SEQ ID NO: 243); (GGGGS) n (SEQ ID NO: 244), wherein n=1, 2, 3, 4 or 5 (Kim et al. Proc. Natl. Acad. Sci. 93:1156-1160. 1996); EGKSSGSGSESKVD (SEQ ID NO: 245) (Chaudhary et al. Proc. Natl. Acad. Sci. 87:1066-1070. 1990); KESGSVSSEQLAQFRSLD (SEQ ID NO: 246) (Bird et al. Science. 242:423-426. 1988), GGRRGGGS (SEQ ID NO: 247); LRQRDGERP (SEQ ID NO: 248); LRQKDGGGSERP (SEQ ID NO: 249); and GSTSGSGKPGSGEGSTKG (SEQ ID NO: 250) (Cooper et al. Blood. 101 (4): 1637-1644. 2003). Preferred linkers are shorter, e.g., consisting of 2, 3, 4 or 5 amino acids.

Additional examples of linkers are provided in Chen et al. (Adv Drug Deliv Rev. 65 (10): 1357-1369. 2013), incorporated herein by reference.

### Compositions

The invention provides a composition comprising one or more nucleic acids of the disclosure. The invention also provides a composition comprising one or more polypeptides of the disclosure. A composition of the invention may be a pharmaceutical composition, e.g. comprising a pharmaceutically acceptable carrier, excipient or diluent. In certain embodiments, the composition of the invention is an immunogenic composition. An “immunogenic composition” means a composition comprising a nucleic acid or protein that, when administered to a subject, elicits an immune response, e.g. an antigen-specific immune response. The immune response may be a humoral (antibody) immune response or a cell-mediated immune response. The composition of the invention may be a vaccine composition. Immunogenic compositions (e.g. vaccine compositions) may elicit immunity (e.g. antibody response) against P. gingivalis infection. The antibody response may include antibodies that bind to the surface of P. gingivalis bacteria or its outer membrane vesicles (OMVs) and neutralise the gingipain activities associated with P. gingivalis pathogenicity. The antibodies may be cross-reactive across a range of P. gingivalis strains.

“Protective immunity” or a “protective immune response”, as used herein, refers to immunity or eliciting an immune response against an infectious agent (e.g., P. gingivalis), which is exhibited by a subject, that prevents or ameliorates an infection or reduces at least one symptom thereof. Specifically, induction of protective immunity or a protective immune response from administration of a composition of the invention is evident by elimination or reduction of the presence of one or more symptoms of the P. gingivalis infection (e.g. periodontitis). As used herein, the term “immune response” refers to both the humoral immune response and the cell-mediated immune response. In some embodiments, treatment with a composition of the invention as described herein provides protective immunity against infection by P. gingivalis.

### Nucleic Acid Compositions

In one aspect the invention provides a composition comprising a nucleic acid as described herein comprising a nucleotide sequence encoding a gingipain-based polypeptide as described herein. For example, in one embodiment the composition comprises a nucleic acid as described herein comprising a nucleotide sequence encoding a Kgp-based polypeptide. In another embodiment the composition comprises a nucleic acid as described herein comprising a nucleotide sequence encoding a RgpA-based polypeptide. In a further embodiment, the composition comprises a nucleic acid as described herein comprising a nucleotide sequence encoding a Kgp and RgpA-based polypeptide

In a further aspect, the invention provides a composition comprising (a) a nucleic acid as described herein that comprises a nucleotide sequence encoding a Kgp-based polypeptide as described herein; (b) a nucleic acid as described herein that comprises a nucleotide sequence encoding a RgpA-based polypeptide as described herein. Exemplary combinations are described in and polypeptides comprising sequences having at least 70% (for example 90% or 95%) identity to the sequences referred to in this table may be used as a combination of Kgp-based polypeptides and RgpA-based polypeptides.

The specific Kgp-based polypeptide and Rgp-based polypeptide sequences used in these combinations specified in may be modified, as described elsewhere herein. For instance, the DUF2436 domain may be replaced with a truncated DUF2436 domain, or a DUF2436 domain having at least 70% (e.g. at least 90% or 95%) identity to the DUF2436 domain.

TABLE 27
Examples of combinations Kgp-based and Rgp-based polypeptides

| Kgp-based | | RgpA-based | |
|---|---|---|---|
| polypeptide | | polypeptide | |
| sequence | SEQ ID NO: | sequence | SEQ ID NO: |
| Kgp_1A_mCat_mGly_SP | SEQ ID NO: 3 | RgpA_1B_mCat_mGly_SP | SEQ ID NO: 73 |
| Kgp_1A_mCat_mGly_SP | SEQ ID NO: 3 | RgpA_2B_mGly_SP | SEQ ID NO: 74 |
| Kgp_1B_mCat_mGly_SP | SEQ ID NO: 8 | RgpA_1B_mCat_mGly_SP | SEQ ID NO: 73 |
| Kgp_1B_mCat_mGly_SP | SEQ ID NO: 8 | RgpA_2B_mGly_SP | SEQ ID NO: 74 |
| Kgp_2A_mGly_SP | SEQ ID NO: 13 | RgpA_1B_mCat_mGly_SP | SEQ ID NO: 73 |
| Kgp_2A_mGly_SP | SEQ ID NO: 13 | RgpA_2B_mGly_SP | SEQ ID NO: 74 |
| Kgp_2B_mGly_SP | SEQ ID NO: 18 | RgpA_1B_mCat_mGly_SP | SEQ ID NO: 73 |
| Kgp_2B_mGly_SP | SEQ ID NO: 18 | RgpA_2B_mGly_SP | SEQ ID NO: 74 |
| Kgp_1A_mCat_mGly_SP_G2 | SEQ ID NO: 279 | RgpA_1B_mCat_mGly_SP | SEQ ID NO: 73 |
| Kgp_1A_mCat_mGly_SP_G2 | SEQ ID NO: 279 | RgpA_2B_mGly_SP | SEQ ID NO: 74 |
| Kgp_2A_mGly_SP_G2 | SEQ ID NO: 280 | RgpA_1B_mCat_mGly_SP | SEQ ID NO: 73 |
| Kgp_2A_mGly_SP_G2 | SEQ ID NO: 280 | RgpA_2B_mGly_SP | SEQ ID NO: 74 |
| Kgp_2A_extKas2_mGly_SP_G2 | SEQ ID NO: 297 | RgpA_1B_mCat_mGly_SP | SEQ ID NO: 73 |
| Kgp_2A_ext_Kas2_mGly_SP_G2 | SEQ ID NO: 297 | RgpA_2B_mGly_SP | SEQ ID NO: 74 |
| Kgp_1A_mCat_mGly | SEQ ID NO: 359 | RgpA_1B_mCat | SEQ ID NO: 369 |
| Kgp_1A_mCat_mGly | SEQ ID NO: 359 | RgpA_2B_mGly | SEQ ID NO: 373 |
| Kgp_1B_mCat_mGly | SEQ ID NO: 361 | RgpA_1B_mCat_mGly | SEQ ID NO: 369 |
| Kgp_1B_mCat_mGly | SEQ ID NO: 361 | RgpA_2B_mGly | SEQ ID NO: 373 |
| Kgp_2A_mGly | SEQ ID NO: 363 | RgpA_1B_mCat_mGly | SEQ ID NO: 369 |
| Kgp_2A_mGly | SEQ ID NO: 363 | RgpA_2B_mGly | SEQ ID NO: 373 |
| Kgp_2B_mGly | SEQ ID NO: 365 | RgpA_1B_mCat_mGly | SEQ ID NO: 369 |
| Kgp_2B_mGly | SEQ ID NO: 365 | RgpA_2B_mGly | SEQ ID NO: 373 |
| Kgp_1A_mCat_mGly_G2 | SEQ ID NO: 387 | RgpA_1B_mCat_mGly | SEQ ID NO: 369 |
| Kgp_1A_mCat_mGly_G2 | SEQ ID NO: 387 | RgpA_2B_mGly | SEQ ID NO: 373 |
| Kgp_2A_mGly_G2 | SEQ ID NO: 389 | RgpA_1B_mCat_mGly | SEQ ID NO: 369 |
| Kgp_2A_mGly_G2 | SEQ ID NO: 389 | RgpA_2B_mGly | SEQ ID NO: 373 |
| Kgp_2A_extKas2_mGly_G2 | SEQ ID NO: 399 | RgpA_1B_mCat_mGly | SEQ ID NO: 369 |
| Kgp_2A_ext_Kas2_mGly_G2 | SEQ ID NO: 399 | RgpA_2B_mGly | SEQ ID NO: 373 |

In a particular embodiment, the invention provides a composition comprising:
(a) a first nucleic acid encoding a polypeptide comprising: i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; and vi) a Kgp portion that comprises ABM3; wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
(b) a second nucleic acid encoding a polypeptide comprising: i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

In a particular embodiment, the invention provides a composition comprising:
(a) a first nucleic acid encoding a polypeptide comprising: i) at least a portion of a Kgp catalytic domain, wherein the at least a portion of a Kgp catalytic domain comprises a full-length Kgp catalytic domain; ii) at least a portion of a Kgp DUF2436; iii) at least a portion of a Kgp K1 adhesin domain, wherein the at least a portion of a Kgp K1 adhesin comprises a full-length Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; and vi) a Kgp portion that comprises ABM3; wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
(b) a second nucleic acid encoding a polypeptide comprising: i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

In a particular embodiment, the invention provides a composition comprising:
(a) a first nucleic acid encoding a polypeptide comprising: i) at least a portion of a Kgp catalytic domain, wherein the at least a portion of a Kgp catalytic domain comprises a full-length Kgp catalytic domain; ii) at least a portion of a Kgp DUF2436; iii) at least a portion of a Kgp K1 adhesin domain, wherein the at least a portion of a Kgp K1 adhesin comprises a full-length Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; and vi) a Kgp portion that comprises ABM3; wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2; and wherein the glycosylation site corresponding to the glycosylation site N691-T693 in the WT Kgp sequence (SEQ ID NO: 157) is mutated by a T693A substitution; and wherein the glycosylation site corresponding to the glycosylation site N950-T952 in the WT Kgp sequence (SEQ ID NO: 157) is mutated by a T952A substitution; and
(b) a second nucleic acid encoding a polypeptide comprising: i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

In a particular embodiment, the invention provides a composition comprising:
(a) a first nucleic acid encoding a Kgp-based polypeptide comprising a sequence according to SEQ ID NO: 279, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto; and
(b) a second nucleic acid encoding a RgpA-based polypeptide comprising a sequence according to SEQ ID NO: 73, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

A composition of the invention may comprise a combination as described herein of the nucleic acids as described herein (e.g. they may be formulated in the same composition). The combinations as described herein of the nucleic acids as described herein may alternatively be in two or more separate compositions (e.g. as a combination of compositions for simultaneous, separate or sequential administration, (e.g. in a therapeutic use as described herein)).

A composition of the present disclosure comprising one or more nucleic acids of the present disclosure can also include one or more additional components such as small molecule immunopotentiators (e.g., TLR agonists). A composition of the present disclosure can also include a delivery system for a nucleic acid described herein (e.g. RNA), such as a liposome, an oil-in-water emulsion, or a microparticle. In some embodiments, the composition comprises a lipid nanoparticle (LNP). In certain embodiments, the composition comprises a nucleic acid molecule of the invention encapsulated within an LNP.

### Polypeptide Compositions

In one aspect the invention provides a composition comprising a gingipain-based polypeptide as described herein.

In one aspect the invention provides a composition comprising a gingipain-based polypeptide as described herein. For example, in one embodiment the composition comprises a Kgp-based polypeptide. In another embodiment the composition comprises a RgpA-based polypeptide. In a further embodiment, the composition comprises a Kgp and RgpA-based polypeptide.

In a further aspect, the invention provides a composition comprising (a) a Kgp-based polypeptide as described herein; (b) a RgpA-based polypeptide as described herein. Exemplary combinations are described in and polypeptides comprising sequences having at least 70% (for example 90% or 95%) identity to the sequences referred to in this table may be used as a combination of Kgp-based polypeptides and RgpA-based polypeptides.

In a particular embodiment, the invention provides a composition comprising:
(a) a first polypeptide comprising: i) at least a portion of a Kgp catalytic domain; ii) at least a portion of a Kgp DUF2436; iii) at least a portion of a Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; and vi) a Kgp portion that comprises ABM3; wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
(b) a second polypeptide comprising: i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

In a particular embodiment, the invention provides a composition comprising:
(a) a first polypeptide comprising: i) at least a portion of a Kgp catalytic domain, wherein the at least a portion of a Kgp catalytic domain comprises a full-length Kgp catalytic domain; ii) at least a portion of a Kgp DUF2436; iii) at least a portion of a Kgp K1 adhesin domain, wherein the at least a portion of a Kgp K1 adhesin comprises a full-length Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; and vi) a Kgp portion that comprises ABM3; wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
(b) a second polypeptide comprising: i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

In a particular embodiment, the invention provides a composition comprising:
(a) a first polypeptide comprising: i) at least a portion of a Kgp catalytic domain, wherein the at least a portion of a Kgp catalytic domain comprises a full-length Kgp catalytic domain; ii) at least a portion of a Kgp DUF2436; iii) at least a portion of a Kgp K1 adhesin domain, wherein the at least a portion of a Kgp K1 adhesin comprises a full-length Kgp K1 adhesin domain; iv) a first Kgp portion that comprises an ABM1 and a first Kgp portion that comprises an ABM2; v) a second Kgp portion that comprises an ABM1 and a second Kgp portion that comprises an ABM2; and vi) a Kgp portion that comprises ABM3; wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2; and wherein the glycosylation site corresponding to the glycosylation site N691-T693 in the WT Kgp sequence (SEQ ID NO: 157) is mutated by a T693A substitution; and wherein the glycosylation site corresponding to the glycosylation site N950-T952 in the WT Kgp sequence (SEQ ID NO: 157) is mutated by a T952A substitution; and
(b) a second polypeptide comprising: i) at least a portion of a RgpA catalytic domain; ii) at least a portion of a RgpA DUF2436; iii) at least a portion of a RgpA K1 adhesin domain; iv) a first RgpA portion that comprises an ABM1 and a first RgpA portion that comprises an ABM2; v) a second RgpA portion that comprises an ABM1 and a second RgpA portion that comprises an ABM2; vi) a RgpA portion that comprises ABM3; vii) at least a portion of a RgpA K2 adhesin domain; wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

In a particular embodiment, the invention provides a composition comprising:
(a) a first polypeptide comprising a sequence according to SEQ ID NO: 279, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto; and
(b) a second polypeptide comprising a sequence according to SEQ ID NO: 73, or a sequence that has at least 70% (e.g. at least 75, 80, 85, 90 or 95%; or e.g. at least 75, 80, 85, 86, 87, 88, 89, 90, 91, 92, 93, 94, 95, 96, 97, 98 or 99%) identity thereto.

The specific Kgp-based polypeptide and Rgp-based polypeptide sequences used in these combinations specified in may be modified, as described elsewhere herein. For instance, the DUF2436 domain may be replaced with a truncated DUF2436 domain, or a DUF2436 domain having at least 70% (e.g. at least 90% or 95%) identity to the DUF2436 domain.

A composition of the invention may comprise a combination as described herein of the polypeptides as described herein (e.g. they may be formulated in the same composition). The combinations as described herein of the polypeptides as described herein may alternatively be in two or more separate compositions (e.g. as a combination of compositions for simultaneous, separate or sequential administration, (e.g. in a therapeutic use as described herein)).

A composition of the present disclosure comprising one or more polypeptides of the present disclosure may comprise an adjuvant. As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists (e.g. SPA14, e.g. as described in WO2022090359) and costimulatory molecules. In some embodiments, the adjuvant is AF03 (an oil-in-water squalene-based emulsion adjuvant).

In some embodiments, the adjuvant is selected from the group consisting of: Aluminum based adjuvant (e.g. AlOOH), Squalene based oil in water emulsion adjuvants (e.g. AF03, AS03, MF59) and Liposome-based adjuvants comprising a saponin and a TLR4 agonist (e.g. SPA14, AS01).

### LNPs

In certain embodiments, the composition of the invention (e.g. the composition comprising a nucleic acid of the invention) further comprises a lipid nanoparticle (LNP). In certain embodiments, the nucleic acid of the invention is encapsulated in the LNP.

The LNPs of the disclosure may comprise four categories of lipids: (i) an ionizable lipid (e.g., a cationic lipid); (ii) a PEGylated lipid; (iii) a cholesterol-based lipid, and (iv) a helper lipid.

### A. Ionizable Lipids

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the cationic lipid is OF-02:

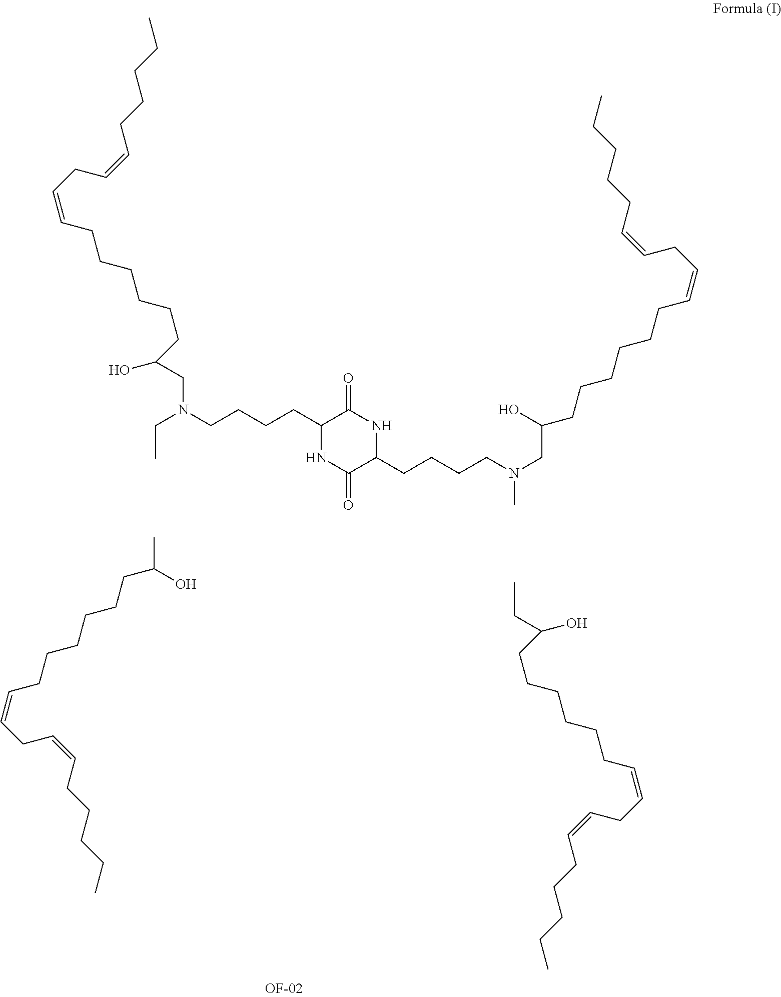 [SMILES: CCCCC/C=C\C/C=C\CCCCCCC(O)CN(C)CCCCC1NC(=O)C(CCCCN(CC)CC(O)CCCCCC/C=C\C/C=C\CCCCC)NC1=O ; CCCCC/C=C\C/C=C\CCCCCCC(C)O ; CCCCC/C=C\C/C=C\CCCCCCC(O)CC]

OF-02 is a non-degradable structural analog of OF-Deg-Lin. OF-Deg-Lin contains degradable ester linkages to attach the diketopiperazine core and the doubly-unsaturated tails, whereas OF-02 contains non-degradable 1,2-amino-alcohol linkages to attach the same diketopiperazine core and the doubly-unsaturated tails (Fenton et al., Adv Mater. (2016) 28:2939; U.S. Pat. No. 10,201,618). An exemplary LNP formulation herein, Lipid A, contains OF-2.

In some embodiments, the cationic lipid is cKK-E10 (Dong et al., PNAS (2014) 111 (11): 3955-60; U.S. Pat. No. 9,512,073):

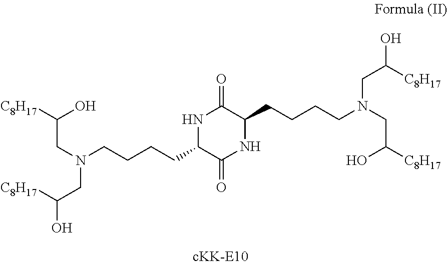

An exemplary LNP formulation herein, Lipid B, contains cKK-E10.

In some embodiments, the cationic lipid is GL-HEPES-E3-E10-DS-3-E18-1 (2-(4-(2-((3-(Bis((Z)-2-hydroxyoctadec-9-en-1-yl)amino)propyl)disulfaneyl)ethyl)piperazin-1-yl)ethyl 4-(bis(2-hydroxydecyl)amino)butanoate) (WO2022/221688), which is a HEPES-based disulfide cationic lipid with a piperazine core, having the Formula III:

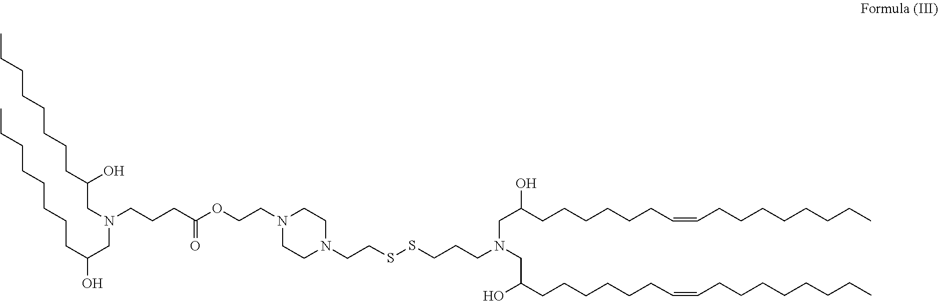 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

An exemplary LNP formulation herein, Lipid C, contains GL-HEPES-E3-E10-DS-3-E18-1. Lipid C has the same composition as Lipid A or Lipid B but for the difference in the cationic lipid.

In some embodiments, the cationic lipid is GL-HEPES-E3-E12-DS-4-E10 (2-(4-(2-((3-(bis(2-hydroxydecyl)amino)butyl)disulfaneyl)ethyl)piperazin-1-yl)ethyl 4-(bis(2-hydroxydodecyl)amino)butanoate) (WO2022/221688), which is a HEPES-based disulfide cationic lipid with a piperazine core, having the Formula IV:

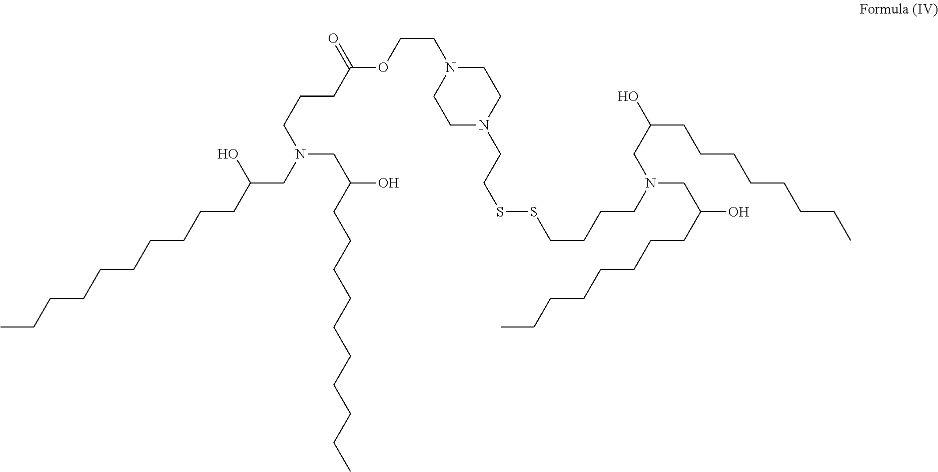 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

An exemplary LNP formulation herein, Lipid D, contains GL-HEPES-E3-E12-DS-4-E10. Lipid D has the same composition as Lipid A or Lipid B but for the difference in the cationic lipid.

In some embodiments, the cationic lipid is GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate) (WO2022/221688), which is a HEPES-based disulfide cationic lipid with a piperazine core, having the Formula V:

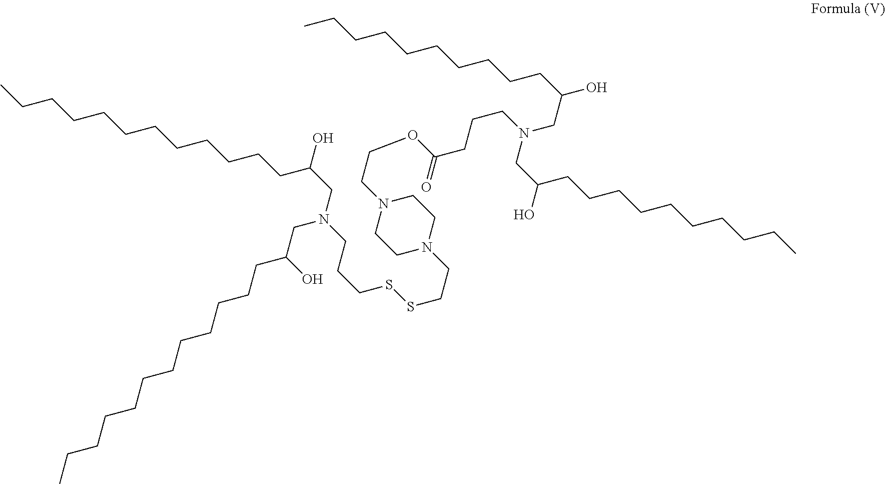 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC]

An exemplary LNP formulation herein, Lipid E, contains GL-HEPES-E3-E12-DS-3-E14. Lipid E has the same composition as Lipid A or Lipid B but for the difference in the cationic lipid.

The cationic lipids GL-HEPES-E3-E10-DS-3-E18-1 (III), GL-HEPES-E3-E12-DS-4-E10 (IV), and GL-HEPES-E3-E12-DS-3-E14 (v) can be synthesized according to the general procedure set out in Scheme 1:

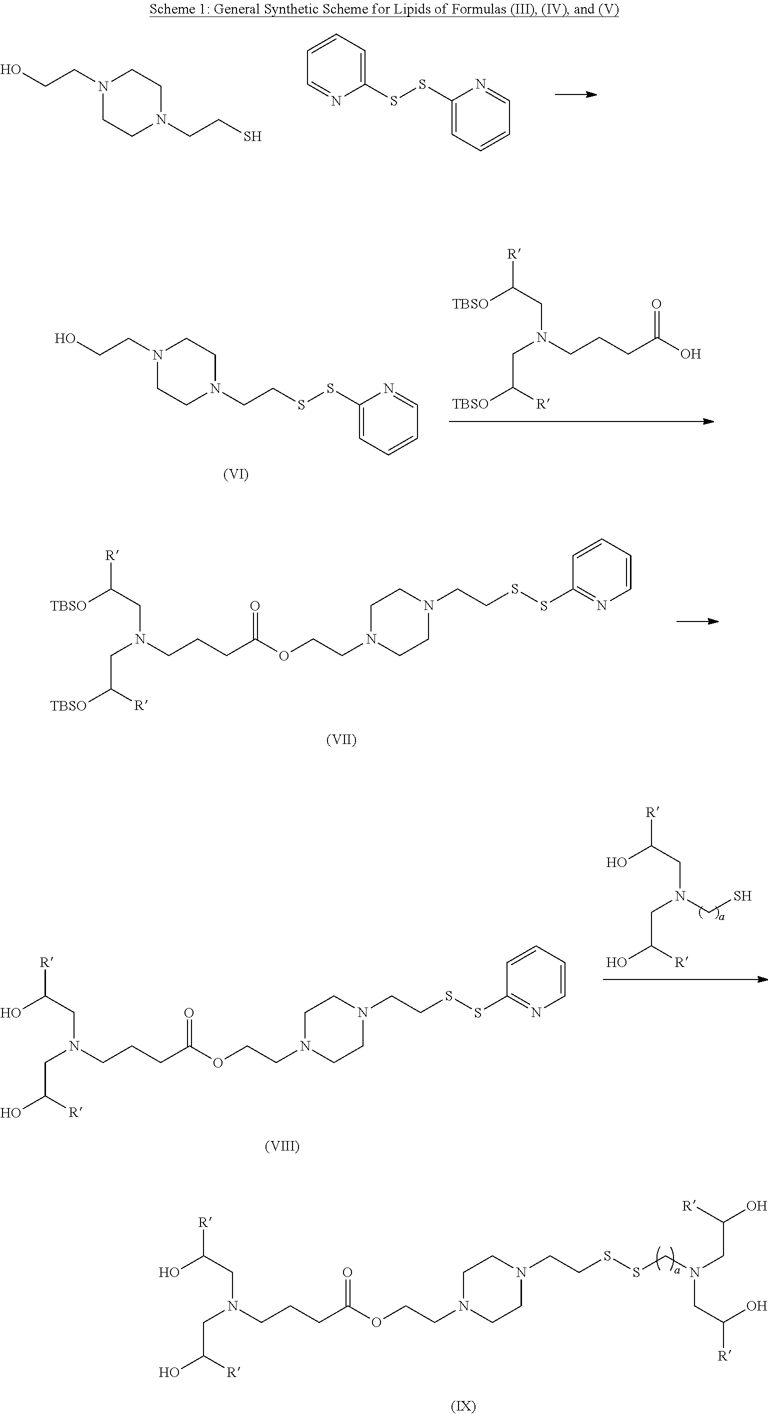

In some embodiments, the cationic lipid is MC3, having the Formula VI:

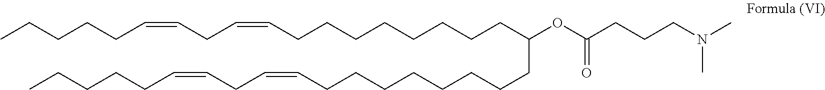 [SMILES: CCCCC/C=C\C/C=C\CCCCCCCCC(CCCCCCCC/C=C\C/C=C\CCCCC)OC(=O)CCCN(C)C]

In some embodiments, the cationic lipid is SM-102 (9-heptadecanyl 8-{(2-hydroxyethyl)[6-oxo-6-(undecyloxy)hexyl]amino}octanoate), having the Formula VII:

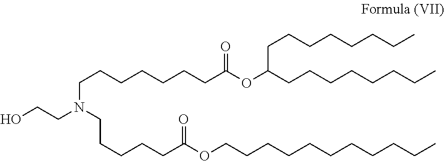 [SMILES: CCCCCCCCCCCOC(=O)CCCCCN(CCO)CCCCCCCC(=O)OC(CCCCCCCC)CCCCCCCC]

In some embodiments, the cationic lipid is ALC-0315 [(4-hydroxybutyl)azanediyl]di(hexane-6,1-diyl) bis(2-hexyldecanoate), having the Formula VIII:

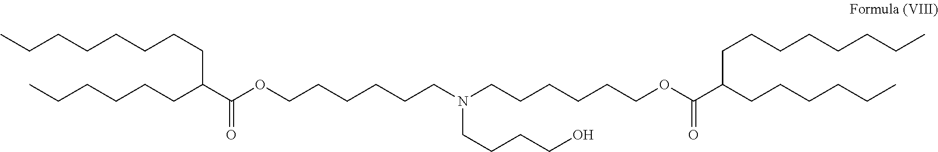 [SMILES: CCCCCCCCC(CCCCCC)C(=O)OCCCCCCN(CCCCO)CCCCCCOC(=O)C(CCCCCC)CCCCCCCC]

In some embodiments, the cationic lipid is cOrn-EE1, having the Formula IX:

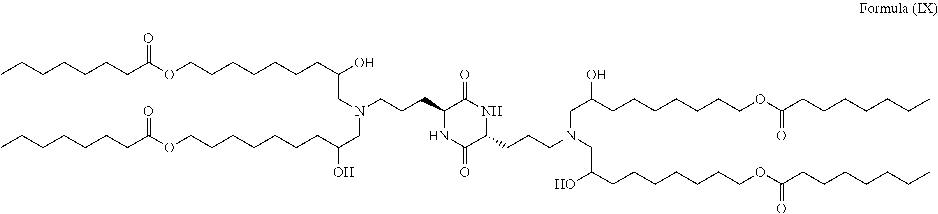 [SMILES: CCCCCCCC(=O)OCCCCCCCC(O)CN(CCC[C@@H]1NC(=O)[C@@H](CCCN(CC(O)CCCCCCCOC(=O)CCCCCCC)CC(O)CCCCCCCOC(=O)CCCCCCC)NC1=O)CC(O)CCCCCCCOC(=O)CCCCCCC]

In some embodiments, the cationic lipid may be selected from the group comprising cKK-E10; OF-02; [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy) heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl] pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3″-(((methylazanediyl) bis(propane-3,1 diyl))bis(azanetriyl))tetrapropionate (306Oi10); decyl(2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); 1,1′-((2-(4-(2-((2-(bis(2-hydroxydodecyl)amino)ethyl) (2-hydroxydodecyl)amino)ethyl)piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa (octan-3-yl) 9,9′,9″,9″,9″ “,9”-((((benzene-1,3,5-tricarbonyl) yris (azanediyl)) tris(propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl) (8-(nonyloxy)-8-oxooctyl)amino) octanoate (Lipid 5); GL-HEPES-E3-E10-DS-3-E18-1; GL-HEPES-E3-E12-DS-4-E10; GL-HEPES-E3-E12-DS-3-E14; and combinations thereof.

In some embodiments, the cationic lipid is IM-001, having the Formula X (EP23306049.0):

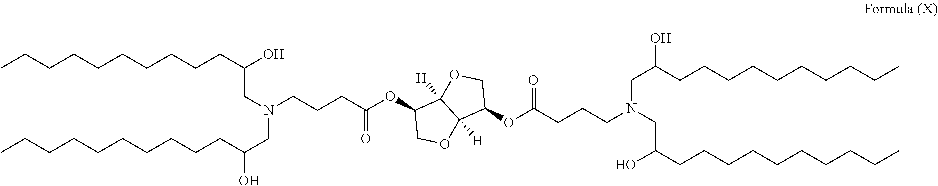 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

An exemplary LNP formulation herein, Lipid G, contains IM-001. Lipid G has the same composition as Lipid A or Lipid B but for the difference in cationic lipid.

The cationic lipid IM-001 (X) can be synthesised according to the general procedure set out in Scheme 2:

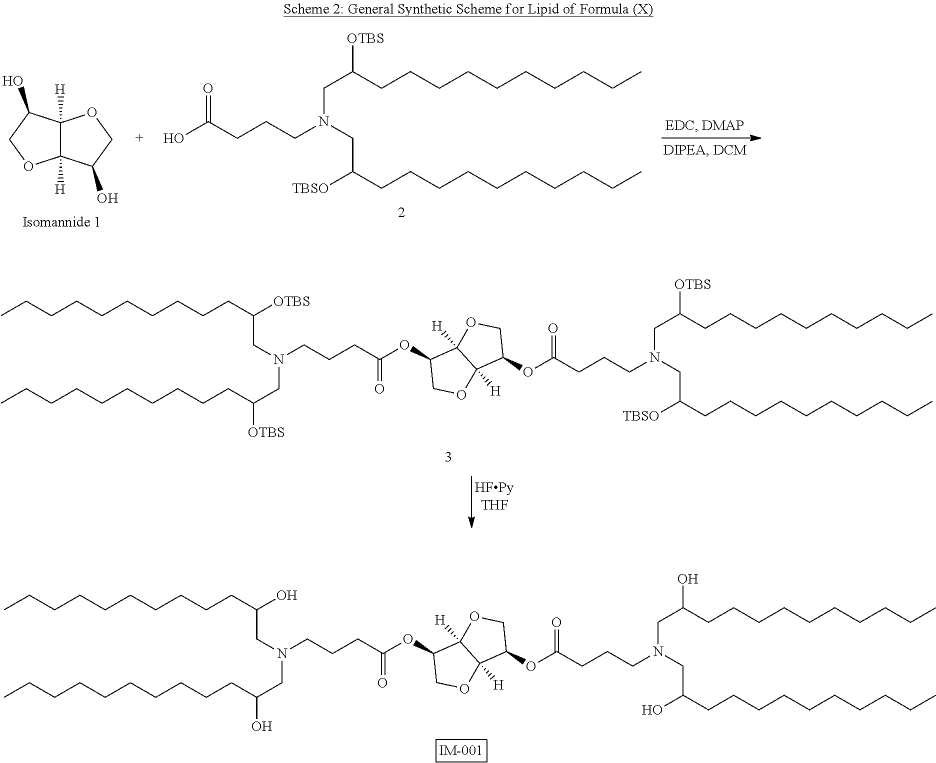

Scheme 2 may be performed as described in Example 2.

In some embodiments, the cationic lipid is IS-001, having the Formula XI (EP23306049.0):

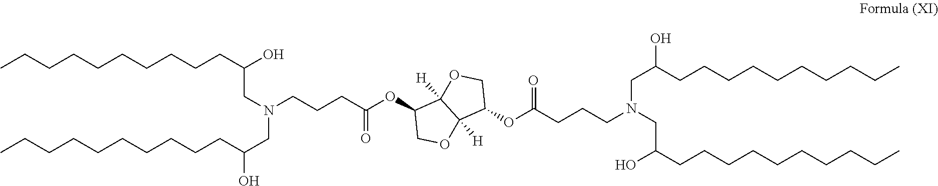 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

An exemplary LNP formulation herein, Lipid H, contains IS-001. Lipid H has the same composition as Lipid A or Lipid B but for the difference in the cationic lipid.

The cationic lipid IS-001 (XI) can be synthesized according to the general procedure set out in Scheme 3:

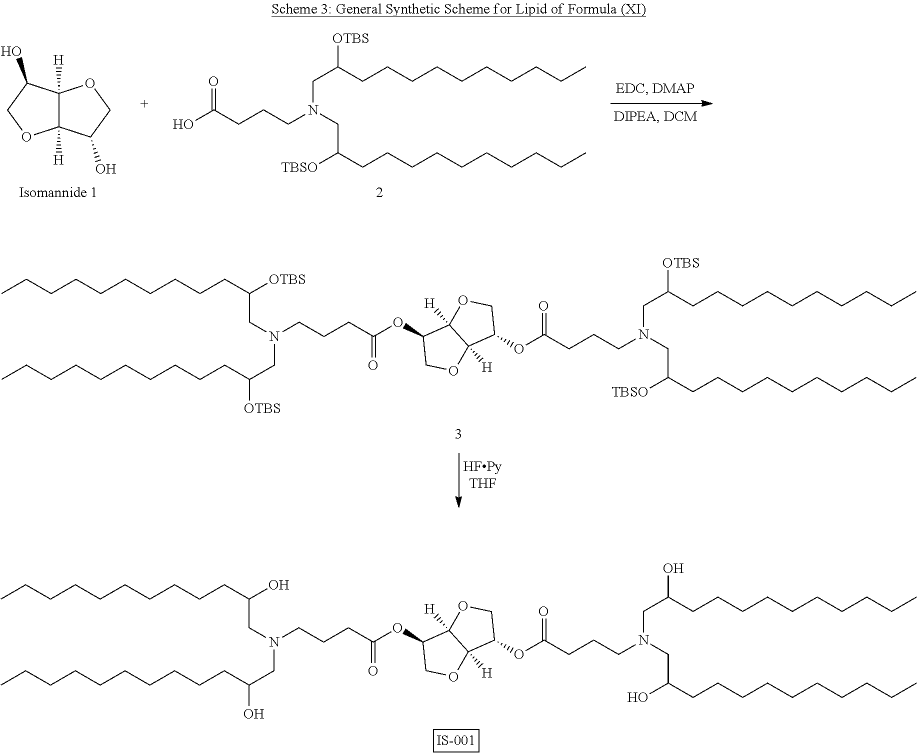

Scheme 3 may be performed as described in Example 3.

In some embodiments, the cationic lipid is biodegradable.

In some embodiments, the cationic lipid is not biodegradable.

In some embodiments, the cationic lipid is cleavable.

In some embodiments, the cationic lipid is not cleavable.

Cationic lipids are described in further detail in Dong et al. (PNAS. 111 (11): 3955-60. 2014); Fenton et al. (Adv Mater. 28:2939. 2016); U.S. Pat. Nos. 9,512,073; and 10,201,618, each of which is incorporated herein by reference.

### B. PEGylated Lipids

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268 (1): 235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or C₁₈) length, such as ceramide a derivatized (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

### C. Cholesterol-Based Lipids

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3ß-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3ß-ol); lathosterol (5a-cholest-7-en-3ß-ol); diosgenin ((3β,25R)-spirost-5-en-3-ol); campesterol (campest-5-en-3ß-ol); campestanol (5a-campestan-3b-ol); 24-methylene cholesterol (5,24 (28)-cholestadien-24-methylen-3ß-ol); cholesteryl margarate (cholest-5-en-3ß-yl heptadecanoate); cholesteryl oleate; cholesteryl stearate and other modified forms of cholesterol. In some embodiments, the cholesterol-based lipid used in the LNPs is cholesterol.

### D. Helper Lipids

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, GL-HEPES-E3-E12-DS-3-E14, IM-001 or IS-001; (ii) DMG-PEG2000; (iii) cholesterol; and (iv) DOPE.

In other embodiments, the present LNPs comprise (i) SM-102; (ii) DMG-PEG2000; (iii) cholesterol; and (iv) DSPC.

In yet other embodiments, the present LNPs comprise (i) ALC-0315; (ii) ALC-0159; (iii) cholesterol; and (iv) DSPC.

### E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A: B: C: D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:
a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);
a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);
a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and
a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),
wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the LNP of the disclosure comprises: a cationic lipid at a molar ratio of 45 to 50%; a PEGylated lipid at a molar ratio of 1.5 to 1.7%; a cholesterol-based lipid at a molar ratio of 38 to 43%; and a helper lipid at a molar ratio of 9 to 10%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: GL-HEPES-E3-E12-DS-3-E14at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises DLin-MC3-DMA (MC3) at a molar ratio of 50%; DMG-PEG2000 at a molar ratio of 1.5%; cholesterol at a molar ratio of 38.5%; and DSPC at a molar ratio of 10%. This LNP formulation is designated “Lipid F” herein. In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

In certain embodiments, the LNP comprises: IM-001 at a molar ratio of 40%; DMG-PEG2000 at a molar ratio of 1.5%; cholesterol at a molar ratio of 28.5%; and DOPE at a molar ratio of 30%. This LNP formulation is designated “Lipid G” herein.

In certain embodiments, the LNP comprises: IS-001 at a molar ratio of 40%; DMG-PEG2000 at a molar ratio of 1.5%; cholesterol at a molar ratio of 28.5%; and DOPE at a molar ratio of 30%. This LNP formulation is designated “Lipid H” herein.

In some embodiments, the LNP formulation is as defined for “Lipid A”, “Lipid B” or “Lipid D”. In some embodiments, the LNP formulation is as defined for “Lipid G” or “Lipid H”.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

F. Nucleic Acids within LNPs

The LNP compositions described herein may comprise a nucleic acid (e.g., a mRNA) of the present invention.

Where desired, the LNP may be multi-valent. In some embodiments, the LNP may carry nucleic acids, such as mRNAs, that encode more than one polypeptide of the present invention, such as two, three, four, five, six, seven, or eight polypeptides. For example, the LNP may carry multiple nucleic acids of the present invention (e.g., mRNA), each encoding a different polypeptide of the invention; or carry a polycistronic mRNA that can be translated into more than one polypeptide of the invention (e.g., each antigen-coding sequence is separated by a nucleotide linker encoding a self-cleaving peptide such as a 2A peptide). An LNP carrying different nucleic acids (e.g., mRNA) typically comprises (encapsulate) multiple copies of each nucleic acid. For example, an LNP carrying or encapsulating two different nucleic acids typically carries multiple copies of each of the two different nucleic acids.

In some embodiments, a single LNP formulation may comprise multiple kinds (e.g., two, three, four, five, six, seven, eight, nine, ten, or more) of LNPs, each kind carrying a different nucleic acid (e.g., mRNA).

When the nucleic acid is mRNA, the mRNA may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridine), 2′-fluoro ribonucleotides, and 2′-methoxy ribonucleotides, and/or phosphorothioate bonds). The mRNA molecule may comprise a 5′ cap and a polyA tail.

### G. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution-thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

### Nucleic Acids

A nucleic acid of the invention may be RNA or DNA. The nucleic acids of the invention may be single or double-stranded. In certain embodiments, the nucleic acid is RNA, e.g. mRNA.

mRNA

In some embodiments, the nucleic acids of the present invention are messenger RNAs (mRNAs). mRNAs can be modified or unmodified. mRNAs may contain one or more coding and non-coding regions. A coding region is alternatively referred to as an open reading frame (ORF). Non-coding regions in an mRNA include the 5′ cap, 5′ untranslated region (UTR), 3′ UTR, and a polyA tail. An mRNA can be purified from natural sources, produced using recombinant expression systems (e.g., in vitro transcription) and optionally purified, or chemically synthesised.

In certain embodiments, the mRNA comprises an ORF encoding an antigen of interest. In certain embodiments, the RNA (e.g., mRNA) further comprises at least one 5′ UTR, 3′ UTR, a poly(A) tail, and/or a 5′ cap.

### 5′ Cap

An mRNA 5′ cap can provide resistance to nucleases found in most eukaryotic cells and promote translation efficiency. Several types of 5′ caps are known. A 7-methylguanosine cap (also referred to as “m′G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, MA; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the invention comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the invention comprises a 5′ cap of:

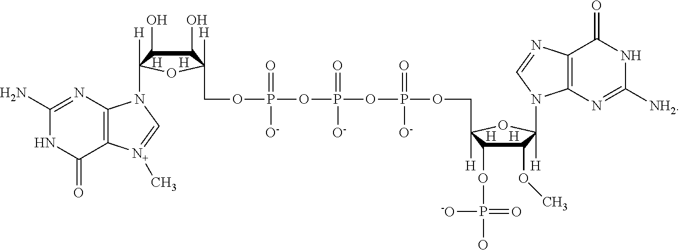

### Untranslated Region (UTR)

In some embodiments, the mRNA of the invention includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 140) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 238 and reproduced below:

(SEQ ID NO: 238)
GGACAGAUCGCCUGGAGACGCCAUCCACGCUGUUUUGACCUCCAUAGAA
GACACCGGGACCGAUCCAGCCUCCGCGGCCGGGAACGGUGCAUUGGAAC
GCGGAUUCCCCGUGCCAAGAGUGACUCACCGUCCUUGACACG.

In some embodiments, the 3′UTR comprises a nucleic acid sequence set forth in SEQ ID NO: 239 and reproduced below:

(SEQ ID NO: 239)
CGGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGCA
UC.

Accordingly, in some instances, the nucleic acid of the present invention comprises a 5′UTR comprising a nucleic acid sequence set forth in SEQ ID NO: 238, a nucleic acid sequence encoding any one of the polypeptides disclosed herein, and a 3′ UTR comprising a nucleic acid sequence set forth in SEQ ID NO: 239.

In some embodiments, the nucleic acid comprises a nucleic acid encoding the polypeptide of SEQ ID NO: 1, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 359, flanked by a 5′ UTR sequence according to SEQ ID NO: 238 and a 3′ UTR sequence according to SEQ ID NO: 239. In some embodiments, the nucleic acid comprises a nucleic acid encoding the polypeptide of SEQ ID NO: 2, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 3, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 4, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 5, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 6, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 361, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 7, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 8, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 9, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 10, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 11, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 363, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 12, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 13, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 14, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 15, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 16, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 365, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 17, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 18, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 19, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 20, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 367, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 369, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 73, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 371, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 373, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 74, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 375, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 377, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 75, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 379, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 381, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 76, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 383, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 385, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 77, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 387, flanked by a 5′ UTR sequence according to SEQ ID NO: 238 and a 3′ UTR sequence according to SEQ ID NO: 239

In some embodiments, the nucleic acid comprises a nucleic acid encoding the polypeptide of SEQ ID NO: 279, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 389, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 280, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 391, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 283, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 393, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 284, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 395, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 285, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 397, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 399, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 297, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 401, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 407, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 405, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 417, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 411, flanked by a 5′ UTR sequence according to SEQ ID NO: 238 and a 3′ UTR sequence according to SEQ ID NO: 239.

In some embodiments, the nucleic acid comprises a nucleic acid encoding the polypeptide of SEQ ID NO: 403, flanked by a 5′ UTR sequence according to SEQ ID NO: 238 and a 3′ UTR sequence according to SEQ ID NO: 239

In some embodiments, the nucleic acid comprises a nucleic acid encoding the polypeptide of SEQ ID NO: 415, flanked by a 5′ UTR sequence according to SEQ ID NO: 238 and a 3′ UTR sequence according to SEQ ID NO: 239

In some embodiments, the nucleic acid comprises a nucleic acid encoding the polypeptide of SEQ ID NO: 409, flanked by a 5′ UTR sequence according to SEQ ID NO: 238 and a 3′ UTR sequence according to SEQ ID NO: 239

The 5′ UTR and 3′UTR are described in further detail in WO2012/075040, incorporated herein by reference.

### Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence

(SEQ ID NO: 240)
AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A) polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

### Chemical Modification

The mRNA disclosed herein may be modified or unmodified. Typically, the mRNA comprises at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25 (2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 rNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### Processes for Making Present LNP Compositions

The present LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and PCT/US2020/043223 (filed Jul. 23, 2020) and can be used to practice the present disclosure. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present disclosure. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm) or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present disclosure is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of between 50% and 99%; or greater than about 60, 65, 70, 75, 80, 85, 90, 92, 95, 98, or 99%. Typically, lipid nanoparticles for use herein have an encapsulation efficiency of at least 90% (e.g., at least 91, 92, 93, 94, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present disclosure contains at least about 0.5 μg, 1 μg, 5 μg, 10 μg, 100 μg, 500 μg, or 1000μ g of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. An exemplary process for making and purifying mRNA is described in Example 1. In this process, in an IVT process, a cDNA template is used to produce an mRNA transcript and the DNA template is degraded by a DNase. The transcript is purified by depth filtration and tangential flow filtration (TFF).

The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 3.5 or 4.5.

An exemplary, nonlimiting process for making an mRNA-LNP composition is described in Example 1. The process involves mixing of a buffered mRNA solution with a solution of lipids in ethanol in a controlled homogeneous manner, where the ratio of lipids: mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

### Vectors

In one aspect, disclosed herein are vectors comprising a nucleic acid disclosed herein. In some embodiments, mRNAs as described herein may be cloned into a vector. Vectors include, but are not limited to, a plasmid, a phagemid, a phage derivative, an animal virus, and a cosmid. Vectors also include expression vectors, replication vectors, probe generation vectors, sequencing vectors, and vectors optimized for in vitro transcription (IVT).

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or nucleic acids disclosed herein. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid or vector disclosed herein.

Vectors can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12 (8): 861-70, or the TransIT-RNA transfection Kit (Mirus, Madison, WI).

Chemical means for introducing a vector into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### Self-Replicating RNA, Trans-Replicating RNA and Non-Replicating RNA

Typically, the nucleic acid molecules described herein are non-replicating RNAs. However, the nucleic acid molecules described herein may alternatively be self-replicating RNAs or trans-replicating RNAs.

### Self-Replicating RNA

Self-replicating (or self-amplifying) RNA can be produced by using replication elements derived from, e.g., alphaviruses, and substituting the structural viral proteins with a nucleotide sequence encoding a protein of interest (e.g., a polypeptide disclosed herein). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) a polypeptide antigen, as disclosed herein. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

### Trans-Replicating RNA

Trans-replicating (or trans-amplifying) RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., a polypeptide described herein). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

### Non-Replicating RNA

Non-replicating (or non-amplifying) RNA is an RNA without the ability to replicate itself.

### Therapeutic Uses

In another aspect, the invention provides the polypeptides, nucleic acids, combinations or compositions of the present invention for use as a medicament. The invention also provides the use of the polypeptides, nucleic acids, combinations or compositions of the present invention for the manufacture of a medicament. The medicament may be used for treating or preventing a disease as described herein. The invention further provides a method of treating or preventing a disease comprising administering the polypeptides, nucleic acids, combinations or compositions of the present invention to a subject in need thereof. The polypeptides, nucleic acids, combinations or compositions of the present invention may, for example, be administered in an amount effective to treat or prevent the disease in the subject. Polypeptides, nucleic acids, combinations or compositions may thus be administered in an effective amount. In some embodiments, the treatment is prophylactic.

In another aspect, the invention provides the polypeptides, nucleic acids, combinations or compositions of the present invention for use in treating or preventing a P. gingivalis infection in a subject. The invention also provides the use of the polypeptides, nucleic acids, combinations or compositions of the present invention for the manufacture of a medicament for treating or preventing a P. gingivalis infection in a subject. The invention further provides a method of treating or preventing a P. gingivalis infection in a subject, the method comprising administering the polypeptides, nucleic acids, combinations or compositions of the present invention to the subject. The polypeptides, nucleic acids, combinations or compositions of the present invention may, for example, be administered in an amount effective to treat or prevent a P. gingivalis infection in the subject (i.e. administered in an effective amount). The polypeptides, nucleic acids, combinations or compositions may be used for generating an immune response against P. gingivalis infection in a subject. In preferred embodiments, the infection is a P. gingivalis infection.

The invention provides the polypeptides, nucleic acids, combinations or compositions of the present invention for use in treating or preventing periodontitis caused by P. gingivalis in a subject. The invention also provides the use of the polypeptides, nucleic acids, combinations or compositions of the present invention for the manufacture of a medicament for treating or preventing periodontitis caused by P. gingivalis in a subject. The invention further provides a method of treating or preventing periodontitis caused by P. gingivalis in a subject, the method comprising administering the polypeptides, nucleic acids, combinations or compositions of the present invention to the subject. The polypeptides, nucleic acids, combinations or compositions of the present invention may, for example, be administered in an amount effective to treat or prevent periodontitis caused by P. gingivalis in the subject (i.e. administered in an effective amount).

The invention provides the polypeptides, nucleic acids, combinations or compositions of the present invention for use in a method of providing protective immunity against a P. gingivalis infection in a subject. The invention also provides the use of the polypeptides, nucleic acids, combinations or compositions of the present invention for the manufacture of a medicament for use in a method of providing protective immunity against a P. gingivalis infection in a subject. The invention further provides a method of providing protective immunity against a P. gingivalis infection in a subject, the method comprising administering the polypeptides, nucleic acids, combinations or compositions of the present invention to the subject. The polypeptides, nucleic acids, combinations or compositions of the present invention may, for example, be administered in an amount effective to providing protective immunity against a P. gingivalis infection in the subject (i.e. administered in an effective amount). In preferred embodiments, the infection is a P. gingivalis infection.

The polypeptides, nucleic acids, combinations or compositions of the invention may elicit an antibody (e.g. IgG) response in a subject, such as a neutralising antibody (IgG) response. The antibodies may be of any isotype (e.g. IgA, IgG, IgM i.e. an a, y or u heavy chain), but will generally be IgG. Within the IgG isotype, antibodies may be IgG1, IgG2, IgG3 or IgG4 subclass. The antibody may have a k or a 2 light chain. A “neutralising antibody” is an antibody which neutralises the biological effects of a P. gingivalis antigen in a subject.

Administration of a polypeptide, nucleic acid, combination or composition of the invention to a subject may enable the subject to produce a P. gingivalis antigen-responsive memory B cell population on exposure to P. gingivalis bacteria or the P. gingivalis antigen.

The polypeptides, nucleic acids, combinations or compositions of the invention may reduce inflammation associated with (e.g. caused by) P. gingivalis infection. The polypeptides, nucleic acids, combinations or compositions of the invention may reduce P. gingivalis-mediated tissue inflammation.

The polypeptides, nucleic acids, combinations or compositions of the invention may inhibit biofilm formation by P. gingivalis. In some embodiments, biofilm formation may be prevented. In some embodiments, biofilm formation may be reduced.

The polypeptides, nucleic acids, combinations or compositions of the invention may be used to induce a primary immune response and/or to boost an immune response.

The polypeptides, nucleic acids, combinations or compositions of the invention may be used in a prime-boost vaccination regime. Protective immunity against a P. gingivalis according to the invention may be provided by administering a priming vaccine, comprising a polypeptide, nucleic acid, combinations or composition of the invention, followed by a booster vaccine. The booster vaccine may be the same as the primer vaccine.

In certain embodiments, the subject is a vertebrate, e.g., a mammal, such as a human or a veterinary mammal (e.g. cat, dog, horse, cow, sheep, cattle, deer, goat, pig, rodents (e.g. mice)). In one aspect, the subject is a mammal such as a primate or a human. In preferred embodiments, the subject is a human. The subject (e.g. the human subject) may be male or female.

### Modes of Administration

The compositions of the present invention can be administered parenterally (e.g., intramuscularly, intradermally, subcutaneously, intraperitoneally, intravenously, or to the interstitial space of a tissue) or by rectal, oral, vaginal, topical, transdermal, intranasal, sublingual, ocular, aural, pulmonary or other mucosal administration. In some embodiments, the compositions of the invention are administered intramuscularly. In some embodiments, the compositions of the invention are delivered by mucosal administration.

In certain embodiments, a composition of the invention is provided for use in intramuscular (IM) injection. The composition can be administered to the thigh or the upper arm of a subject at, e.g., their deltoid muscle in the upper arm. In some embodiments, the composition is provided in a pre-filled syringe or injector (e.g., single-chambered or multi-chambered). Injection may be via a needle (e.g. a hypodermic needle), but needle-free injection may alternatively be used. A typical intramuscular dose is 0.5 ml. In some embodiments, the composition is provided for use in inhalation and is provided in a pre-filled pump, aerosolizer, or inhaler.

The compositions of the invention may be used to elicit systemic and/or mucosal immunity.

Dosage treatment can be a single dose schedule or a multiple dose schedule. Multiple doses (e.g. two or three) may be used in a primary immunisation schedule and/or in a booster immunisation schedule. A primary dose schedule may be followed by a booster dose schedule. Multiple doses (e.g., two doses or three) will typically be administered at least 1 week apart (e.g. about 2 weeks, about 3 weeks, about 4 weeks, about 6 weeks, about 8 weeks, about 10 weeks, about 12 weeks, about 16 weeks, etc.). to subjects in need thereof to achieve the desired prophylactic effects. The doses (e.g., prime and booster doses) may be separated by an interval of e.g., 1 week, 2 weeks, 3 weeks, 4 weeks, one month, two months, three months, four months, five months, six months, one year, two years, five years, or ten years.

A composition of the invention may be in the form of an extemporaneous formulation, e.g. a composition of the invention may be lyophilised. Such compositions may be reconstituted with a physiological buffer (e.g., PBS) just before use. The compositions of the invention may be provided in the form of an aqueous solution or a frozen aqueous solution and can be directly administered to subjects without reconstitution (after thawing, if previously frozen).

In some embodiments of a composition comprising a mRNA as described herein, a single dose of the composition contains 1-50 μg of a mRNA as described herein (e.g., monovalent or multivalent). For example, a single dose may contain about 2.5 μg, about 5 μg, about 7.5 μg, about 10 μg, about 12.5 μg, or about 15 μg of a mRNA described herein e.g. for intramuscular (IM) injection. In further embodiments, a composition of the invention may be provided as a multi-valent single dose contains multiple (e.g., 2, 3, or 4) kinds of LNPs, each for a different antigen, and each kind of LNP has an mRNA amount of, e.g., 2.5 μg, about 5 μg, about 7.5 μg, about 10 μg, about 12.5 μg, or about 15 μg.

In some embodiments, the subject is administered one or more nucleic acid compositions of the present invention. The nucleic acid compositions may comprise a nucleic acid comprising a nucleotide sequence encoding a polypeptide antigen as described herein. The nucleic acid compositions may be administered simultaneously, separately or sequentially. In some embodiments, the subject is administered a nucleic acid combination of the present invention. The nucleic acid combinations include combinations of two or more nucleic acids as described herein. The nucleic acids within a combination may be administered simultaneously, separately or sequentially.

In some embodiments, the subject is administered one or more polypeptide compositions of the present invention. The polypeptide compositions may comprise a polypeptide antigen as described herein. The polypeptide compositions may be administered simultaneously, separately or sequentially. In some embodiments, the subject is administered a polypeptide combination of the present invention. The polypeptide combinations include combinations of two or more polypeptides as described herein. The nucleic acids within a combination may be administered simultaneously, separately or sequentially

In some embodiments, the subject is administered one or more nucleic acid compositions of the present invention and one or more polypeptide compositions of the invention. In some embodiments, the subject is administered a nucleic acid composition comprising a nucleotide sequence encoding a polypeptide of the invention and a polypeptide composition comprising one more polypeptides of the invention. In some embodiments, the subject is administered two or more polypeptide compositions each comprising a polypeptide of the invention. The nucleic acid composition and the one or more polypeptide compositions may be administered simultaneously, separately or sequentially.

Compositions administered separately or sequentially may be administered within 12 months of each other, within six months of each other, or within one month or less of each other (e.g. within 10 days). Compositions may be administered within 7 days, within 3 days, within 2 days, or within 24 hours of each other. Simultaneous administration may involve administering the compositions of the invention at the same time. Simultaneous administration may include administration of the compositions of the invention to a patient within 12 hours of each other, within 6 hours, within 3 hours, within 2 hours or within 1 hour of each other, typically within the same visit to a clinical centre.

The present invention also provides a kit comprising one or more compositions described herein in one or more containers or provides one or more composition as described herein in one or more containers and a physiological buffer for reconstitution in another container. The container(s) may contain a single-use dosage or multi-use dosage. The containers may be pre-treated glass vials or ampules. The kit may include instructions for use.

### Definitions

The term “comprising” encompasses “including” as well as “consisting” e.g. a composition “comprising” X may consist exclusively of X or may include something additional e.g. X+Y.

As used herein, the term “messenger RNA” or “mRNA” refers to a polynucleotide that encodes at least one polypeptide. mRNA as used herein encompasses both modified and unmodified RNA. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a polyA tail. mRNA can be purified from natural sources, produced using recombinant expression systems (e.g., in vitro transcription) and optionally purified, or chemically synthesized.

As used herein, the term “viral secretion signal peptide” or “SS” refers to an amino acid sequence derived from a virus that directs a polypeptide sequence to which it is attached through the cellular secretory pathway. Polypeptides with SS sequences are transited through one or more organelles in the cell until secretion outside of the cell through a secretory vesicle.

As used herein, the term “transmembrane domain” or “TMB” refers to an amino acid sequence that possesses a cell membrane-spanning property. A TMB triggers anchoring of a polypeptide to which it is attached to the cell membrane.

The terms “truncated”, “fragment” or “variant” when referring to the polypeptides of the present disclosure include any polypeptides which retain at least some of the properties (e.g., specific antigenic property of the polypeptide or the ability of polypeptide to contribute to the induction of antibody binding) of the reference polypeptide. Fragments or truncations of polypeptides include N-terminally and/or C-terminally truncated fragments, e.g. C-terminal fragments and N-terminal fragments, as well as deletion fragments but do not include the naturally occurring full-length polypeptide (or mature polypeptide). A deletion fragment or a truncated polypeptide refers to a polypeptide with 1 or more internal amino acids deleted from the full-length polypeptide. Variants of polypeptides include fragments as described above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions. Such variations (i.e. truncations and/or amino acid substitutions, deletions, or insertions) may occur either on the amino acid level or correspondingly on the nucleic acid level.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

As used herein, the term “effective amount” refers to an amount (e.g., of a nucleic acid, a polypeptide, a combination or a composition as described herein) sufficient to effect beneficial or desired results. An effective amount can be administered in one or more administrations, applications or dosages, and is not intended to be limited to a particular formulation or administration route. The term “effective amount” includes, e.g., therapeutically effective amount and/or prophylactically effective amount. The term “effective amount” as used herein refers to an amount (e.g., of a nucleic acid, a polypeptide, a combination or a composition as described herein) which is effective for producing some desired therapeutic or prophylactic effects in the treatment or prevention of an infection, disease, disorder and/or condition at a reasonable benefit/risk ratio applicable to any medical treatment.

Identity with respect to a sequence is defined herein as the percentage of nucleic acid or amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides or the nucleic acids of two polynucleotides. For example, using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. The percent identity can be calculated as: the total number of identical matches multiplied by 100 and then divided by the sum of the length of the longer sequence within the matched span and the number of gaps introduced into the shorter sequences in order to align the two sequences.

As used herein, the term “kit” refers to a packaged set of related components, such as one or more compounds or compositions and one or more related materials such as solvents, solutions, buffers, instructions, or desiccants.

As used herein, the terms “N-terminally” and “C-terminally” are used to describe the position of a first polypeptide sequence or domain relative to a second polypeptide sequence or domain within the same polypeptide chain.

A first polypeptide sequence or domain that is positioned “N-terminally” from a second polypeptide sequence or domain is positioned towards the N-terminus of the full polypeptide chain relative to the second polypeptide sequence or domain. In situations where the first polypeptide sequence or domain overlaps with the second polypeptide sequence or domain, the first polypeptide sequence or domain is positioned N-terminally of the second polypeptide sequence or domain when the N-terminus of the first polypeptide sequence or domain is located towards the N-terminus of the full polypeptide sequence relative to the N-terminus of the second polypeptide sequence or domain.

A first polypeptide sequence or domain that is positioned “C-terminally” from a second polypeptide sequence or domain is positioned towards the C-terminus of the full polypeptide chain relative to the second polypeptide sequence or domain. In situations where the first polypeptide sequence or domain overlaps with the second polypeptide sequence or domain, the first polypeptide sequence or domain is positioned C-terminally of the second polypeptide sequence or domain when the N-terminus of the first polypeptide sequence or domain is located towards the C-terminus of the full polypeptide sequence relative to the N-terminus of the second polypeptide sequence or domain.

The term “adjacent to” means that a first polypeptide sequence or domain is positioned N-terminally or C-terminally to a second polypeptide sequence or domain without any other domains or modules being positioned between the first polypeptide sequence or domain and the second polypeptide sequence or domain. For example, a Kgp catalytic domain may be positioned N-terminally and adjacent to a DUF2436. This means that no other domains or modules as described herein are positioned between the Kgp catalytic domain and the DUF2436. A first polypeptide sequence that is positioned adjacent to a second polypeptide sequence may be joined by a linker sequence.

The term “linked” or “attached” as used herein refers to a first amino acid sequence or nucleotide sequence covalently joined to a second amino acid sequence or nucleotide sequence, respectively (e.g., a secretion signal peptide amino acid sequence and/or a heterologous transmembrane domain amino acid sequence linked to a polypeptide of the invention). The first amino acid or nucleotide sequence can be directly joined to the second amino acid or nucleotide sequence or alternatively an intervening sequence can covalently join the first sequence to the second sequence. The term “linked” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be linked to a second amino acid sequence by a peptide bond or a linker. The first nucleotide sequence can be linked to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The term “linked” is also indicated by a hyphen (-).

### NUMBERED EMBODIMENTS

1. A nucleic acid comprising a nucleotide sequence encoding a polypeptide, wherein the polypeptide comprises:
i) at least a portion of a Porphyromonas gingivalis Lys-specific proteinase (Kgp) catalytic domain;
ii) at least a portion of a Porphyromonas gingivalis Kgp domain of unknown function 2436 (DUF2436);
iii) at least a portion of a Porphyromonas gingivalis Kgp K1 adhesin domain;
iv) a first Porphyromonas gingivalis Kgp portion that comprises an adhesin binding motif 1 (ABM1) and a first Porphyromonas gingivalis Kgp portion that comprises an adhesin binding motif 2 (ABM2);
v) a second Porphyromonas gingivalis Kgp portion that comprises an ABM1 and a second Porphyromonas gingivalis Kgp portion that comprises an ABM2; and
vi) a Porphyromonas gingivalis Kgp portion that comprises an ABM3.

2. The nucleic acid of embodiment 1, wherein the first Kgp portion that comprises an ABM1 comprises a sequence according to SEQ ID NO: 120 or a sequence that has at least 70% (e.g. at least 90 or 95%) identity thereto.

3. The nucleic acid of embodiment 1 or embodiment 2, wherein the first Kgp portion that comprises an ABM2 comprises a sequence according to SEQ ID NO: 130 or a sequence that has at least 70% (e.g. at least 90 or 95%) identity thereto.

4. The nucleic acid of any preceding embodiment, wherein the second Kgp portion that comprises an ABM1 comprises a sequence according to SEQ ID NO: 120 or a sequence that has at least 70% (e.g. at least 90 or 95%) identity thereto.

5. The nucleic acid of any preceding embodiment, wherein the second Kgp portion that comprises an ABM2 comprises a sequence according to SEQ ID NO: 130 or a sequence that has at least 70% (e.g. at least 90 or 95%) identity thereto.

6 The nucleic acid of any preceding embodiment, wherein the first Kgp portion that comprises an ABM1 comprises a sequence according to SEQ ID NO: 89 or a sequence that has at least 70% (e.g. at least 90 or 95%) identity thereto.

7. The nucleic acid of any preceding embodiment, wherein the first Kgp portion that comprises an ABM2 comprises a sequence according to SEQ ID NO: 91 or a sequence that has at least 70% (e.g. at least 90 or 95%) identity thereto.

8. The nucleic acid of any preceding embodiment, wherein the second Kgp portion that comprises an ABM1 comprises a sequence according to SEQ ID NO: 92 or a sequence that has at least 70% (e.g. at least 90 or 95%) identity thereto.

9. The nucleic acid of any preceding embodiment, wherein the second Kgp portion that comprises an ABM2 comprises a sequence according to SEQ ID NO: 95 or a sequence that has at least 70% (e.g. at least 90 or 95%) identity thereto.

10. The nucleic acid of any preceding embodiment, wherein the first Kgp portion that comprises an ABM1 and the first domain Kgp that comprises ABM2 are positioned N-terminally of the second Kgp portion that comprises ABM1 and the second Kgp portion that comprises ABM2.

11. The nucleic acid of any preceding embodiment, wherein the first Kgp portion that comprises an ABM1 is positioned N-terminally of the at least a portion of the Kgp DUF2436 and the first Kgp portion that comprises an ABM2 is positioned C-terminally of the at least a portion of the Kgp DUF2436, for example wherein the Kgp portion that comprises an ABM1 is positioned N-terminally and adjacent to the at least a portion of the Kgp DUF2436 and the first Kgp portion that comprises an ABM2 is positioned C-terminally and adjacent to the at least a portion of the Kgp DUF2436.

12. The nucleic acid of any preceding embodiment, wherein the second Kgp portion that comprises an ABM1 is positioned N-terminally of the at least a portion of the Kgp K1 adhesin domain and the second Kgp portion that comprises an ABM2 is positioned C-terminally of the at least a portion of the Kgp K1 adhesin domain.

13. The nucleic acid of any preceding embodiment, wherein the at least a portion of Kgp DUF2436 is a full-length Kgp DUF2436.

14. The nucleic acid of embodiment 13, wherein the full-length Kgp DUF2436 comprises a sequence according to SEQ ID NO: 90 or a sequence that has at least 70% (e.g. at least 90 or 95%) identity thereto.

15. The nucleic acid of any preceding embodiment, wherein the Kgp DUF2436 is positioned C-terminally of the Kgp catalytic domain.

16. The nucleic acid of any preceding embodiment, wherein the Kgp DUF2436 is positioned N-terminally of the Kgp K1 adhesin domain.

17. The nucleic acid of any preceding embodiment, wherein the at least a portion of Kgp catalytic domain is a full-length Kgp catalytic domain.

18. The nucleic acid of embodiment 17, wherein the full-length Kgp catalytic domain comprises a sequence according to SEQ ID NO: 64 or a sequence that has at least 70% (e.g. at least 90 or 95%) identity thereto.

19. The nucleic acid of any one of embodiments 1-16, wherein the at least a portion of Kgp catalytic domain is a truncated Kgp catalytic domain.

20. The nucleic acid of embodiment 19, wherein the truncated Kgp catalytic domain comprises a Lys-gingipain active site peptide (KAS peptide).

21. The nucleic acid of embodiment 20, wherein the KAS peptide comprises a Kas2 peptide according to SEQ ID NO: 61 or a sequence that has at least 70% (e.g. at least 90 or 95%) identity thereto.

22. The nucleic acid of embodiment 20, wherein the KAS peptide comprises an extended Kas2 peptide according to SEQ ID NO: 88 or a sequence that has at least 70% (e.g. at least 90 or 95%) identity thereto.

23. The nucleic acid of any preceding embodiment, wherein the Kgp catalytic domain comprises a mutation that inactivates proteinase activity, wherein optionally the mutation that inactivates proteinase activity is a cysteine to serine mutation at position 477 (C₄₇₇S), wherein the mutation position corresponds to position 477 of the wild-type Kgp sequence of SEQ ID NO: 157.

24. The nucleic acid of any preceding embodiment, wherein the polypeptide further comprises at least a portion of the Porphyromonas gingivalis Arg-specific proteinase A (RgpA) or Arg-specific proteinase B (RgpB) catalytic domain, for example at least a portion of the Porphyromonas gingivalis Arg-specific proteinase A (RgpA).

25. The nucleic acid of embodiment 24, wherein the at least a portion of RgpA or RgpB catalytic domain is a truncated RgpA or RgpB catalytic domain.

26. The nucleic acid of embodiment 25, wherein the truncated RgpA or RgpB catalytic domain comprises of a Arg-gingipain active site peptide (RAS peptide).

27. The nucleic acid of embodiment 26, wherein the RAS peptide comprises a Ras2 peptide according to SEQ ID NO: 166 or a sequence that has at least 70% (e.g. at least 90 or 95%) identity thereto 28. The nucleic acid of embodiment 26, wherein the RAS peptide comprises an extended Ras2 peptide according to SEQ ID NO: 97 or a sequence that has at least 70% (e.g. at least 90 or 95%) identity thereto.

29. The nucleic acid of any one of embodiments 24-28, wherein the at least a portion of the RgpA or RgpB catalytic domain is positioned at the C-terminus of the polypeptide.

30. The nucleic acid of any preceding embodiment, wherein the polypeptide further comprises at least a portion of a Porphyromonas gingivalis Kgp K2 adhesin domain.

31. The nucleic acid of embodiment 30, wherein the at least a portion of Kgp K2 adhesin domain is a full-length K2 adhesin domain.

32. The nucleic acid of embodiment 31, wherein the full-length Kgp K2 adhesin domain comprises a sequence according to SEQ ID NO: 96 or a sequence that has at least 70% (e.g. at least 90 or 95%) identity thereto.

33. The nucleic acid of any one of embodiments 30-32, wherein the at least a portion of Kgp K2 adhesin domain is positioned C-terminally of the Kgp K1 adhesin domain, for example C-terminally and adjacent to the Kgp K1 adhesin domain.

34. The nucleic acid of any one of embodiments 30-33, wherein the at least a portion of Kgp K2 adhesin domain is positioned between the second Kgp portion that comprises an ABM1 and the second Kgp portion that comprises an ABM2.

35. The nucleic acid of 1-29, wherein the polypeptide further comprises at least a portion of a Porphyromonas gingivalis RgpA K2 adhesin domain.

36. The nucleic acid of embodiment 35, wherein the at least a portion of RgpA K2 adhesin domain is a full-length K2 adhesin domain.

37. The nucleic acid of embodiment 36, wherein the full-length RgpA K2 adhesin domain comprises a sequence according to SEQ ID NO: 104 or a sequence that has at least 70% (e.g. at least 90 or 95%) identity thereto.

38. The nucleic acid of any one of embodiments 35-37, wherein the at least a portion of RgpA K2 adhesin domain is positioned C-terminally of the Kgp K1 adhesin domain, for example C-terminally and adjacent to the Kgp K1 adhesin domain.

39. The nucleic acid of any one of embodiments 35-38, wherein the at least a portion of RgpA K2 adhesin domain is positioned between the second Kgp portion that comprises an ABM1 and the second Kgp portion that comprises an ABM2.

40. The nucleic acid of embodiment any one of embodiments 24-39, wherein the polypeptide comprises at least a portion of an RgpA DUF2436.

41. The nucleic acid of embodiment 40, wherein the RgpA DUF2436 is a full-length RgpA DUF2436.

42. The nucleic acid of embodiment 41, wherein the full-length RgpA DUF2436 comprises a sequence according to SEQ ID NO: 100 or a sequence that has at least 70% (e.g. at least 90 or 95%) identity thereto.

43. The nucleic acid of any one of embodiments 40-42, wherein the polypeptide comprises a first Porphyromonas gingivalis RgpA portion that comprises an ABM1, and a first Porphyromonas gingivalis RgpA portion that comprises an ABM2.

44. The nucleic acid of embodiment 43, wherein the first RgpA portion that comprises an ABM1 comprises a sequence according to SEQ ID NO: 99 or a sequence that has at least 70% (e.g. at least 90 or 95%) identity thereto.

45. The nucleic acid of embodiment 43 or embodiment 44, wherein the first RgpA portion that comprises an ABM2 comprises a sequence according to SEQ ID NO: 101 or a sequence that has at least 70% (e.g. at least 90 or 95%) identity thereto.

46. The nucleic acid of any one of embodiments 43-45, wherein the first RgpA portion that comprises an ABM1 is positioned N-terminally of the at least a portion of the RgpA DUF2436 and the first RgpA portion that comprises an ABM2 is positioned C-terminally of the at least a portion of the RgpA DUF2436, for example wherein the first RgpA portion that comprises an ABM1 is positioned N-terminally and adjacent to the at least a portion of the RgpA DUF2436 and the first RgpA portion that comprises an ABM2 is positioned C-terminally and adjacent to the at least a portion of the RgpA DUF2436.

47. The nucleic acid of any preceding embodiment, wherein the Kgp portion comprising ABM3 comprises a sequence according to SEQ ID NO: 139 or a sequence that has at least 70% (e.g. at least 90 or 95%) identity thereto.

48. The nucleic acid of any preceding embodiment, wherein the at least a portion of the Kgp K1 adhesin domain is a full-length Kgp K1 adhesin domain.

49. The nucleic acid of embodiment 48, wherein the full-length Kgp K1 adhesin domain and Kgp portion comprising ABM3 together comprise a sequence according to SEQ ID NO: 93 or a sequence that has at least 70% (e.g. at least 90 or 95%) identity thereto.

50. The nucleic acid of any one of embodiments 1-48, wherein the at least a portion of the Kgp K1 adhesin domain is a truncated Kgp K1 adhesin domain.

51. The nucleic acid of embodiment 50, wherein the truncated Kgp K1 adhesin domain and Kgp portion comprising ABM3 together comprise a sequence according to SEQ ID NO: 94 or a sequence that has at least 70% (e.g. at least 90 or 95%) identity thereto.

52. The nucleic acid of any one of embodiments 1-51, wherein the polypeptide comprises a sequence according to SEQ ID NO:1 or a sequence that has at least 70% (e.g. at least 90 or 95%) identity thereto.

53. The nucleic acid of any one of embodiments 1-51, wherein the polypeptide comprises a sequence according to SEQ ID NO: 6 or a sequence that has at least 70% (e.g. at least 90 or 95%) identity thereto.

54. The nucleic acid of any one of embodiments 1-51, wherein the polypeptide comprises a sequence according to SEQ ID NO: 11 or a sequence that has at least 70% (e.g. at least 90 or 95%) identity thereto.

55. The nucleic acid of any one of embodiments 1-51, wherein the polypeptide comprises a sequence according to SEQ ID NO: 16 or a sequence that has at least 70% (e.g. at least 90 or 95%) identity thereto.

56. The nucleic acid of any one of embodiments 1-51, wherein the polypeptide comprises a sequence according to SEQ ID NO: 375 or a sequence that has at least 70% (e.g. at least 90 or 95%) identity thereto.

57. The nucleic acid of any one of embodiments 1-51, wherein the polypeptide comprises a sequence according to SEQ ID NO: 379 or a sequence that has at least 70% (e.g. at least 90 or 95%) identity thereto.

58. The nucleic acid of any one of embodiments 1-51, wherein the polypeptide comprises a sequence according to SEQ ID NO: 383 or a sequence that has at least 70% (e.g. at least 90 or 95%) identity thereto.

59. The nucleic acid of any one of embodiments 1-51, wherein the polypeptide comprises a sequence according to SEQ ID NO: 397 or a sequence that has at least 70% (e.g. at least 90 or 95%) identity thereto.

60. The nucleic acid of any one of embodiments 1-51, wherein the polypeptide comprises a sequence according to SEQ ID NO: 403 or a sequence that has at least 70% (e.g. at least 90 or 95%) identity thereto.

61. The nucleic acid of any one of embodiments 1-51, wherein the polypeptide comprises a sequence according to SEQ ID NO: 415 or a sequence that has at least 70% (e.g. at least 90 or 95%) identity thereto.

62. The nucleic acid of any one of embodiments 1-51, wherein the polypeptide comprises a sequence according to SEQ ID NO: 409 or a sequence that has at least 70% (e.g. at least 90 or 95%) identity thereto.

63. A nucleic acid comprising a nucleotide sequence encoding a polypeptide, wherein the polypeptide comprises:
at least a portion of a Porphyromonas gingivalis Arg-specific proteinase A (RgpA) or Arg-specific proteinase B (RgpB) catalytic domain, for example at least a portion of the Porphyromonas gingivalis Arg-specific proteinase A (RgpA) catalytic domain;
ii) at least a portion of a Porphyromonas gingivalis RgpA DUF2436;
iii) at least a portion of a Porphyromonas gingivalis RgpA K1 adhesin domain;
iv) a first Porphyromonas gingivalis RgpA portion that comprises an ABM1, and a first Porphyromonas gingivalis RgpA portion that comprises an ABM2;
v) a second Porphyromonas gingivalis RgpA portion that comprises an ABM1, and a second Porphyromonas gingivalis RgpA portion that comprises an ABM2; and
vi) a Porphyromonas gingivalis RgpA portion that comprises an ABM3.

64. The nucleic acid of embodiment 63, wherein the first RgpA portion that comprises an ABM1 comprises a sequence according to SEQ ID NO: 120 or a sequence that has at least 70% (e.g. at least 90 or 95%) identity thereto.

65. The nucleic acid of embodiment 63 or embodiment 64, wherein the first RgpA portion that comprises an ABM2 comprises a sequence according to SEQ ID NO: 130 or a sequence that has at least 70% (e.g. at least 90 or 95%) identity thereto.

66. The nucleic acid of any one of embodiments 63-65, wherein the second RgpA portion that comprises an ABM1 comprises a sequence according to SEQ ID NO: 120 or a sequence that has at least 70% (e.g. at least 90 or 95%) identity thereto.

67. The nucleic acid of any one of embodiments 63-66, wherein the second RgpA portion that comprises an ABM2 comprises a sequence according to SEQ ID NO: 130 or a sequence that has at least 70% (e.g. at least 90 or 95%) identity thereto.

68. The nucleic acid of any one of embodiments 63-67, wherein the first RgpA portion that comprises an ABM1 comprises a sequence according to SEQ ID NO: 99 or a sequence that has at least 70% (e.g. at least 90 or 95%) identity thereto.

69. The nucleic acid of any one of embodiments 63-68, wherein the first RgpA portion that comprises an ABM2 comprises a sequence according to SEQ ID NO: 101 or a sequence that has at least 70% (e.g. at least 90 or 95%) identity thereto.

70. The nucleic acid of any one of embodiments 63-69, wherein the second RgpA portion that comprises an ABM1 comprises a sequence according to SEQ ID NO: 102 or a sequence that has at least 70% (e.g. at least 90 or 95%) identity thereto.

71. The nucleic acid of any one of embodiments 63-70, wherein the second RgpA portion that comprises an ABM2 comprises a sequence according to SEQ ID NO: 105 or a sequence that has at least 70% (e.g. at least 90 or 95%) identity thereto.

72. The nucleic acid of any one of embodiments 63-71, wherein the first RgpA portion that comprises an ABM1 and the first domain RgpA that comprises an ABM2 are positioned N-terminally of the second RgpA portion that comprises an ABM1 and the second RgpA portion that comprises an ABM2.

73. The nucleic acid of any one of embodiments 63-72, wherein the first RgpA portion that comprises an ABM1 is positioned N-terminally of the at least a portion of the RgpA DUF2436 and the first RgpA portion that comprises an ABM2 is positioned C-terminally of the at least a portion of the RgpA DUF2436, for example wherein the RgpA portion that comprises an ABM1 is positioned N-terminally and adjacent to the at least a portion of the RgpA DUF2436 and the first RgpA portion that comprises an ABM2 is positioned C-terminally and adjacent to the at least a portion of the RgpA DUF2436.

74. The nucleic acid of any one of embodiments 63-73, wherein the second RgpA portion that comprises an ABM1 is positioned N-terminally of the at least a portion of the RgpA K1 adhesin domain and the second RgpA portion that comprises an ABM2 is positioned C-terminally of the at least a portion of the RgpA K1 adhesin domain.

75. The nucleic acid of any one of embodiments 63-74, wherein the at least a portion of RgpA DUF2436 is a full-length RgpA DUF2436.

76. The nucleic acid of embodiment 75, wherein the full-length RgpA DUF2436 comprises a sequence according to SEQ ID NO: 100 or a sequence that has at least 70% (e.g. at least 90 or 95%) identity thereto.

77. The nucleic acid of any one of embodiments 63-76, wherein the RgpA DUF2436 is positioned C-terminally of the at least a portion of RgpA or RgpB catalytic domain.

78. The nucleic acid of any one of embodiments 63-76, wherein the at least a portion of the RgpA or RgpB catalytic domain is positioned at the C-terminus of the polypeptide.

79. The nucleic acid of any one of embodiments 63-78, wherein the RgpA DUF2436 is positioned N-terminally of the RgpA K1 adhesin domain.

80. The nucleic acid of any one of embodiments 63-79, wherein the at least a portion of RgpA or RgpB catalytic domain is a full-length RgpA or RgpB catalytic domain.

81. The nucleic acid of embodiment 80, wherein the full-length RgpA catalytic domain comprises a sequence according to SEQ ID NO: 98 or a sequence that has at least 70% (e.g. at least 90 or 95%) identity thereto.

82. The nucleic acid of any one of embodiments 63-79, wherein the at least a portion of RgpA or RgpB catalytic domain is a truncated RgpA or RgpB catalytic domain.

83. The nucleic acid of embodiment 82, wherein the truncated RgpA or RgpB catalytic domain comprises a Arg-gingipain active site peptide (RAS peptide).

84. The nucleic acid of embodiment 83, wherein the RAS peptide comprises a Ras2 peptide according to SEQ ID NO: 166 or a sequence that has at least 70% (e.g. at least 90 or 95%) identity thereto.

85. The nucleic acid of embodiment 83, wherein the RAS peptide comprises an extended Ras2 peptide according to SEQ ID NO: 97 or a sequence that has at least 70% (e.g. at least 90 or 95%) identity thereto.

86. The nucleic acid of any one of embodiments 63-85, wherein the RgpA or RgpB catalytic domain comprises a mutation that inactivates proteinase activity, wherein optionally the mutation that inactivates proteinase activity is a cysteine to serine mutation at position 471 (C₄₇₁S), wherein the mutation position corresponds to position 471 of the wild-type RgpA sequence of SEQ ID NO: 158.

87. The nucleic acid of any one of embodiments 63-86, wherein the polypeptide further comprises at least a portion of a Porphyromonas gingivalis RgpA K2 adhesin domain.

88. The nucleic acid of embodiment 87, wherein the at least a portion of RgpA K2 adhesin domain is a full-length K2 adhesin domain.

89. The nucleic acid of embodiment 88, wherein the full-length RgpA K2 adhesin domain comprises a sequence according to SEQ ID NO: 104 or a sequence that has at least 70% (e.g. at least 90 or 95%) identity thereto.

90. The nucleic acid of embodiment 88 or embodiment 89, wherein the at least a portion of RgpA K2 adhesin domain is positioned C-terminally of the RgpA K1 adhesin domain, for example C-terminally and adjacent to the RgpA K1 adhesin domain.

91. The nucleic acid of any one of embodiments 88-90, wherein the at least a portion of RgpA K2 adhesin domain is positioned between the second RgpA portion that comprises an ABM1 and the second RgpA portion that comprises an ABM2.

92. The nucleic acid of any one of embodiments 63-91, wherein the RgpA portion comprising an ABM3 comprises a sequence according to SEQ ID NO: 139 or a sequence that has at least 70% (e.g. at least 90 or 95%) identity thereto.

93. The nucleic acid of any one of embodiments 63-92, wherein the at least a portion of the RgpA K1 adhesin domain is a truncated RgpA K1 adhesin domain.

94. The nucleic acid of embodiment 93, wherein the truncated RgpA K1 adhesin domain and RgpA portion comprising an ABM3 together comprise a sequence according to SEQ ID NO: 103 or a sequence that has at least 70% (e.g. at least 90 or 95%) identity thereto.

95. The nucleic acid of any one of embodiments 63-94, wherein the polypeptide comprises a sequence according to SEQ ID NO: 367 or a sequence that has at least 70% (e.g. at least 90 or 95%) identity thereto.

96. The nucleic acid of any one of embodiments 63-94, wherein the polypeptide comprises a sequence according to SEQ ID NO: 371 or a sequence that has at least 70% (e.g. at least 90 or 95%) identity thereto.

97. The nucleic acid of any preceding embodiment, wherein the polypeptide further comprises a secretion signal peptide sequence.

98. The nucleic acid of embodiment 97, wherein the secretion signal peptide sequence is a viral secretion signal peptide sequence, optionally selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, and a smallpox 6 kDa IC protein secretion signal peptide sequence, optionally wherein the secretion signal peptide sequence comprises an amino acid sequence according to one of the SEQ ID NOs in Table 23.

99. The nucleic acid of embodiment 97 or embodiment 98, wherein the secretion signal peptide sequence comprises a secretion signal peptide sequence of HA protein of influenza A virus, e.g, wherein the secretion signal peptide sequence comprises a sequence according to SEQ ID NO: 67

100. The nucleic acid of embodiment 97, wherein the polypeptide comprises a sequence according to SEQ ID NO: 2 or a sequence that has at least 70% (e.g. at least 90 or 95%) identity thereto.

101. The nucleic acid of embodiment 97, wherein the polypeptide comprises a sequence according to SEQ ID NO: 7 or a sequence that has at least 70% (e.g. at least 90 or 95%) identity thereto.

102. The nucleic acid of embodiment 97, wherein the polypeptide comprises a sequence according to SEQ ID NO: 12 or a sequence that has at least 70% (e.g. at least 90 or 95%) identity thereto.

103. The nucleic acid of embodiment 97, wherein the polypeptide comprises a sequence according to SEQ ID NO: 17 or a sequence that has at least 70% (e.g. at least 90 or 95%) identity thereto.

104. The nucleic acid of any preceding embodiment, wherein the polypeptide further comprises a heterologous transmembrane domain.

105. The nucleic acid of embodiment 104, wherein the transmembrane domain sequence is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence and an Ebola GP protein transmembrane domain sequence, optionally wherein the transmembrane domain comprises an amino acid sequence according to one of the SEQ ID NOs in Table 25.

106. The nucleic acid of embodiment 104 or embodiment 105, wherein the transmembrane domain comprises the sequence of a transmembrane domain of HA protein of influenza A virus, e.g, wherein the transmembrane domain comprises a sequence according to SEQ ID NO: 70.

107. The nucleic acid of embodiment 104, wherein the polypeptide comprises a sequence according to SEQ ID NO: 4 or a sequence that has at least 70% (e.g. at least 90 or 95%) identity thereto.

108. The nucleic acid of embodiment 104, wherein the polypeptide comprises a sequence according to SEQ ID NO: 9 or a sequence that has at least 70% (e.g. at least 90 or 95%) identity thereto.

109. The nucleic acid of embodiment 104, wherein the polypeptide comprises a sequence according to SEQ ID NO: 14 or a sequence that has at least 70% (e.g. at least 90 or 95%) identity thereto.

110. The nucleic acid of embodiment 104, wherein the polypeptide comprises a sequence according to SEQ ID NO: 19 or a sequence that has at least 70% (e.g. at least 90 or 95%) identity thereto.

111. The nucleic acid of any preceding embodiment, wherein the polypeptide comprises a mutation at one or more (for example, all) positions corresponding to a glycosylation site, optionally an N-glycosylation site in a native Porphyromonas gingivalis polypeptide, optionally wherein the mutation is a single amino acid substitution.

112. The nucleic acid of embodiment 111 wherein the polypeptide comprises a sequence according to SEQ ID NO: 359 or a sequence that has at least 70% (e.g. at least 90 or 95%) identity thereto.

113. The nucleic acid of embodiments 111, wherein the polypeptide comprises a sequence according to SEQ ID NO: 361 or a sequence that has at least 70% (e.g. at least 90 or 95%) identity thereto.

114. The nucleic acid of embodiment 111, wherein the polypeptide comprises a sequence according to SEQ ID NO: 363 or a sequence that has at least 70% (e.g. at least 90 or 95%) identity thereto.

115. The nucleic acid of embodiment 111, wherein the polypeptide comprises a sequence according to SEQ ID NO: 365 or a sequence that has at least 70% (e.g. at least 90 or 95%) identity thereto.

116. The nucleic acid of embodiment 111, wherein the polypeptide comprises a sequence according to SEQ ID NO: 369 or a sequence that has at least 70% (e.g. at least 90 or 95%) identity thereto.

117. The nucleic acid of embodiment 111, wherein the polypeptide comprises a sequence according to SEQ ID NO: 373 or a sequence that has at least 70% (e.g. at least 90 or 95%) identity thereto.

118. The nucleic acid of embodiment 111, wherein the polypeptide comprises a sequence according to SEQ ID NO: 377 or a sequence that has at least 70% (e.g. at least 90 or 95%) identity thereto.

119. The nucleic acid of embodiment 111, wherein the polypeptide comprises a sequence according to SEQ ID NO: 381 or a sequence that has at least 70% (e.g. at least 90 or 95%) identity thereto.

120. The nucleic acid of embodiment 111, wherein the polypeptide comprises a sequence according to SEQ ID NO: 385 or a sequence that has at least 70% (e.g. at least 90 or 95%) identity thereto.

121. The nucleic acid of embodiment 111, wherein the polypeptide comprises a sequence according to SEQ ID NO: 387 or a sequence that has at least 70% (e.g. at least 90 or 95%) identity thereto.

122. The nucleic acid of embodiment 111, wherein the polypeptide comprises a sequence according to SEQ ID NO: 389 or a sequence that has at least 70% (e.g. at least 90 or 95%) identity thereto.

123. The nucleic acid of embodiment 111, wherein the polypeptide comprises a sequence according to SEQ ID NO: 391 or a sequence that has at least 70% (e.g. at least 90 or 95%) identity thereto.

124. The nucleic acid of embodiment 111, wherein the polypeptide comprises a sequence according to SEQ ID NO: 393 or a sequence that has at least 70% (e.g. at least 90 or 95%) identity thereto.

125. The nucleic acid of embodiment 111, wherein the polypeptide comprises a sequence according to SEQ ID NO: 395 or a sequence that has at least 70% (e.g. at least 90 or 95%) identity thereto.

126. The nucleic acid of embodiment 111, wherein the polypeptide comprises a sequence according to SEQ ID NO: 399 or a sequence that has at least 70% (e.g. at least 90 or 95%) identity thereto.

127. The nucleic acid of embodiment 111, wherein the polypeptide comprises a sequence according to SEQ ID NO: 405 or a sequence that has at least 70% (e.g. at least 90 or 95%) identity thereto.

128. The nucleic acid of embodiment 111, wherein the polypeptide comprises a sequence according to SEQ ID NO: 417 or a sequence that has at least 70% (e.g. at least 90 or 95%) identity thereto.

129. The nucleic acid of embodiment 111, wherein the polypeptide comprises a sequence according to SEQ ID NO: 411 or a sequence that has at least 70% (e.g. at least 90 or 95%) identity thereto.

130 The nucleic acid of embodiment 111, wherein the polypeptide comprises a sequence according to SEQ ID NO: 3 or a sequence that has at least 70% (e.g. at least 90 or 95%) identity thereto.

131. The nucleic acid of embodiment 111, wherein the polypeptide comprises a sequence according to SEQ ID NO: 279 or a sequence that has at least 70% (e.g. at least 90 or 95%) identity thereto.

132. The nucleic acid of embodiment 111, wherein the polypeptide comprises a sequence according to SEQ ID NO: 8 or a sequence that has at least 70% (e.g. at least 90 or 95%) identity thereto.

133. The nucleic acid of embodiment 111, wherein the polypeptide comprises a sequence according to SEQ ID NO: 13 or a sequence that has at least 70% (e.g. at least 90 or 95%) identity thereto.

134. The nucleic acid of embodiment 111, wherein the polypeptide comprises a sequence according to SEQ ID NO: 280 or a sequence that has at least 70% (e.g. at least 90 or 95%) identity thereto.

135. The nucleic acid of embodiment 111, wherein the polypeptide comprises a sequence according to SEQ ID NO: 297 or a sequence that has at least 70% (e.g. at least 90 or 95%) identity thereto.

136 The nucleic acid of embodiment 111, wherein the polypeptide comprises a sequence according to SEQ ID NO: 18 or a sequence that has at least 70% (e.g. at least 90 or 95%) identity thereto.

137. The nucleic acid of embodiment 111, wherein the polypeptide comprises a sequence according to SEQ ID NO: 73 or a sequence that has at least 70% (e.g. at least 90 or 95%) identity thereto.

138. The nucleic acid of embodiment 111, wherein the polypeptide comprises a sequence according to SEQ ID NO: 74 or a sequence that has at least 70% (e.g. at least 90 or 95%) identity thereto.

139. The nucleic acid of embodiment 111, wherein the polypeptide comprises a sequence according to SEQ ID NO: 75 or a sequence that has at least 70% (e.g. at least 90 or 95%) identity thereto.

140. The nucleic acid of embodiment 111, wherein the polypeptide comprises a sequence according to SEQ ID NO: 283 or a sequence that has at least 70% (e.g. at least 90 or 95%) identity thereto.

141. The nucleic acid of embodiment 111, wherein the polypeptide comprises a sequence according to SEQ ID NO: 76 or a sequence that has at least 70% (e.g. at least 90 or 95%) identity thereto.

142. The nucleic acid of embodiment 111, wherein the polypeptide comprises a sequence according to SEQ ID NO: 284 or a sequence that has at least 70% (e.g. at least 90 or 95%) identity thereto.

143. The nucleic acid of embodiment 111, wherein the polypeptide comprises a sequence according to SEQ ID NO: 401 or a sequence that has at least 70% (e.g. at least 90 or 95%) identity thereto.

144. The nucleic acid of embodiment 111, wherein the polypeptide comprises a sequence according to SEQ ID NO: 413 or a sequence that has at least 70% (e.g. at least 90 or 95%) identity thereto.

145. The nucleic acid of embodiment 111, wherein the polypeptide comprises a sequence according to SEQ ID NO: 77 or a sequence that has at least 70% (e.g. at least 90 or 95%) identity thereto.

146. The nucleic acid of embodiment 111, wherein the polypeptide comprises a sequence according to SEQ ID NO: 285 or a sequence that has at least 70% (e.g. at least 90 or 95%) identity thereto.

147. The nucleic acid of embodiment 111, wherein the polypeptide comprises a sequence according to SEQ ID NO: 407 or a sequence that has at least 70% (e.g. at least 90 or 95%) identity thereto.

148. The nucleic acid of embodiment 111, wherein the polypeptide comprises a sequence according to SEQ ID NO: 5 or a sequence that has at least 70% (e.g. at least 90 or 95%) identity thereto.

149. The nucleic acid of embodiment 111, wherein the polypeptide comprises a sequence according to SEQ ID NO: 10 or a sequence that has at least 70% (e.g. at least 90 or 95%) identity thereto.

150. The nucleic acid of embodiment 111, wherein the polypeptide comprises a sequence according to SEQ ID NO: 15 or a sequence that has at least 70% (e.g. at least 90 or 95%) identity thereto.

151. The nucleic acid of embodiment 111, wherein the polypeptide comprises a sequence according to SEQ ID NO: 20 or a sequence that has at least 70% (e.g. at least 90 or 95%) identity thereto.

152. The nucleic acid of any preceding embodiment, wherein the nucleic acid is a messenger RNA (mRNA), optionally wherein
(i) the mRNA comprises at least one 5′ untranslated portion (5′ UTR), at least one 3′ untranslated portion (3′ UTR), and/or at least one polyadenylation (poly(A)) sequence;
(ii) the mRNA is unmodified or comprises at least one chemical modification, optionally wherein the mRNA comprises at least one chemical modification, for example wherein the chemical modification comprises N1-methylpseudouridine; and/or
(iii) the mRNA is a self-replicating mRNA or a non-replicating mRNA, e.g. a non-replicating mRNA.

153. A polypeptide comprising:
i) at least a portion of a Porphyromonas gingivalis Lys-specific proteinase (Kgp) catalytic domain;
ii) at least a portion of a Porphyromonas gingivalis Kgp domain of unknown function 2436 (DUF2436);
iii) at least a portion of a Porphyromonas gingivalis Kgp K1 adhesin domain;
iv) a first Porphyromonas gingivalis Kgp portion that comprises an adhesin binding motif 1 (ABM1) and a first Porphyromonas gingivalis Kgp portion that comprises an adhesin binding motif 2 (ABM2);
v) a second Porphyromonas gingivalis Kgp portion that comprises an ABM1 and a second Porphyromonas gingivalis Kgp portion that comprises an ABM2; and
vi) a Porphyromonas gingivalis Kgp portion that comprises ABM3.

154. The polypeptide of embodiment 153, wherein the first Kgp portion that comprises an ABM1 comprises a sequence according to SEQ ID NO: 120 or a sequence that has at least 70% (e.g. at least 90 or 95%) identity thereto.

155. The polypeptide of embodiment 153 or 154, wherein the first Kgp portion that comprises an ABM2 comprises a sequence according to SEQ ID NO: 130 or a sequence that has at least 70% (e.g. at least 90 or 95%) identity thereto.

156. The polypeptide of any one of embodiments 153-155, wherein the second Kgp portion that comprises an ABM1 comprises a sequence according to SEQ ID NO: 120 or a sequence that has at least 70% (e.g. at least 90 or 95%) identity thereto.

157. The polypeptide of any one of embodiments 153-156, wherein the second Kgp portion that comprises an ABM2 comprises a sequence according to SEQ ID NO: 130 or a sequence that has at least 70% (e.g. at least 90 or 95%) identity thereto.

158. The polypeptide of any one of embodiments 153-157, wherein the first Kgp portion that comprises an ABM1 comprises a sequence according to SEQ ID NO: 89 or a sequence that has at least 70% (e.g. at least 90 or 95%) identity thereto.

159. The polypeptide of any one of embodiments 153-158, wherein the first Kgp portion that comprises an ABM2 comprises a sequence according to SEQ ID NO: 91 or a sequence that has at least 70% (e.g. at least 90 or 95%) identity thereto.

160. The polypeptide of any one of embodiments 153-159, wherein the second Kgp portion that comprises an ABM1 comprises a sequence according to SEQ ID NO: 92 or a sequence that has at least 70% (e.g. at least 90 or 95%) identity thereto.

161. The polypeptide of any one of embodiments 153-160, wherein the second Kgp portion that comprises an ABM2 comprises a sequence according to SEQ ID NO: 95 or a sequence that has at least 70% (e.g. at least 90 or 95%) identity thereto.

162. The polypeptide of any one of embodiments 153-161, wherein the first Kgp portion that comprises an ABM1 and the first domain Kgp that comprises ABM2 are positioned N-terminally of the second Kgp portion that comprises ABM1 and the second Kgp portion that comprises ABM2.

163. The polypeptide of any one of embodiments 153-162, wherein the first Kgp portion that comprises an ABM1 is positioned N-terminally of the at least a portion of the Kgp DUF2436 and the first Kgp portion that comprises an ABM2 is positioned C-terminally of the at least a portion of the Kgp DUF2436, for example wherein the Kgp portion that comprises an ABM1 is positioned N-terminally and adjacent to the at least a portion of the Kgp DUF2436 and the first Kgp portion that comprises an ABM2 is positioned C-terminally and adjacent to the at least a portion of the Kgp DUF2436.

164. The polypeptide of any one of embodiments 153-163, wherein the second Kgp portion that comprises an ABM1 is positioned N-terminally of the at least a portion of the Kgp K1 adhesin domain and the second Kgp portion that comprises an ABM2 is positioned C-terminally of the at least a portion of the Kgp K1 adhesin domain.

165. The polypeptide of any one of embodiments 153-164, wherein the at least a portion of Kgp DUF2436 is a full-length Kgp DUF2436.

166. The polypeptide of embodiment 165, wherein the full-length Kgp DUF2436 comprises a sequence according to SEQ ID NO: 90 or a sequence that has at least 70% (e.g. at least 90 or 95%) identity thereto.

167. The polypeptide of any one of embodiments 153-166, wherein the Kgp DUF2436 is positioned C-terminally of the Kgp catalytic domain.

168. The polypeptide of any one of embodiments 153-167, wherein the Kgp DUF2436 is positioned N-terminally of the Kgp K1 adhesin domain.

169. The polypeptide of any one of embodiments 153-168, wherein the at least a portion of Kgp catalytic domain is a full-length Kgp catalytic domain.

170. The polypeptide of embodiment 169, wherein the full-length Kgp catalytic domain comprises a sequence according to SEQ ID NO: 64 or a sequence that has at least 70% (e.g. at least 90 or 95%) identity thereto.

171. The polypeptide of any one of embodiments 153-168, wherein the at least a portion of Kgp catalytic domain is a truncated Kgp catalytic domain.

172. The polypeptide of embodiment 171, wherein the truncated Kgp catalytic domain comprises a Lys-gingipain active site peptide (KAS peptide).

173. The polypeptide of embodiment 172, wherein the KAS peptide comprises a Kas2 peptide according to SEQ ID NO: 61 or a sequence that has at least 70% (e.g. at least 90 or 95%) identity thereto.

174. The polypeptide of embodiment 172, wherein the KAS peptide comprises an extended Kas2 peptide according to SEQ ID NO: 88 or a sequence that has at least 70% (e.g. at least 90 or 95%) identity thereto.

175. The polypeptide of any one of embodiments 153-174, wherein Kgp catalytic domain comprises a mutation that inactivates proteinase activity, wherein optionally the mutation that inactivates proteinase activity is a cysteine to serine mutation at position 477 (C₄₇₇S), wherein the mutation position corresponds to position 477 of the wild-type Kgp sequence of SEQ ID NO: 157.

176. The polypeptide of any one of embodiments 153-175, wherein the polypeptide further comprises at least a portion of a Porphyromonas gingivalis Arg-specific proteinase A (RgpA) or Arg-specific proteinase B (RgpB) catalytic domain, for example at least a portion of the Porphyromonas gingivalis Arg-specific proteinase A (RgpA).

177. The polypeptide of embodiment 176, wherein the at least a portion of RgpA or RgpB catalytic domain is a truncated RgpA or RgpB catalytic domain.

178. The polypeptide of embodiment 177, wherein the truncated RgpA or RgpB catalytic domain comprises of a Arg-gingipain active site peptide (RAS peptide).

179. The polypeptide of embodiment 178, wherein the RAS peptide comprises a Ras2 peptide according to SEQ ID NO: 166 or a sequence that has at least 70% (e.g. at least 90 or 95%) identity thereto.

180. The polypeptide of embodiment 178, wherein the RAS peptide comprises an extended Ras2 peptide according to SEQ ID NO: 97 or a sequence that has at least 70% (e.g. at least 90 or 95%) identity thereto.

181. The polypeptide of any one of embodiments 176-180, wherein the at least a portion of the RgpA or RgpB catalytic domain is positioned at the C-terminus of the polypeptide.

182. The polypeptide of any one of embodiments 153-181, wherein the polypeptide further comprises at least a portion of a Porphyromonas gingivalis Kgp K2 adhesin domain.

183. The polypeptide of embodiment 182, wherein the at least a portion of Kgp K2 adhesin domain is a full-length K2 adhesin domain.

184. The polypeptide of embodiment 183, wherein the full-length Kgp K2 adhesin domain comprises a sequence according to SEQ ID NO: 96 or a sequence that has at least 70% (e.g. at least 90 or 95%) identity thereto.

185. The polypeptide of any one of embodiments 182-184, wherein the at least a portion of Kgp K2 adhesin domain is positioned C-terminally of the Kgp K1 adhesin domain, for example C-terminally and adjacent to the Kgp K1 adhesin domain.

186. The polypeptide of any one of embodiments 182-185, wherein the at least a portion of Kgp K2 adhesin domain is positioned between the second Kgp portion that comprises an ABM1 and the second Kgp portion that comprises an ABM2.

187. The polypeptide of any one of embodiments 176-181, wherein the polypeptide further comprises at least a portion of a Porphyromonas gingivalis RgpA K2 adhesin domain.

188. The polypeptide of embodiment 187, wherein the at least a portion of RgpA K2 adhesin domain is a full-length K2 adhesin domain.

189. The polypeptide of embodiment 187 or 188, wherein the full-length RgpA K2 adhesin domain comprises a sequence according to SEQ ID NO: 104 or a sequence that has at least 70% (e.g. at least 90 or 95%) identity thereto.

190. The polypeptide of any one of embodiments 187-189, wherein the at least a portion of RgpA K2 adhesin domain is positioned C-terminally of the Kgp K1 adhesin domain, for example C-terminally and adjacent to the Kgp K1 adhesin domain.

191. The polypeptide of any one of embodiments 187-190, wherein the at least a portion of RgpA K2 adhesin domain is positioned between the second Kgp portion that comprises an ABM1 and the second Kgp portion that comprises an ABM2.

192. The polypeptide of embodiment any one of embodiments 176-191, wherein the polypeptide comprises at least a portion of an RgpA DUF2436.

193. The polypeptide of embodiment 192, wherein the RgpA DUF2436 is a full-length RgpA DUF2436.

194. The polypeptide of embodiment 193, wherein the full-length RgpA DUF2436 comprises a sequence according to SEQ ID NO: 100 or a sequence that has at least 70% (e.g. at least 90 or 95%) identity thereto.

195. The polypeptide of any one of embodiments 192-194, wherein the polypeptide comprises a first Porphyromonas gingivalis RgpA portion that comprises an ABM1, and a first Porphyromonas gingivalis RgpA portion that comprises an ABM2.

196. The polypeptide of embodiment 195, wherein the first RgpA portion that comprises an ABM1 comprises a sequence according to SEQ ID NO: 99 or a sequence that has at least 70% (e.g. at least 90 or 95%) identity thereto.

197. The polypeptide of embodiment 195 or embodiment 196, wherein the first RgpA portion that comprises an ABM2 comprises a sequence according to SEQ ID NO: 101 or a sequence that has at least 70% (e.g. at least 90 or 95%) identity thereto.

198. The polypeptide of any one of embodiments 195-197, wherein the first RgpA portion that comprises an ABM1 is positioned N-terminally of the at least a portion of the RgpA DUF2436 and the first RgpA portion that comprises an ABM2 is positioned C-terminally of the at least a portion of the RgpA DUF2436, for example wherein the first RgpA portion that comprises an ABM1 is positioned N-terminally and adjacent to the at least a portion of the RgpA DUF2436 and the first RgpA portion that comprises an ABM2 is positioned C-terminally and adjacent to the at least a portion of the RgpA DUF2436.

199. The polypeptide of any one of embodiments 153-198, wherein the Kgp portion comprising ABM3 comprises a sequence according to SEQ ID NO: 139 or a sequence that has at least 70% (e.g. at least 90 or 95%) identity thereto.

200. The polypeptide of any one of embodiments 153-199, wherein the at least a portion of the Kgp K1 adhesin domain is a full-length Kgp K1 adhesin domain.

201. The polypeptide of embodiment 200, wherein the full-length Kgp K1 adhesin domain and Kgp portion comprising ABM3 together comprise a sequence according to SEQ ID NO: 93 or a sequence that has at least 70% (e.g. at least 90 or 95%) identity thereto.

202. The polypeptide of any one of embodiments 153-201, wherein the at least a portion of the Kgp K1 adhesin domain is a truncated Kgp K1 adhesin domain.

203. The polypeptide of embodiment 202, wherein the truncated Kgp K1 adhesin domain and Kgp portion comprising ABM3 together comprise a sequence according to SEQ ID NO: 94 or a sequence that has at least 70% (e.g. at least 90 or 95%) identity thereto.

204. The polypeptide of any one of embodiments 153-203, wherein the polypeptide comprises a sequence according to SEQ ID NO:1 or a sequence that has at least 70% (e.g. at least 90 or 95%) identity thereto.

205. The polypeptide of any one of embodiments 153-203, wherein the polypeptide comprises a sequence according to SEQ ID NO: 6 or a sequence that has at least 70% (e.g. at least 90 or 95%) identity thereto.

206. The polypeptide of any one of embodiments 153-203, wherein the polypeptide comprises a sequence according to SEQ ID NO: 11 or a sequence that has at least 70% (e.g. at least 90 or 95%) identity thereto.

207. The polypeptide of any one of embodiments 153-203, wherein the polypeptide comprises a sequence according to SEQ ID NO: 16 or a sequence that has at least 70% (e.g. at least 90 or 95%) identity thereto.

208. The polypeptide of any one of embodiments 153-203, wherein the polypeptide comprises a sequence according to SEQ ID NO: 375 or a sequence that has at least 70% (e.g. at least 90 or 95%) identity thereto.

209. The polypeptide of any one of embodiments 153-203, wherein the polypeptide comprises a sequence according to SEQ ID NO: 379 or a sequence that has at least 70% (e.g. at least 90 or 95%) identity thereto.

210. The polypeptide of any one of embodiments 153-203, wherein the polypeptide comprises a sequence according to SEQ ID NO: 383 or a sequence that has at least 70% (e.g. at least 90 or 95%) identity thereto.

211. The polypeptide of any one of embodiments 153-203, wherein the polypeptide comprises a sequence according to SEQ ID NO: 397 or a sequence that has at least 70% (e.g. at least 90 or 95%) identity thereto.

212. The polypeptide of any one of embodiments 153-203, wherein the polypeptide comprises a sequence according to SEQ ID NO: 403 or a sequence that has at least 70% (e.g. at least 90 or 95%) identity thereto.

213. The polypeptide of any one of embodiments 153-203, wherein the polypeptide comprises a sequence according to SEQ ID NO: 415 or a sequence that has at least 70% (e.g. at least 90 or 95%) identity thereto.

214. The polypeptide of any one of embodiments 153-203, wherein the polypeptide comprises a sequence according to SEQ ID NO: 409 or a sequence that has at least 70% (e.g. at least 90 or 95%) identity thereto.

215. A polypeptide comprising:
i) at least a portion of a Porphyromonas gingivalis Arg-specific proteinase A (RgpA) or Arg-specific proteinase B (RgpB) catalytic domain, for example at least a portion of the Porphyromonas gingivalis Arg-specific proteinase A (RgpA);
ii) at least a portion of a Porphyromonas gingivalis RgpA DUF2436;
iii) at least a portion of a Porphyromonas gingivalis RgpA K1 adhesin domain;
iv) a first Porphyromonas gingivalis RgpA portion that comprises an ABM1, and a first Porphyromonas gingivalis RgpA portion that comprises an ABM2;
v) a second Porphyromonas gingivalis RgpA portion that comprises an ABM1, and a second Porphyromonas gingivalis RgpA portion that comprises an ABM2; and
vi) a Porphyromonas gingivalis RgpA portion that comprises an ABM3.

216. The polypeptide of embodiment 215, wherein the first RgpA portion that comprises an ABM1 comprises a sequence according to SEQ ID NO: 120 or a sequence that has at least 70% (e.g. at least 90 or 95%) identity thereto.

217. The polypeptide of embodiment 215 or embodiment 216, wherein the first RgpA portion that comprises an ABM2 comprises a sequence according to SEQ ID NO: 130 or a sequence that has at least 70% (e.g. at least 90 or 95%) identity thereto.

218. The polypeptide of any one of embodiments 215-217, wherein the second RgpA portion that comprises an ABM1 comprises a sequence according to SEQ ID NO: 120 or a sequence that has at least 70% (e.g. at least 90 or 95%) identity thereto.

219. The polypeptide of any one of embodiments 215-218, wherein the second RgpA portion that comprises an ABM2 comprises a sequence according to SEQ ID NO: 130 or a sequence that has at least 70% (e.g. at least 90 or 95%) identity thereto.

220. The polypeptide of any one of embodiments 215-219, wherein the first RgpA portion that comprises an ABM1 comprises a sequence according to SEQ ID NO: 99 or a sequence that has at least 70% (e.g. at least 90 or 95%) identity thereto.

221. The polypeptide of any one of embodiments 215-220, wherein the first RgpA portion that comprises an ABM2 comprises a sequence according to SEQ ID NO: 101 or a sequence that has at least 70% (e.g. at least 90 or 95%) identity thereto.

222. The polypeptide of any one of embodiments 215-221, wherein the second RgpA portion that comprises an ABM1 comprises a sequence according to SEQ ID NO: 102 or a sequence that has at least 70% (e.g. at least 90 or 95%) identity thereto.

223. The polypeptide of any one of embodiments 215-222, wherein the second RgpA portion that comprises an ABM2 comprises a sequence according to SEQ ID NO: 105 or a sequence that has at least 70% (e.g. at least 90 or 95%) identity thereto.

224. The polypeptide of any one of embodiments 215-223, wherein the first RgpA portion that comprises an ABM1 and the first domain RgpA that comprises an ABM2 are positioned N-terminally of the second RgpA portion that comprises an ABM1 and the second RgpA portion that comprises an ABM2.

225. The polypeptide of any one of embodiments 215-224, wherein the first RgpA portion that comprises an ABM1 is positioned N-terminally of the at least a portion of the RgpA DUF2436 and the first RgpA portion that comprises an ABM2 is positioned C-terminally of the at least a portion of the RgpA DUF2436, for example wherein the RgpA portion that comprises an ABM1 is positioned N-terminally and adjacent to the at least a portion of the RgpA DUF2436 and the first RgpA portion that comprises an ABM2 is positioned C-terminally and adjacent to the at least a portion of the RgpA DUF2436.

226. The polypeptide of any one of embodiments 215-225, wherein the second RgpA portion that comprises an ABM1 is positioned N-terminally of the at least a portion of the RgpA K1 adhesin domain and the second RgpA portion that comprises an ABM2 is positioned C-terminally of the at least a portion of the RgpA K1 adhesin domain.

227. The polypeptide of any one of embodiments 215-226, wherein the at least a portion of RgpA DUF2436 is a full-length RgpA DUF2436.

228. The polypeptide of embodiment 227, wherein the full-length RgpA DUF2436 comprises a sequence according to SEQ ID NO: 100 or a sequence that has at least 70% (e.g. at least 90 or 95%) identity thereto.

229. The polypeptide of any one of embodiments 215-228, wherein the RgpA DUF2436 is positioned C-terminally of the at least a portion of the RgpA or RgpB catalytic domain.

230. The polypeptide of any one of embodiments 215-228, wherein the at least a portion of the RgpA or RgpB catalytic domain is positioned at the C-terminus of the polypeptide.

231. The polypeptide of any one of embodiments 215-230, wherein the RgpA DUF2436 is positioned N-terminally of the RgpA K1 adhesin domain.

232. The polypeptide of any one of embodiments 215-231, wherein the at least a portion of RgpA or RgpB catalytic domain is a full-length RgpA or RgpB catalytic domain.

233. The polypeptide of embodiment 232, wherein the full-length RgpA catalytic domain comprises a sequence according to SEQ ID NO: 98 or a sequence that has at least 70% (e.g. at least 90 or 95%) identity thereto.

234. The polypeptide of any one of embodiments 215-231, wherein the at least a portion of RgpA or RgpB catalytic domain is a truncated RgpA or RgpB catalytic domain.

235. The polypeptide of embodiment 234, wherein the truncated RgpA or RgpB catalytic domain comprises a Arg-gingipain active site peptide (RAS peptide).

236. The polypeptide of embodiment 235, wherein the RAS peptide comprises a Ras2 peptide according to SEQ ID NO: 166 or a sequence that has at least 70% (e.g. at least 90 or 95%) identity thereto.

237. The polypeptide of embodiment 235, wherein the RAS peptide comprises an extended Ras2 peptide according to SEQ ID NO: 97 or a sequence that has at least 70% (e.g. at least 90 or 95%) identity thereto.

238. The polypeptide of any one of embodiments 215-237, wherein the RgpA or RgpB catalytic domain comprises a mutation that inactivates proteinase activity, wherein optionally the mutation that inactivates proteinase activity is a cysteine to serine mutation at position 471 (C₄₇₁S), wherein the mutation position corresponds to position 471 of the wild-type RgpA sequence of SEQ ID NO: 158.

239. The polypeptide of any one of embodiments 215-238, wherein the polypeptide further comprises at least a portion of a Porphyromonas gingivalis RgpA K2 adhesin domain.

240. The polypeptide of embodiment 239, wherein the at least a portion of RgpA K2 adhesin domain is a full-length K2 adhesin domain.

241. The polypeptide of embodiment 240, wherein the full-length RgpA K2 adhesin domain comprises a sequence according to SEQ ID NO: 104 or a sequence that has at least 70% (e.g. at least 90 or 95%) identity thereto.

242. The polypeptide of embodiment 240or embodiment 241, wherein the at least a portion of RgpA K2 adhesin domain is positioned C-terminally of the RgpA K1 adhesin domain, for example C-terminally and adjacent to the RgpA K1 adhesin domain.

243. The polypeptide of any one of embodiments 240-242, wherein the at least a portion of RgpA K2 adhesin domain is positioned between the second RgpA portion that comprises an ABM1 and the second RgpA portion that comprises an ABM2.

244. The polypeptide of any one of embodiments 215-243, wherein the RgpA portion comprising an ABM3 motif comprises a sequence according to SEQ ID NO: 139 or a sequence that has at least 70% (e.g. at least 90 or 95%) identity thereto.

245. The polypeptide of any one of embodiments 215-244, wherein the at least a portion of the RgpA K1 adhesin domain is a truncated RgpA K1 adhesin domain.

246. The polypeptide of embodiment 245, wherein the truncated RgpA K1 adhesin domain and RgpA portion comprising an ABM3 together comprise a sequence according to SEQ ID NO: 103 or a sequence that has at least 70% (e.g. at least 90 or 95%) identity thereto.

247. The polypeptide of any one of embodiments 215-246, wherein the polypeptide comprises a sequence according to SEQ ID NO: 367 or a sequence that has at least 70% (e.g. at least 90 or 95%) identity thereto.

248. The nucleic acid of any one of embodiments 215-246, wherein the polypeptide comprises a sequence according to SEQ ID NO: 371 or a sequence that has at least 70% (e.g. at least 90 or 95%) identity thereto.

249. The polypeptide of any of embodiments 153-248, wherein the polypeptide further comprises a secretion signal peptide sequence.

250. The polypeptide of embodiment 249, wherein the secretion signal peptide sequence is a viral secretion signal peptide sequence, optionally selected from the group consisting of: an influenza hemagglutinin (HA) secretion signal peptide sequence, a SARS CoV-2 spike secretion signal peptide sequence, a VZV gB secretion signal peptide sequence, a VZV gE secretion signal peptide sequence, a VZV gI secretion signal peptide sequence, a VZV gK secretion signal peptide sequence, a measles F-protein secretion signal peptide sequence, a rubella E1 protein secretion signal peptide sequence, a rubella E2 protein secretion signal peptide sequence, a mumps F-protein secretion signal peptide sequence, an Ebola GP protein secretion signal peptide sequence, and a smallpox 6 kDa IC protein secretion signal peptide sequence, optionally wherein the secretion signal peptide sequence comprises an amino acid sequence according to one of the SEQ ID NOs in Table 23.

251. The polypeptide of embodiment 249 or embodiment 250, wherein the secretion signal peptide sequence comprises a secretion signal peptide sequence of HA protein of influenza A virus, e.g, wherein the secretion signal peptide sequence comprises a sequence according to SEQ ID NO: 67

252. The polypeptide of embodiment 249, wherein the polypeptide comprises a sequence according to SEQ ID NO: 2 or a sequence that has at least 70% (e.g. at least 90 or 95%) identity thereto.

253. The polypeptide of embodiment 249, wherein the polypeptide comprises a sequence according to SEQ ID NO: 7 or a sequence that has at least 70% (e.g. at least 90 or 95%) identity thereto.

254. The polypeptide of embodiment 249, wherein the polypeptide comprises a sequence according to SEQ ID NO: 12 or a sequence that has at least 70% (e.g. at least 90 or 95%) identity thereto.

255. The polypeptide of embodiment 249, wherein the polypeptide comprises a sequence according to SEQ ID NO: 17 or a sequence that has at least 70% (e.g. at least 90 or 95%) identity thereto.

256. The polypeptide of any one of embodiments 153-255, wherein the polypeptide further comprises a heterologous transmembrane domain.

257. The polypeptide of embodiment 256, wherein the transmembrane domain sequence is selected from the group consisting of: an influenza hemagglutinin (HA) transmembrane domain sequence, a SARS CoV-2 spike transmembrane domain sequence, a VZV gB transmembrane domain sequence, a VZV gE transmembrane domain sequence, a VZV gI transmembrane domain sequence, a VZV gK transmembrane domain sequence, a measles F-protein transmembrane domain sequence, a rubella E1 protein transmembrane domain sequence, a rubella E2 protein transmembrane domain sequence, a mumps F-protein transmembrane domain sequence and an Ebola GP protein transmembrane domain sequence, optionally wherein the transmembrane domain comprises an amino acid sequence according to one of the SEQ ID NOs in Table 25.

258. The polypeptide of embodiment 256 or embodiment 257, wherein the transmembrane domain comprises the sequence of a transmembrane domain of HA protein of influenza A virus, e.g, wherein the transmembrane domain comprises a sequence according to SEQ ID NO: 70.

259. The polypeptide of embodiment 256, wherein the polypeptide comprises a sequence according to SEQ ID NO: 4 or a sequence that has at least 70% (e.g. at least 90 or 95%) identity thereto.

260. The polypeptide of embodiment 256, wherein the polypeptide comprises a sequence according to SEQ ID NO: 9 or a sequence that has at least 70% (e.g. at least 90 or 95%) identity thereto.

261. The polypeptide of embodiment 256, wherein the polypeptide comprises a sequence according to SEQ ID NO: 14 or a sequence that has at least 70% (e.g. at least 90 or 95%) identity thereto.

262. The polypeptide of embodiment 256, wherein the polypeptide comprises a sequence according to SEQ ID NO: 19 or a sequence that has at least 70% (e.g. at least 90 or 95%) identity thereto.

263. The polypeptide of any one of embodiments 153-262, wherein the polypeptide comprises a mutation at one or more (for example, all) positions corresponding to a glycosylation site, optionally an N-glycosylation site in a native Porphyromonas gingivalis polypeptide, optionally wherein the mutation is a single amino acid substitution.

264. The polypeptide of embodiment 263, wherein the polypeptide comprises a sequence according to SEQ ID NO: 359 or a sequence that has at least 70% (e.g. at least 90 or 95%) identity thereto.

265. The polypeptide of embodiment 263, wherein the polypeptide comprises a sequence according to SEQ ID NO: 361 or a sequence that has at least 70% (e.g. at least 90 or 95%) identity thereto.

266. The polypeptide of embodiment 263, wherein the polypeptide comprises a sequence according to SEQ ID NO: 363 or a sequence that has at least 70% (e.g. at least 90 or 95%) identity thereto.

267. The polypeptide of embodiment 263, wherein the polypeptide comprises a sequence according to SEQ ID NO: 365 or a sequence that has at least 70% (e.g. at least 90 or 95%) identity thereto.

268. The polypeptide of embodiment 263, wherein the polypeptide comprises a sequence according to SEQ ID NO: 369 or a sequence that has at least 70% (e.g. at least 90 or 95%) identity thereto.

269. The polypeptide of embodiment 263, wherein the polypeptide comprises a sequence according to SEQ ID NO: 373 or a sequence that has at least 70% (e.g. at least 90 or 95%) identity thereto.

270. The polypeptide of embodiment 263, wherein the polypeptide comprises a sequence according to SEQ ID NO: 377 or a sequence that has at least 70% (e.g. at least 90 or 95%) identity thereto.

271. The polypeptide of embodiment 263, wherein the polypeptide comprises a sequence according to SEQ ID NO: 381 or a sequence that has at least 70% (e.g. at least 90 or 95%) identity thereto.

272. The polypeptide of embodiment 263, wherein the polypeptide comprises a sequence according to SEQ ID NO: 385 or a sequence that has at least 70% (e.g. at least 90 or 95%) identity thereto.

273. The polypeptide of embodiment 263, wherein the polypeptide comprises a sequence according to SEQ ID NO: 387 or a sequence that has at least 70% (e.g. at least 90 or 95%) identity thereto.

274. The polypeptide of embodiment 263, wherein the polypeptide comprises a sequence according to SEQ ID NO: 389 or a sequence that has at least 70% (e.g. at least 90 or 95%) identity thereto.

275. The polypeptide of embodiment 263, wherein the polypeptide comprises a sequence according to SEQ ID NO: 391 or a sequence that has at least 70% (e.g. at least 90 or 95%) identity thereto.

276. The polypeptide of embodiment 263, wherein the polypeptide comprises a sequence according to SEQ ID NO: 393 or a sequence that has at least 70% (e.g. at least 90 or 95%) identity thereto.

277. The polypeptide of embodiment 263, wherein the polypeptide comprises a sequence according to SEQ ID NO: 395 or a sequence that has at least 70% (e.g. at least 90 or 95%) identity thereto.

278. The polypeptide of embodiment 263, wherein the polypeptide comprises a sequence according to SEQ ID NO: 399 or a sequence that has at least 70% (e.g. at least 90 or 95%) identity thereto.

279. The polypeptide of embodiment 263, wherein the polypeptide comprises a sequence according to SEQ ID NO: 405 or a sequence that has at least 70% (e.g. at least 90 or 95%) identity thereto.

280. The polypeptide of embodiment 263, wherein the polypeptide comprises a sequence according to SEQ ID NO: 417 or a sequence that has at least 70% (e.g. at least 90 or 95%) identity thereto.

281. The polypeptide of embodiment 263, wherein the polypeptide comprises a sequence according to SEQ ID NO: 411 or a sequence that has at least 70% (e.g. at least 90 or 95%) identity thereto.

282. The polypeptide of embodiment 263, wherein the polypeptide comprises a sequence according to SEQ ID NO: 3 or a sequence that has at least 70% (e.g. at least 90 or 95%) identity thereto.

283. The polypeptide of embodiment 263, wherein the polypeptide comprises a sequence according to SEQ ID NO: 279 or a sequence that has at least 70% (e.g. at least 90 or 95%) identity thereto.

284. The polypeptide of embodiment 263, wherein the polypeptide comprises a sequence according to SEQ ID NO: 8 or a sequence that has at least 70% (e.g. at least 90 or 95%) identity thereto.

285. The polypeptide of embodiment 263, wherein the polypeptide comprises a sequence according to SEQ ID NO: 13 or a sequence that has at least 70% (e.g. at least 90 or 95%) identity thereto.

286. The polypeptide of embodiment 263, wherein the polypeptide comprises a sequence according to SEQ ID NO: 280 or a sequence that has at least 70% (e.g. at least 90 or 95%) identity thereto.

287. The polypeptide of embodiment 263, wherein the polypeptide comprises a sequence according to SEQ ID NO: 297 or a sequence that has at least 70% (e.g. at least 90 or 95%) identity thereto.

288. The polypeptide of embodiment 263, wherein the polypeptide comprises a sequence according to SEQ ID NO: 18 or a sequence that has at least 70% (e.g. at least 90 or 95%) identity thereto.

289. The polypeptide of embodiment 263, wherein the polypeptide comprises a sequence according to SEQ ID NO: 73 or a sequence that has at least 70% (e.g. at least 90 or 95%) identity thereto.

290. The polypeptide of embodiment 263, wherein the polypeptide comprises a sequence according to SEQ ID NO: 74 or a sequence that has at least 70% (e.g. at least 90 or 95%) identity thereto.

291. The polypeptide of embodiment 263, wherein the polypeptide comprises a sequence according to SEQ ID NO: 75 or a sequence that has at least 70% (e.g. at least 90 or 95%) identity thereto.

292. The polypeptide of embodiment 263, wherein the polypeptide comprises a sequence according to SEQ ID NO: 283 or a sequence that has at least 70% (e.g. at least 90 or 95%) identity thereto.

293. The polypeptide of embodiment 263, wherein the polypeptide comprises a sequence according to SEQ ID NO: 76 or a sequence that has at least 70% (e.g. at least 90 or 95%) identity thereto.

294. The polypeptide of embodiment 263, wherein the polypeptide comprises a sequence according to SEQ ID NO: 284 or a sequence that has at least 70% (e.g. at least 90 or 95%) identity thereto.

295. The polypeptide of embodiment 263, wherein the polypeptide comprises a sequence according to SEQ ID NO: 401 or a sequence that has at least 70% (e.g. at least 90 or 95%) identity thereto.

296. The polypeptide of embodiment 263, wherein the polypeptide comprises a sequence according to SEQ ID NO: 413 or a sequence that has at least 70% (e.g. at least 90 or 95%) identity thereto.

297. The polypeptide of embodiment 263, wherein the polypeptide comprises a sequence according to SEQ ID NO: 77 or a sequence that has at least 70% (e.g. at least 90 or 95%) identity thereto.

298. The polypeptide of embodiment 263, wherein the polypeptide comprises a sequence according to SEQ ID NO: 285 or a sequence that has at least 70% (e.g. at least 90 or 95%) identity thereto.

299. The polypeptide of embodiment 263, wherein the polypeptide comprises a sequence according to SEQ ID NO: 407 or a sequence that has at least 70% (e.g. at least 90 or 95%) identity thereto.

300. The polypeptide of embodiment 263, wherein the polypeptide comprises a sequence according to SEQ ID NO: 5 or a sequence that has at least 70% (e.g. at least 90 or 95%) identity thereto.

301. The polypeptide of embodiment 263, wherein the polypeptide comprises a sequence according to SEQ ID NO: 10 or a sequence that has at least 70% (e.g. at least 90 or 95%) identity thereto.

302. The polypeptide of embodiment 263, wherein the polypeptide comprises a sequence according to SEQ ID NO: 15 or a sequence that has at least 70% (e.g. at least 90 or 95%) identity thereto.

303. The polypeptide of embodiment 263, wherein the polypeptide comprises a sequence according to SEQ ID NO: 20 or a sequence that has at least 70% (e.g. at least 90 or 95%) identity thereto.

304. A composition comprising the nucleic acid of any one of embodiments 1-152, preferably wherein the composition is an immunogenic composition.

305. A composition comprising a first nucleic acid according to any one of embodiments 1-23, 47-59, 100-103, 107-110, 112-115, 121-122, 126 or 130-136 and second nucleic acid according to any one of embodiments 63-110, 116-117 or 137-138, for example a first nucleic acid and second nucleic acid as defined in Table 27.

306. A composition comprising:
(a) a first nucleic acid encoding a polypeptide comprising:
   i) at least a portion of a Kgp catalytic domain, optionally wherein the at least a portion of a Kgp catalytic domain comprises a full-length Kgp catalytic domain;
   ii) at least a portion of a Kgp DUF2436;
   iii) at least a portion of a Kgp K1 adhesin domain, optionally wherein the at least a portion of a Kgp K1 adhesin comprises a full-length Kgp K1 adhesin domain;
   iv) a first Kgp portion that comprises an ABM1 and a first Kgp portion that comprises an ABM2;
   v) aa second Kgp portion that comprises an ABM1 and a second Kgp portion that comprises an ABM2; and
   vi) a Kgp portion that comprises ABM3;
   wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
   optionally wherein the glycosylation site corresponding to the glycosylation site N691-T693 in the WT Kgp sequence (SEQ ID NO: 157) is mutated by a T693A substitution; and wherein the glycosylation site corresponding to the glycosylation site N950-T952 in the WT Kgp sequence (SEQ ID NO: 157) is mutated by a T952A substitution; and
(b) a second nucleic acid encoding a polypeptide comprising:
   i) at least a portion of a RgpA catalytic domain;
   ii) at least a portion of a RgpA DUF2436;
   iii) at least a portion of a RgpA K1 adhesin domain;
   iv) a first RgpA portion that comprises an ABM1 and a first RgpA portion that comprises an ABM2;
   v) a second RgpA portion that comprises an ABM1 and a second RgpA portion that comprises an ABM2;
   vi) a RgpA portion that comprises ABM3;
   vii) at least a portion of a RgpA K2 adhesin domain;
   wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

307. A composition comprising a first nucleic acid according to embodiment 131 and second nucleic acid according to embodiment 137.

308. The composition of embodiment 307, wherein the first nucleic acid encodes a polypeptide comprising a sequence according to SEQ ID NO: 279, and the second nucleic acid encodes a polypeptide comprising a sequence according to SEQ ID NO: 73.

309. The composition of embodiment 307, wherein:
(a) the first nucleic acid comprises a sequence has at least 70% (e.g. at least 90 or 95%) identity to SEQ ID NO: 289, and the second nucleic acid comprises a sequence has at least 70% (e.g. at least 90 or 95%) identity to SEQ ID NO: 78; or
(b) the first nucleic acid comprises a sequence has at least 70% (e.g. at least 90 or 95%) identity to SEQ ID NO: 289, and the second nucleic acid comprises a sequence has at least 70% (e.g. at least 90 or 95%) identity to SEQ ID NO: 79.

310. The composition of embodiment 307, wherein:
(a) the first nucleic acid comprises a sequence according to SEQ ID NO: 289, and the second nucleic acid comprises a sequence according to SEQ ID NO: 78; or
(b) the first nucleic acid comprises a sequence according to SEQ ID NO: 289, and the second nucleic acid comprises a sequence according to SEQ ID NO: 79.

311. A composition comprising a first nucleic acid and second nucleic acid, wherein the first nucleic acid encodes a polypeptide comprising a sequence that has at least 70% (e.g. at least 90 or 95%) identity to SEQ ID NO: 388, and the second nucleic acid encodes a polypeptide comprising a sequence that has at least 70% sequence identity (e.g. at least 90 or 95%) to SEQ ID NO: 370.

312. A composition comprising a first nucleic acid and second nucleic acid, wherein the first nucleic acid encodes a polypeptide comprising a sequence according to SEQ ID NO: 388, and the second nucleic acid encodes a polypeptide comprising a sequence according to SEQ ID NO: 370.

313. The composition of any one of embodiments 306-312, wherein the first nucleic acid and the second nucleic acid comprise a 5′ UTR according to SEQ ID NO: 238, and a 3′UTR according to SEQ ID NO: 239.

314. The composition of any one of embodiments 304-313, wherein the composition further comprises a lipid nanoparticle (LNP), wherein optionally the nucleic acid is encapsulated in the LNP.

315. The composition of embodiment 314, wherein the LNP comprises at least one cationic lipid, optionally wherein:
(i) the cationic lipid is selected from the group consisting of OF-02, cKK-E10, OF-Deg-Lin, GL-HEPES-E3-E10-DS-3-E18-1, GL-HEPES-E3-E12-DS-4-E10, GL-HEPES-E3-E12-DS-3-E14, SM-102, ALC-0315, IM-001 and IS-001; and/or
(ii) the LNP further comprises a polyethylene glycol (PEG) conjugated (PEGylated) lipid, a cholesterol-based lipid, and a helper lipid.

316. A composition comprising the polypeptide of any one of embodiments 153-303, preferably wherein the composition is an immunogenic composition.

317. A composition comprising a first polypeptide according to any one of embodiments 153-175, 199-207, 211, 252-255, 259-262, 264-267, 273-274, 278 or 282-288 and second polypeptide according to any one of embodiments 215-248, 269-270, 289-290, for example a first polypeptide and second polypeptide as defined in Table 27.

318. A composition comprising:
(a) a first polypeptide comprising:
   i) at least a portion of a Kgp catalytic domain, optionally wherein the at least a portion of a Kgp catalytic domain comprises a full-length Kgp catalytic domain;
   ii) at least a portion of a Kgp DUF2436;
   iii) at least a portion of a Kgp K1 adhesin domain, optionally wherein the at least a portion of a Kgp K1 adhesin comprises a full-length Kgp K1 adhesin domain;
   iv) a first Kgp portion that comprises an ABM1 and a first Kgp portion that comprises an ABM2;
   v) aa second Kgp portion that comprises an ABM1 and a second Kgp portion that comprises an ABM2; and
   vi) a Kgp portion that comprises ABM3;
   wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
   optionally wherein the glycosylation site corresponding to the glycosylation site N691-T693 in the WT Kgp sequence (SEQ ID NO: 157) is mutated by a T693A substitution; and wherein the glycosylation site corresponding to the glycosylation site N950-T952 in the WT Kgp sequence (SEQ ID NO: 157) is mutated by a T952A substitution; and
(b) a second polypeptide comprising:
   i) at least a portion of a RgpA catalytic domain;
   ii) at least a portion of a RgpA DUF2436;
   iii) at least a portion of a RgpA K1 adhesin domain;
   iv) a first RgpA portion that comprises an ABM1 and a first RgpA portion that comprises an ABM2;
   v) a second RgpA portion that comprises an ABM1 and a second RgpA portion that comprises an ABM2;
   vi) a RgpA portion that comprises ABM3;
   vii) at least a portion of a RgpA K2 adhesin domain;
   wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

319. A composition comprising a first polypeptide according to embodiment 283 and a second polypeptide according to embodiment 289.

320. The composition of embodiment 319, wherein the first polypeptide comprises a sequence according to SEQ ID NO: 279, and the second polypeptide comprises a sequence according to SEQ ID NO: 73.

321. A composition comprising a first polypeptide and a second polypeptide, wherein the first polypeptide comprises a sequence that has at least 70% (e.g. at least 90 or 95%) identity to SEQ ID NO: 388, and the second polypeptide comprises a sequence that has at least 70% sequence identity (e.g. at least 90 or 95%) to SEQ ID NO: 370.

322. A composition comprising a first polypeptide and a second polypeptide, wherein the first polypeptide comprises a sequence according to SEQ ID NO: 388, and the second polypeptide comprises a sequence according to SEQ ID NO: 370.

323. The composition of any one of embodiments 316-322, wherein the composition further comprises an adjuvant.

324. The nucleic acid of any one of embodiments 1-152, the polypeptide of any one of embodiments 153-303 or the composition of any one of embodiments 304-323 for use as a medicament.

325. The nucleic acid of any one of embodiments 1-152, the polypeptide of any one of embodiments 153-303 or the composition of any one of embodiments 304-323 for use in treating or preventing a Porphyromonas gingivalis infection, for example periodontitis.

326. Use of the nucleic acid of any one of embodiments 1-152, the polypeptide of any one of embodiments 153-303 or the composition of any one of the embodiments 304-323 for the manufacture of a medicament for treating or preventing a Porphyromonas gingivalis infection, for example periodontitis.

327. A method of treating or preventing a Porphyromonas gingivalis infection, for example periodontitis, in a subject in need thereof, wherein the method comprises administering to the subject the nucleic acid of any one of embodiments 1-152, the polypeptide of any one of embodiments 153-303 or the composition of any one of the embodiments 304-323.

328. A vaccine comprising the nucleic acid of any one of embodiments 1-152, the polypeptide of any one of embodiments 153-303 or the composition of any one of embodiments 304-323.

329. The nucleic acid of any one of embodiments 1-152, the polypeptide of any one of embodiments 153-303, the composition of any one of embodiments 304-323, the nucleic acid, polypeptide or composition for use according to embodiment 324 or embodiment 325, the use according to embodiment 3256, the method of embodiment 327, or the vaccine of embodiment 328, wherein the polypeptide is not a full-length naturally occurring Kgp, RgpA or RgpB polypeptide.

330. The nucleic acid of any one of embodiments 1-152, the polypeptide of any one of embodiments 152-303, the composition of any one of embodiments 304-323, the nucleic acid, polypeptide or composition for use according to embodiment 324 or embodiment 325, the use according to embodiment 326, the method of embodiment 327, or the vaccine of embodiment 328, wherein the polypeptide is a modified polypeptide.

### BRIEF DESCRIPTION OF THE DRAWINGS

FIG. 1 shows the arrangement of the different domains of Kgp and RgpA from P. gingivalis strain W50. Each domain is annotated according to residue position within the wild-type sequences of Kgp (on the top) and RgpA (on the bottom).

FIGS. 2A-2B show the wild-type sequences of Kgp and RgpA from P. gingivalis strain W50. FIG. 2A shows the wild-type sequence of Kgp. FIG. 2B shows the wild-type sequence of RgpA. The sequence of each domain is annotated according to residue position within the wild-type sequence in line with FIG. 1. The wild-type sequences corresponding to the extended Kas2 peptide and the extended Ras2 peptide are underlined. The wild-type sequences corresponding to the Kas2 peptide and the Ras2 peptide are shown in bold. The overlap between the ABM3 sequence and the K1 adhesin domain sequence is highlighted. In particular, the last eight residues of the ABM3 sequence overlap with the first eight residues of the K1 domain. These residues are GTTLSESF.

FIG. 3 shows a model of the three-dimensional structure of the wild-type Kgp (amino acids 229-1732) from P. gingivalis strain W50 produced by AlphaFold2. Each domain is highlighted and annotated. The model suggests that the first portion of Kgp comprising an ABM1 and the first portion of Kgp comprising an ABM2 may associate to form a first fibronectin type III-like domain, while the second portion of Kgp comprising an ABM1 and the second portion of Kgp comprising an ABM2 may associate to form a second fibronectin type III-like domain.

FIGS. 4A-4C show a schematic of the exemplary constructs of the invention. FIG. 4A shows the Kgp constructs Kgp_Kas2-A1, Kgp_1A, Kgp_1B, Kgp_2A and Kgp_2B. FIG. 4B shows the RgpA constructs RgpA_1B and RgpA_2B, as well as the constructs that comprise domains from both Kgp and RgpA domains (Kgp_2A_Ras2, Kgp_2A_K2_Ras2 and Kgp_2A_K2_RgpA_DUF_Ras2). FIG. 4C shows constructs that comprise domains from both Kgp and RgpA (Kgp_2A_K2 Ras2_G3 and Kgp_2A_K2_RgpA_DUF_Ras2_G3). The RgpA domains are highlighted in italics and underlined.

FIG. 5 is a chart depicting anti-HKWC IgG sera of mice immunized with the new antigen designs (Kgp_1A (1A), Kgp_1B (1B), Kgp_2A (2A) and Kgp_2B (2B)) and positive and negative controls. For each construct, the bars from left to right correspond to intracytoplasmic (IC), secreted (Sec), secreted deglycosylated (SecG-), transmembranar (Tm) and transmembranar deglycosylated (TmG-). For the controls, the bars from left to right correspond to empty LNP, Kas2-A1+Alum, 2A (HEK)+Alum, 2A (E. coli)+Alum, P. gingivalis OMV, Buffer+Alum, OMV Buffer.

FIG. 6 is a chart depicting inhibition of Kgp gingipain protease activity in the presence of sera of mice immunized with the new antigen designs and positive and negative controls. For each construct, the bars from left to right correspond to intracytoplasmic (IC), secreted (Sec), secreted deglycosylated (SecG-), transmembranar (Tm) and transmembranar deglycosylated (TmG-). For the controls, the bars from left to right correspond to empty LNP, Kas2-A1+AIOOH, 2A (HEK)+AIOOH, 2A (E. coli)+AIOOH, P. gingivalis OMV, Buffer+Alum, OMV Buffer.

FIG. 7 is a chart depicting inhibition of hemagglutinin activity in the presence of sera of mice immunized with the new antigen designs and positive and negative controls. For each construct, the bars from left to right correspond to intracytoplasmic (IC), secreted (Sec), secreted deglycosylated (SecG-), transmembranar (Tm) and transmembranar deglycosylated (TmG-). For the controls, the bars from left to right correspond to empty LNP, Kas2-A1+Alum, 2A (HEK)+Alum, 2A (E. coli)+Alum, P. gingivalis OMV, Buffer+Alum, OMV Buffer.

FIG. 8 is a chart depicting anti-HKWC IgG sera of mice immunized with the new antigen designs or controls. The new antigen designs include Kgp_1A (A), Kgp_2A (B), Rgp_1B (C), Rgp_2B (D), Kgp_2A_K2_Ras2 (E), or Kgp_2A_K2_RgpA_DUF_Ras2 (F). Also tested were antigen design combinations including Kgp_1A+Rgp_1B (G), Kgp_1A+Rgp_2B (H), Kgp_2A+Rgp_1B (I), Kgp_2A+Rgp_2B (J). The Kas2-A1 protein antigen (K) was used as a benchmark. Protein versions of Rgp_1B (L), Rgp_2B, Kgp_2A_K2_Ras2 (M) and Kgp_2A_K2_RgpA_DUF_Ras2 (N), and P. gingivalis outer membrane vesicles as a source of native gingipain (OMv) were used as positive controls. Empty LNP (LNP alone) served as a negative control.

FIG. 9 is a chart depicting inhibition of hemagglutinin activity in the presence of sera of mice immunized with the new antigen designs or controls. The new antigen designs include Kgp_1A (A), Kgp_2A (B), Rgp_1B (C), Rgp_2B (D), Kgp_2A_K2_Ras2 (E), or Kgp_2A_K2_RgpA_DUF_Ras2 (F). Also tested were antigen design combinations including Kgp_1A+Rgp_1B (G), Kgp_1A+Rgp_2B (H), Kgp_2A+Rgp_1B (I), Kgp_2A+Rgp_2B (J). The Kas2-A1 protein antigen (K) was used as a benchmark. Protein versions of Rgp_1B (L), Rgp_2B, Kgp_2A_K2_Ras2 (M) and Kgp_2A_K2_RgpA_DUF_Ras2 (N), and P. gingivalis outer membrane vesicles as a source of native gingipain (OMv) were used as positive controls. Empty LNP (LNP alone) served as a negative control.

FIG. 10 is a chart depicting inhibition of gingipain protease activity in the presence of sera of mice immunized with the new antigen designs or controls. The new antigen designs include Kgp_1A (A), Kgp_2A (B), Rgp_1B (C), Rgp_2B (D), Kgp_2A_K2_Ras2 (E), or Kgp_2A_K2_RgpA_DUF_Ras2 (F). Also tested were antigen design combinations including Kgp_1A+Rgp_1B (G), Kgp_1A+Rgp_2B (H), Kgp_2A+Rgp_1B (I), Kgp_2A+Rgp_2B (J). The Kas2-A1 protein antigen (K) was used as a benchmark. Protein versions of Rgp_1B (L), Rgp_2B, Kgp_2A_K2_Ras2 (M) and Kgp_2A_K2_RgpA_DUF_Ras2 (N), and P. gingivalis outer membrane vesicles as a source of native gingipain (OMv) were used as positive controls. Empty LNP (LNP alone) served as a negative control.

FIG. 11 is a chart depicting anti-HKWC IgG sera of mice immunized with the new antigen designs or controls. The new antigen designs include Rgp_1B (A), Kgp_1A (B), Kgp_1A_G2 (C), Kgp_2A (D), Kgp_ext_2A_G2 (E). Also tested were antigen design combinations including Kgp_1A_G2+Rgp_1B (G), Kgp_2A+Rgp_1B (H), and Kgp_ext_2A_G2+Rgp_1B (I), Kgp_2A+Rgp_2B (J). The Kas2-A1 protein antigen was used as a benchmark. P. gingivalis outer membrane vesicles as a source of native gingipain (OMv) were used as positive controls. Empty LNP (LNP alone) served as a negative control.

FIG. 12 is a chart depicting inhibition of hemagglutinin activity in the presence of sera of mice immunized with the new antigen designs or controls. The new antigen designs include Rgp_1B (A), Kgp_1A (B), Kgp_1A_G2 (C), Kgp_2A (D), Kgp_ext_2A_G2 (E). Also tested were antigen design combinations including Kgp_LA_G2+Rgp_1B (G), Kgp_2A+Rgp_1B (H), and Kgp_ext_2A_G2+Rgp_1B (I), Kgp_2A+Rgp_2B (J). The Kas2-A1 protein antigen was used as a benchmark. P. gingivalis outer membrane vesicles as a source of native gingipain (OMv) were used as positive controls. Empty LNP (LNP alone) served as a negative control.

FIG. 13 is a chart depicting inhibition of gingipain protease activity in the presence of sera of mice immunized with the new antigen designs or controls. The new antigen designs include Rgp_1B (A), Kgp_1A (B), Kgp_1A_G2 (C), Kgp_2A (D), Kgp_ext_2A_G2 (E). Also tested were antigen design combinations including Kgp_1A_G2+Rgp_1B (G), Kgp_2A+Rgp_1B (H), and Kgp_ext_2A_G2+Rgp_1B (I), Kgp_2A+Rgp_2B (J). The Kas2-A1 protein antigen was used as a benchmark. P. gingivalis outer membrane vesicles as a source of native gingipain (OMv) were used as positive controls. Empty LNP (LNP alone) served as a negative control.

### MODES FOR CARRYING OUT THE INVENTION

### Examples

It will be understood that the invention has been described by way of example only and modifications may be made whilst remaining within the scope and spirit of the invention.

### Example 1—Immunogenicity of the Antigen Designs

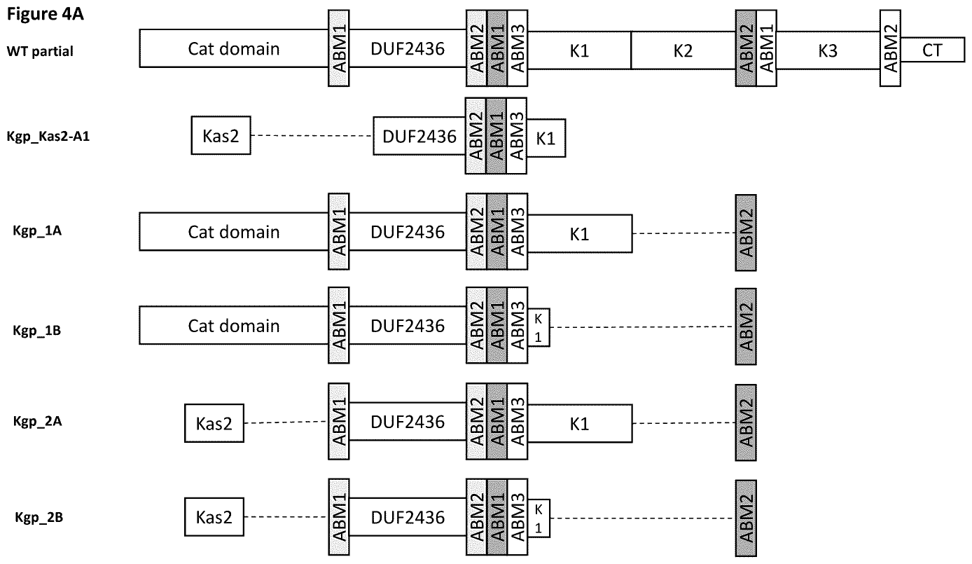

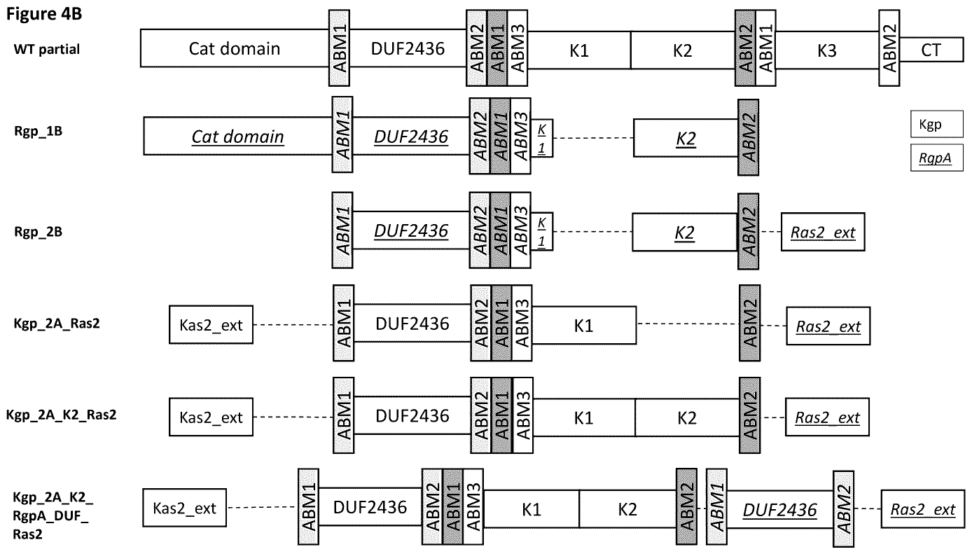

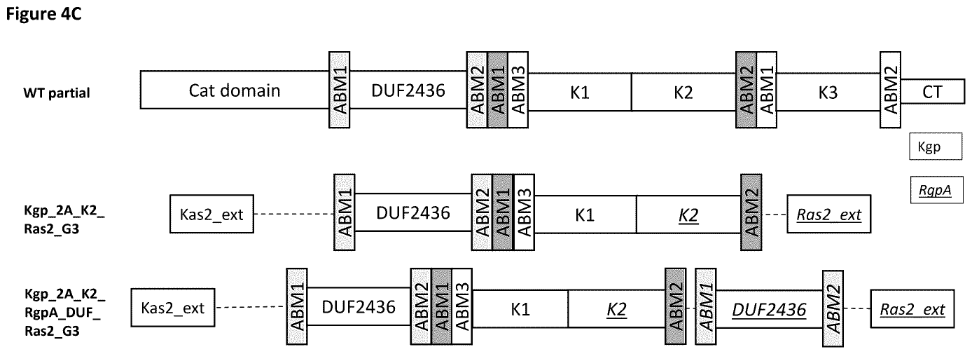

The new antigen designs were tested for their ability to induce a functional immune response. In the present study, 24 different mRNA constructs were evaluated (FIG. 4 and): 4 different Kgp mRNA designs with different sequences of protease domain fused to different sequences of adhesin domain, (i) the entire Cat domain associated with either a full DUF-K1 adhesin domains (Kgp_1A), or with a truncated DUF-K1 adhesin domain (Kgp_1B), or (ii) Kas2 peptide (catalytic site from the Cat domain) associated to the longer (Kgp_2A) or shorter (Kgp_2B) adhesin domains. Each of these mRNA designs were evaluated through 5 different constructs for intracellular, secreted or transmembrane expression location as well as glycosylation modifications (secreted and transmembrane forms were evaluated with and without mutated N-glycosylation sites).

TABLE 28
The mRNA constructs evaluated in the present study.

| | Targeted | | Exemplary nucleic |
|---|---|---|---|
| Construct | expression | SEQ ID NO: | acids (SEQ ID NO:) |
| Kgp_Kas2-A1 | Intracytoplasmic | SEQ ID NO: 142 | SEQ ID NO: 147 |
| | | | SEQ ID NO: 148 |
| Kgp_Kas2-A1_SP | Secreted | SEQ ID NO: 143 | SEQ ID NO: 149 |
| | | | SEQ ID NO: 150 |
| Kgp_Kas2-Al_mGly_SP | Secreted and | SEQ ID NO: 144 | SEQ ID NO: 151 |
| | deglycosylated | | SEQ ID NO: 152 |
| Kgp_Kas2-A1_SP_TM | Transmembranar | SEQ ID NO: 145 | SEQ ID NO: 153 |
| | | | SEQ ID NO: 154 |
| Kgp_Kas2-A1_mGly_SP_TM | Transmembranar | SEQ ID NO: 146 | SEQ ID NO: 155 |
| | and deglycosylated | | SEQ ID NO: 156 |
| Kgp_1A_mCat | Intracytoplasmic | SEQ ID NO: 1 | SEQ ID NO: 21 |
| | | | SEQ ID NO: 22 |
| Kgp_1A_mCat_SP | Secreted | SEQ ID NO: 2 | SEQ ID NO: 23 |
| | | | SEQ ID NO: 24 |
| Kgp_1A_mCat_mGly_SP | Secreted and | SEQ ID NO: 3 | SEQ ID NO: 25 |
| | deglycosylated | | SEQ ID NO: 26 |
| Kgp_1A_mCat_SP_TM | Transmembranar | SEQ ID NO: 4 | SEQ ID NO: 27 |
| | | | SEQ ID NO: 28 |
| Kgp_1A_mCat_mGly_SP_TM | Transmembranar | SEQ ID NO: 5 | SEQ ID NO: 29 |
| | and deglycosylated | | SEQ ID NO: 30 |
| Kgp_1B_mCat | Intracytoplasmic | SEQ ID NO: 6 | SEQ ID NO: 31 |
| | | | SEQ ID NO: 32 |
| Kgp_1B_mCat_SP | Secreted | SEQ ID NO: 7 | SEQ ID NO: 33 |
| | | | SEQ ID NO: 34 |
| Kgp_1B_mCat_mGly_SP | Secreted and | SEQ ID NO: 8 | SEQ ID NO: 35 |
| | deglycosylated | | SEQ ID NO: 36 |
| Kgp_1B_mCat_SP_TM | Transmembranar | SEQ ID NO: 9 | SEQ ID NO: 37 |
| | | | SEQ ID NO: 38 |
| Kgp_1B_mCat_mGly_SP_TM | Transmembranar | SEQ ID NO: 10 | SEQ ID NO: 39 |
| | and deglycosylated | | SEQ ID NO: 40 |
| Kgp_2A | Intracytoplasmic | SEQ ID NO: 11 | SEQ ID NO: 41 |
| | | | SEQ ID NO: 42 |
| Kgp_2A_SP | Secreted | SEQ ID NO: 12 | SEQ ID NO: 43 |
| | | | SEQ ID NO: 44 |
| Kgp_2A_mGly_SP | Secreted and | SEQ ID NO: 13 | SEQ ID NO: 45 |
| | deglycosylated | | SEQ ID NO: 46 |
| Kgp_2A_SP_TM | Transmembranar | SEQ ID NO: 14 | SEQ ID NO: 47 |
| | | | SEQ ID NO: 48 |
| Kgp_2A_mGly_SP_TM | Transmembranar | SEQ ID NO: 15 | SEQ ID NO: 49 |
| | and deglycosylated | | SEQ ID NO: 50 |
| Kgp_2B | Intracytoplasmic | SEQ ID NO: 16 | SEQ ID NO: 51 |
| | | | SEQ ID NO: 52 |
| Kgp_2B_SP | Secreted | SEQ ID NO: 17 | SEQ ID NO: 53 |
| | | | SEQ ID NO: 54 |
| Kgp_2B_mGly_SP | Secreted and | SEQ ID NO: 18 | SEQ ID NO: 55 |
| | deglycosylated | | SEQ ID NO: 56 |
| Kgp_2B_SP_TM | Transmembranar | SEQ ID NO: 19 | SEQ ID NO: 57 |
| | | | SEQ ID NO: 58 |

Preparation of mRNA-LNPs

mRNAs expressing the new antigen designs were synthesized for encapsulation in lipid nanoparticles (LNPs). Each mRNA comprised a cap, identical 5′ and 3′ UTRs (SEQ ID NO: 238 and SEQ ID NO: 239, respectively), and a poly-A tail. Each synthesized mRNA was tested for percentage of capping and poly-A tail length. The percentage of capping exceeded 90% in all constructs apart from four where capping ranged from 82% to 89%. Similarly, the poly-A tail exceeded 100 nucleotides in length in all constructs with a few exceptions. The synthesized mRNAs were also tested for their ability to express the new antigens when transfected in vitro in HEK-293 mammalian cells. All new antigens were expressed at the expected molecular weight (with and without N-glycosylation) and at the targeted localization cell compartments.

Each mRNA was encapsulated in LNPs composed of four lipids: OF-02/DOPE/cholesterol/DMG-PEG, at the molar ratio of 40:30: 28.5:1.5.

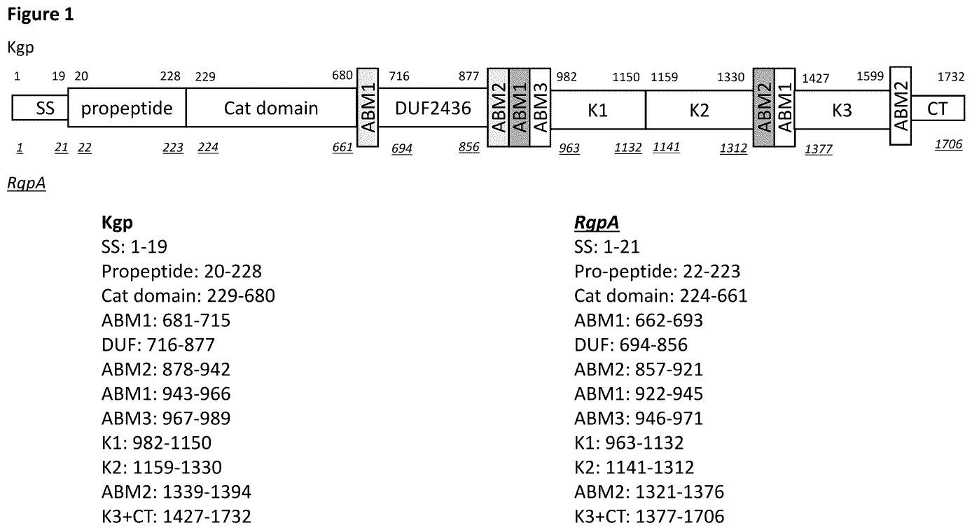

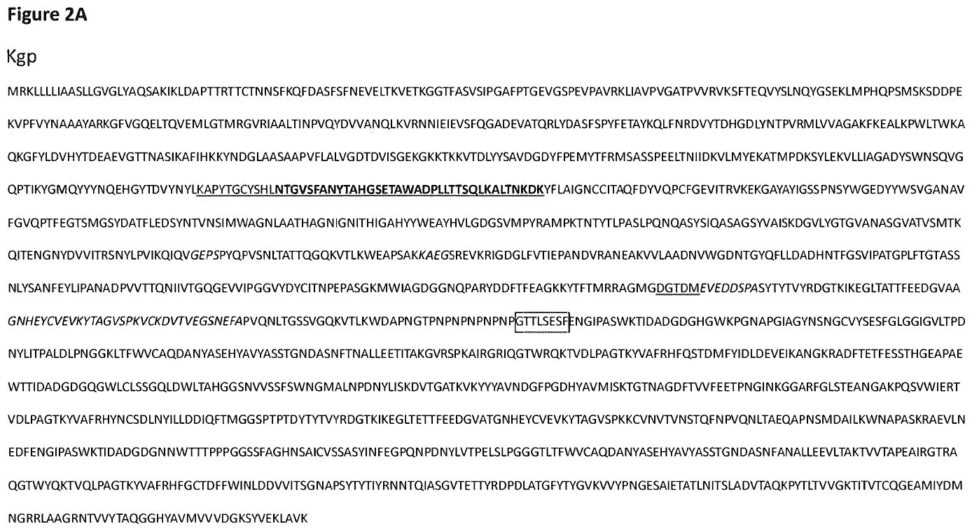

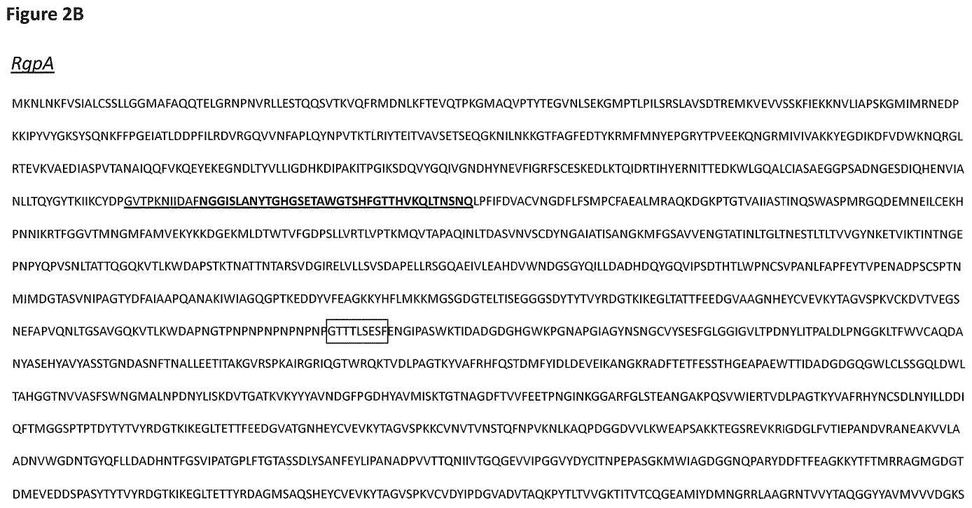

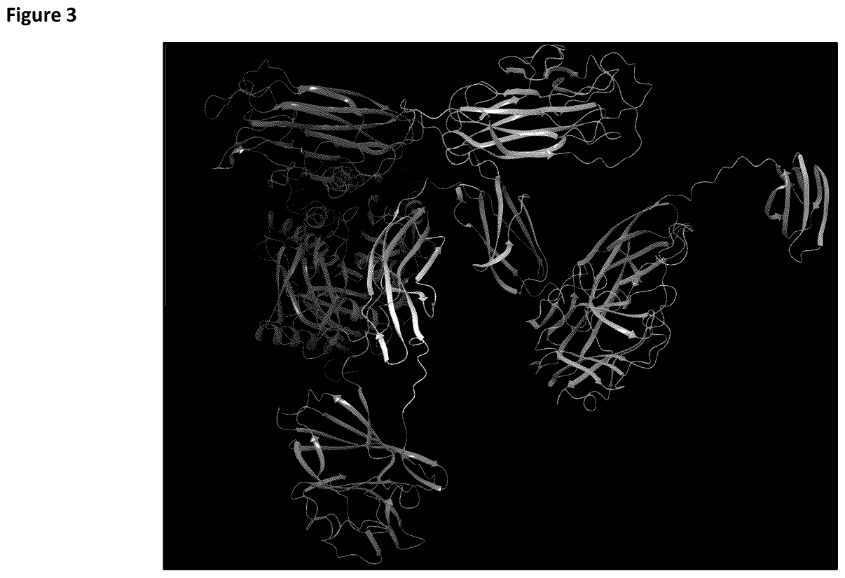

Process A of WO 2018/089801 (see, e.g., Example 1 and FIG. 1 of WO 2018/089801). Process A (“A”) relates to a conventional method of encapsulating mRNA by mixing mRNA with a mixture of lipids, without first pre-forming the lipids into lipid nanoparticles. In an exemplary process, an ethanolic solution of a mixture of lipids (cationic lipid, phosphatidylethanolamine, cholesterol, and polyethylene glycol-lipid) at a fixed lipid to mRNA ratio were combined with an aqueous buffered solution of target mRNA at an acidic pH under controlled conditions to yield a suspension of uniform LNPs. After ultrafiltration and diafiltration into a suitable diluent system, the resulting nanoparticle suspensions were diluted to final concentration of 500 μg/ml mRNA in 10% trehalose, filtered, and stored frozen at −80° C. until use. The LNPs were tested for mRNA integrity, encapsulation efficiency and particle size. The LNPs used in further testing had an average particle size between 103 nm and 130 nm, and encapsulation efficiency between 92% and 98%.

### Immunogenicity Testing

The immunogenicity of the different mRNA constructs formulated in LNP was evaluated in several studies in mice. Representative results are reported hereafter for one study.

Groups of 8 naïve BALB/c female mice received two immunizations via the intramuscular route on days 0 and 21 of mRNA (1 μg/dose) constructs formulated with LNP as described above. As benchmark or positive controls, groups of mice were administered either with 25 μg/dose of recombinant Kas2-A 1 protein antigen (O'Brien-Simpson et al., 2011 and WO2011014947A) adjuvanted in Alum or with P. gingivalis outer membrane vesicles (OMVs, 10 μg/dose) as source of native gingipain. Additionally, one of the mRNA constructs, specifically the Kgp_2A construct, was expressed in both E. coli and in HEK293 mammalian cells. The resulting two Kgp_2A recombinant proteins were purified from the cell supernatants using a 6-His tag, adjuvanted in Alum and administered to two groups of mice (25 μg/dose).

For negative control, mice were administered with empty LNP, OMV buffer or Alum adjuvant alone. Blood samples were collected at day 21 and 35 to assess the induced humoral response.

The P. gingivalis and gingipain specific IgG antibody binding were measured by ELISA using heat killed whole cell bacteria (HKWC) or recombinant protease and adhesin domains as coating antigens.

The functional ability of antibodies to neutralize the gingipain activities were measured by protease activity inhibition (PAI) and hemagglutination inhibition (HAI) assays.

### Hemagglutination Inhibition Assay

In 96-well round-bottom plates, 25 μl of a PBS solution containing P. gingivalis W50 heat killed whole cell bacteria (2.5×10⁷ cells/well) or purified OMVs (100 ng/well) were mixed with increasing amounts of purified IgG (0.25-50 μg prepared in 25 μl PBS) and incubated for one hour at room temperature. Fifty microliters of a 0.5% hen or sheep erythrocytes solution prepared in PBS were then added and the reactions incubated for 3 to 4 hours at room temperature. After incubation, the plates were scanned on Biotek Cytation 7 cell imaging multimode reader and images processed. For each sample the HAI titer was determined through a regression curve to determine the 50% reduction of red blood size pellet and the corresponding reverse dilution of purified IgG.

### Protease Inhibition Assays

Protease inhibition methods were adapted from Nagano et al, Periodontal pathogens-Methods and Protocols 2021, Humana, New York, NY. doi.org/10.1007/978-1-0716-0939-2_10. Briefly, P. gingivalis W50 purified OMVs were diluted to 1 μg/ml in assay buffer (50 mM Tris-HCl, pH 8.0; 100 mM NaCl; 5 mM CaCl₂)) containing 5 mM L-Cysteine (Sigma) and pre-incubated for 10 minutes at 37° C. for enzyme activation. Purified polyclonal IgGs were serial-diluted two-fold in assay buffer (50 μl/well), in 96-well flat-bottom black plates. 50 μl of activated enzyme were then added to each well and the reactions incubated overnight at 4° C. Lysine and Arginine protease activity were evaluated by adding 50 μl of the synthetic substrates Z-His-Glu-Lys-4-methylcoumaryl-7-amide (Peptides International; 30 μM final) and Boc-Phe-Ser-Arg-4-methylcoumaryl-7-amide (Peptides International; 20 μM final), respectively. After 30 minutes of incubation, the release of 4-methylcoumaryl-7-amide was determined by measuring the fluorescence emitted at 440 nm following excitation of the fluorophore at 380 nm. Results were expressed as the percent inhibition compared to the relative fluorescence measured in a reaction with no antibody.

### Results

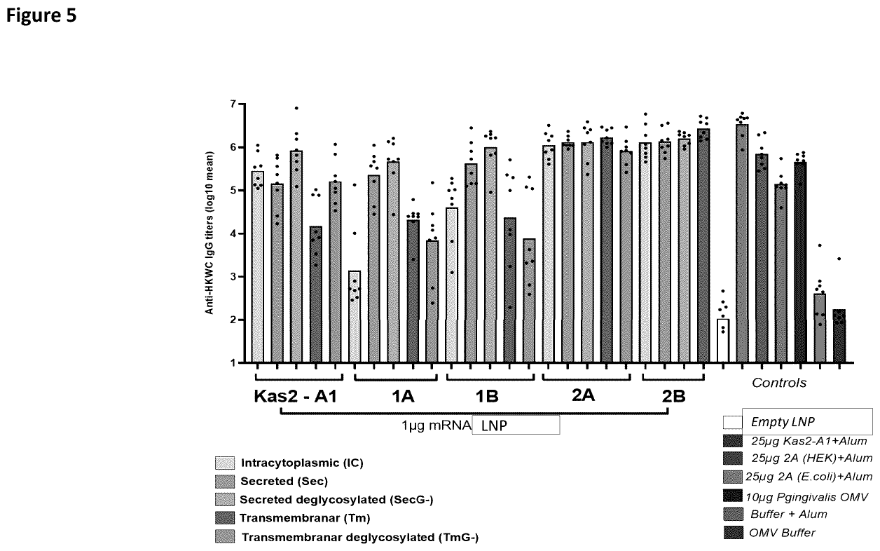

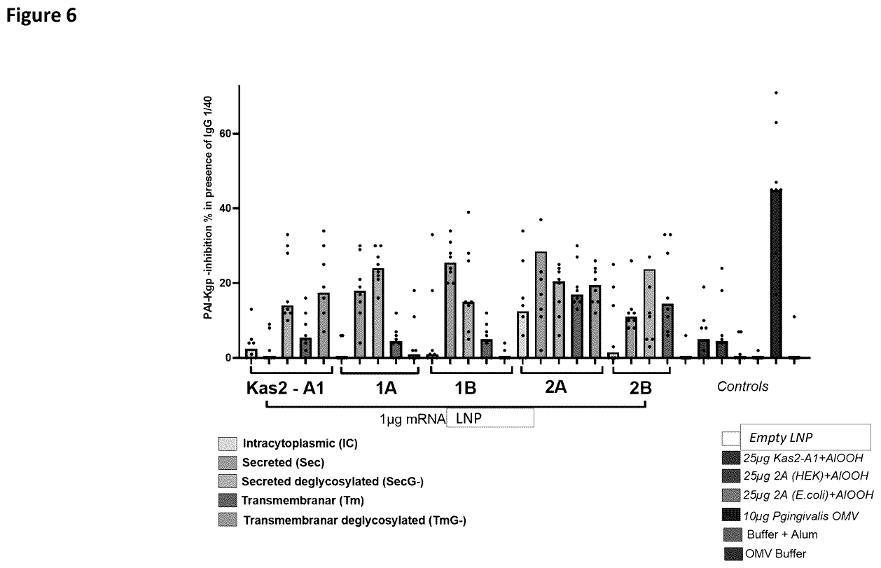

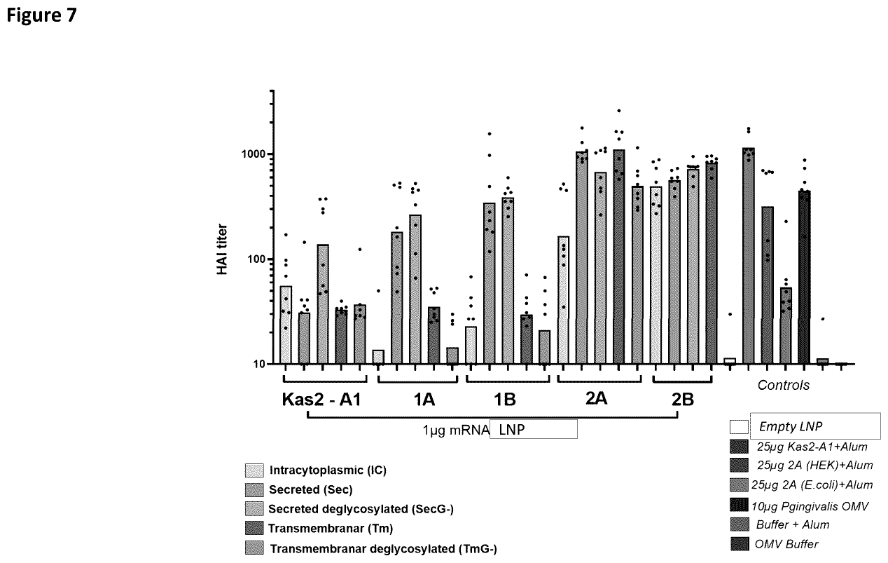

Results showed that all the tested mRNA/LNP constructs were immunogenic and able to induce IgG binding to P. gingivalis HKWC and to the Kgp gingipain protease and adhesin domains. mRNA/LNP from Kgp_2A and Kgp_2B designs as well as some from Kgp_1A and Kgp_1B designs were able to induce IgG titers comparable or higher compared to the OMV positive control or to the Kas2-A1 protein benchmark (FIG. 5). With respect to the Kgp_2A mRNA constructs, the induced IgG titers were comparable or higher than the Kgp_2A recombinant proteins. For all the mRNA constructs, the secreted forms, irrespective of the glycosylation site mutations, were shown to be either at the same level or superior to other expression location. We investigated the ability of mRNA/LNP-induced antibodies (purified IgG from polyclonal serum, pAb) compared with non-specific (empty LNP, PBS/alum)-pAb to inhibit each of these gingipain activities associated with P. gingivalis pathogenicity. mRNA-induced pAb inhibited P. gingivalis proteolytic activity with at least a 9-fold significantly higher (all p-values <0.05, ANOVA for mRNA/LNP secreted forms) anti-protease inhibition activity compared to Kas2-A1 recombinant protein (FIG. 6). With respect to the neutralizing activity of adhesin functions, the mRNA induced-pAb inhibited P. gingivalis hemagglutination activity as well at the same level of the OMVs- or Kas2-A1 induced pAb (FIG. 7). This example demonstrates that the new antigen designs elicit immune responses and the production of functional antibodies that could affect pathogenic activities of P. gingivalis. Further antigen designs, as presented in FIGS. 4A-4C may also be effective at eliciting similar immune responses and the production of functional antibodies that could affect pathogenic activities of P. gingivalis.

### Example 2—Synthesis of IM-001 According to Scheme 2

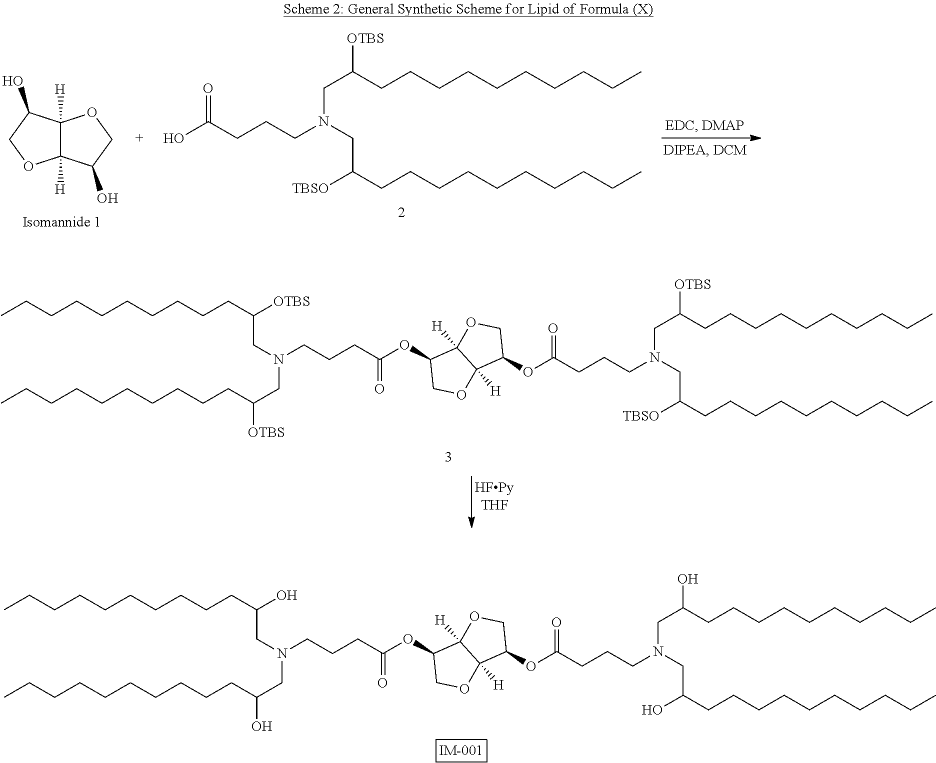

### Abbreviations

DCM: Dichloromethane
DIPEA: N,N-Diisopropylethylamine
DMAP: 4-Dimethylaminopyridine
EDC: 1-Ethyl-3-(3-dimethylaminopropyl)carbodiimide
EtOAc: Ethyl acetate
NaHCO₃: Sodium hydrogencarbonate
Py: Pyridine
Na₂SO₄: Sodium Sulfate
TEA: Triethylamine
TFA: Trifluoroacetic Acid
MS: Mass spectrometry
ESI-MS: Electrospray ionization mass spectrometry
TLC: Thin Layer Chromatography

### Step 1: Synthesis of Intermediate (3)

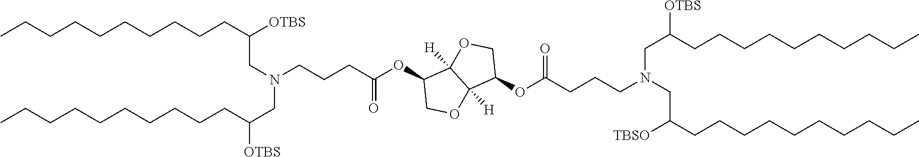 [SMILES: CCCCCCCCCCC(CN(CCCC(=O)O[C@@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 2: To a solution of acid (2) (4.58 g, 6.55 mmol) and isomannide (1) (0.38 g, 2.62 mmol) in dichloromethane (40 mL) were added DIPEA (3.65 mL, 20.96 mmol), DMAP (0.32 g, 2.62 mmol) and EDC (1.5 g, 7.86 mmol). The resulting mixture was stirred at room temperature for overnight. After 16 h, MS and TLC (30% EtOAc in hexanes) analysis indicated completion of the reaction. The reaction mixture was diluted with dichloromethane and washed with saturated NaHCO₃ solution, water and brine solution. The organic layer was dried over anhydrous Na₂SO₄ and concentrated. The crude residue was purified, and the desired product was eluted at 6% EtOAc in hexanes. The product containing fractions were concentrated to obtain 2.58 g (65%) of pure product.

### Results:

ESI-MS: Calculated C₈₆H₁₇₇N₂O₁₀Si₄, [M+H⁺]=1510.25, Observed=1510.3

### Step 2: Synthesis of IM-001

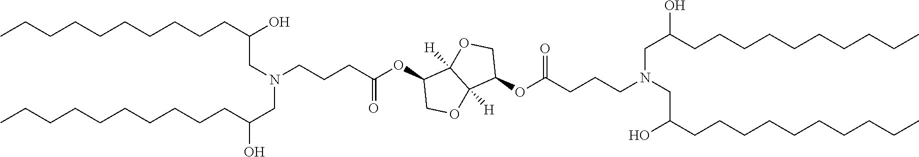 [SMILES: CCCCCCCCCCC(O)CN(CCCC(=O)O[C@@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 2: To a solution of Intermediate (3) (2.58 g, 1.70 mmol) in tetrahydrofuran (14 mL) was added hydrogen fluoride (70% HF.py complex, 7 mL, 51.23 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO₄ and concentrated. The crude residue was purified, and the desired product was eluted at 67% EtOAc in hexanes. The purest fractions were concentrated to obtain 1.1 g (61%) of pure product.

### Results:

¹H NMR (400 MHZ, CDCl₃) δ 5.13-5.03 (m, 2H), 4.73-4.65 (m, 2H), 4.29-3.83 (m, 8H), 3.52-2.98 (m, 12H), 2.69-2.49 (m, 4H), 2.32-2.09 (m, 4H), 1.73-1.12 (m, 72H), 0.88 (t, J=6.6 Hz, 12H).

ESI-MS: Calculated C₆₂H₁₂₁N₂O₁₀, [M+H⁺]=1053.90, Observed=1053.2 and 527.2 [M/2+H+].

### Example 3—Synthesis of IS-001 According to Scheme 3

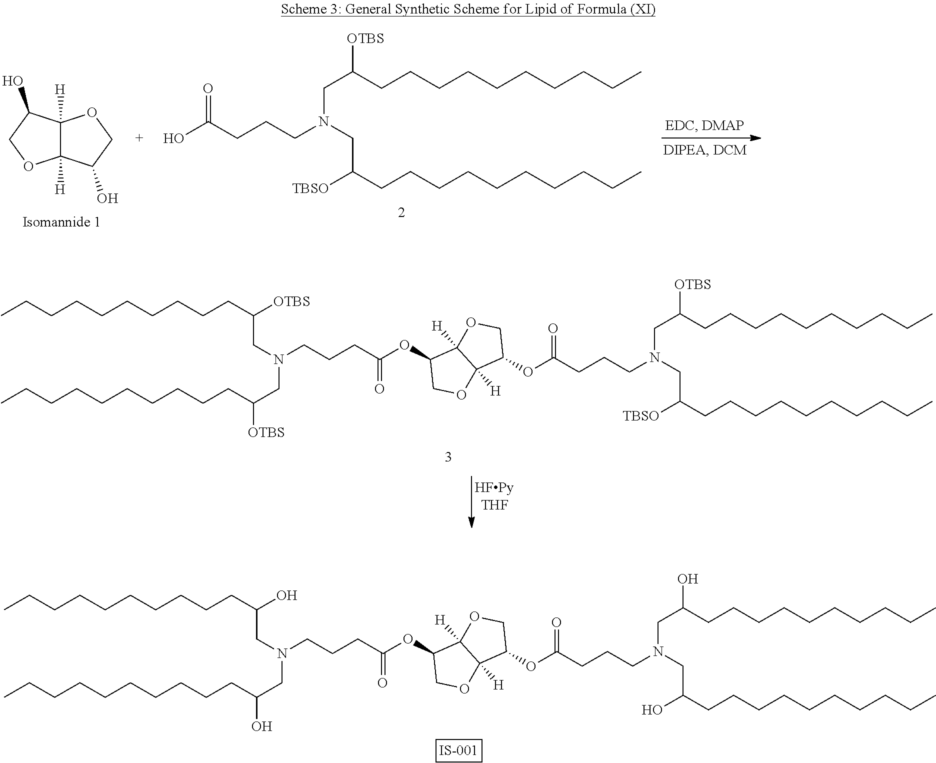

### Abbreviations

DCM: Dichloromethane
DIPEA: N,N-Diisopropylethylamine
DMAP: 4-Dimethylaminopyridine
EDC: 1-Ethyl-3-(3-dimethylaminopropyl)carbodiimide
EtOAc: Ethyl acetate
NaHCO₃: Sodium hydrogencarbonate
Py: Pyridine
Na₂SO₄: Sodium Sulfate
TEA: Triethylamine
TFA: Trifluoroacetic Acid
MS: Mass spectrometry
ESI-MS: Electrospray ionization mass spectrometry
TLC: Thin Layer Chromatography

### Step 1: Synthesis of Intermediate (3)

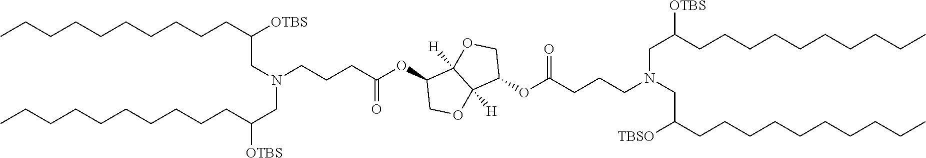 [SMILES: CCCCCCCCCCC(CN(CCCC(=O)O[C@H]1CO[C@H]2[C@@H]1OC[C@H]2OC(=O)CCCN(CC(CCCCCCCCCC)O[Si](C)(C)C(C)(C)C)CC(CCCCCCCCCC)O[Si](C)(C)C(C)(C)C)CC(CCCCCCCCCC)O[Si](C)(C)C(C)(C)C)O[Si](C)(C)C(C)(C)C]

As depicted in Scheme 3: To a solution of acid (2) (1.2 g, 1.71 mmol) and isosorbide (1) (0.100 g, 0.68 mmol) in dichloromethane (10 mL) were added DIPEA (0.95 mL, 5.47 mmol), DMAP (0.084 g, 0.68 mmol) and EDC (0.393 g, 2.05 mmol). The resulting mixture was stirred at room temperature for overnight. After 16 h, MS and TLC (30% EtOAc in hexanes) analysis indicated completion of the reaction. The reaction mixture was diluted with dichloromethane and washed with saturated NaHCO₃ solution, water and brine solution. The organic layer was dried over anhydrous Na₂SO₄ and concentrated. The crude residue was purified, and the desired product was eluted at 6% EtOAc in hexanes. The product containing fractions were concentrated to obtain 0.72 g (69%) of pure product.

### Results:

ESI-MS: Calculated C₈₆H₁₇₇N₂O₁₀Si₄, [M+H⁺]=1510.25, Observed=1510.3 and 755.4 [M/2+H⁺]

### Step 2: Synthesis of IS-001

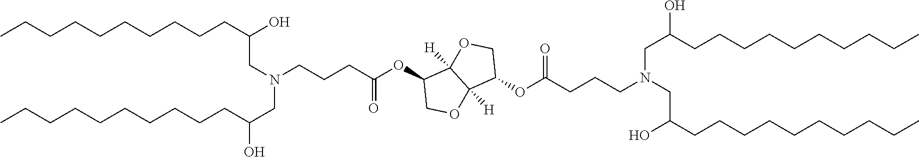 [SMILES: CCCCCCCCCCC(O)CN(CCCC(=O)O[C@H]1CO[C@H]2[C@@H]1OC[C@H]2OC(=O)CCCN(CC(O)CCCCCCCCCC)CC(O)CCCCCCCCCC)CC(O)CCCCCCCCCC]

As depicted in Scheme 3: To a solution of Intermediate (3) (0.72 g, 0.476 mmol) in tetrahydrofuran (4 mL) was added hydrogen fluoride (70% HF.py complex, 2 mL, 14.298 mmol) at 0° C. and stirred at the same temperature for 5 minutes. Then reaction mixture was warmed to room temperature and stirred for 16 h. MS analysis indicated completion of the reaction. The reaction mixture was diluted with ethyl acetate, quenched by slow addition of solid NaHCO₃ at 0° C., followed by saturated NaHCO₃ solution. The organic layer was washed with sat. NaHCO₃ solution, water and brine. Then dried over anhydrous Na₂SO₄ and concentrated. The crude residue was purified, and the desired product was eluted at 65% EtOAc in hexanes. The purest fractions were concentrated to obtain 0.120 g (24%) of pure product.

### Results:

¹H NMR (400 MHZ, CDCl₃) δ 5.30-5.00 (m, 2H), 4.97-4.68 (m, 2H), 4.55-3.71 (m, 8H), 3.57-2.92 (m, 8H), 2.84-2.04 (m, 8H), 1.99-1.01 (m, 76H), 0.88 (t, J=6.8 Hz, 12H).

ESI-MS: Calculated C₆₂H₁₂₁N₂O₁₀, [M+H⁺]=1053.90, Observed=1053.2 and 527.3 [M/2+H⁺]

### Example 4—Immunogenicity of Additional Antigen Designs

Additional antigen designs and antigen combinations were tested for their ability to induce a functional immune response. In the present study, six mRNA constructs were evaluated singly and in combinations (FIG. 4 and). Two of the constructs (Kgp_1A and Kgp_2A) were Kgp constructs tested in Example 1. The remaining four constructs included sequences from RgpA (Rgp_1B and Rgp_2B) or combined sequences from both Kgp and RgpA (Kgp_2A_K2_Ras2 andKgp_2A_K2_RgpA_DUF_Ras2). The mRNA constructs were compared against adjuvanted protein versions of certain antigens, including KAS2-A1, and purified P. gingivalis OMV.

TABLE 29
The mRNA constructs evaluated in the present study.

| | | Exemplary nucleic |
|---|---|---|
| Construct | SEQ ID NO: | acids (SEQ ID NO:) |
| Kgp_1A | SEQ ID NO: 3 | SEQ ID NO: 25 |
| | | SEQ ID NO: 26 |
| Kgp_2A | SEQ ID NO: 13 | SEQ ID NO: 45 |
| | | SEQ ID NO: 46 |
| Rgp_1B | SEQ ID NO: 73 | SEQ ID NO: 78 |
| | | SEQ ID NO: 79 |
| Rgp_2B | SEQ ID NO: 74 | SEQ ID NO: 80 |
| | | SEQ ID NO: 81 |
| Kgp_2A_K2_Ras2 | SEQ ID NO: 401 | |
| Kgp_2A_K2_RgpA_DUF_Ras2 | SEQ ID NO: 407 | |

Preparation of mRNA-LNPs

mRNAs expressing the new antigen designs were synthesized for encapsulation in lipid nanoparticles (LNPs) as described in Example 1.

Immunogenicity testing

The immunogenicity of the different mRNA constructs formulated in LNP was evaluated in mice ( )

Groups of 7 or 8 naïve BALB/c female mice received two immunizations via the intramuscular route on days 0 and 21 of mRNA (2.5 μg/dose) constructs formulated with LNP as described above. When two constructs were administered simultaneously, each was injected at 2.5 μg/dose. As benchmark or positive controls, groups of mice were administered either with 25 μg/dose of recombinant Kas2-A1 protein antigen (O'Brien-Simpson et al., 2011 and WO2011014947A), or protein versions of antigens Rgp_1B, Rgp_2B, Kgp_2A_K2_Ras2 and Kgp_2A_K2_RgpA_DUF_Ras2 adjuvanted in Alum or with P. gingivalis outer membrane vesicles (OMVs, 10 μg/dose) as source of native gingipain.

TABLE 30
The groups evaluated in the present study.

| | | Immunogen | | |
|---|---|---|---|---|
| Sample | Construct(s) | type | Dose | n |
| A | Kgp_1A | mRNA LNP | 2.5 μg | 7 |
| B | Kgp_2A | mRNA LNP | 2.5 μg | 8 |
| C | Rgp_1B | mRNA LNP | 2.5 μg | 8 |
| D | Rgp_2B | mRNA LNP | 2.5 μg | 8 |
| E | Kgp_2A_K2_Ras2 | mRNA LNP | 2.5 μg | 8 |
| F | Kgp_2A_K2_RgpA_DUF_Ras2 | mRNA LNP | 2.5 μg | 7 |
| G | Kgp_1A + Rgp_1B | mRNA LNP | 2.5 μg + | 8 |
| | | | 2.5 μg | |
| H | Kgp_1A + Rgp_2B | mRNA LNP | 2.5 μg + | 8 |
| | | | 2.5 μg | |
| I | Kgp_2A + Rgp_1B | mRNA LNP | 2.5 μg + | 8 |
| | | | 2.5 μg | |
| J | Kgp_2A + Rgp_2B | mRNA LNP | 2.5 μg + | 8 |
| | | | 2.5 μg | |
| K | KAS2-A1 | Protein | 25 | 8 |
| L | Rgp_2B ( | Protein | 25 | 8 |
| M | Kgp_2A_K2_Ras2 | Protein | 25 | 8 |
| N | Kgp_2A_K2_RgpA_DUF_Ras2 | Protein | 25 | 7 |
| OMV | | OMV | 10 | 8 |
| LNP alone | | Empty LNP | — | 8 |

For negative control, mice were administered empty LNP. Blood samples were collected at days 20 and 35 to assess the induced humoral response.

P. gingivalis specific IgG antibody binding was measured by ELISA using heat killed whole cell bacteria (HKWC) as coating antigen.

The functional ability of antibodies to neutralize the gingipain activities were measured by protease activity inhibition (PAI) and hemagglutination inhibition (HAI) assays.

### Hemagglutination Inhibition Assay

In 96-well round-bottom plates, 25 μl of a PBS solution containing P. gingivalis W50 purified OMVs was mixed with increasing amounts of purified IgG (0.25-50 μg prepared in 25 μl PBS) and incubated for one hour at room temperature. Fifty microliters of a 0.5% hen erythrocytes solution prepared in PBS were then added and the reactions incubated for 3 to 4 hours at room temperature. After incubation, the plates were scanned on Biotek Cytation 7 cell imaging multimode reader and images processed. For each sample the HAI titer was determined through a regression curve to determine the 50% reduction of red blood size pellet and the corresponding reverse dilution of purified IgG.

### Protease Inhibition Assays

Protease inhibition methods were adapted from Nagano et al, Periodontal pathogens-Methods and Protocols 2021, Humana, New York, NY. doi.org/10.1007/978-1-0716-0939-2_10. Briefly, P. gingivalis W50 purified OMVs were diluted to 5 μg/ml in assay buffer (50 mM Tris-HCl, pH 8.0; 100 mM NaCl; 5 mM CaCl₂)) containing 5 mM L-Cysteine (Sigma) and pre-incubated for 10 minutes at 37° C. for enzyme activation. Purified polyclonal IgGs were serial-diluted two-fold in assay buffer (50 μl/well), in 96-well flat-bottom black plates. 50 μl of activated enzyme was then added to each well and the reactions incubated overnight at 4° C. Casein protease activity was evaluated by adding 50 μl of the synthetic substrates EnzChek Protease Assay Kits (Molecular Probes; 2.5 ug/mL) after 4 hours of incubation, the fluorescent cleavage product, was determined by measuring the fluorescence emitted at 530 nm following excitation of the fluorophore at 485 nm. Casein PAI titers were determined through the regression curve and correspond to the inverse of dilution which induce 50% of fluorescence of the OMV with no antibody.

### Results

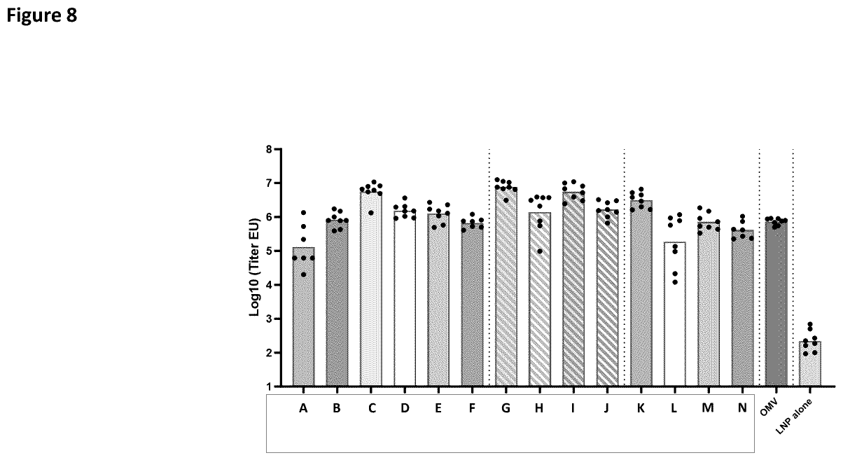

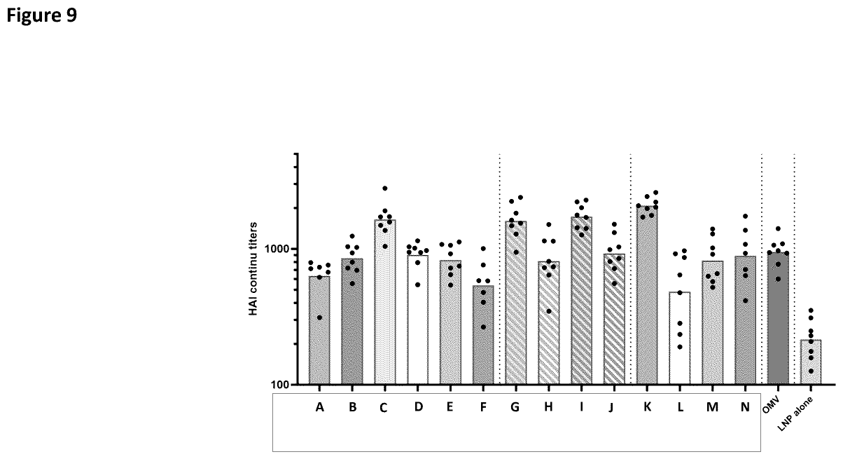

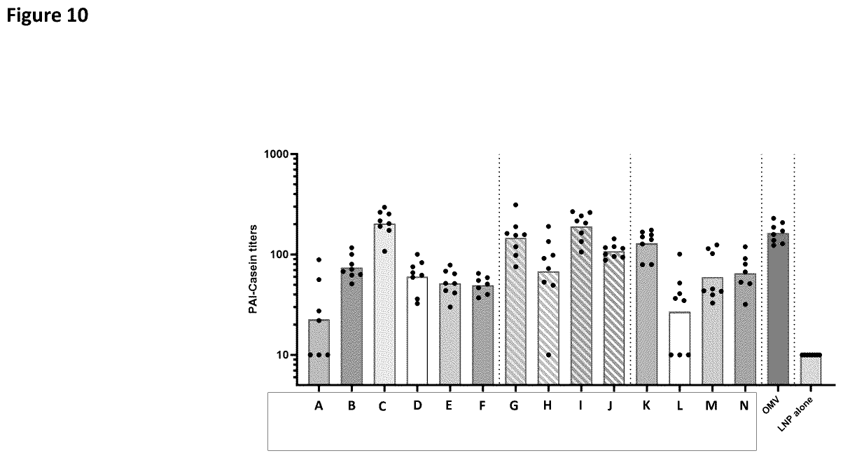

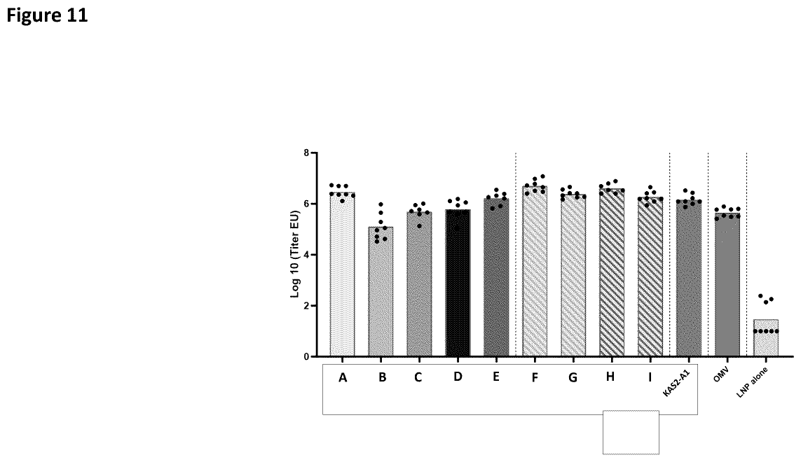

Results showed that all the tested mRNA/LNP constructs and combinations were immunogenic and able to induce IgG binding to P. gingivalis HKWC. The highest titers were observed for mRNA construct Rgp_1B and its combinations Kgp_1A+Rgp_1B, and Kgp_2A+Rgp_1B (FIG. 8). We investigated the ability of mRNA/LNP-induced antibodies (purified IgG from polyclonal serum, pAb) to inhibit each of the gingipain activities associated with P. gingivalis pathogenicity. The highest protease inhibition activities were observed for mRNA construct Rgp_1B and its combinations Kgp_1A+Rgp_1B, and Kgp_2A+Rgp_1B (FIG. 9). With respect to the neutralizing activity of adhesin functions, the mRNA induced-pAb of Rgp_1B and its combinations Kgp_1A+Rgp_1B, and Kgp_2A+Rgp_1B inhibited P. gingivalis hemagglutination activity as well or at the same level of the OMVs-induced pAb (FIG. 10). This example demonstrates that the new antigen designs elicit immune responses and the production of functional antibodies that could affect the pathogenic activities of P. gingivalis.

### Example 5—Immunogenicity of Alternative Antigen Designs

Antigen designs with alternative glycosylation site mutations were compared to the constructs from Example 4 for their ability to induce a functional immune response. In the present study, six mRNA constructs were evaluated singly and in combinations (FIG. 4 and). Two of the constructs (Kgp_1A_G2 and Kgp_ext_2A_G2) were versions of the Kgp constructs tested in Examples 1 and 4 having alternative glycosylation site mutations. The mRNA constructs were compared against adjuvanted protein versions of certain antigens, including KAS2-A1, and purified P. gingivalis OMV.

TABLE 31
The mRNA constructs evaluated in the present study.

| | | Exemplary nucleic |
|---|---|---|
| Construct | SEQ ID NO: | acids (SEQ ID NO:) |
| Kgp_1A | SEQ ID NO: 3 | SEQ ID NO: 25 |
| | | SEQ ID NO: 26 |
| Kgp_1A_G2 | SEQ ID NO: 279 | SEQ ID NO: 289 |
| Kgp_2A | SEQ ID NO: 13 | SEQ ID NO: 45 |
| | | SEQ ID NO: 46 |
| Kgp_ext_2A_G2 | SEQ ID NO: 297 | SEQ ID NO: 298 |
| Rgp_1B | SEQ ID NO: 73 | SEQ ID NO: 78 |
| | | SEQ ID NO: 79 |

Preparation of mRNA-LNPs

mRNAs expressing the new antigen designs were synthesized for encapsulation in lipid nanoparticles (LNPs) as described in Example 4.

### Immunogenicity Testing

The immunogenicity of the different mRNA constructs formulated in LNP was evaluated in mice ( )

Groups of 7 or 8 naïve BALB/c female mice received two immunizations via the intramuscular route on days 0 and 21 of mRNA (2.5 μg/dose) constructs formulated with LNP as described above. When two constructs were administered simultaneously, each was injected at 2.5 μg/dose. As benchmark or positive controls, groups of mice were administered either with 25 μg/dose of recombinant Kas2-A1 protein antigen (O'Brien-Simpson et al., 2011 and WO2011014947A) adjuvanted in Alum or with P. gingivalis outer membrane vesicles (OMVs, 10 μg/dose) as source of native gingipain.

TABLE 32
The groups evaluated in the present study.

| | | Immunogen | | |
|---|---|---|---|---|
| Sample | Construct(s) | type | Dose | n |
| A | Rgp_1B | mRNA LNP | 2.5 μg | 8 |
| B | Kgp_1A | mRNA LNP | 2.5 μg | 8 |
| C | Kgp_1A_G2 | mRNA LNP | 2.5 μg | 8 |
| D | Kgp_2A | mRNA LNP | 2.5 μg | 7 |
| E | Kgp_ext_2A_G2 | mRNA LNP | 2.5 μg | 7 |
| F | Kgp_1A + Rgp_1B | mRNA LNP | 2.5 μg + | 8 |
| | | | 2.5 μg | |
| G | Kgp_1A_G2 + Rgp_1B | mRNA LNP | 2.5 μg + | 8 |
| | | | 2.5 μg | |
| H | Kgp_2A + Rgp_1B | mRNA LNP | 2.5 μg + | 8 |
| | | | 2.5 μg | |
| I | Kgp_ext_2A_G2 + | mRNA LNP | 2.5 μg + | 7 |
| | Rgp_1B | | 2.5 μg | |
| Kas2-A1 | KAS2-A1 | Protein | 25 | 8 |
| OMV | | OMV | 10 | 8 |
| LNP alone | | Empty LNP | — | 8 |

For negative control, mice were administered empty LNP. Blood samples were collected at days 20 and 35 to assess the induced humoral response.

P. gingivalis specific IgG antibody binding was measured by ELISA using heat killed whole cell bacteria (HKWC) as coating antigen.

The functional ability of antibodies to neutralize the gingipain activities were measured by protease activity inhibition (PAI) and hemagglutination inhibition (HAI) assays as described in Example 1.

### Results

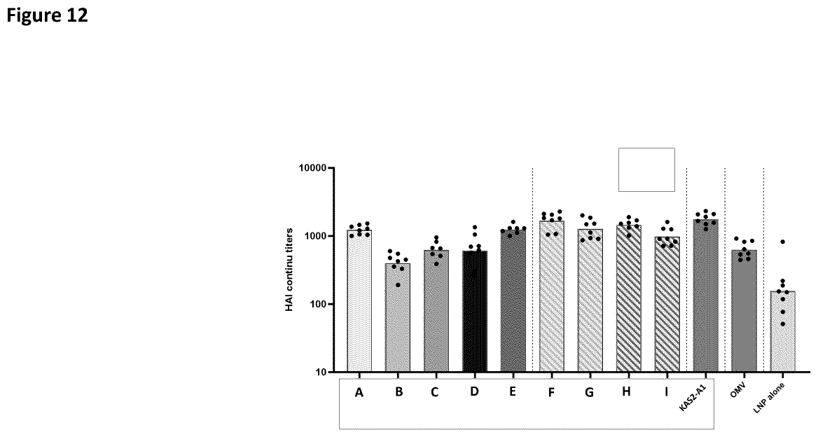

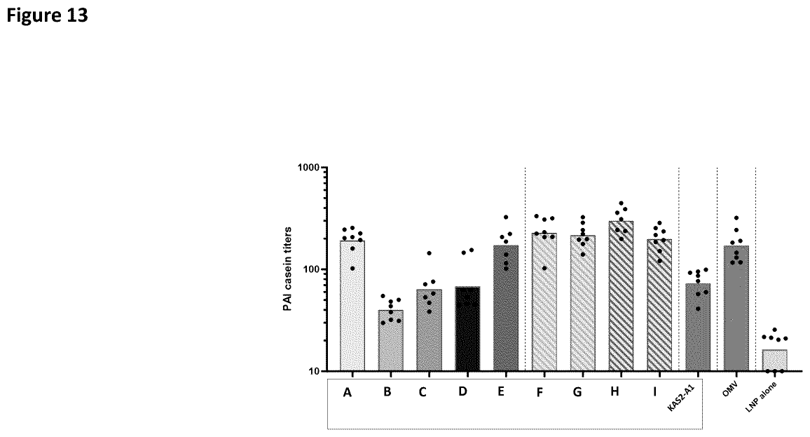

Results showed that all the tested mRNA/LNP constructs and combinations were immunogenic and able to induce IgG binding to P. gingivalis HKWC (FIG. 10), as well as antibodies that provided protease activity inhibition (FIG. 13) and hemagglutination inhibition (FIG. 12). The constructs with alternative glycosylation site mutations showed higher activities when administered singly than the constructs with the original glycosylation site mutations. When administered in combination with Rgp_1B, the constructs with alternative glycosylation site mutations provided generally equivalent activity as those having the original glycosylation site mutations. This example demonstrates that the new antigen designs elicit immune responses and the production of functional antibodies that could affect pathogenic activities of P. gingivalis.

### REFERENCES

Aleksijevic' L H et al. (2022) Porphyromonas gingivalis virulence factors and clinical significance in periodontal disease and coronary artery disease. Pathogens. 11:1173.
Bostanci N and Belibasakis G N (2012) Porphyromonas gingivalis: an invasive and evasive opportunistic oral pathogen. FEMS Microbiology Letters 333:1-9.
Dashper S G et al. (2017) Porphyromonas gingivalis uses specific domain rearrangements and allelic exchange to generate diversity in surface virulence factors. Frontiers in Microbiology 8:48.
Ganuelas L A et al. (2013) The lysine gingipain adhesin domains from Porphyromonas gingivalis interact with erythrocytes and albumin: structures correlate to functions. European Journal of Microbiology and Immunology3: 152-162.
How K et al. (2016) Porphyromonas gingivalis: An overview of periodontopathic pathogen below the gum line. Frontiers in Microbiology 7:53.
Kinane D et al. (2017) Periodontal diseases. Nature Reviews Disease Primers 3:17038.
Konkel M E et al. (2010) Campylobacter jejuni FlpA binds fibronectin and is required for maximal host cell adherence. Journal of Bacteriology 192:68-76.
Li N and Collyer C A (2011). Gingipains from Porphyromonas gingivalis-complex domain structures confer diverse functions. European Journal of Microbiology and Immunology 1:41-58.
Li N et al. (2011) The modular structure of haemagglutinin/adhesin regions in gingipains in Porphyromonas gingivalis. Molecular Microbiology 81:1358-1373.
Mei F et al. (2020) Porphyromonas gingivalis and its systematic impact: current status. Pathogens 9:944.
Nagano et al, Periodontal pathogens-Methods and Protocols 2021, Humana, New York, NY. doi.org/10.1007/978-1-0716-0939-2_10
O'Brien-Simpson N M et al. (2005) An immune response directed to proteinase and adhesin functional epitopes protects against Porphyromonas gingivalis-induced periodontal bone loss. Journal of Immunology 175:3980-3989.
O'Brien-Simpson N M et al. (2016) A therapeutic Porphyromonas gingivalis gingipain vaccine induces neutralising IgG1 antibodies that protect against experimental periodontitis. Npj Vaccines 1:16022.
Paysan-Lafosse et al. (2023) InerPro in 2022. Nucleic Acids Research 6:51.
Slakeski N et al. (1998) Characterization of a second cell-associated Arg-specific cysteine proteinase of Porphyromonas gingivalis and identification of an adhesin-binding motif involved in association of the prtR and prtK proteinases and adhesins into large complexes. Microbiology 144:1583-1592.
World Health Organization (2022) Global oral health status report: towards universal health coverage for oral health by 2030. Licence: CC BY-NC-SA 3.0 IGO. ISBN 978-92-4-006148-4
Xu W et al. (2020) Roles of therapeutic Porphyromonas gingivalis and its virulence factors in periodontitis. Advances in Protein Chemistry and Structural Biology 120:45-84.

1. A nucleic acid comprising a nucleotide sequence encoding a polypeptide, wherein the polypeptide comprises:
i) at least a portion of a Porphyromonas gingivalis Lys-specific proteinase (Kgp) catalytic domain;
ii) at least a portion of a Porphyromonas gingivalis Kgp domain of unknown function 2436 (DUF2436);
iii) at least a portion of a Porphyromonas gingivalis Kgp K1 adhesin domain;
iv) a first Porphyromonas gingivalis Kgp portion that comprises an adhesin binding motif 1 (ABM1) and a first Porphyromonas gingivalis Kgp portion that comprises an adhesin binding motif 2 (ABM2);
v) a second Porphyromonas gingivalis Kgp portion that comprises an ABM1 and a second Porphyromonas gingivalis Kgp portion that comprises an ABM2; and
vi) a Porphyromonas gingivalis Kgp portion that comprises an adhesin binding motif 3 (ABM3).

2. The nucleic acid of claim 1, wherein:
(a) the first Kgp portion that comprises an ABM1 comprises a sequence according to SEQ ID NO: 120 or a sequence that has at least 70% (e.g. at least 90 or 95%) identity thereto;
(b) the first Kgp portion that comprises an ABM2 comprises a sequence according to SEQ ID NO: 130 or a sequence that has at least 70% (e.g. at least 90 or 95%) identity thereto;
(c) the second Kgp portion that comprises an ABM1 comprises a sequence according to SEQ ID NO: 120 or a sequence that has at least 70% (e.g. at least 90 or 95%) identity thereto; and/or
(d) the second Kgp portion that comprises an ABM2 comprises a sequence according to SEQ ID NO: 130 or a sequence that has at least 70% (e.g. at least 90 or 95%) identity thereto.

3. The nucleic acid of claim 1, wherein:
(a) the first Kgp portion that comprises an ABM1 comprises a sequence according to SEQ ID NO: 89 or a sequence that has at least 70% (e.g. at least 90 or 95%) identity thereto;
(b) the first Kgp portion that comprises an ABM2 comprises a sequence according to SEQ ID NO: 91 or a sequence that has at least 70% (e.g. at least 90 or 95%) identity thereto;
(c) the second Kgp portion that comprises an ABM1 comprises a sequence according to SEQ ID NO: 92 or a sequence that has at least 70% (e.g. at least 90 or 95%) identity thereto; and/or
(d) the second Kgp portion that comprises an ABM2 comprises a sequence according to SEQ ID NO: 95 or a sequence that has at least 70% (e.g. at least 90 or 95%) identity thereto.

4. The nucleic acid of claim 1, wherein:
(I) the at least a portion of Kgp DUF2436 is a full-length Kgp DUF2436, wherein optionally the full-length Kgp DUF2436 comprises a sequence according to SEQ ID NO: 90 or a sequence that has at least 70% (e.g. at least 90 or 95%) identity thereto;
(II) the at least a portion of Kgp catalytic domain is:
(a) a full-length Kgp catalytic domain, wherein optionally the full-length Kgp catalytic domain comprises a sequence according to SEQ ID NO: 64 or a sequence that has at least 70% (e.g. at least 90 or 95%) identity thereto; or
(b) a truncated Kgp catalytic domain, wherein optionally the truncated Kgp catalytic domain comprises a Lys-gingipain active site peptide (KAS peptide), for example wherein:
(i) the KAS peptide comprises a Kas2 peptide according to SEQ ID NO: 61 or a sequence that has at least 70% (e.g. at least 90 or 95%) identity thereto; or
(ii) the KAS peptide comprises an extended Kas2 peptide according to SEQ ID NO: 88 or a sequence that has at least 70% (e.g. at least 90 or 95%) identity thereto;
(III) the polypeptide further comprises at least a portion of a Porphyromonas gingivalis Arg-specific proteinase A (RgpA) or Arg-specific proteinase B (RgpB) catalytic domain, wherein optionally the at least a portion of RgpA or RgpB catalytic domain is a truncated RgpA or RgpB catalytic domain, for example wherein the truncated RgpA or RgpB catalytic domain comprises an Arg-gingipain active site peptide (RAS peptide), such as:
(i) the RAS peptide comprises a Ras2 peptide according to SEQ ID NO: 166 or a sequence that has at least 70% (e.g. at least 90 or 95%) identity thereto; or
(ii) the RAS peptide comprises an extended Ras2 peptide according to SEQ ID NO: 97 or a sequence that has at least 70% (e.g. at least 90 or 95%) identity thereto; and/or
(IV) the polypeptide further comprises at least a portion of a Porphyromonas gingivalis Kgp K2 adhesin domain, wherein optionally the at least a portion of Kgp K2 adhesin domain is a full-length K2 adhesin domain, for example wherein the full-length Kgp K2 adhesin domain comprises a sequence according to SEQ ID NO: 96 or a sequence that has at least 70% (e.g. at least 90 or 95%) identity thereto.

5-7. (canceled)

8. The nucleic acid of claim 4, wherein:
(I) the polypeptide further comprises at least a portion of a Porphyromonas gingivalis RgpA K2 adhesin domain,
wherein optionally the at least a portion of RgpA K2 adhesin domain is a full-length K2 adhesin domain, for example wherein the full-length RgpA K2 adhesin domain comprises a sequence according to SEQ ID NO: 104 or a sequence that has at least 70% (e.g. at least 90 or 95%) identity thereto; and/or
(II) the polypeptide comprises at least a portion of an RgpA DUF2436, wherein optionally the RgpA DUF2436 is a full-length RgpA DUF2436, for example, wherein the full-length RgpA DUF2436 comprises a sequence according to SEQ ID NO: 100 or a sequence that has at least 70% (e.g. at least 90 or 95%) identity thereto.

9. (canceled)

10. The nucleic acid of claim 1, wherein:
(a) the at least a portion of the Kgp K1 adhesin domain is a full-length Kgp K1 adhesin domain, wherein optionally the full-length Kgp K1 adhesin domain and Kgp portion comprising ABM3 together comprise a sequence according to SEQ ID NO: 93 or a sequence that has at least 70% (e.g. at least 90 or 95%) identity thereto; or
(b) the at least a portion of the Kgp K1 adhesin domain is a truncated Kgp K1 adhesin domain, wherein optionally the truncated Kgp K1 adhesin domain and Kgp portion comprising ABM3 together comprise a sequence according to SEQ ID NO: 94 or a sequence that has at least 70% (e.g. at least 90 or 95%) identity thereto.

11. A nucleic acid comprising a nucleotide sequence encoding a polypeptide, wherein the polypeptide comprises:
i) at least a portion of a Porphyromonas gingivalis Arg-specific proteinase A (RgpA) or Arg-specific proteinase B (RgpB) catalytic domain, for example at least a portion of the Porphyromonas gingivalis Arg-specific proteinase A (RgpA) catalytic domain;
ii) at least a portion of a Porphyromonas gingivalis RgpA DUF2436;
iii) at least a portion of a Porphyromonas gingivalis RgpA K1 adhesin domain;
iv) a first Porphyromonas gingivalis RgpA portion that comprises an ABM1, and a first Porphyromonas gingivalis RgpA portion that comprises an ABM2;
v) a second Porphyromonas gingivalis RgpA portion that comprises an ABM1, and a second Porphyromonas gingivalis RgpA portion that comprises an ABM2; and
vi) a Porphyromonas gingivalis RgpA portion that comprises an ABM3.

12. The nucleic acid of claim 11, wherein:
(a) the first RgpA portion that comprises an ABM1 comprises a sequence according to SEQ ID NO: 120 or a sequence that has at least 70% (e.g. at least 90 or 95%) identity thereto;
(b) the first RgpA portion that comprises an ABM2 comprises a sequence according to SEQ ID NO: 130 or a sequence that has at least 70% (e.g. at least 90 or 95%) identity thereto;
(c) the second RgpA portion that comprises an ABM1 comprises a sequence according to SEQ ID NO: 120 or a sequence that has at least 70% (e.g. at least 90 or 95%) identity thereto; and/or
(d) the second RgpA portion that comprises an ABM2 comprises a sequence according to SEQ ID NO: 130 or a sequence that has at least 70% (e.g. at least 90 or 95%) identity thereto.

13. The nucleic acid of claim 11, wherein:
(a) the first RgpA portion that comprises an ABM1 comprises a sequence according to SEQ ID NO: 99 or a sequence that has at least 70% (e.g. at least 90 or 95%) identity thereto;
(b) the first RgpA portion that comprises an ABM2 comprises a sequence according to SEQ ID NO: 101 or a sequence that has at least 70% (e.g. at least 90 or 95%) identity thereto;
(c) the second RgpA portion that comprises an ABM1 comprises a sequence according to SEQ ID NO: 102 or a sequence that has at least 70% (e.g. at least 90 or 95%) identity thereto; and/or
(d) the second RgpA portion that comprises an ABM2 comprises a sequence according to SEQ ID NO: 105 or a sequence that has at least 70% (e.g. at least 90 or 95%) identity thereto.

14. The nucleic acid of claim 11, wherein:
(I) the at least a portion of RgpA DUF2436 is a full-length RgpA DUF2436, wherein optionally the full-length RgpA DUF2436 comprises a sequence according to SEQ ID NO: 100 or a sequence that has at least 70% (e.g., at least 90 or 95%) identity thereto;
(II) the at least a portion of RgpA catalytic domain is:
(a) a full-length RgpA catalytic domain, wherein optionally the full-length RgpA catalytic domain comprises a sequence according to SEQ ID NO: 98 or a sequence that has at least 70% (e.g. at least 90 or 95%) identity thereto; or
(b) a truncated RgpA catalytic domain, wherein optionally the truncated RgpA catalytic domain comprises a Arg-gingipain active site peptide (RAS peptide), for example wherein:
(i) the RAS peptide comprises a Ras2 peptide according to SEQ ID NO: 166 or a sequence that has at least 70% (e.g. at least 90 or 95%) identity thereto; or
(ii) the RAS peptide comprises an extended Ras2 peptide according to SEQ ID NO: 97 or a sequence that has at least 70% (e.g. at least 90 or 95%) identity thereto;
(III) the at least a portion of RgpA K1 adhesin domain is a truncated RgpA K1 adhesin domain, wherein optionally the truncated RgpA K1 adhesin domain and RgpA potion comprising ABM3 together comprise a sequence according to SEQ ID NO: 103 or a sequence that has at least 70% (e.g. at least 90 or 95%) identity thereto; and/or
(IV) the polypeptide comprises at least a portion of a Porphyromonas gingivalis RgpA K2 adhesin domain, wherein optionally the at least a portion of RgpA K2 adhesin domain is a full-length K2 adhesin domain, for example wherein the full-length RgpA K2 adhesin domain comprises a sequence according to SEQ ID NO: 104 or a sequence that has at least 70% (e.g. at least 90 or 95%) identity thereto.

15-17. (canceled)

18. The nucleic acid of claim 1, wherein;
(I) the polypeptide further comprises a secretion signal peptide sequence, optionally wherein the secretion signal peptide comprises a secretion signal peptide sequence of HA protein of influenza A virus, further optionally wherein the secretion signal peptide sequence comprises a sequence according to SEQ ID NO: 67;
(II) the polypeptide comprises a sequence according to any one of SEQ ID NOs: 1-20, 73-77, 279, 280, 283-285, 297, 359, 361, 363, 365, 367, 369, 371, 373, 375, 377, 379, 381, 383, 385, 387, 389, 391, 393, 395, 397, 399, 401, 403, 405, 407, 409 or 411; or a sequence that has at least 70% (e.g. at least 90 or 95%) identity thereto; and/or
(III) the nucleic acid is a messenger RNA (mRNA), optionally wherein
(i) the mRNA comprises at least one 5′ untranslated portion (5′ UTR), at least one 3′ untranslated portion (3′ UTR), and/or at least one polyadenylation (poly(A)) sequence;
(ii) the mRNA is unmodified or comprises at least one chemical modification, optionally wherein the mRNA comprises at least one chemical modification, for example wherein the chemical modification comprises N1-methylpseudouridine; and/or
(iii) the mRNA is a self-replicating mRNA or a non-replicating mRNA, e.g. a non-replicating mRNA.

19-21. (canceled)

22. A polypeptide as defined in claim 1.

23. A composition comprising the nucleic acid of claim 1, preferably wherein the composition is an immunogenic composition.

24. A composition comprising:
(a) a first nucleic acid encoding a polypeptide comprising:
i) at least a portion of a Kgp catalytic domain, optionally wherein the at least a portion of a Kgp catalytic domain comprises a full-length Kgp catalytic domain;
ii) at least a portion of a Kgp DUF2436;
iii) at least a portion of a Kgp K1 adhesin domain, optionally wherein the at least a portion of a Kgp K1 adhesin comprises a full-length Kgp K1 adhesin domain;
iv) a first Kgp portion that comprises an ABM1 and a first Kgp portion that comprises an ABM2;
v) a second Kgp portion that comprises an ABM1 and a second Kgp portion that comprises an ABM2; and
vi) a Kgp portion that comprises ABM3;
wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
optionally wherein the glycosylation site corresponding to the glycosylation site N691-T693 in the WT Kgp sequence (SEQ ID NO: 157) is mutated by a T693A substitution; and wherein the glycosylation site corresponding to the glycosylation site N950-T952 in the WT Kgp sequence (SEQ ID NO: 157) is mutated by a T952A substitution; and
(b) a second nucleic acid encoding a polypeptide comprising:
i) at least a portion of a RgpA catalytic domain;
ii) at least a portion of a RgpA DUF2436;
iii) at least a portion of a RgpA K1 adhesin domain;
iv) a first RgpA portion that comprises an ABM1 and a first RgpA portion that comprises an ABM2;
v) a second RgpA portion that comprises an ABM1 and a second RgpA portion that comprises an ABM2;
vi) a RgpA portion that comprises ABM3;
vii) at least a portion of a RgpA K2 adhesin domain;
wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

25. The composition of claim 24, wherein the composition comprises:
i) a first nucleic acid encoding a polypeptide which comprises a sequence according to SEQ ID NO: 279, or a sequence that has at least 70% (e.g. at least 90% or 95%) identity thereto; and
ii) a second nucleic acid encoding a polypeptide which comprises a sequence according to SEQ ID NO: 73, or a sequence that has at least 70% (e.g. at least 90% or 95%) identity thereto.

26. A composition comprising:
(a) a first polypeptide comprising:
i) at least a portion of a Kgp catalytic domain, optionally wherein the at least a portion of a Kgp catalytic domain comprises a full-length Kgp catalytic domain;
ii) at least a portion of a Kgp DUF2436;
iii) at least a portion of a Kgp K1 adhesin domain, optionally wherein the at least a portion of a Kgp K1 adhesin comprises a full-length Kgp K1 adhesin domain;
iv) a first Kgp portion that comprises an ABM1 and a first Kgp portion that comprises an ABM2;
v) aa second Kgp portion that comprises an ABM1 and a second Kgp portion that comprises an ABM2; and
vi) a Kgp portion that comprises ABM3;
wherein the domains are positioned from the N-terminus to the C-terminus of the polypeptide in the following order: Kgp catalytic domain; first Kgp portion comprising ABM1; Kgp DUF2436; first Kgp portion comprising ABM2; second Kgp portion comprising ABM1; Kgp portion comprising ABM3; Kgp K1; second Kgp portion comprising ABM2;
optionally wherein the glycosylation site corresponding to the glycosylation site N691-T693 in the WT Kgp sequence (SEQ ID NO: 157) is mutated by a T693A substitution; and wherein the glycosylation site corresponding to the glycosylation site N950-T952 in the WT Kgp sequence (SEQ ID NO: 157) is mutated by a T952A substitution; and
(b) a second polypeptide comprising:
i) at least a portion of a RgpA catalytic domain;
ii) at least a portion of a RgpA DUF2436;
iii) at least a portion of a RgpA K1 adhesin domain;
iv) a first RgpA portion that comprises an ABM1 and a first RgpA portion that comprises an ABM2;
v) a second RgpA portion that comprises an ABM1 and a second RgpA portion that comprises an ABM2;
vi) a RgpA portion that comprises ABM3;
vii) at least a portion of a RgpA K2 adhesin domain;
wherein the domains are positioned from the N-terminus to the C-terminus of the polypeptide in the following order: RgpA catalytic domain; first RgpA portion comprising ABM1; RgpA DUF2436; first RgpA portion comprising ABM2; second RgpA portion comprising ABM1; RgpA portion comprising ABM3; RgpA K1; RgpA K2; second RgpA portion comprising ABM2.

27. The composition of claim 26, wherein the composition comprises:
i) a first polypeptide which comprises a sequence according to SEQ ID NO: 279, or a sequence that has at least 70% (e.g. at least 90% or 95%) identity thereto; and
ii) a second polypeptide which comprises a sequence according to SEQ ID NQ: 73, or a sequence that has at least 70% (e.g. at least 90% or 95%) identity thereto.

28. A method of treating or preventing a disease in a subject in need thereof, wherein the method comprises administering to the subject the nucleic acid of claim 1.

29. A method of treating or preventing a Porphyromonas gingivalis infection, for example periodontitis, in a subject in need thereof, wherein the method comprises administering to the subject the nucleic acid of claim 1.

30. A vaccine comprising the nucleic acid of claim 1.

31. A polypeptide as defined in claim 11.


SEQUENCE LISTING (418 sequences)

SEQ ID NO: 1 - protein, 989 aa, synthetic construct
MDVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 2 - protein, 1005 aa, synthetic construct
MKAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 3 - protein, 1005 aa, synthetic construct
MKAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 4 - protein, 1033 aa, synthetic construct
MKAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 5 - protein, 1033 aa, synthetic construct
MKAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 6 - protein, 819 aa, synthetic construct
MDVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 7 - protein, 835 aa, synthetic construct
MKAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 8 - protein, 835 aa, synthetic construct
MKAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 9 - protein, 863 aa, synthetic construct
MKAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 10 - protein, 863 aa, synthetic construct
MKAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 11 - protein, 569 aa, synthetic construct
MNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 12 - protein, 585 aa, synthetic construct
MKAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 13 - protein, 585 aa, synthetic construct
MKAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 14 - protein, 613 aa, synthetic construct
MKAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 15 - protein, 613 aa, synthetic construct
MKAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 16 - protein, 399 aa, synthetic construct
MNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 17 - protein, 415 aa, synthetic construct
MKAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 18 - protein, 415 aa, synthetic construct
MKAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 19 - protein, 443 aa, synthetic construct
MKAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 20 - protein, 443 aa, synthetic construct
MKAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 21 - RNA, 2970 nt, synthetic construct
  source 1..2970: mol_type = other RNA
atggacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacaaacgcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacactgcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcacacacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccagcggagttgccacagtgagcatgaccaagcagatcactgagaacggcaattacgatgtggtcatcaccagatccaactatctgcctgtgattaagcaaatccaggtgggcgagccatctccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttaccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 22 - RNA, 2970 nt, synthetic construct
  source 1..2970: mol_type = other RNA
atggacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaaccaatgccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacaccgctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattacacatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgcttccggggtcgccaccgtctctatgacaaagcagattaccgaaaatggcaactatgacgtcgtgatcacccgcagcaattacctgccagtgattaagcagattcaggtgggagagcctagcccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcacaaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 23 - RNA, 3018 nt, synthetic construct
  source 1..3018: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacaaacgcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacactgcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcacacacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccagcggagttgccacagtgagcatgaccaagcagatcactgagaacggcaattacgatgtggtcatcaccagatccaactatctgcctgtgattaagcaaatccaggtgggcgagccatctccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttaccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 24 - RNA, 3018 nt, synthetic construct
  source 1..3018: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaaccaatgccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacaccgctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattacacatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgcttccggggtcgccaccgtctctatgacaaagcagattaccgaaaatggcaactatgacgtcgtgatcacccgcagcaattacctgccagtgattaagcagattcaggtgggagagcctagcccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcacaaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 25 - RNA, 3018 nt, synthetic construct
  source 1..3018: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacacaggcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacgtggcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcgtgcacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccgccggagttgccacagtgagcatgaccaagcagatcactgagaacggcaattacgatgtggtcatcaccagatccaactatctgcctgtgattaagcaaatccaggtgggcgagccatctccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttgccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaactga

SEQ ID NO: 26 - RNA, 3018 nt, synthetic construct
  source 1..3018: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaacccaggccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacgtggctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattgtgcatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgctgccggggtcgccaccgtctctatgacaaagcagattaccgaaaatggcaactatgacgtcgtgatcacccgcagcaattacctgccagtgattaagcagattcaggtgggagagcctagcccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcgccaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaattga

SEQ ID NO: 27 - RNA, 3102 nt, synthetic construct
  source 1..3102: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacaaacgcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacactgcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcacacacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccagcggagttgccacagtgagcatgaccaagcagatcactgagaacggcaattacgatgtggtcatcaccagatccaactatctgcctgtgattaagcaaatccaggtgggcgagccatctccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttaccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaacggcggaagcatcctggctatctacagcaccgtggcctccagcctggtgctggtggtttccctgggcgccattagcttcggcggctga

SEQ ID NO: 28 - RNA, 3102 nt, Synthetic construct
  source 1..3102: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaaccaatgccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacaccgctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattacacatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgcttccggggtcgccaccgtctctatgacaaagcagattaccgaaaatggcaactatgacgtcgtgatcacccgcagcaattacctgccagtgattaagcagattcaggtgggagagcctagcccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcacaaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaatggaggctccatcctggccatctacagcaccgtggcctcctccctggtgctggtggtgtctctcggagccatctccttcggcgggtga

SEQ ID NO: 29 - RNA, 3102 nt, synthetic construct
  source 1..3102: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacacaggcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacgtggcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcgtgcacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccgccggagttgccacagtgagcatgaccaagcagatcactgagaacggcaattacgatgtggtcatcaccagatccaactatctgcctgtgattaagcaaatccaggtgggcgagccatctccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttgccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaacggcggaagcatcctggctatctacagcaccgtggcctccagcctggtgctggtggtttccctgggcgccattagcttcggcggctga

SEQ ID NO: 30 - RNA, 3102 nt, synthetic construct
  source 1..3102: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaacccaggccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacgtggctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattgtgcatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgctgccggggtcgccaccgtctctatgacaaagcagattaccgaaaatggcaactatgacgtcgtgatcacccgcagcaattacctgccagtgattaagcagattcaggtgggagagcctagcccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcgccaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaatggaggctccatcctggccatctacagcaccgtggcctcctccctggtgctggtggtgtctctcggagccatctccttcggcgggtga

SEQ ID NO: 31 - RNA, 2460 nt, synthetic construct
  source 1..2460: mol_type = other RNA
atggacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacaaacgcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacactgcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcacacacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccagcggagttgccacagtgagcatgaccaagcagatcactgagaacggcaattacgatgtggtcatcaccagatccaactatctgcctgtgattaagcaaatccaggtgggcgagccatctccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttccccaccgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 32 - RNA, 2460 nt, synthetic construct
  source 1..2460: mol_type = other RNA
atggacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaaccaatgccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacaccgctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattacacatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgcttccggggtcgccaccgtctctatgacaaagcagattaccgaaaatggcaactatgacgtcgtgatcacccgcagcaattacctgccagtgattaagcagattcaggtgggagagcctagcccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 33 - RNA, 2508 nt, synthetic construct
  source 1..2508: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacaaacgcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacactgcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcacacacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccagcggagttgccacagtgagcatgaccaagcagatcactgagaacggcaattacgatgtggtcatcaccagatccaactatctgcctgtgattaagcaaatccaggtgggcgagccatctccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttccccaccgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 34 - RNA, 2508 nt, synthetic construct
  source 1..2508: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaaccaatgccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacaccgctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattacacatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgcttccggggtcgccaccgtctctatgacaaagcagattaccgaaaatggcaactatgacgtcgtgatcacccgcagcaattacctgccagtgattaagcagattcaggtgggagagcctagcccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 35 - RNA, 2508 nt, synthetic construct
  source 1..2508: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacacaggcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacgtggcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcgtgcacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccgccggagttgccacagtgagcatgaccaagcagatcactgagaacggcaattacgatgtggtcatcaccagatccaactatctgcctgtgattaagcaaatccaggtgggcgagccatctccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttccccactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaactga

SEQ ID NO: 36 - RNA, 2508 nt, synthetic construct
  source 1..2508: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaacccaggccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacgtggctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattgtgcatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgctgccggggtcgccaccgtctctatgacaaagcagattaccgaaaatggcaactatgacgtcgtgatcacccgcagcaattacctgccagtgattaagcagattcaggtgggagagcctagcccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaattga

SEQ ID NO: 37 - RNA, 2592 nt, synthetic construct
  source 1..2592: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacaaacgcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacactgcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcacacacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccagcggagttgccacagtgagcatgaccaagcagatcactgagaacggcaattacgatgtggtcatcaccagatccaactatctgcctgtgattaagcaaatccaggtgggcgagccatctccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttccccaccgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaacggcggaagcatcctggctatctacagcaccgtggcctccagcctggtgctggtggtttccctgggcgccattagcttcggcggctga

SEQ ID NO: 38 - RNA, 2592 nt, synthetic construct
  source 1..2592: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaaccaatgccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacaccgctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattacacatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgcttccggggtcgccaccgtctctatgacaaagcagattaccgaaaatggcaactatgacgtcgtgatcacccgcagcaattacctgccagtgattaagcagattcaggtgggagagcctagcccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaatggaggctccatcctggccatctacagcaccgtggcctcctccctggtgctggtggtgtctctcggagccatctccttcggcgggtga

SEQ ID NO: 39 - RNA, 2592 nt, synthetic construct
  source 1..2592: mol_type = other RNA
atgaaggccattctggtggtgctgctgtacacattcgccaccgccaatgccgacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacacaggcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacgtggcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcgtgcacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccgccggagttgccacagtgagcatgaccaagcagatcactgagaacggcaattacgatgtggtcatcaccagatccaactatctgcctgtgattaagcaaatccaggtgggcgagccatctccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttccccactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaacggcggaagcatcctggctatctacagcaccgtggcctccagcctggtgctggtggtttccctgggcgccattagcttcggcggctga

SEQ ID NO: 40 - RNA, 2592 nt, synthetic construct
  source 1..2592: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaacccaggccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacgtggctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattgtgcatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgctgccggggtcgccaccgtctctatgacaaagcagattaccgaaaatggcaactatgacgtcgtgatcacccgcagcaattacctgccagtgattaagcagattcaggtgggagagcctagcccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaatggaggctccatcctggccatctacagcaccgtggcctcctccctggtgctggtggtgtctctcggagccatctccttcggcgggtga

SEQ ID NO: 41 - RNA, 1710 nt, synthetic construct
  source 1..1710: mol_type = other RNA
atgaacaccggagtgagcttcgccaactacactgcacacggaagcgagacagcctgggctgatccactgctgaccaccagccagctgaaggctctgacaaataaggataaaccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttaccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 42 - RNA, 1710 nt, synthetic construct
  source 1..1710: mol_type = other RNA
atgaataccggagtgtccttcgccaactacaccgctcatggatctgaaactgcctgggctgatcccctgctgacaacaagccagctgaaggccctgaccaataaggacaagccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcacaaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 43 - RNA, 1758 nt, synthetic construct
  source 1..1758: mol_type = other RNA
atgaaggccattctggtggtgctgctgtacacattcgccaccgccaatgccaacaccggagtgagcttcgccaactacactgcacacggaagcgagacagcctgggctgatccactgctgaccaccagccagctgaaggctctgacaaataaggataaaccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttaccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 44 - RNA, 1758 nt, synthetic construct
  source 1..1758: mol_type = other RNA
atgaaagccatcctggtggtgctgctgtacactttcgccaccgctaacgccaataccggagtgtccttcgccaactacaccgctcatggatctgaaactgcctgggctgatcccctgctgacaacaagccagctgaaggccctgaccaataaggacaagccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcacaaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 45 - RNA, 1758 nt, synthetic construct
  source 1..1758: mol_type = other RNA
atgaaggccattctggtggtgctgctgtacacattcgccaccgccaatgccaacaccggagtgagcttcgccaactacgtggcacacggaagcgagacagcctgggctgatccactgctgaccaccagccagctgaaggctctgacaaataaggataaaccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttgccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaactga

SEQ ID NO: 46 - RNA, 1758 nt, synthetic construct
  source 1..1758: mol_type = other RNA
atgaaagccatcctggtggtgctgctgtacactttcgccaccgctaacgccaataccggagtgtccttcgccaactacgtggctcatggatctgaaactgcctgggctgatcccctgctgacaacaagccagctgaaggccctgaccaataaggacaagccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcgccaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaattga

SEQ ID NO: 47 - RNA, 1842 nt, synthetic construct
  source 1..1842: mol_type = other RNA
atgaaggccattctggtggtgctgctgtacacattcgccaccgccaatgccaacaccggagtgagcttcgccaactacactgcacacggaagcgagacagcctgggctgatccactgctgaccaccagccagctgaaggctctgacaaataaggataaaccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttaccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaacggcggaagcatcctggctatctacagcaccgtggcctccagcctggtgctggtggtttccctgggcgccattagcttcggcggctga

SEQ ID NO: 48 - RNA, 1842 nt, synthetic construct
  source 1..1842: mol_type = other RNA
atgaaagccatcctggtggtgctgctgtacactttcgccaccgctaacgccaataccggagtgtccttcgccaactacaccgctcatggatctgaaactgcctgggctgatcccctgctgacaacaagccagctgaaggccctgaccaataaggacaagccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcacaaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaatggaggctccatcctggccatctacagcaccgtggcctcctccctggtgctggtggtgtctctcggagccatctccttcggcgggtga

SEQ ID NO: 49 - RNA, 1842 nt, synthetic construct
  source 1..1842: mol_type = other RNA
atgaaggccattctggtggtgctgctgtacacattcgccaccgccaatgccaacaccggagtgagcttcgccaactacgtggcacacggaagcgagacagcctgggctgatccactgctgaccaccagccagctgaaggctctgacaaataaggataaaccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttcgaaaatggcatcccagcaagctggaagacaattgacgctgatggcgatgggcacggctggaagcctggaaacgcccccggcatcgctggctataactctaatggatgcgtgtatagcgagagctttggcctgggggggatcggggtgctgaccccagataactatctcattacaccagccctcgatctgcccaatggcgggaagctgacattctgggtgtgcgcccaggacgccaattatgcctctgagcactacgccgtctacgcatcctctactgggaatgatgcctctaactttgccaacgccctgctggaggaaacaatcactgcaaagggagtgagaagcccaaaggccattcggggcagaattcagggaacttggagacagaagaccgtggatctgcccgcaggcactaagtacgtggcctttcgccacttccagtctaccgacatgttctatatcgacctggatgaggttgaaatcaaggcaaacgggggcggctcccctaccccaactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaacggcggaagcatcctggctatctacagcaccgtggcctccagcctggtgctggtggtttccctgggcgccattagcttcggcggctga

SEQ ID NO: 50 - RNA, 1842 nt, synthetic construct
  source 1..1842: mol_type = other RNA
atgaaagccatcctggtggtgctgctgtacactttcgccaccgctaacgccaataccggagtgtccttcgccaactacgtggctcatggatctgaaactgcctgggctgatcccctgctgacaacaagccagctgaaggccctgaccaataaggacaagccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcgccaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaatggaggctccatcctggccatctacagcaccgtggcctcctccctggtgctggtggtgtctctcggagccatctccttcggcgggtga

SEQ ID NO: 51 - RNA, 1200 nt, synthetic construct
  source 1..1200: mol_type = other RNA
atgaacaccggagtgagcttcgccaactacactgcacacggaagcgagacagcctgggctgatccactgctgaccaccagccagctgaaggctctgacaaataaggataaaccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttccccactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 52 - RNA, 1200 nt, synthetic construct
  source 1..1200: mol_type = other RNA
atgaataccggagtgtccttcgccaactacaccgctcatggatctgaaactgcctgggctgatcccctgctgacaacaagccagctgaaggccctgaccaataaggacaagccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 53 - RNA, 1248 nt, synthetic construct
  source 1..1248: mol_type = other RNA
atgaaggccattctggtggtgctgctgtacacattcgccaccgccaatgccaacaccggagtgagcttcgccaactacactgcacacggaagcgagacagcctgggctgatccactgctgaccaccagccagctgaaggctctgacaaataaggataaaccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttccccactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaactga

SEQ ID NO: 54 - RNA, 1248 nt, synthetic construct
  source 1..1248: mol_type = other RNA
atgaaagccatcctggtggtgctgctgtacactttcgccaccgctaacgccaataccggagtgtccttcgccaactacaccgctcatggatctgaaactgcctgggctgatcccctgctgacaacaagccagctgaaggccctgaccaataaggacaagccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaattga

SEQ ID NO: 55 - RNA, 1248 nt, synthetic construct
  source 1..1248: mol_type = other RNA
atgaaggccattctggtggtgctgctgtacacattcgccaccgccaatgccaacaccggagtgagcttcgccaactacgtggcacacggaagcgagacagcctgggctgatccactgctgaccaccagccagctgaaggctctgacaaataaggataaaccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttccccactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaactga

SEQ ID NO: 56 - RNA, 1248 nt, synthetic construct
  source 1..1248: mol_type = other RNA
atgaaagccatcctggtggtgctgctgtacactttcgccaccgctaacgccaataccggagtgtccttcgccaactacgtggctcatggatctgaaactgcctgggctgatcccctgctgacaacaagccagctgaaggccctgaccaataaggacaagccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaattga

SEQ ID NO: 57 - RNA, 1332 nt, synthetic construct
  source 1..1332: mol_type = other RNA
atgaaggccattctggtggtgctgctgtacacattcgccaccgccaatgccaacaccggagtgagcttcgccaactacactgcacacggaagcgagacagcctgggctgatccactgctgaccaccagccagctgaaggctctgacaaataaggataaaccataccagccagtttccaatctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagaacctcactggatcttccgtgggacagaaggtgaccctgaagtgggacgctcctaacggaactccaaaccctaaccccaaccctaaccctaaccctgggaccactctgagcgagtccttccccactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtgaatgtgaccgtgaacggcggaagcatcctggctatctacagcaccgtggcctccagcctggtgctggtggtttccctgggcgccattagcttcggcggctga

SEQ ID NO: 58 - RNA, 1332 nt, synthetic construct
  source 1..1332: mol_type = other RNA
atgaaagccatcctggtggtgctgctgtacactttcgccaccgctaacgccaataccggagtgtccttcgccaactacaccgctcatggatctgaaactgcctgggctgatcccctgctgacaacaagccagctgaaggccctgaccaataaggacaagccttaccagccagtcagcaatctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctgaccggcagcagcgtgggccagaaagtgactctgaaatgggatgccccaaacggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtgaatgtgaccgtgaatggaggctccatcctggccatctacagcaccgtggcctcctccctggtgctggtggtgtctctcggagccatctccttcggcgggtga

SEQ ID NO: 59 - RNA, 1332 nt, synthetic construct
  source 1..1332: mol_type = other RNA
atgaaggccattctggtggtgctgctgtacacattcgccaccgccaatgccaacaccggagtgagcttcgccaactacgtggcacacggaagcgagacagcctgggctgatccactgctgaccaccagccagctgaaggctctgacaaataaggataaaccataccagccagtttcccagctgaccgcaaccacccagggccagaaggtgactctgaagtgggaggcaccaagcgcaaagaagtctgagggctcaagggaggtcaagaggatcggggatggcctgtttgtgacaattgagcctgccaatgacgtgcgggctaatgaggccaaagtggttctggctgccgataatgtgtggggggacaacactgggtaccagtttctgctggacgccgaccacaatacatttggctctgtcattccagccaccggaccactgttcaccggaacagcctctagcaatctttattccgctaactttgaatacctgatcccagccaacgccgatcctgtggtgactactcagaatatcatcgtgaccggacagggagaagttgtgattccaggaggggtgtatgactattgcattaccaaccctgagcccgctagcggcaaaatgtggatcgccggagatggcggaaatcagcctgccagatacgacgattttacattcgaagccggaaagaagtacactttcaccatgcggagagccggaatgggcgatggcaccgacatggaggccgaggatgacagcccagcatcctacacctacacagtgtatagagacggaactaaaatcaaagaagggctgacagcaacaaccttcgaggaagatggcgtggccgccggaaatcacgaatattgcgtggaagtgaagtatactgccggagtgtcccccaaggtgtgcaaggacgtgaccgttgaggggtccaatgagttcgctcctgtgcagcagctcactggatcttccgtgggacagaaggtgaccctgaagtgggacgctcctcagggaactccaaaccctaaccccaaccctaaccctaaccctgggaccactctgagcgagtccttccccactgactatacatacacagtgtacagagatgggaccaagatcaaggaaggcctgacagagactaccttcgaggaggatggcgtggcaacaggaaatcacgaatactgcgtggaggtgaagtataccgccggggtgtccccaaagaagtgcgtggacgtgaccgtgaacggcggaagcatcctggctatctacagcaccgtggcctccagcctggtgctggtggtttccctgggcgccattagcttcggcggctga

SEQ ID NO: 60 - RNA, 1332 nt, synthetic construct
  source 1..1332: mol_type = other RNA
atgaaagccatcctggtggtgctgctgtacactttcgccaccgctaacgccaataccggagtgtccttcgccaactacgtggctcatggatctgaaactgcctgggctgatcccctgctgacaacaagccagctgaaggccctgaccaataaggacaagccttaccagccagtcagccagctgaccgctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagcagctgaccggcagcagcgtgggccagaaagtgactctgaaatgggatgccccacagggaactcccaatcctaaccctaatccaaaccctaaccctggcaccacactgtctgagtctttccccaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaatggaggctccatcctggccatctacagcaccgtggcctcctccctggtgctggtggtgtctctcggagccatctccttcggcgggtga

SEQ ID NO: 61 - protein, 36 aa, Porphyromonas gingivalis
NTGVSFANYTAHGSETAWADPLLTTSQLKALTNKDK

SEQ ID NO: 62 - RNA, 108 nt, synthetic construct
  source 1..108: mol_type = other RNA
aacaccggagtgagcttcgccaactacactgcacacggaagcgagacagcctgggctgatccactgctgaccaccagccagctgaaggctctgacaaataaggataaa

SEQ ID NO: 63 - RNA, 108 nt, synthetic construct
  source 1..108: mol_type = other RNA
aataccggagtgtccttcgccaactacaccgctcatggatctgaaactgcctgggctgatcccctgctgacaacaagccagctgaaggccctgaccaataaggacaag

SEQ ID NO: 64 - protein, 456 aa, synthetic construct
DVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPS

SEQ ID NO: 65 - RNA, 1368 nt, synthetic construct
  source 1..1368: mol_type = other RNA
gacgtgtacactgaccacggggacctgtacaacactccagttcgcatgctggtggtggccggggcaaagttcaaagaggccctgaagccatggctgacttggaaagcccagaagggcttctacctggacgtgcactacaccgacgaggcagaagtcggcacaacaaacgcttccatcaaggccttcatccataagaagtacaacgatggactggctgctagcgctgccccagttttcctggccctggtgggcgatacagacgtgatttccggcgaaaagggcaagaagtccaagaaggtgactgacctgtactactcagccgtggatggcgactacttcccagagatgtacacattcaggatgtccgccagctcacctgaggagctgaccaatatcattgacaaagtgctgatgtacgaaaaggcaaccatgcctgacaagtcctatctggagaaggtgctgctgatcgccggagctgattattcttggaactcccaggtggggcagcccacaattaagtacgggatgcagtactattacaaccaggagcatgggtacaccgatgtgtataattacctgaaggccccatacaccggctgctacagccatctgaacaccggagtgagcttcgccaactacactgcacacggaagcgagacagcctgggctgatccactgctgaccaccagccagctgaaggctctgacaaataaggataaatactttctggccatcggcaactgcagcattacagcccagtttgactacgtgcagccttgctttggcgaagtgatcacacgggtgaaagagaaaggagcttacgcttacattggcagctcccctaactcttactggggagaggactattattggagcgtgggcgccaatgcagtgttcggcgtgcagcctacatttgagggcactagcatgggcagctacgacgccacattcctggaggacagctataacaccgtcaactccatcatgtgggctgggaacctggccgcaactcacgccgggaatatcggcaatatcacacacattggggcccattactactgggaagcctatcacgtgctgggcgacgggagcgtgatgccttacagagccatgcctaagactaatacatacaccctgccagcctcactgcctcagaaccaggccagttattctatccaggcctctgcaggaagctacgtggcaatttctaaggatggcgtgctgtatgggactggagtggccaacgccagcggagttgccacagtgagcatgaccaagcagatcactgagaacggcaattacgatgtggtcatcaccagatccaactatctgcctgtgattaagcaaatccaggtgggcgagccatct

SEQ ID NO: 66 - RNA, 1368 nt, synthetic construct
  source 1..1368: mol_type = other RNA
gacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaaccaatgccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacaccgctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattacacatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgcttccggggtcgccaccgtctctatgacaaagcagattaccgaaaatggcaactatgacgtcgtgatcacccgcagcaattacctgccagtgattaagcagattcaggtgggagagcctagc

SEQ ID NO: 67 - protein, 17 aa, synthetic construct
MKAILVVLLYTFATANA

SEQ ID NO: 68 - RNA, 51 nt, synthetic construct
  source 1..51: mol_type = other RNA
atgaaggccattctggtggtgctgctgtacacattcgccaccgccaatgcc

SEQ ID NO: 69 - RNA, 51 nt, synthetic construct
  source 1..51: mol_type = other RNA
atgaaagccatcctggtggtgctgctgtacactttcgccaccgctaacgcc

SEQ ID NO: 70 - protein, 28 aa, synthetic construct
GGSILAIYSTVASSLVLVVSLGAISFGG

SEQ ID NO: 71 - RNA, 84 nt, synthetic construct
  source 1..84: mol_type = other RNA
ggcggaagcatcctggctatctacagcaccgtggcctccagcctggtgctggtggtttccctgggcgccattagcttcggcggc

SEQ ID NO: 72 - RNA, 84 nt, synthetic construct
  source 1..84: mol_type = other RNA
ggaggctccatcctggccatctacagcaccgtggcctcctccctggtgctggtggtgtctctcggagccatctccttcggcggg

SEQ ID NO: 73 - protein, 1006 aa, synthetic construct
MKAILVVLLYTFATANAEPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNIATEDKWLGQALCIASAEGGPSADNGESDIQHENVIANLLTQYGYTKIIKCYDPGVTPKNIIDAFNGGISLANYAGHGSETAWGTSHFGTTHVKQLTNSNQLPFIFDVASVNGDFLFSMPCFAEALMRAQKDGKPTGTVAIIASTIQQSWASPMRGQDEMNEILCEKHPNNIKRTFGGVTMNGMFAMVEKYKKDGEKMLDTWTVFGDPSLLVRTLVPTKMQVTAPAQINLADASVQVSCDYNGAIATISANGKMFGSAVVEQGTATIQLTGLTQESTLTLTVVGYNKETVIKTINTNGEPNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVN

SEQ ID NO: 74 - protein, 615 aa, synthetic construct
MKAILVVLLYTFATANANPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVNGVTPKNIIDAFNGGISLAQYTGHGSETAWGTSHFGTTHVKQLTNSNQ

SEQ ID NO: 75 - protein, 643 aa, synthetic construct
MKAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 76 - protein, 821 aa, synthetic construct
MKAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 77 - protein, 1081 aa, synthetic construct
MKAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 78 - RNA, 3024 nt, synthetic construct
  source 1..3024: mol_type = other RNA
atgaaggccatcctggtggtgctgctgtacaccttcgccaccgccaacgccgagcccggcaggtatacccccgtggaggagaagcagaatggtcggatgatcgtgatcgtggccaagaagtacgagggggacatcaaagacttcgtggactggaagaaccagaggggcctgcggaccgaggtgaaggtggccgaggacatcgccagccccgtgacagccaacgccatccagcagtttgtgaagcaggagtacgagaaggagggcaacgacctgacctatgtgctgctgatcggcgaccacaaagacatcccagccaagatcacccccgggatcaagtccgaccaggtgtatggccagatcgtgggcaacgatcactacaacgaggtgtttatcggcagattctcctgcgagagcaaggaggacctgaagacacagatcgacaggaccatccactacgagcggaacatcgccactgaggataagtggctgggccaggccctgtgcatcgccagcgccgagggcggcccctccgccgacaacggcgagtccgacatccagcacgagaatgtgatcgccaacctcctgacccagtacggctacaccaagatcatcaagtgctacgaccccggggtgacccccaagaatatcatcgacgccttcaacggcggcatttccctggccaattacgccggccacggctccgagacagcctggggcacaagccactttggcaccactcacgtgaagcagctgaccaacagcaatcagctgcccttcatcttcgacgtggccagcgtgaatggagacttcctgttcagcatgccctgcttcgccgaggccctgatgcgggcccagaaggatggcaagcctaccggcaccgtggccatcatcgcctcaaccatccagcagagctgggcctcccccatgaggggccaggacgagatgaacgagatcctgtgcgagaagcaccccaacaacattaaaaggactttcggcggcgtgaccatgaacgggatgtttgccatggtggagaagtacaagaaggacggggaaaagatgctggatacctggaccgtgttcggcgacccctccctgctggtgaggacactggtgcccaccaagatgcaggtcaccgcccccgcccagattaatctggccgacgccagcgttcaggtgtcctgcgactacaacggcgccatcgccaccatctccgccaatggcaaaatgttcggctccgccgtggtggagcaggggactgccacaatccagctgaccggactgacccaggagtccaccctgactctgaccgtggtgggctacaacaaggaaaccgtgatcaagaccatcaacaccaatggcgagccaaatccataccagcccgtgagcaacctggccgccaccacccagggccagaaggtgaccctgaagtgggacgcccctagcaccaagacccaggccaccaccaacaccgccagaagcgtggacggcattagagagctggtgctgctgagcgtgagcgatgcccctgagctgctgaggtccggccaggccgagatcgtgctggaggcccacgatgtgtggaacgacggcagcggctaccagatcctgctggacgccgatcacgatcagtacggccaggtgatcccctccgacacccataccctgtggcccaactgcgccgtgcccgccaacctgtttgcccccttcgagtacaccgtgcccgagaatgccgatccatcttgctcccccaccaacatgatcatggatggcaccgccagcgtgaacatccccgccggcacctacgacttcgccatcgccgccccccaggccaatgccaagatctggatcgccggtcagggccccacaaaggaggacgactacgtgtttgaggccggcaagaagtaccactttctgatgaagaagatgggcagcggcgacggaaccgagctgaccatcagcgaggggggcggatctgactacacctacaccgtgtacagagacggcacaaagatcaaggagggcctgaccgccaccaccttcgaggaggatggcgtggccgccggcaaccacgagtactgcgtggaggtgaagtacaccgccggcgtgagccccaaggtgtgcaaggacgtgaccgtggagggctccaacgagttcgccccagtgcagaacctggccggaagcgccgtgggccagaaggtgactctgaagtgggacgcccctcagggcacccccaaccctaaccccaatcccaaccccaaccccaatccaggaacaacaaccctgtccgagagcttcggcaagagggccgacttcaccgaaaccttcgagagctccacccacggagaggcccccgccgagtggaccacaatcgacgccgacggcgacggccagggctggctgtgcctgtcctctggccagctggactggctgaccgcccacggcggcaccaacgtggtggcttccttctcctggaacggaatggccctgaaccctgataactacctgattagcaaggacgtgaccggcgccaccaaagtgaagtactactacgccgtgaacgacggattccccggcgatcactacgccgtgatgattagcaagaccggcaccaatgccggcgatttcaccgtggtgtttgaggagactcccaatggcatcaacaagggcggcgccagattcggcctgtccacagaggccaacggcgccaagccacagagcgtgtggatcgagagaaccgtggacctgcctgccgggaccaagtacgtggccttcaggcactaccagtgttctgacctgaactacatcctgctggacgacatccagttcaccatgggcggtagccccacccccaccgactacacctacaccgtgtaccgcgatggcaccaagatcaaggagggcctgaccgagacaacctttgaggaggacggcgtggcaaccggcaaccacgagtactgcgtggaggtgaagtacaccgccggcgtgagcccaaagaagtgcgtgcaggtcaccgtgaattaataa

SEQ ID NO: 79 - RNA, 3024 nt, synthetic construct
  source 1..3024: mol_type = other RNA
atgaaggccatcctggtggtgctgctgtacaccttcgccaccgccaacgccgagcccggcaggtatacccccgtggaggagaagcagaatggtcggatgatcgtgatcgtggccaagaagtacgagggggacatcaaagacttcgtggactggaagaaccagaggggcctgcggaccgaggtgaaggtggccgaggacatcgccagccccgtgacagccaacgccatccagcagtttgtgaagcaggagtacgagaaggagggcaacgacctgacctatgtgctgctgatcggcgaccacaaagacatcccagccaagatcacccccgggatcaagtccgaccaggtgtatggccagatcgtgggcaacgatcactacaacgaggtgtttatcggcagattctcctgcgagagcaaggaggacctgaagacacagatcgacaggaccatccactacgagcggaacatcgccactgaggataagtggctgggccaggccctgtgcatcgccagcgccgagggcggcccctccgccgacaacggcgagtccgacatccagcacgagaatgtgatcgccaacctcctgacccagtacggctacaccaagatcatcaagtgctacgaccccggggtgacccccaagaatatcatcgacgccttcaacggcggcatttccctggccaattacgccggccacggctccgagacagcctggggcacaagccactttggcaccactcacgtgaagcagctgaccaacagcaatcagctgcccttcatcttcgacgtggccagcgtgaatggagacttcctgttcagcatgccctgcttcgccgaggccctgatgcgggcccagaaggatggcaagcctaccggcaccgtggccatcatcgcctcaaccatccagcagagctgggcctcccccatgaggggccaggacgagatgaacgagatcctgtgcgagaagcaccccaacaacattaaaaggactttcggcggcgtgaccatgaacgggatgtttgccatggtggagaagtacaagaaggacggggaaaagatgctggatacctggaccgtgttcggcgacccctccctgctggtgaggacactggtgcccaccaagatgcaggtcaccgcccccgcccagattaatctggccgacgccagcgttcaggtgtcctgcgactacaacggcgccatcgccaccatctccgccaatggcaaaatgttcggctccgccgtggtggagcaggggactgccacaatccagctgaccggactgacccaggagtccaccctgactctgaccgtggtgggctacaacaaggaaaccgtgatcaagaccatcaacaccaatggcgagccaaatccataccagcccgtgagcaacctggccgccaccacccagggccagaaggtgaccctgaagtgggacgcccctagcaccaagacccaggccaccaccaacaccgccagaagcgtggacggcattagagagctggtgctgctgagcgtgagcgatgcccctgagctgctgaggtccggccaggccgagatcgtgctggaggcccacgatgtgtggaacgacggcagcggctaccagatcctgctggacgccgatcacgatcagtacggccaggtgatcccctccgacacccataccctgtggcccaactgcgccgtgcccgccaacctgtttgcccccttcgagtacaccgtgcccgagaatgccgatccatcttgctcccccaccaacatgatcatggatggcaccgccagcgtgaacatccccgccggcacctacgacttcgccatcgccgccccccaggccaatgccaagatctggatcgccggtcagggccccacaaaggaggacgactacgtgtttgaggccggcaagaagtaccactttctgatgaagaagatgggcagcggcgacggaaccgagctgaccatcagcgaggggggcggatctgactacacctacaccgtgtacagagacggcacaaagatcaaggagggcctgaccgccaccaccttcgaggaggatggcgtggccgccggcaaccacgagtactgcgtggaggtgaagtacaccgccggcgtgagccccaaggtgtgcaaggacgtgaccgtggagggctccaacgagttcgccccagtgcagaacctggccggaagcgccgtgggccagaaggtgactctgaagtgggacgcccctcagggcacccccaaccctaaccccaatcccaaccccaaccccaatccaggaacaacaaccctgtccgagagcttcggcaagcgggccgatttcaccgagacattcgagagcagcacccacggcgaggcccctgccgagtggaccacaatcgacgccgacggcgacggccagggctggctgtgcctgagtagcggccagctggactggctgaccgcccatgggggcaccaacgtggtggccagcttcagctggaatggcatggccctgaaccccgataactacctgatcagcaaggacgtgaccggcgccaccaaggtgaagtactactacgccgtgaacgacgggttccccggcgaccactacgccgtgatgatcagcaaaaccggcacaaacgccggggacttcaccgtggtgttcgaggaaacccccaacggcatcaataaaggcggcgccaggttcggcctgagcaccgaggccaacggcgccaagccccagagcgtgtggatcgagagaaccgtggacctgccagccggcaccaagtacgtggcctttagacactaccagtgctctgacctgaactacatcctgctggacgatatccagttcaccatgggcggcagccccacccccaccgactacacctacaccgtgtaccgcgatggcaccaagatcaaggagggcctgaccgagacaacctttgaggaggacggcgtggcaaccggcaaccacgagtactgcgtggaggtgaagtacaccgccggcgtgagcccaaagaagtgcgtgcaggtcaccgtgaattaataa

SEQ ID NO: 80 - RNA, 1851 nt, synthetic construct
  source 1..1851: mol_type = other RNA
atgaaggccatcctggtggtgctgctgtacaccttcgccaccgccaacgccaatccataccagcccgtgagcaacctggccgccaccacccagggccagaaggtgaccctgaagtgggacgcccctagcaccaagacccaggccaccaccaacaccgccagaagcgtggacggcattagagagctggtgctgctgagcgtgagcgatgcccctgagctgctgaggtccggccaggccgagatcgtgctggaggcccacgatgtgtggaacgacggcagcggctaccagatcctgctggacgccgatcacgatcagtacggccaggtgatcccctccgacacccataccctgtggcccaactgcgccgtgcccgccaacctgtttgcccccttcgagtacaccgtgcccgagaatgccgatccatcttgctcccccaccaacatgatcatggatggcaccgccagcgtgaacatccccgccggcacctacgacttcgccatcgccgccccccaggccaatgccaagatctggatcgccggtcagggccccacaaaggaggacgactacgtgtttgaggccggcaagaagtaccactttctgatgaagaagatgggcagcggcgacggaaccgagctgaccatcagcgaggggggcggatctgactacacctacaccgtgtacagagacggcacaaagatcaaggagggcctgaccgccaccaccttcgaggaggatggcgtggccgccggcaaccacgagtactgcgtggaggtgaagtacaccgccggcgtgagccccaaggtgtgcaaggacgtgaccgtggagggctccaacgagttcgccccagtgcagaacctggccggaagcgccgtgggccagaaggtgactctgaagtgggacgcccctcagggcacccccaaccctaaccccaatcccaaccccaaccccaatccaggaacaacaaccctgtccgagagcttcggcaagagggccgacttcaccgaaaccttcgagagctccacccacggagaggcccccgccgagtggaccacaatcgacgccgacggcgacggccagggctggctgtgcctgtcctctggccagctggactggctgaccgcccacggcggcaccaacgtggtggcttccttctcctggaacggaatggccctgaaccctgataactacctgattagcaaggacgtgaccggcgccaccaaagtgaagtactactacgccgtgaacgacggattccccggcgatcactacgccgtgatgattagcaagaccggcaccaatgccggcgatttcaccgtggtgtttgaggagactcccaatggcatcaacaagggcggcgccagattcggcctgtccacagaggccaacggcgccaagccacagagcgtgtggatcgagagaaccgtggacctgcctgccgggaccaagtacgtggccttcaggcactaccagtgttctgacctgaactacatcctgctggacgacatccagttcaccatgggcggtagccccacccccaccgactacacctacaccgtgtaccgcgatggcaccaagatcaaggagggcctgaccgagacaacctttgaggaggacggcgtggcaaccggcaaccacgagtactgcgtggaggtgaagtacaccgccggcgtgagcccaaagaagtgcgtgcaggtcaccgtgaatggcgtgacccccaagaacattatcgacgccttcaatggcggaatctctctggcccagtataccggccacggcagcgaaaccgcctggggcacctctcactttggcaccacccacgtgaagcagctgaccaatagcaaccagtaataa

SEQ ID NO: 81 - RNA, 1851 nt, synthetic construct
  source 1..1851: mol_type = other RNA
atgaaggccatcctggtggtgctgctgtacaccttcgccaccgccaacgccaatccataccagcccgtgagcaacctggccgccaccacccagggccagaaggtgaccctgaagtgggacgcccctagcaccaagacccaggccaccaccaacaccgccagaagcgtggacggcattagagagctggtgctgctgagcgtgagcgatgcccctgagctgctgaggtccggccaggccgagatcgtgctggaggcccacgatgtgtggaacgacggcagcggctaccagatcctgctggacgccgatcacgatcagtacggccaggtgatcccctccgacacccataccctgtggcccaactgcgccgtgcccgccaacctgtttgcccccttcgagtacaccgtgcccgagaatgccgatccatcttgctcccccaccaacatgatcatggatggcaccgccagcgtgaacatccccgccggcacctacgacttcgccatcgccgccccccaggccaatgccaagatctggatcgccggtcagggccccacaaaggaggacgactacgtgtttgaggccggcaagaagtaccactttctgatgaagaagatgggcagcggcgacggaaccgagctgaccatcagcgaggggggcggatctgactacacctacaccgtgtacagagacggcacaaagatcaaggagggcctgaccgccaccaccttcgaggaggatggcgtggccgccggcaaccacgagtactgcgtggaggtgaagtacaccgccggcgtgagccccaaggtgtgcaaggacgtgaccgtggagggctccaacgagttcgccccagtgcagaacctggccggaagcgccgtgggccagaaggtgactctgaagtgggacgcccctcagggcacccccaaccctaaccccaatcccaaccccaaccccaatccaggaacaacaaccctgtccgagagcttcggcaagcgggccgatttcaccgagacattcgagagcagcacccacggcgaggcccctgccgagtggaccacaatcgacgccgacggcgacggccagggctggctgtgcctgagtagcggccagctggactggctgaccgcccatgggggcaccaacgtggtggccagcttcagctggaatggcatggccctgaaccccgataactacctgatcagcaaggacgtgaccggcgccaccaaggtgaagtactactacgccgtgaacgacgggttccccggcgaccactacgccgtgatgatcagcaaaaccggcacaaacgccggggacttcaccgtggtgttcgaggaaacccccaacggcatcaataaaggcggcgccaggttcggcctgagcaccgaggccaacggcgccaagccccagagcgtgtggatcgagagaaccgtggacctgccagccggcaccaagtacgtggcctttagacactaccagtgctctgacctgaactacatcctgctggacgatatccagttcaccatgggcggcagccccacccccaccgactacacctacaccgtgtaccgcgatggcaccaagatcaaggagggcctgaccgagacaacctttgaggaggacggcgtggcaaccggcaaccacgagtactgcgtggaggtgaagtacaccgccggcgtgagcccaaagaagtgcgtgcaggtcaccgtgaatggcgtgacccccaagaacattatcgacgccttcaatggcggaatctctctggcccagtataccggccacggcagcgaaaccgcctggggcacctctcactttggcaccacccacgtgaagcagctgaccaatagcaaccagtaataa

SEQ ID NO: 82 - RNA, 1935 nt, synthetic construct
  source 1..1935: mol_type = other RNA
atgaaggccatccttgtggtgctgctgtatacctttgccaccgccaacgccaaggccccttacaccggctgctacagccacctgaacaccggcgtgagcttcgccaactacgtggcccacggctctgagactgcctgggccgacccactgctgaccacatcccagctgaaggccctgaccaataaggacaagccctaccagcccgtgagccagctgaccgccaccacacagggccagaaggtgaccctgaaatgggaggcccccagcgccaagaagtccgagggctccagagaggtgaagagaatcggcgacggcctgttcgtgactatcgagcccgccaacgatgtgagagccaatgaggccaaggtggtgctggccgccgacaacgtgtggggcgataacaccggctaccagttcctgctggacgccgaccacaatacatttggcagcgtgatccccgccaccggcccactgttcaccggcacagcctccagcaacctgtactccgccaactttgagtacctgatccccgccaacgccgacccagtggtgaccacccagaacatcatcgtgaccggccagggcgaagtggtgatccccggcggcgtgtacgactattgtatcaccaacccagagccagccagcggaaagatgtggatcgccggcgatggcggcaaccagcctgcccggtacgacgatttcaccttcgaggccggcaaaaagtacacctttaccatgaggagagccggcatgggcgacggcaccgacatggaggccgaggacgatagccccgccagctacacctacacagtgtaccgggacggcaccaagatcaaggaaggcctgaccgccaccaccttcgaggaagacggcgtggccgccggcaaccacgaatactgtgtggaggtgaagtacacagccggcgtgagccccaaggtgtgcaaggacgtgaccgtggagggctccaacgagtttgccccagtgcagcagctgaccggcagcagcgtgggccagaaggtgaccctgaagtgggacgccccacagggtacccccaaccctaaccccaaccccaaccccaacccaggcaccaccctgagcgagtccttcgaaaacgggattcccgcctcctggaagaccatcgacgccgacggcgacggccacggctggaagccaggcaacgcccccggcatcgccggctataacagcaacggctgtgtgtatagcgagagcttcggactgggcggcatcggggtgctgacccccgataactacctgatcacccccgccctggacctgcctaacggcggcaagctgaccttctgggtgtgcgcccaggacgccaattatgccagcgagcactacgccgtgtacgccagcagcaccggcaacgacgccagcaacttcgccaatgccctgctggaagaaaccatcaccgccaaaggcgtgagaagccccaaggccatcagaggcagaatccagggcacctggcgccaaaaaaccgtggacctgcccgccggcaccaagtacgtggcctttagacactttcagtcaacggacatgttttacatcgacctggacgaagttgaaatcaaagcaaatgggggaggcagccctacccccaccgactacacatacaccgtgtacagggacggcaccaagatcaaggaggggctgaccgagactacctttgaggaggacggcgtggccaccggaaaccacgagtactgcgtggaggtgaagtataccgccggcgtgagtcccaagaagtgcgtggacgtgaccgtgaacggcgtgacccccaagaatatcatcgacgccttcaatggcggcatcagcctggcccagtacaccgggcacggctccgagacagcctggggcacatcccacttcggaaccacccacgtgaagcagctgaccaatagcaaccagtaataa

SEQ ID NO: 83 - RNA, 1935 nt, synthetic construct
  source 1..1935: mol_type = other RNA
atgaaggccatccttgtggtgctgctgtatacctttgccaccgccaacgccaaggccccttacaccggctgctacagccacctgaacaccggcgtgagcttcgccaactacgtggcccacggctctgagactgcctgggccgacccactgctgaccacatcccagctgaaggccctgaccaataaggacaagccctaccagcccgtgagccagctgaccgccaccacacagggccagaaggtgaccctgaaatgggaggcccccagcgccaagaagtccgagggctccagagaggtgaagagaatcggcgacggcctgtttgtgacaatcgagcccgccaacgacgtgagggccaatgaggccaaggtggtgctggccgccgacaacgtctggggcgataacaccggctatcagttcctgctggacgccgaccacaatacctttggctctgtgatccccgccaccggccccctgttcaccggcaccgccagcagcaacctgtacagcgccaacttcgagtacctgatccccgccaacgccgacccagtggtgacaacccagaacatcatcgtgaccggccagggcgaggtggtgatccccgggggcgtgtacgactactgcatcaccaaccccgagcccgcctccggcaagatgtggatcgccggcgacggcggcaaccagcccgccagatacgatgacttcaccttcgaggctggaaagaagtacaccttcaccatgaggagggccggcatgggcgacggaacagacatggaggccgaggacgactcccccgccagctacacctataccgtgtacagggacggcaccaagatcaaagagggcctgaccgccaccacctttgaagaggacggcgtggccgccggaaaccacgagtactgcgtggaggtgaagtacaccgccggcgtgtccccaaaggtgtgcaaggacgtgacagtggaggggagcaacgagttcgcccccgtgcagcagctgaccggcagcagcgtgggccagaaagtgacactgaagtgggacgccccacagggtacccccaaccctaaccccaaccccaaccccaacccaggcaccaccctgagcgagtccttcgaaaacgggattcccgcctcctggaagaccatcgacgccgacggcgacggccacggctggaagccaggcaacgcccccggcatcgccggctataacagcaacggctgtgtgtatagcgagagcttcggactgggcggcatcggggtgctgacccccgataactacctgatcacccccgccctggacctgcctaacggcggcaagctgaccttctgggtgtgcgcccaggacgccaattatgccagcgagcactacgccgtgtacgccagcagcaccggcaacgacgccagcaacttcgccaatgccctgctggaagaaaccatcaccgccaaaggcgtgagaagccccaaggccatcagaggcagaatccagggcacctggcgccaaaaaaccgtggacctgcccgccggcaccaagtacgtggcctttagacactttcagtcaacggacatgttttacatcgacctggacgaagttgaaatcaaagcaaatgggggaggcagccctacccccaccgactacacatacaccgtgtacagggacggcaccaagatcaaggaggggctgaccgagactacctttgaggaggacggcgtggccaccggaaaccacgagtactgcgtggaggtgaagtataccgccggcgtgagtcccaagaagtgcgtggacgtgaccgtgaacggcgtgacccccaagaatatcatcgacgccttcaatggcggcatcagcctggcccagtacaccgggcacggctccgagacagcctggggcacatcccacttcggaaccacccacgtgaagcagctgaccaatagcaaccagtaataa

SEQ ID NO: 84 - RNA, 2469 nt, synthetic construct
  source 1..2469: mol_type = other RNA
atgaaggccatccttgtggtgctgctgtatacctttgccaccgccaacgccaaggccccttacaccggctgctacagccacctgaacaccggcgtgagcttcgccaactacgtggcccacggctctgagactgcctgggccgacccactgctgaccacatcccagctgaaggccctgaccaataaggacaagccctaccagcccgtgagccagctgaccgccaccacacagggccagaaggtgaccctgaaatgggaggcccccagcgccaagaagtccgagggctccagagaggtgaagagaatcggcgacggcctgttcgtgactatcgagcccgccaacgatgtgagagccaatgaggccaaggtggtgctggccgccgacaacgtgtggggcgataacaccggctaccagttcctgctggacgccgaccacaatacatttggcagcgtgatccccgccaccggcccactgttcaccggcacagcctccagcaacctgtactccgccaactttgagtacctgatccccgccaacgccgacccagtggtgaccacccagaacatcatcgtgaccggccagggcgaagtggtgatccccggcggcgtgtacgactattgtatcaccaacccagagccagccagcggaaagatgtggatcgccggcgatggcggcaaccagcctgcccggtacgacgatttcaccttcgaggccggcaaaaagtacacctttaccatgaggagagccggcatgggcgacggcaccgacatggaggccgaggacgatagccccgccagctacacctacacagtgtaccgggacggcaccaagatcaaggaaggcctgaccgccaccaccttcgaggaagacggcgtggccgccggcaaccacgaatactgtgtggaggtgaagtacacagccggcgtgagccccaaggtgtgcaaggacgtgaccgtggagggctccaacgagtttgccccagtgcagcagctgaccggcagcagcgtgggccagaaggtgaccctgaagtgggacgccccacagggtacccccaaccctaaccccaaccccaaccccaacccaggcaccaccctgagcgagtccttcgaaaacgggattcccgcctcctggaagaccatcgacgccgacggcgacggccacggctggaagccaggcaacgcccccggcatcgccggctataacagcaacggctgtgtgtatagcgagagcttcggactgggcggcatcggggtgctgacccccgataactacctgatcacccccgccctggacctgcctaacggcggcaagctgaccttctgggtgtgcgcccaggacgccaattatgccagcgagcactacgccgtgtacgccagcagcaccggcaacgacgccagcaacttcgccaatgccctgctggaagaaaccatcaccgccaaaggcgtgagaagccccaaggccatcagaggcagaatccagggcacctggagacagaagaccgtggacctgcccgccggcaccaagtacgtggccttcagacacttccagtccaccgacatgttctacatcgacctggatgaggtggagatcaaggccaacggcaagagggccgacttcacagaaaccttcgagagctccacccacggcgaggcccctgccgagtggacaaccatcgacgccgatggcgacggccagggctggctgtgcctgagctccgggcagctggactggctgaccgcccacggcggcagcaacgtggtgagctccttcagttggaacggcatggccctgaacccagacaactatctgatcagcaaggacgtgaccggcgccaccaaggtgaaatactactatgctgtgaacgacggatttcccggcgaccactacgccgtgatgatcagcaagaccggcaccaatgccggagacttcaccgtggtgtttgaggaaacccccaacggcatcaataagggcggcgccagattcggcctgtccaccgaggccaacggcgccaagcctcagtccgtgtggattgagcgcaccgtggacctgcccgccggcaccaagtacgtggcctttagacactaccagtgtagcgacctgaattacatcctgctggacgacatccagttcaccatgggaggcagccctacccccaccgactacacatacaccgtgtacagggacggcaccaagatcaaggaggggctgaccgagactacctttgaggaggacggcgtggccaccggaaaccacgagtactgcgtggaggtgaagtataccgccggcgtgagtcccaagaagtgcgtggacgtgaccgtgaacggcgtgacccccaagaatatcatcgacgccttcaatggcggcatcagcctggcccagtacaccgggcacggctccgagacagcctggggcacatcccacttcggaaccacccacgtgaagcagctgaccaatagcaaccagtaataa

SEQ ID NO: 85 - RNA, 2469 nt, synthetic construct
  source 1..2469: mol_type = other RNA
atgaaggccatccttgtggtgctgctgtatacctttgccaccgccaacgccaaggccccttacaccggctgctacagccacctgaacaccggcgtgagcttcgccaactacgtggcccacggctctgagactgcctgggccgacccactgctgaccacatcccagctgaaggccctgaccaataaggacaagccctaccagcccgtgagccagctgaccgccaccacacagggccagaaggtgaccctgaaatgggaggcccccagcgccaagaagtccgagggctccagagaggtgaagagaatcggcgacggcctgtttgtgacaatcgagcccgccaacgacgtgagggccaatgaggccaaggtggtgctggccgccgacaacgtctggggcgataacaccggctatcagttcctgctggacgccgaccacaatacctttggctctgtgatccccgccaccggccccctgttcaccggcaccgccagcagcaacctgtacagcgccaacttcgagtacctgatccccgccaacgccgacccagtggtgacaacccagaacatcatcgtgaccggccagggcgaggtggtgatccccgggggcgtgtacgactactgcatcaccaaccccgagcccgcctccggcaagatgtggatcgccggcgacggcggcaaccagcccgccagatacgatgacttcaccttcgaggctggaaagaagtacaccttcaccatgaggagggccggcatgggcgacggaacagacatggaggccgaggacgactcccccgccagctacacctataccgtgtacagggacggcaccaagatcaaagagggcctgaccgccaccacctttgaagaggacggcgtggccgccggaaaccacgagtactgcgtggaggtgaagtacaccgccggcgtgtccccaaaggtgtgcaaggacgtgacagtggaggggagcaacgagttcgcccccgtgcagcagctgaccggcagcagcgtgggccagaaagtgacactgaagtgggacgccccacagggtacccccaaccctaaccccaaccccaaccccaacccaggcaccaccctgagcgagtccttcgaaaacgggattcccgcctcctggaagaccatcgacgccgacggcgacggccacggctggaagccaggcaacgcccccggcatcgccggctataacagcaacggctgtgtgtatagcgagagcttcggactgggcggcatcggggtgctgacccccgataactacctgatcacccccgccctggacctgcctaacggcggcaagctgaccttctgggtgtgcgcccaggacgccaattatgccagcgagcactacgccgtgtacgccagcagcaccggcaacgacgccagcaacttcgccaatgccctgctggaagaaaccatcaccgccaaaggcgtgagaagccccaaggccatcagaggcagaatccagggcacctggagacagaagaccgtggacctgcccgccggcaccaagtacgtggccttcagacacttccagtccaccgacatgttctacatcgacctggatgaggtggagatcaaggccaacggcaagagggccgacttcacagaaaccttcgagagctccacccacggcgaggcccctgccgagtggacaaccatcgacgccgatggcgacggccagggctggctgtgcctgagctccgggcagctggactggctgaccgcccacggcggcagcaacgtggtgagctccttcagttggaacggcatggccctgaacccagacaactatctgatcagcaaggacgtgaccggcgccaccaaggtgaaatactactatgctgtgaacgacggatttcccggcgaccactacgccgtgatgatcagcaagaccggcaccaatgccggagacttcaccgtggtgtttgaggaaacccccaacggcatcaataagggcggcgccagattcggcctgtccaccgaggccaacggcgccaagcctcagtccgtgtggattgagcgcaccgtggacctgcccgccggcaccaagtacgtggcctttagacactaccagtgtagcgacctgaattacatcctgctggacgacatccagttcaccatgggaggcagccctacccccaccgactacacatacaccgtgtacagggacggcaccaagatcaaggaggggctgaccgagactacctttgaggaggacggcgtggccaccggaaaccacgagtactgcgtggaggtgaagtataccgccggcgtgagtcccaagaagtgcgtggacgtgaccgtgaacggcgtgacccccaagaatatcatcgacgccttcaatggcggcatcagcctggcccagtacaccgggcacggctccgagacagcctggggcacatcccacttcggaaccacccacgtgaagcagctgaccaatagcaaccagtaataa

SEQ ID NO: 86 - RNA, 3249 nt, synthetic construct
  source 1..3249: mol_type = other RNA
atgaaggccatccttgtggtgctgctgtatacctttgccaccgccaacgccaaggccccttacaccggctgctacagccacctgaacaccggcgtgagcttcgccaactacgtggcccacggctctgagactgcctgggccgacccactgctgaccacatcccagctgaaggccctgaccaataaggacaagccctaccagcccgtgagccagctgaccgccaccacacagggccagaaggtgaccctgaaatgggaggcccccagcgccaagaagtccgagggctccagagaggtgaagagaatcggcgacggcctgttcgtgactatcgagcccgccaacgatgtgagagccaatgaggccaaggtggtgctggccgccgacaacgtgtggggcgataacaccggctaccagttcctgctggacgccgaccacaatacatttggcagcgtgatccccgccaccggcccactgttcaccggcacagcctccagcaacctgtactccgccaactttgagtacctgatccccgccaacgccgacccagtggtgaccacccagaacatcatcgtgaccggccagggcgaagtggtgatccccggcggcgtgtacgactattgtatcaccaacccagagccagccagcggaaagatgtggatcgccggcgatggcggcaaccagcctgcccggtacgacgatttcaccttcgaggccggcaaaaagtacacctttaccatgaggagagccggcatgggcgacggcaccgacatggaggccgaggacgatagccccgccagctacacctacacagtgtaccgggacggcaccaagatcaaggaaggcctgaccgccaccaccttcgaggaagacggcgtggccgccggcaaccacgaatactgtgtggaggtgaagtacacagccggcgtgagccccaaggtgtgcaaggacgtgaccgtggagggctccaacgagtttgccccagtgcagcagctgaccggcagcagcgtgggccagaaggtgaccctgaagtgggacgccccacagggtacccccaaccctaaccccaaccccaaccccaacccaggcaccaccctgagcgagtccttcgaaaacgggattcccgcctcctggaagaccatcgacgccgacggcgacggccacggctggaagccaggcaacgcccccggcatcgccggctataacagcaacggctgtgtgtatagcgagagcttcggactgggcggcatcggggtgctgacccccgataactacctgatcacccccgccctggacctgcctaacggcggcaagctgaccttctgggtgtgcgcccaggacgccaattatgccagcgagcactacgccgtgtacgccagcagcaccggcaacgacgccagcaacttcgccaatgccctgctggaagaaaccatcaccgccaaaggcgtgagaagccccaaggccatcagaggcagaatccagggcacctggagacagaagaccgtggacctgcccgccggcaccaagtacgtggccttcagacacttccagtccaccgacatgttctacatcgacctggatgaggtggagatcaaggccaacggcaagagggccgacttcacagaaaccttcgagagctccacccacggcgaggcccctgccgagtggacaaccatcgacgccgatggcgacggccagggctggctgtgcctgagctccgggcagctggactggctgaccgcccacggcggcagcaacgtggtgagctccttcagttggaacggcatggccctgaacccagacaactatctgatcagcaaggacgtgaccggcgccaccaaggtgaaatactactatgctgtgaacgacggatttcccggcgaccactacgccgtgatgatcagcaagaccggcaccaatgccggagacttcaccgtggtgtttgaggaaacccccaacggcatcaataagggcggcgccagattcggcctgtccaccgaggccaacggcgccaagcctcagtccgtgtggattgagcgcaccgtggacctgcccgccggcaccaagtacgtggcctttagacactaccagtgtagcgacctgaattacatcctgctggacgacatccagttcaccatgggaggcagccctacccccaccgactacacatacaccgtgtacagggacggcaccaagatcaaggaggggctgaccgagactacctttgaggaggacggcgtggccaccggaaaccacgagtactgcgtggaggtgaagtataccgccggcgtgagtcccaagaagtgcgtggacgtgaccgtgaacaacccctaccagcccgtgtctaacctggccgccaccacccagggccagaaggtgaccctgaagtgggacgcccccagcaccaagacacaggccaccaccaacacagcccgcagcgtggacggcattcgggagctggtgctgctgagcgtgagcgacgcccctgagctgctgagaagcggccaggcagagattgtgctggaggcccacgacgtgtggaacgacggcagcggctaccagatcctgctggacgccgaccacgaccagtacggccaggtgatcccctccgacacccacaccctgtggcctaactgcgccgtgcccgccaacctgttcgcccccttcgagtacaccgtgcctgagaacgccgaccctagctgctctccaaccaacatgatcatggacggcaccgccagcgtgaacatccccgccgggacatacgacttcgccattgccgccccccaggccaatgccaagatctggatcgccggccagggccccaccaaggaggacgactacgtgttcgaggccggcaagaagtaccacttcctgatgaagaagatgggctccggggacggcaccgagctgaccatcagcgagggcggcggaagcgactacacctataccgtgtacagagacggcaccaagatcaaggagggcctgaccgcaaccacctttgaggaggatggcgtggccgctggcaaccacgagtactgcgtggaggtgaagtacaccgccggcgtgagccccaaggtgtgcaaggacgtgaccgtggagggctccggcgtgacccccaagaatatcatcgacgccttcaatggcggcatcagcctggcccagtacaccgggcacggctccgagacagcctggggcacatcccacttcggaaccacccacgtgaagcagctgaccaatagcaaccagtaataa

SEQ ID NO: 87 - RNA, 3249 nt, synthetic construct
  source 1..3249: mol_type = other RNA
atgaaggccatccttgtggtgctgctgtatacctttgccaccgccaacgccaaggccccttacaccggctgctacagccacctgaacaccggcgtgagcttcgccaactacgtggcccacggctctgagactgcctgggccgacccactgctgaccacatcccagctgaaggccctgaccaataaggacaagccctaccagcccgtgagccagctgaccgccaccacacagggccagaaggtgaccctgaaatgggaggcccccagcgccaagaagtccgagggctccagagaggtgaagagaatcggcgacggcctgtttgtgacaatcgagcccgccaacgacgtgagggccaatgaggccaaggtggtgctggccgccgacaacgtctggggcgataacaccggctatcagttcctgctggacgccgaccacaatacctttggctctgtgatccccgccaccggccccctgttcaccggcaccgccagcagcaacctgtacagcgccaacttcgagtacctgatccccgccaacgccgacccagtggtgacaacccagaacatcatcgtgaccggccagggcgaggtggtgatccccgggggcgtgtacgactactgcatcaccaaccccgagcccgcctccggcaagatgtggatcgccggcgacggcggcaaccagcccgccagatacgatgacttcaccttcgaggctggaaagaagtacaccttcaccatgaggagggccggcatgggcgacggaacagacatggaggccgaggacgactcccccgccagctacacctataccgtgtacagggacggcaccaagatcaaagagggcctgaccgccaccacctttgaagaggacggcgtggccgccggaaaccacgagtactgcgtggaggtgaagtacaccgccggcgtgtccccaaaggtgtgcaaggacgtgacagtggaggggagcaacgagttcgcccccgtgcagcagctgaccggcagcagcgtgggccagaaagtgacactgaagtgggacgccccacagggtacccccaaccctaaccccaaccccaaccccaacccaggcaccaccctgagcgagtccttcgaaaacgggattcccgcctcctggaagaccatcgacgccgacggcgacggccacggctggaagccaggcaacgcccccggcatcgccggctataacagcaacggctgtgtgtatagcgagagcttcggactgggcggcatcggggtgctgacccccgataactacctgatcacccccgccctggacctgcctaacggcggcaagctgaccttctgggtgtgcgcccaggacgccaattatgccagcgagcactacgccgtgtacgccagcagcaccggcaacgacgccagcaacttcgccaatgccctgctggaagaaaccatcaccgccaaaggcgtgagaagccccaaggccatcagaggcagaatccagggcacctggagacagaagaccgtggacctgcccgccggcaccaagtacgtggccttcagacacttccagtccaccgacatgttctacatcgacctggatgaggtggagatcaaggccaacggcaagagggccgacttcacagaaaccttcgagagctccacccacggcgaggcccctgccgagtggacaaccatcgacgccgatggcgacggccagggctggctgtgcctgagctccgggcagctggactggctgaccgcccacggcggcagcaacgtggtgagctccttcagttggaacggcatggccctgaacccagacaactatctgatcagcaaggacgtgaccggcgccaccaaggtgaaatactactatgctgtgaacgacggatttcccggcgaccactacgccgtgatgatcagcaagaccggcaccaatgccggagacttcaccgtggtgtttgaggaaacccccaacggcatcaataagggcggcgccagattcggcctgtccaccgaggccaacggcgccaagcctcagtccgtgtggattgagcgcaccgtggacctgcccgccggcaccaagtacgtggcctttagacactaccagtgtagcgacctgaattacatcctgctggacgacatccagttcaccatgggaggcagccctacccccaccgactacacatacaccgtgtacagggacggcaccaagatcaaggaggggctgaccgagactacctttgaggaggacggcgtggccaccggaaaccacgagtactgcgtggaggtgaagtataccgccggcgtgagtcccaagaagtgcgtggacgtgaccgtgaacaacccctaccagcccgtgtctaacctggccgccaccacccagggccagaaggtgaccctgaagtgggacgcccccagcaccaagacacaggccaccaccaacacagcccgcagcgtggacggcattcgggagctggtgctgctgagcgtgagcgacgcccctgagctgctgagaagcggccaggcagagattgtgctggaggcccacgacgtgtggaacgacggcagcggctaccagatcctgctggacgccgaccacgaccagtacggccaggtgatcccctccgacacccacaccctgtggcctaactgcgccgtgcccgccaacctgttcgcccccttcgagtacaccgtgcctgagaacgccgaccctagctgctctccaaccaacatgatcatggacggcaccgccagcgtgaacatccccgccgggacatacgacttcgccattgccgccccccaggccaatgccaagatctggatcgccggccagggccccaccaaggaggacgactacgtgttcgaggccggcaagaagtaccacttcctgatgaagaagatgggctccggggacggcaccgagctgaccatcagcgagggcggcggaagcgactacacctataccgtgtacagagacggcaccaagatcaaggagggcctgaccgcaaccacctttgaggaggatggcgtggccgctggcaaccacgagtactgcgtggaggtgaagtacaccgccggcgtgagccccaaggtgtgcaaggacgtgaccgtggagggctccggcgtgacccccaagaatatcatcgacgccttcaatggcggcatcagcctggcccagtacaccgggcacggctccgagacagcctggggcacatcccacttcggaaccacccacgtgaagcagctgaccaatagcaaccagtaataa

SEQ ID NO: 88 - protein, 47 aa, synthetic construct
KAPYTGCYSHLNTGVSFANYVAHGSETAWADPLLTTSQLKALTNKDK

SEQ ID NO: 89 - protein, 31 aa, synthetic construct
PYQPVSQLTATTQGQKVTLKWEAPSAKKSEG

SEQ ID NO: 90 - protein, 162 aa, synthetic construct
SREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDM

SEQ ID NO: 91 - protein, 65 aa, synthetic construct
EAEDDSPASYTYTVYRDGTKIKEGLTATTFEEDGVAAGNHEYCVEVKYTAGVSPKVCKDVTVEGS

SEQ ID NO: 92 - protein, 24 aa, synthetic construct
NEFAPVQQLTGSSVGQKVTLKWDA

SEQ ID NO: 93 - protein, 187 aa, synthetic construct
PQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKAN

SEQ ID NO: 94 - protein, 23 aa, synthetic construct
PQGTPNPNPNPNPNPGTTLSESF

SEQ ID NO: 95 - protein, 56 aa, synthetic construct
TDYTYTVYRDGTKIKEGLTETTFEEDGVATGNHEYCVEVKYTAGVSPKKCVDVTVN

SEQ ID NO: 96 - protein, 178 aa, synthetic construct
KRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTM

SEQ ID NO: 97 - protein, 47 aa, synthetic construct
GVTPKNIIDAFNGGISLAQYTGHGSETAWGTSHFGTTHVKQLTNSNQ

SEQ ID NO: 98 - protein, 438 aa, synthetic construct
EPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNIATEDKWLGQALCIASAEGGPSADNGESDIQHENVIANLLTQYGYTKIIKCYDPGVTPKNIIDAFNGGISLANYAGHGSETAWGTSHFGTTHVKQLTNSNQLPFIFDVASVNGDFLFSMPCFAEALMRAQKDGKPTGTVAIIASTIQQSWASPMRGQDEMNEILCEKHPNNIKRTFGGVTMNGMFAMVEKYKKDGEKMLDTWTVFGDPSLLVRTLVPTKMQVTAPAQINLADASVQVSCDYNGAIATISANGKMFGSAVVEQGTATIQLTGLTQESTLTLTVVGYNKETVIKTINTNGEP

SEQ ID NO: 99 - protein, 32 aa, synthetic construct
NPYQPVSNLAATTQGQKVTLKWDAPSTKTQAT

SEQ ID NO: 100 - protein, 163 aa, synthetic construct
TNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTEL

SEQ ID NO: 101 - protein, 65 aa, synthetic construct
TISEGGGSDYTYTVYRDGTKIKEGLTATTFEEDGVAAGNHEYCVEVKYTAGVSPKVCKDVTVEGS

SEQ ID NO: 102 - protein, 24 aa, synthetic construct
NEFAPVQNLAGSAVGQKVTLKWDA

SEQ ID NO: 103 - protein, 26 aa, synthetic construct
PQGTPNPNPNPNPNPNPGTTTLSESF

SEQ ID NO: 104 - protein, 176 aa, synthetic construct
DFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMG

SEQ ID NO: 105 - protein, 56 aa, synthetic construct
TDYTYTVYRDGTKIKEGLTETTFEEDGVATGNHEYCVEVKYTAGVSPKKCVQVTVN

SEQ ID NO: 106 - protein, 35 aa, Porphyromonas gingivalis
GEPSPYQPVSNLTATTQGQKVTLKWEAPSAKKAEG

SEQ ID NO: 107 - protein, 24 aa, Porphyromonas gingivalis
PVSNLTATTQGQKVTLKWEAPSAK

SEQ ID NO: 108 - protein, 30 aa, Porphyromonas gingivalis
GEPSPYPQPVSNLTATTQGQKVTLKWEAPS

SEQ ID NO: 109 - protein, 23 aa, Porphyromonas gingivalis
PVSNLTATTQGQKVTLKWEAPSA

SEQ ID NO: 110 - protein, 26 aa, Porphyromonas gingivalis
NEFAPVQNLTGSSVGQKVTLKWDAPN

SEQ ID NO: 111 - protein, 22 aa, Porphyromonas gingivalis
PVQNLTGSSVGQKVTLKWDAPN

SEQ ID NO: 112 - protein, 15 aa, Porphyromonas gingivalis
VGQKVTLKWDAPNGT

SEQ ID NO: 113 - protein, 23 aa, Porphyromonas gingivalis
PVQNLTGSSVGQKVTLKWDAPST

SEQ ID NO: 114 - protein, 32 aa, Porphyromonas gingivalis
NPYQPVSNLTATTQGQKVTLKWDAPSTKTNAT

SEQ ID NO: 115 - protein, 30 aa, Porphyromonas gingivalis
GEPNPYPQPVSNLTATTQGQKVTLKWDAPS

SEQ ID NO: 116 - protein, 23 aa, Porphyromonas gingivalis
PVSNLTATTQGQKVTLKWDAPST

SEQ ID NO: 117 - protein, 15 aa, Porphyromonas gingivalis
VGQKVTLKWDAPNGT

SEQ ID NO: 118 - protein, 23 aa, Porphyromonas gingivalis
PVQNLTGSAVGQKVTLKWDAPNG

SEQ ID NO: 119 - protein, 27 aa, Porphyromonas gingivalis
NEFAPVQNLTGSAVGQKVTLKWDAPNG

SEQ ID NO: 120 - protein, 11 aa, synthetic construct
  VARIANT 10: note = E/D
GQKVTLKWEXA

SEQ ID NO: 121 - protein, 57 aa, Porphyromonas gingivalis
SYTYTVYRDGTKIKEGLTATTFEEDGVAAGNHEYCVEVKYTAGVSPKVCKDVTVEGS

SEQ ID NO: 122 - protein, 29 aa, Porphyromonas gingivalis
SYTYTVYRDGTKIKEGLTATTFEEDGVAA

SEQ ID NO: 123 - protein, 29 aa, Porphyromonas gingivalis
SYTYTVYRDGTKIKEGLTATTFEEDGVAA

SEQ ID NO: 124 - protein, 56 aa, Porphyromonas gingivalis
TDYTYTVYRDGTKIKEGLTETTFEEDGVATGNHEYCVEVKYTAGVSPKKCVNVTVN

SEQ ID NO: 125 - protein, 15 aa, Porphyromonas gingivalis
EGLTATTFEEDGVAA

SEQ ID NO: 126 - protein, 56 aa, Porphyromonas gingivalis; identical to SEQ ID NO: 124

SEQ ID NO: 127 - protein, 27 aa, Porphyromonas gingivalis
YTYTVYRDGTKIKEGLTETTFEEDGVA

SEQ ID NO: 128 - protein, 29 aa, Porphyromonas gingivalis
SYTYTVYRDGTKIKEGLTATTFEEDGVAA

SEQ ID NO: 129 - protein, 15 aa, Porphyromonas gingivalis
EGLTATTFEEDGVAA

SEQ ID NO: 130 - protein, 14 aa, synthetic construct
  VARIANT 5: note = X=E/A
EGLTXTTFEEDGVA

SEQ ID NO: 131 - protein, 21 aa, Porphyromonas gingivalis
GTPNPNPNPNPNPGTTLSESF

SEQ ID NO: 132 - protein, 30 aa, Porphyromonas gingivalis
VTLKWDAPNGTPNPNPNPNPNPGTTLSESF

SEQ ID NO: 133 - protein, 17 aa, Porphyromonas gingivalis
GTPNPNPNPNPNPNPGT

SEQ ID NO: 134 - protein, 16 aa, Porphyromonas gingivalis
  REPEAT 6..7: note = 1-6
  VARIANT 11: note = X=T; note = X may be deleted
PNGTPNPGTTXLSESF

SEQ ID NO: 135 - protein, 26 aa, Porphyromonas gingivalis
PNGTPNPNPNPNPNPNPGTTTLSESF

SEQ ID NO: 136 - protein, 30 aa, Porphyromonas gingivalis; identical to SEQ ID NO: 132

SEQ ID NO: 137 - protein, 17 aa, Porphyromonas gingivalis
GTPNPNPNPNPNPNPGT

SEQ ID NO: 138 - protein, 16 aa, Porphyromonas gingivalis
  REPEAT 6..7: note = 1-6
  VARIANT 11: note = X=T; note = X may be deleted
PNGTPNPGTTXLSESF

SEQ ID NO: 139 - protein, 15 aa, synthetic construct
  REPEAT 12..13: note = 1-2
GTPNPNPNPNPNPGT

SEQ ID NO: 140 - RNA, 11 nt, Cytomegalovirus (CMV)
  source 1..11: mol_type = viral cRNA
gggatcctacc

SEQ ID NO: 141 - protein, 17 aa, Influenza virus
  VARIANT 3: note = X=A/V
  VARIANT 4: note = X=I/K
  VARIANT 6: note = X=V/L
  VARIANT 8: note = X=L/M
  VARIANT 10: note = X=Y/C
  VARIANT 13: note = X=T/A
  VARIANT 14: note = X=T/A
  VARIANT 15: note = X=A/T
  VARIANT 16: note = X=N/Y
MKXXLXVXLXTFXXXXA

SEQ ID NO: 142 - protein, 343 aa, synthetic construct
MNTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGG

SEQ ID NO: 143 - protein, 359 aa, synthetic construct
MKAILVVLLYTFATANANTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGG

SEQ ID NO: 144 - protein, 359 aa, synthetic construct
MKAILVVLLYTFATANANTGVSFANYVAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGG

SEQ ID NO: 145 - protein, 386 aa, synthetic construct
MKAILVVLLYTFATANANTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGGSILAIYSTVASSLVLVVSLGAISFGG

SEQ ID NO: 146 - protein, 386 aa, synthetic construct
MKAILVVLLYTFATANANTGVSFANYVAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGGSILAIYSTVASSLVLVVSLGAISFGG

SEQ ID NO: 147 - RNA, 1032 nt, synthetic construct
  source 1..1032: mol_type = other RNA
atgaacaccggagtgagcttcgccaactacactgcacacggaagcgagacagcctgggctgatccactgctgaccaccagccagctgaaggctctgacaaataaggataaatggggcgacaacacaggctaccagttcctgctggacgctgatcacaacacatttggaagcgtgattccagccaccgggccactgttcaccggcaccgcttctagcaacctgtattctgctaattttgagtacctgattcccgccaacgctgatcctgtggtgactacacagaacattatcgtgacaggccagggcgaggtggtcatccctggaggagtgtacgactactgcatcactaatcccgagcccgcttctggcaagatgtggatcgccggggatggggggaaccagccagccaggtatgatgactttaccttcgaagccggcaagaaatatacctttacaatgaggcgcgccggaatgggagatggcacagacatggaggccgaggacgacagcccagcctcctacacttacaccgtgtacagggacggcactaagatcaaggaaggactgaccgccacaaccttcgaggaagatggcgtggctgctggaaaccacgagtattgtgtggaggtgaaatacaccgccggagtgagcccaaaggtgtgtaaagacgtgaccgtggaaggcagcaacgagttcgcccccgtgcagaacctgactgggagctccgtgggccagaaggtgactctgaagtgggacgcccccaacggaactccaaaccccaaccctaaccccaaccctaaccctggaaccacactgtccgagtctttcgagaatgggatcccagcctcctggaagactatcgacgccgacggagacggacacggctggaaaccaggaaatgccccaggcatcgccggctacaatagcaacggctgcgtctattctgagtcctttggactgggcgggatcggcgtgctgactcccgacaactacctgatcacccctgccctggatctgccaaacggcggctga

SEQ ID NO: 148 - RNA, 1032 nt, synthetic construct
  source 1..1032: mol_type = other RNA
atgaataccggagtgtccttcgccaactacaccgctcatggatctgaaactgcctgggctgatcccctgctgacaacaagccagctgaaggccctgaccaataaggacaagtggggagataataccggctaccagttcctgctggatgccgaccacaatactttcggaagtgtgattcctgctaccggccctctctttacaggcaccgcctcatccaacctgtattctgccaatttcgaatatctcattccagctaacgccgacccagtggtgacaacacagaacatcattgtgacagggcagggcgaagtggttatcccagggggggtgtacgactattgcattacaaaccccgagccagcctccgggaaaatgtggatcgctggagatggagggaaccagcccgccagatacgatgactttactttcgaagccggcaaaaagtatacctttaccatgagaagggctggaatgggagacggcactgacatggaggcagaggatgacagccctgcctcctacacatacacagtgtaccgggacggcactaagatcaaggaggggctgactgcaactacctttgaggaagatggcgtggctgctggcaaccacgaatattgtgtggaggtgaagtatacagccggagtgtctcctaaggtttgtaaggacgtgaccgtggaaggctccaacgagtttgccccagtgcagaacctgacaggatcaagtgtcggccaaaaagtgactctgaagtgggacgcccctaatggcacccccaatcctaatcctaaccctaaccccaatcctggcaccaccctgtccgagagcttcgagaatggcattcccgcctcctggaagactatcgacgccgacggggatggacatggatggaagcccggcaatgccccagggattgcaggctacaatagcaacggttgcgtgtactccgagagcttcgggctgggagggatcggagtcctgacacctgataattacctgattacccccgccctggatctgcctaacgggggctga

SEQ ID NO: 149 - RNA, 1080 nt, synthetic construct
  source 1..1080: mol_type = other RNA
atgaaggccattctggtggtgctgctgtacacattcgccaccgccaatgccaacaccggagtgagcttcgccaactacactgcacacggaagcgagacagcctgggctgatccactgctgaccaccagccagctgaaggctctgacaaataaggataaatggggcgacaacacaggctaccagttcctgctggacgctgatcacaacacatttggaagcgtgattccagccaccgggccactgttcaccggcaccgcttctagcaacctgtattctgctaattttgagtacctgattcccgccaacgctgatcctgtggtgactacacagaacattatcgtgacaggccagggcgaggtggtcatccctggaggagtgtacgactactgcatcactaatcccgagcccgcttctggcaagatgtggatcgccggggatggggggaaccagccagccaggtatgatgactttaccttcgaagccggcaagaaatatacctttacaatgaggcgcgccggaatgggagatggcacagacatggaggccgaggacgacagcccagcctcctacacttacaccgtgtacagggacggcactaagatcaaggaaggactgaccgccacaaccttcgaggaagatggcgtggctgctggaaaccacgagtattgtgtggaggtgaaatacaccgccggagtgagcccaaaggtgtgtaaagacgtgaccgtggaaggcagcaacgagttcgcccccgtgcagaacctgactgggagctccgtgggccagaaggtgactctgaagtgggacgcccccaacggaactccaaaccccaaccctaaccccaaccctaaccctggaaccacactgtccgagtctttcgagaatgggatcccagcctcctggaagactatcgacgccgacggagacggacacggctggaaaccaggaaatgccccaggcatcgccggctacaatagcaacggctgcgtctattctgagtcctttggactgggcgggatcggcgtgctgactcccgacaactacctgatcacccctgccctggatctgccaaacggcggctga

SEQ ID NO: 150 - RNA, 1080 nt, synthetic construct
  source 1..1080: mol_type = other RNA
atgaaagccatcctggtggtgctgctgtacactttcgccaccgctaacgccaataccggagtgtccttcgccaactacaccgctcatggatctgaaactgcctgggctgatcccctgctgacaacaagccagctgaaggccctgaccaataaggacaagtggggagataataccggctaccagttcctgctggatgccgaccacaatactttcggaagtgtgattcctgctaccggccctctctttacaggcaccgcctcatccaacctgtattctgccaatttcgaatatctcattccagctaacgccgacccagtggtgacaacacagaacatcattgtgacagggcagggcgaagtggttatcccagggggggtgtacgactattgcattacaaaccccgagccagcctccgggaaaatgtggatcgctggagatggagggaaccagcccgccagatacgatgactttactttcgaagccggcaaaaagtatacctttaccatgagaagggctggaatgggagacggcactgacatggaggcagaggatgacagccctgcctcctacacatacacagtgtaccgggacggcactaagatcaaggaggggctgactgcaactacctttgaggaagatggcgtggctgctggcaaccacgaatattgtgtggaggtgaagtatacagccggagtgtctcctaaggtttgtaaggacgtgaccgtggaaggctccaacgagtttgccccagtgcagaacctgacaggatcaagtgtcggccaaaaagtgactctgaagtgggacgcccctaatggcacccccaatcctaatcctaaccctaaccccaatcctggcaccaccctgtccgagagcttcgagaatggcattcccgcctcctggaagactatcgacgccgacggggatggacatggatggaagcccggcaatgccccagggattgcaggctacaatagcaacggttgcgtgtactccgagagcttcgggctgggagggatcggagtcctgacacctgataattacctgattacccccgccctggatctgcctaacgggggctga

SEQ ID NO: 151 - RNA, 1080 nt, synthetic construct
  source 1..1080: mol_type = other RNA
atgaaggccattctggtggtgctgctgtacacattcgccaccgccaatgccaacaccggagtgagcttcgccaactacgtggcacacggaagcgagacagcctgggctgatccactgctgaccaccagccagctgaaggctctgacaaataaggataaatggggcgacaacacaggctaccagttcctgctggacgctgatcacaacacatttggaagcgtgattccagccaccgggccactgttcaccggcaccgcttctagcaacctgtattctgctaattttgagtacctgattcccgccaacgctgatcctgtggtgactacacagaacattatcgtgacaggccagggcgaggtggtcatccctggaggagtgtacgactactgcatcactaatcccgagcccgcttctggcaagatgtggatcgccggggatggggggaaccagccagccaggtatgatgactttaccttcgaagccggcaagaaatatacctttacaatgaggcgcgccggaatgggagatggcacagacatggaggccgaggacgacagcccagcctcctacacttacaccgtgtacagggacggcactaagatcaaggaaggactgaccgccacaaccttcgaggaagatggcgtggctgctggaaaccacgagtattgtgtggaggtgaaatacaccgccggagtgagcccaaaggtgtgtaaagacgtgaccgtggaaggcagcaacgagttcgcccccgtgcagcagctgactgggagctccgtgggccagaaggtgactctgaagtgggacgccccccagggaactccaaaccccaaccctaaccccaaccctaaccctggaaccacactgtccgagtctttcgagaatgggatcccagcctcctggaagactatcgacgccgacggagacggacacggctggaaaccaggaaatgccccaggcatcgccggctacaatagcaacggctgcgtctattctgagtcctttggactgggcgggatcggcgtgctgactcccgacaactacctgatcacccctgccctggatctgccaaacggcggctga

SEQ ID NO: 152 - RNA, 1080 nt, synthetic construct
  source 1..1080: mol_type = other RNA
atgaaagccatcctggtggtgctgctgtacactttcgccaccgctaacgccaataccggagtgtccttcgccaactacgtggctcatggatctgaaactgcctgggctgatcccctgctgacaacaagccagctgaaggccctgaccaataaggacaagtggggagataataccggctaccagttcctgctggatgccgaccacaatactttcggaagtgtgattcctgctaccggccctctctttacaggcaccgcctcatccaacctgtattctgccaatttcgaatatctcattccagctaacgccgacccagtggtgacaacacagaacatcattgtgacagggcagggcgaagtggttatcccagggggggtgtacgactattgcattacaaaccccgagccagcctccgggaaaatgtggatcgctggagatggagggaaccagcccgccagatacgatgactttactttcgaagccggcaaaaagtatacctttaccatgagaagggctggaatgggagacggcactgacatggaggcagaggatgacagccctgcctcctacacatacacagtgtaccgggacggcactaagatcaaggaggggctgactgcaactacctttgaggaagatggcgtggctgctggcaaccacgaatattgtgtggaggtgaagtatacagccggagtgtctcctaaggtttgtaaggacgtgaccgtggaaggctccaacgagtttgccccagtgcagcagctgacaggatcaagtgtcggccaaaaagtgactctgaagtgggacgcccctcagggcacccccaatcctaatcctaaccctaaccccaatcctggcaccaccctgtccgagagcttcgagaatggcattcccgcctcctggaagactatcgacgccgacggggatggacatggatggaagcccggcaatgccccagggattgcaggctacaatagcaacggttgcgtgtactccgagagcttcgggctgggagggatcggagtcctgacacctgataattacctgattacccccgccctggatctgcctaacgggggctga

SEQ ID NO: 153 - RNA, 1161 nt, synthetic construct
  source 1..1161: mol_type = other RNA
atgaaggccattctggtggtgctgctgtacacattcgccaccgccaatgccaacaccggagtgagcttcgccaactacactgcacacggaagcgagacagcctgggctgatccactgctgaccaccagccagctgaaggctctgacaaataaggataaatggggcgacaacacaggctaccagttcctgctggacgctgatcacaacacatttggaagcgtgattccagccaccgggccactgttcaccggcaccgcttctagcaacctgtattctgctaattttgagtacctgattcccgccaacgctgatcctgtggtgactacacagaacattatcgtgacaggccagggcgaggtggtcatccctggaggagtgtacgactactgcatcactaatcccgagcccgcttctggcaagatgtggatcgccggggatggggggaaccagccagccaggtatgatgactttaccttcgaagccggcaagaaatatacctttacaatgaggcgcgccggaatgggagatggcacagacatggaggccgaggacgacagcccagcctcctacacttacaccgtgtacagggacggcactaagatcaaggaaggactgaccgccacaaccttcgaggaagatggcgtggctgctggaaaccacgagtattgtgtggaggtgaaatacaccgccggagtgagcccaaaggtgtgtaaagacgtgaccgtggaaggcagcaacgagttcgcccccgtgcagaacctgactgggagctccgtgggccagaaggtgactctgaagtgggacgcccccaacggaactccaaaccccaaccctaaccccaaccctaaccctggaaccacactgtccgagtctttcgagaatgggatcccagcctcctggaagactatcgacgccgacggagacggacacggctggaaaccaggaaatgccccaggcatcgccggctacaatagcaacggctgcgtctattctgagtcctttggactgggcgggatcggcgtgctgactcccgacaactacctgatcacccctgccctggatctgccaaacggcggcggaagcatcctggctatctacagcaccgtggcctccagcctggtgctggtggtttccctgggcgccattagcttcggcggctga

SEQ ID NO: 154 - RNA, 1161 nt, synthetic construct
  source 1..1161: mol_type = other RNA
atgaaagccatcctggtggtgctgctgtacactttcgccaccgctaacgccaataccggagtgtccttcgccaactacaccgctcatggatctgaaactgcctgggctgatcccctgctgacaacaagccagctgaaggccctgaccaataaggacaagtggggagataataccggctaccagttcctgctggatgccgaccacaatactttcggaagtgtgattcctgctaccggccctctctttacaggcaccgcctcatccaacctgtattctgccaatttcgaatatctcattccagctaacgccgacccagtggtgacaacacagaacatcattgtgacagggcagggcgaagtggttatcccagggggggtgtacgactattgcattacaaaccccgagccagcctccgggaaaatgtggatcgctggagatggagggaaccagcccgccagatacgatgactttactttcgaagccggcaaaaagtatacctttaccatgagaagggctggaatgggagacggcactgacatggaggcagaggatgacagccctgcctcctacacatacacagtgtaccgggacggcactaagatcaaggaggggctgactgcaactacctttgaggaagatggcgtggctgctggcaaccacgaatattgtgtggaggtgaagtatacagccggagtgtctcctaaggtttgtaaggacgtgaccgtggaaggctccaacgagtttgccccagtgcagaacctgacaggatcaagtgtcggccaaaaagtgactctgaagtgggacgcccctaatggcacccccaatcctaatcctaaccctaaccccaatcctggcaccaccctgtccgagagcttcgagaatggcattcccgcctcctggaagactatcgacgccgacggggatggacatggatggaagcccggcaatgccccagggattgcaggctacaatagcaacggttgcgtgtactccgagagcttcgggctgggagggatcggagtcctgacacctgataattacctgattacccccgccctggatctgcctaacgggggcggctccatcctggccatctacagcaccgtggcctcctccctggtgctggtggtgtctctcggagccatctccttcggcgggtga

SEQ ID NO: 155 - RNA, 1161 nt, synthetic construct
  source 1..1161: mol_type = other RNA
atgaaggccattctggtggtgctgctgtacacattcgccaccgccaatgccaacaccggagtgagcttcgccaactacgtggcacacggaagcgagacagcctgggctgatccactgctgaccaccagccagctgaaggctctgacaaataaggataaatggggcgacaacacaggctaccagttcctgctggacgctgatcacaacacatttggaagcgtgattccagccaccgggccactgttcaccggcaccgcttctagcaacctgtattctgctaattttgagtacctgattcccgccaacgctgatcctgtggtgactacacagaacattatcgtgacaggccagggcgaggtggtcatccctggaggagtgtacgactactgcatcactaatcccgagcccgcttctggcaagatgtggatcgccggggatggggggaaccagccagccaggtatgatgactttaccttcgaagccggcaagaaatatacctttacaatgaggcgcgccggaatgggagatggcacagacatggaggccgaggacgacagcccagcctcctacacttacaccgtgtacagggacggcactaagatcaaggaaggactgaccgccacaaccttcgaggaagatggcgtggctgctggaaaccacgagtattgtgtggaggtgaaatacaccgccggagtgagcccaaaggtgtgtaaagacgtgaccgtggaaggcagcaacgagttcgcccccgtgcagcagctgactgggagctccgtgggccagaaggtgactctgaagtgggacgccccccagggaactccaaaccccaaccctaaccccaaccctaaccctggaaccacactgtccgagtctttcgagaatgggatcccagcctcctggaagactatcgacgccgacggagacggacacggctggaaaccaggaaatgccccaggcatcgccggctacaatagcaacggctgcgtctattctgagtcctttggactgggcgggatcggcgtgctgactcccgacaactacctgatcacccctgccctggatctgccaaacggcggcggaagcatcctggctatctacagcaccgtggcctccagcctggtgctggtggtttccctgggcgccattagcttcggcggctga

SEQ ID NO: 156 - RNA, 1161 nt, synthetic construct
  source 1..1161: mol_type = other RNA
atgaaagccatcctggtggtgctgctgtacactttcgccaccgctaacgccaataccggagtgtccttcgccaactacgtggctcatggatctgaaactgcctgggctgatcccctgctgacaacaagccagctgaaggccctgaccaataaggacaagtggggagataataccggctaccagttcctgctggatgccgaccacaatactttcggaagtgtgattcctgctaccggccctctctttacaggcaccgcctcatccaacctgtattctgccaatttcgaatatctcattccagctaacgccgacccagtggtgacaacacagaacatcattgtgacagggcagggcgaagtggttatcccagggggggtgtacgactattgcattacaaaccccgagccagcctccgggaaaatgtggatcgctggagatggagggaaccagcccgccagatacgatgactttactttcgaagccggcaaaaagtatacctttaccatgagaagggctggaatgggagacggcactgacatggaggcagaggatgacagccctgcctcctacacatacacagtgtaccgggacggcactaagatcaaggaggggctgactgcaactacctttgaggaagatggcgtggctgctggcaaccacgaatattgtgtggaggtgaagtatacagccggagtgtctcctaaggtttgtaaggacgtgaccgtggaaggctccaacgagtttgccccagtgcagcagctgacaggatcaagtgtcggccaaaaagtgactctgaagtgggacgcccctcagggcacccccaatcctaatcctaaccctaaccccaatcctggcaccaccctgtccgagagcttcgagaatggcattcccgcctcctggaagactatcgacgccgacggggatggacatggatggaagcccggcaatgccccagggattgcaggctacaatagcaacggttgcgtgtactccgagagcttcgggctgggagggatcggagtcctgacacctgataattacctgattacccccgccctggatctgcctaacgggggcggctccatcctggccatctacagcaccgtggcctcctccctggtgctggtggtgtctctcggagccatctccttcggcgggtga

SEQ ID NO: 157 - protein, 1732 aa, Porphyromonas gingivalis
MRKLLLLIAASLLGVGLYAQSAKIKLDAPTTRTTCTNNSFKQFDASFSFNEVELTKVETKGGTFASVSIPGAFPTGEVGSPEVPAVRKLIAVPVGATPVVRVKSFTEQVYSLNQYGSEKLMPHQPSMSKSDDPEKVPFVYNAAAYARKGFVGQELTQVEMLGTMRGVRIAALTINPVQYDVVANQLKVRNNIEIEVSFQGADEVATQRLYDASFSPYFETAYKQLFNRDVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKTKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCCITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNSTQFNPVQNLTAEQAPNSMDAILKWNAPASKRAEVLNEDFENGIPASWKTIDADGDGNNWTTTPPPGGSSFAGHNSAICVSSASYINFEGPQNPDNYLVTPELSLPGGGTLTFWVCAQDANYASEHYAVYASSTGNDASNFANALLEEVLTAKTVVTAPEAIRGTRAQGTWYQKTVQLPAGTKYVAFRHFGCTDFFWINLDDVVITSGNAPSYTYTIYRNNTQIASGVTETTYRDPDLATGFYTYGVKVVYPNGESAIETATLNITSLADVTAQKPYTLTVVGKTITVTCQGEAMIYDMNGRRLAAGRNTVVYTAQGGHYAVMVVVDGKSYVEKLAVK

SEQ ID NO: 158 - protein, 1706 aa, Porphyromonas gingivalis
MKNLNKFVSIALCSSLLGGMAFAQQTELGRNPNVRLLESTQQSVTKVQFRMDNLKFTEVQTPKGMAQVPTYTEGVNLSEKGMPTLPILSRSLAVSDTREMKVEVVSSKFIEKKNVLIAPSKGMIMRNEDPKKIPYVYGKSYSQNKFFPGEIATLDDPFILRDVRGQVVNFAPLQYNPVTKTLRIYTEITVAVSETSEQGKNILNKKGTFAGFEDTYKRMFMNYEPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLANYTGHGSETAWGTSHFGTTHVKQLTNSNQLPFIFDVACVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEPNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPNPNPGTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNSTQFNPVKNLKAQPDGGDVVLKWEAPSAKKTEGSREVKRIGDGLFVTIEPANDVRANEAKVVLAADNVWGDNTGYQFLLDADHNTFGSVIPATGPLFTGTASSDLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTETTYRDAGMSAQSHEYCVEVKYTAGVSPKVCVDYIPDGVADVTAQKPYTLTVVGKTITVTCQGEAMIYDMNGRRLAAGRNTVVYTAQGGYYAVMVVVDGKSYVEKLAIK

SEQ ID NO: 159 - protein, 736 aa, Porphyromonas gingivalis
MKKNFSRIVSIVAFSSLLGGMAFAQPAERGRNPQVRLLSAEQSMSKVQFRMDNLQFTDVQTSKGVAQVPTFTEGVNISEKGTPILPILSRSLAVSETRAMKVEVVSSKFIEKKDVLIAPSKGVISRAENPDQIPYVYGQSYNEDKFFPGEIATLSDPFILRDVRGQVVNFAPLQYNPVTKTLRIYTEIVVAVSETAEAGQNTISLVKNSTFTGFEDIYKSVFMNYEATRYTPVEEKENGRMIVIVAKKYEGDIKDFVDWKNQRGLRTEVKVAEDIASPVTANAIQQFVKQEYEKEGNDLTYVLLVGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLVNYTGHGSETAWGTSHFGTTHVKQLTNSNQLPFIFDVACVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTEMQVTAPANISASAQTFEVACDYNGAIATLSDDGDMVGTAIVKDGKAIIKLNESIADETNLTLTVVGYNKVTVIKDVKVEGTSIADVANDKPYTVAVSGKTITVESPAAGLTIFDMNGRRVATAKNRMVFEAQNGVYAVRIATEGKTYTEKVIVK

SEQ ID NO: 160 - DNA, 6026 bp, Porphyromonas gingivalis
  source 1..6026: mol_type = genomic DNA
ggatcctacgcccgatacccatactcgaagcctttgctcagtaccatcctgcagaaggttactctttcgcatatagtgaccctcttttctctcagcataatggtacctatcatatcagtaaggggcgtattgtcttttcgaacaatgtacagcccgagaactctttacttccacatcacacccccgactccttagtcaaggatcttttttcccctttcccctccgctctcttcctcatgctggactgacttaaccttggtctgctctacttttcggttgtaaatacatgcaacacaataactttaattgttgttagacaacacttttacaagactctgacttttaatgaggtggagcatgaaccttttcctctttcatcttctccttcagattacagtcaatattttggcaaaaggctaattgacagccttttataagggttaatcccttgtcgcttatattgaaaacatgttctttataatccgatactcttcttaaatcgaattttttctctaaattgcgccgcaacaaaactccttgagaaaagtaccaatagaaatagaaggtagcattttgcctttaaattccttttcttttcttggattgttcttgaaatgaatcttatttgtggattttttttgtttttttaacccggccgtggttctctgaatcacgaccataaattgttttaaagtatgaggaaattattattgctgatcgcggcgtcccttttgggagttggtctttacgcccaaagcgccaagattaagcttgatgctccgactactcgaacgacatgtacgaacaatagcttcaagcagttcgatgcaagcttttcgttcaatgaagtcgagctgacaaaggtggagaccaaaggtggtactttcgcctcagtgtcaattccgggtgcattcccgaccggtgaggttggttctcccgaagtgccagcagttaggaagttgattgctgtgcctgtcggagccacacctgttgttcgcgtgaaaagttttaccgagcaagtttactctctgaaccaatacggttccgaaaaactcatgccacatcaaccctctatgagcaagagtgatgatcccgaaaaggttcccttcgtttacaatgctgctgcttatgcacgcaaaggttttgtcggacaagaactgacccaagtagaaatgttggggacaatgcgtggtgttcgcattgcagctcttaccattaatcctgttcagtatgatgtggttgcaaaccaattgaaggttagaaacaacatcgaaattgaagtaagctttcaaggagctgatgaagtagctacacaacgtttgtatgatgcttcttttagcccttatttcgaaacagcttataaacagctcttcaatagagatgtttatacagatcatggcgacttgtataatacgccggttcgtatgcttgttgttgcaggtgcaaaattcaaagaagctctcaagccttggctcacttggaaggctcaaaagggcttctatctggatgtgcattacacagacgaagctgaagtaggaacgacaaacgcctctatcaaggcatttattcacaagaaatacaatgatggattggcagctagtgctgctccggtcttcttggctttggttggtgacactgacgttattagcggagaaaaaggaaagaaaacaaaaaaagttaccgacttgtattacagtgcagtcgatggcgactatttccctgaaatgtatactttccgtatgtctgcttcttccccagaagaactgacgaacatcattgataaggtattgatgtatgaaaaggctactatgccagataagagttatttggagaaagttctcttgattgcaggtgcagattatagctggaattcccaggtaggtcagccaaccattaaatacggtatgcagtactactacaaccaagagcatggttataccgacgtgtacaactatctcaaagccccttatacaggttgctacagtcatttgaataccggagtcagctttgcaaactatacagcgcatggatctgagaccgcatgggctgatccacttctgactacttctcaactgaaagcactcactaataaggacaaatacttcttagctattggcaactgctgtattacagctcaattcgattatgtacagccttgcttcggagaggtaataactcgcgttaaggagaaaggggcttatgcctatatcggttcatctccaaattcttattggggcgaggactactattggagtgtgggtgctaatgccgtatttggtgttcagcctacttttgaaggtacgtctatgggttcttatgatgctacattcttggaggattcgtacaacacagtgaattctattatgtgggcaggtaatcttgccgctactcatgctggaaatatcggcaatattacccatattggtgctcattactattgggaagcttatcatgtccttggcgatggttcggttatgccttatcgtgcaatgcctaagaccaatacttatacgcttcctgcctctttgcctcagaatcaggcttcttatagcattcaggcttctgccggttcttacgtagctatttctaaagatggagttttgtatggaacaggtgttgctaatgccagcggtgttgcgactgtgagtatgactaagcagattacggaaaatggtaattatgatgtagttatcactcgctctaattatcttcctgtgatcaagcaaattcaggtaggtgagcctagcccctaccagcccgtttccaacttgacagctacaacgcagggtcagaaagtaacgctcaagtgggaagcaccgagcgcaaagaaggcagaaggttcccgtgaagtaaaacggatcggagacggtcttttcgttacgatcgaacctgcaaacgatgtacgtgccaacgaagccaaggttgtgcttgcggcagacaacgtatggggagacaatacgggttaccagttcttgttggatgccgatcacaatacattcggaagtgtcattccggcaaccggtcctctctttaccggaacagcttcttccaatctttacagtgcgaacttcgagtatttgatcccggccaatgccgatcctgttgttactacacagaatattatcgttacaggacagggtgaagttgtaatccccggtggtgtttacgactattgcattacgaacccggaacctgcatccggaaagatgtggatcgcaggagatggaggcaaccagcctgcacgttatgacgatttcacattcgaagcaggcaagaagtacaccttcacgatgcgtcgcgccggaatgggagatggaactgatatggaagtcgaagacgattcacctgcaagctatacctacacggtgtatcgtgacggcacgaagatcaaggaaggtctgacagctacgacattcgaagaagacggtgtagctgcaggcaatcatgagtattgcgtggaagttaagtacacagccggcgtatctccgaaggtatgtaaagacgttacggtagaaggatccaatgaatttgctcctgtacagaacctgaccggtagttcagtaggtcagaaagtaacgcttaagtgggatgcacctaatggtaccccgaatccgaatccaaatccgaatccgaatccgggaacaacactttccgaatcattcgaaaatggtattccggcatcttggaagacgatcgatgcagacggtgacgggcatggctggaaacctggaaatgctcccggaatcgctggctacaatagcaatggttgtgtatattcagagtcattcggtcttggtggtataggagttcttacccctgacaactatctgataacaccggcattggatttgcctaacggaggtaagttgactttctgggtatgcgcacaggatgctaattatgcatccgagcactatgcggtgtatgcatcttcgaccggtaacgatgcatccaacttcacgaatgctttgttggaagagacgattacggcaaaaggtgttcgctcgccgaaagctattcgtggtcgtatacagggtacttggcgccagaagacggtagaccttcccgcaggtacgaaatatgttgctttccgtcacttccaaagcacggatatgttctacatcgaccttgatgaggttgagatcaaggccaatggcaagcgcgcagacttcacggaaacgttcgagtcttctactcatggagaggcaccagcggaatggactactatcgatgccgatggcgatggtcagggttggctctgtctgtcttccggacaattggactggctgacagctcatggcggcagcaacgtagtaagctctttctcatggaatggaatggctttgaatcctgataactatctcatctcaaaggatgttacaggcgcaacgaaggtaaagtactactatgcagtcaacgacggttttcccggggatcactatgcggtgatgatctccaagacgggcacgaacgccggagacttcacggttgttttcgaagaaacgcctaacggaataaataagggcggagcaagattcggtctttccacggaagccaatggcgccaaacctcaaagtgtatggatcgagcgtacggtagatttgcctgcaggcacgaagtatgttgctttccgtcactacaattgctcggatttgaactacattcttttggatgatattcagttcaccatgggtggcagccccaccccgaccgattatacctacacggtgtatcgtgatggtacgaagatcaaggaaggtttgaccgaaacgaccttcgaagaagacggcgtagctacgggcaatcatgagtattgcgtggaagtgaagtacacagccggcgtatctccgaagaaatgtgtaaacgtaactgttaattcgacacagttcaatcctgtacagaacctgacggcagaacaagctcctaacagcatggatgcaatccttaaatggaatgcaccggcatctaagcgtgcggaagttctgaacgaagacttcgaaaatggtattcctgcctcatggaagacgatcgatgcagacggtgacggcaacaattggacgacgacccctcctcccggaggctcctcttttgcaggtcacaacagtgcgatctgtgtctcttcagcttcttatatcaactttgaaggtcctcagaaccctgataactatctggttacaccggagctttctcttcctggcggaggaacgcttactttctgggtatgtgcacaagatgccaattatgcatcagagcactatgccgtgtacgcatcttctacgggtaacgacgcttccaacttcgccaacgctttgttggaagaagtgctgacggccaagacagttgttacggcacctgaagccattcgtggtactcgtgctcagggcacctggtatcaaaagacggtacagttgcctgcgggtactaagtatgttgccttccgtcacttcggctgtacggacttcttctggatcaaccttgatgatgttgtaatcacttcagggaacgctccgtcttacacctatacgatctatcgtaataatacacagatagcatcaggcgtaacggagactacttaccgagatccggacttggctaccggtttttacacgtacggtgtaaaggttgtttacccgaacggagaatcagctatcgaaactgctacgttgaatatcacttcgttggcagacgtaacggctcagaagccttacacgctgacagttgtaggaaagacgatcacggtaacttgccaaggcgaagctatgatctacgacatgaacggtcgtcgtctggcagcgggtcgcaacacggttgtttacacggctcagggcggccactatgcagtcatggttgtcgttgacggcaagtcttacgtagagaaactcgctgtaaagtaaatctgtcttggactcggagactttgtgcagacacttttaagataggtctgtaattgtctcagagtatgaatcggtcgcccgacttccttaaaaggaggtcgggcgacttcgtttgtattattgctgtccggt

SEQ ID NO: 161 - DNA, 5121 bp, Porphyromonas gingivalis
  source 1..5121: mol_type = genomic DNA
ttacttgatagcgagtttctctacgtaagacttgccgtcaacgacaaccataactgcatagtagccgccctgagccgtgtaaacaaccgtgttgcgaccggctgccagacgacgaccgttcatgtcgtagatcatagcttcgccttggcaagttaccgtgatcgtctttcctacaactgtcagcgtgtaaggcttctgagccgttacgtctgccactccgtcaggaatataatccacacaaaccttcggagatacgccggctgtgtacttaacttccacgcaatactcatgagattgtgcactcattcctgcatcgcggtaggtcgtttcggtcagaccttccttgatcttcgtgccgtcacgatagactgtataggtatagcttgcaggtgaatcgtcttcgacttccatatcagttccatctcccattccggcgcgacgcatcgtgaaggtgtacttcttgcctgcttcgaatgtgaaatcgtcataacgtgcaggctggttgcctccatctcctgcgatccacatctttccggatgcaggttccgggttcgtaatgcaatagtcgtaaacaccaccggggattacaacttcaccctgtcctgtaacgataatattctgtgtagtaacaacaggatcggcattggccgggatcaaatactcgaagttcgcactgtaaagatcggaagaagctgttccggtaaagagaggaccggttgccggaatgacacttccgaatgtattgtgatcggcatccaacaagaactggtaacccgtattgtctccccatacgttgtctgctgcgagcacaaccttggcttcgttggcacgtacatcgtttgcaggttcgatcgtaacgaaaagaccgtctccgatccgttttacttcacgagaaccttctgtcttctttgcgctcggggcttcccacttgagaaccacgtcgccgccatccggttgtgccttcaggttctttacaggattgaactgtgtcgaattaacagttacgtttacacatttcttcggagatacgccggctgtgtacttcacttccacgcaatactcatgattgcccgtagctacgccgtcttcttcgaaggtcgtttcggtcaaaccttccttgatcttcgtaccatcacgatacaccgtgtaggtataatcggtcggggtggggctgccacccatggtgaactgaatatcatccaaaagaatgtagttcaaatccgagcaattgtagtgacggaaagcaacatacttcgtgcccgcaggcaaatctaccgtacgctcgatccatacactttgaggtttggcgccattggcttccgtggaaagaccgaatcttgctccgcccttatttattccgttaggcgtttcttcgaaaacgaccgtgaagtctccggcgttcgtgcccgtcttggagatcatcaccgcatagtgatccccgggaaaaccgtcgttgactgcatagtagtactttaccttcgttgcgcctgtaacatcctttgagatgagatagttatcaggattcaaagccattccattccatgagaaagaggctactacgttggtgccgccatgagctgtcagccagtccaattgtccggaagacagacagagccaaccctgaccatcgccatcggcatcgatagtagtccattccgctggtgcctctccatgagtagaagactcgaacgtttccgtgaagtctgcgcgcttgccattggccttgatctcaacctcatcaaggtcgatgtagaacatatccgtgctttggaagtgacggaaagcaacatatttcgtacctgcgggaaggtctaccgtcttctggcgccaagtaccctgtatacgaccacgaatagctttcggcgagcgaacaccttttgccgtaatcgtctcttccaacaaagcattcgtgaagttggatgcatcgttaccggtcgaagatgcatacaccgcatagtgctcggatgcataattagcatcctgtgcgcatacccagaaagtcaacttacctccgttaggcaaatccaatgccggtgttatcagatagttgtcaggggtaagaactcctataccaccaagaccgaatgactctgaatatacacaaccattgctattgtagccagcgattccgggagcatttccaggcttccagccatgcccgtcaccgtctgcatcgatcgtcttccatgaggcaggaataccattttcgaatgattcggaaagtgtagttgttccgggattcggatttggattcggatttggatttggattcggggtaccattaggtgcatcccacttaagcgttactttctggccgactgcactaccggtcaggttctgtacaggagcaaattcattggatccttctaccgtaacgtctttacataccttcggagatacgccggctgtgtacttaacttccacgcaatactcatgattgcctgcagctacaccgtcttcttcgaatgtcgtagccgtcagaccttccttgatcttcgtgccgtcacgatagacagtataggtgtaatcgcttccaccaccttcgcttatagtcaattcagttccatcaccgctacccatcttcttcataaggaaatggtattttttaccggcttcaaatacataatcatcttctttcgtcggtccttgtccggcaatccaaatctttgcatttgcttgaggagcagcaattgcaaagtcataagttccggccggtatattaacggatgcagtaccatccattatcatattggtaggggaacaagaaggatctgcattttccggaaccgtatattcgaacggagcgaacagattggccgggacactacagttcggccaaagagtatgggtatcactgggtataacctgtccatattgatcatggtctgcatccaaaagaatctgataaccggatccatcattccaaacatcgtgagcttcaagaacaatctcggcctgaccgctgcgaagaagttcgggggcatcgctgactgacagaagaaccagttctcgtatgccatccacgctgcgagcggtattagtggttgcattggttttcgtgctcggtgcatcccacttgagcgttactttctgaccctgcgttgtagcagtcaagttggaaacaggctggtaggggttaggctcaccattagtgttgatggtcttaataaccgtctctttgttgtaaccaactactgtaagggtaagcgtgctttcatttgtcagacctgtcagattgattgtagctgttccattttcgacaactgcagaaccgaacatctttccattggctgaaatggtagcaatagcaccattataatcgcaagatacgttgactgaagcatccgtcaaattaatctgagccggagccgtaacctgcattttggtcgggacaagtgtacgaacgagcagcgaggggtcgccgaatacagtccatgtgtcgagcatcttctcaccatcctttttatacttttccaccatagcaaacataccgttcatggtgacaccaccgaaagtacgcttgatgttgttcgggtgtttttcgcacagaatttcgttcatctcatcctgcccgcgcataggagaagcccaagactggttgatcgtagacgctatgatagcaacagtacctgtcggcttaccatctttttgtgcacgcatcaatgcttctgcgaaacaaggcatgctgaataggaaatcgccattcacacaagctacgtcgaaaataaacggtagctggttgctgttggtaagctgcttcacatgagtggtgccgaagtgagacgtaccccaagctgtttcgctaccgtggcccgtatagttggccaacgagattcctccgttgaaagcatcaataatgtttttaggagttactcccggatcataacatttgataatcttggtataaccatactgggtaagcagattggcgattacattctcatgctggatatcactttcaccattgtctgcggatgggcctccttcagccgaagcaatacaaagagcctgaccgagccatttgtcttccgtggttatattgcgctcatagtgaatagtccgatcgatttgtgtcttcagatcctctttgctctcacatgagaaacgaccgatgaagacttcgttgtagtggtcattacctactatttgtccatatacctggtcggatttgatccccggagtaattttggcaggaatatctttgtgatcgccaatcaaaagaacataggtcaaatcattaccttctttctcgtattcttgcttaacgaattgctgaatagcattagctgtaacgggagaagcaatatcttctgccactttcacctcggtacggagaccgcgttggtttttccaatcaacgaaatctttaatatctccctcatactttttggctacgatgacgatcatacgaccattttgtttttcctctaccggtgtgtaacgccctggctcgtagttcatgaacatgcgcttgtatgtgtcttcaaagccggcaaatgtacctttcttgttcagaatatttttgccttgctccgaagtttcgctcactgccacagtgatttccgtatagatgcgcaacgtctttgtcacagggttatactgcaaaggcgcaaagtttacaacctgtccacgcacatcacgaaggataaaaggatcatcaagcgtggcgatctctcccgggaagaatttgttttgcgagtagctctttccataaacgtaagggatctttttcggatcttcgttacgcataatcatgcccttggagggtgcaatcaggacatttttcttttcgatgaactttgaggaaacaacctctaccttcatctcacgagtgtctgaaaccgccaaagagcgtgatagaatgggaagcgtaggcatccctttttcagaaagattaaccccttctgtataggtcggcacttgtgccattcccttaggggtttgaacttcggtgaacttgaggttgtccatacggaactgaacctttgtcaccgattgctgagtggattcgagcaatctcacattcggattgcgtcccaactctgtctgctgcgcaaatgccattcctcctaataaggaagagcaaagagcaatcgaaacaaacttgttcaagtttttcat

SEQ ID NO: 162 - DNA, 2211 bp, Porphyromonas gingivalis
  source 1..2211: mol_type = genomic DNA
atgaaaaagaattttagcaggatcgtttcgatcgtagcattctcctctctgttgggaggaatggcgtttgcacagccggcagagcgcggtcgcaacccacaagtacgactgctctccgctgagcagtctatgtccaaggtgcaattccgtatggacaatcttcagttcacagacgtacagacctccaaaggggtagctcaggtgcctaccttcaccgaaggtgtcaatatctcggagaaaggtacgcctatattgcccattctttcccgttctcttgccgtttctgaaacacgtgcgatgaaggtagaggtagtttcttccaaattcatcgaaaagaaggatgtgctgatcgctccctctaaaggtgttatctctcgcgctgaaaatcctgatcaaatcccctacgtgtacggacagagctataatgaagacaagttcttcccgggcgaaatcgctacgctgagcgatcctttcatcctgcgtgatgtacgtggtcaggtagtaaactttgctccgctccagtataaccctgttacaaagaccctccgcatctatacggaaatcgttgtagctgtgagcgaaacagctgaagcgggccagaatacgattagcttggttaagaacagcactttcacaggcttcgaagatatctataagagcgtcttcatgaattatgaagctacgcgctatacgcctgttgaagaaaaggagaatggtcgtatgatcgtcatcgtagccaaaaagtatgagggagatattaaagatttcgttgattggaaaaaccaacgcggtctccgtaccgaggtgaaagtggcagaagatattgcttctcccgttacagctaatgctattcagcagttcgttaagcaagaatacgagaaagaaggtaatgatttgacctatgttcttttggttggcgatcacaaagatattcctgccaaaattactccggggatcaaatccgaccaggtatatggacaaatagtaggtaatgaccactacaacgaagtcttcatcggtcgtttctcatgtgagagcaaagaggatctgaagacacaaatcgatcggactattcactatgagcgcaatataaccacggaagacaaatggctcggtcaggctctttgtattgcttcggctgaaggaggcccatccgcagacaatggtgaaagtgatatccagcatgagaatgtaatcgccaatctgcttacccagtatggctataccaagattatcaaatgttatgatccgggagtaactcctaaaaacattattgatgctttcaacggaggaatctcgttggtcaactatacgggccacggtagcgaaacagcttggggtacgtctcacttcggcaccactcatgtgaagcagcttaccaacagcaaccagctaccgtttattttcgacgtagcttgtgtgaatggcgatttcctattcagcatgccttgcttcgcagaagccctgatgcgtgcacaaaaagatggtaagccgacaggtactgttgctatcatagcgtctacgatcaaccagtcttgggcttctcctatgcgcgggcaggatgagatgaacgaaattctgtgcgaaaaacacccgaacaacatcaagcgtactttcggtggtgtcaccatgaacggtatgtttgctatggtggaaaagtataaaaaggatggtgagaagatgctcgacacatggactgtattcggcgacccctcgctgctcgttcgtacacttgtcccgaccgaaatgcaggttacggctccggcaaatatctctgcctctgcacagacattcgaagtagcttgcgactataacggtgctatcgctacgctctctgacgatggtgatatggtcggcactgctatcgtgaaagacggtaaggctatcatcaaattaaatgagagtatcgctgatgaaacgaacttgacgctcaccgtagtaggatacaataaggttactgtgataaaggatgtgaaagtggaaggtacatctattgccgacgtagccaatgataagccttatactgtagctgtatcaggtaagacgataactgtagaaagtcctgctgccgggctgacgatcttcgatatgaacggccgtcgtgtagctactgctaagaaccgcatggtattcgaagcacaaaacggcgtgtatgccgttcgcatcgctactgaaggcaagacgtatacagaaaaggttatagtgaagtaa

SEQ ID NO: 163 - protein, 36 aa, synthetic construct
NTGVSFANYVAHGSETAWADPLLTTSQLKALTNKDK

SEQ ID NO: 164 - RNA, 108 nt, synthetic construct
  source 1..108: mol_type = other RNA
aacaccggagtgagcttcgccaactacgtggcacacggaagcgagacagcctgggctgatccactgctgaccaccagccagctgaaggctctgacaaataaggataaa

SEQ ID NO: 165 - RNA, 108 nt, synthetic construct
  source 1..108: mol_type = other RNA
aataccggagtgtccttcgccaactacgtggctcatggatctgaaactgcctgggctgatcccctgctgacaacaagccagctgaaggccctgaccaataaggacaag

SEQ ID NO: 166 - protein, 36 aa, synthetic construct
NGGISLAQYTGHGSETAWGTSHFGTTHVKQLTNSNQ

SEQ ID NO: 167 - RNA, 108 nt, synthetic construct
  source 1..108: mol_type = other RNA
aatggcggaatctctctggcccagtataccggccacggcagcgaaaccgcctggggcacctctcactttggcaccacccacgtgaagcagctgaccaatagcaaccag

SEQ ID NO: 168 - protein, 162 aa, Porphyromonas gingivalis; identical to SEQ ID NO: 90

SEQ ID NO: 169 - protein, 162 aa, Porphyromonas gingivalis
SREVKRIGDGLFVTIEPANDVRANEAKVVLAADNVWGDNTGYQFLLDADHNTFGSVIPATGPLFTGTASSNLYSANFEYLVPANADPVVTTQNIIVTGQGEVVIPGGVYDYCITNPEPASGKMWIAGDGGNQPARYDDFTFEAGKKYTFTMRRAGMGDGTDM

SEQ ID NO: 170 - protein, 162 aa, Porphyromonas gingivalis; identical to SEQ ID NO: 90

SEQ ID NO: 171 - protein, 163 aa, Porphyromonas gingivalis
TNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTEL

SEQ ID NO: 172 - protein, 160 aa, Porphyromonas gingivalis
ARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTEL

SEQ ID NO: 173 - protein, 127 aa, synthetic construct
WGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDM

SEQ ID NO: 174 - protein, 452 aa, Porphyromonas gingivalis
DVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKTKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCCITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQV

SEQ ID NO: 175 - protein, 47 aa, synthetic construct
KAPYTGCYSHLNTGVSFANYTAHGSETAWADPLLTTSQLKALTNKDK

SEQ ID NO: 176 - protein, 452 aa, Porphyromonas gingivalis; identical to SEQ ID NO: 174

SEQ ID NO: 177 - protein, 451 aa, Porphyromonas gingivalis
DVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKTKKVTDLYYSAVDGDYFPEMYTFRMSASSPEELTNIIDKVLMYEKATMPDKSYLEKALLIAGADSYWNPKIGQQTIKYAVQYYYNQDHGYTDVYSYPKAPYTGCYSHLNTGVGFANYTAHGSETSWADPSLTATQVKALTNKDKYFLAIGNCCVTAQFDYPQPCFGEVMTRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVNMTKQITENGNYDVVITRSNYLPVIKQIQ

SEQ ID NO: 178 - protein, 438 aa, synthetic construct
EPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLANYTGHGSETAWGTSHFGTTHVKQLTNSNQLPFIFDVACVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEP

SEQ ID NO: 179 - protein, 47 aa, synthetic construct
GVTPKNIIDAFNGGISLANYTGHGSETAWGTSHFGTTHVKQLTNSNQ

SEQ ID NO: 180 - protein, 36 aa, Porphyromonas gingivalis
NGGISLANYTGHGSETAWGTSHFGTTHVKQLTNSNQ

SEQ ID NO: 181 - protein, 439 aa, Porphyromonas gingivalis
EPGRYTPVEEKQNGRMIVIVAKKYEGDIKDFVDWKNQRGLRTEVKVAEDIASPVTANAIQQFVKQEYEKEGNDLTYVLLVGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLVNYTGHGSETAWGTSHFGTTHVKQLTNSNQLPFIFDVACVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEPN

SEQ ID NO: 182 - protein, 169 aa, Porphyromonas gingivalis
GTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEI

SEQ ID NO: 183 - protein, 169 aa, Porphyromonas gingivalis; identical to SEQ ID NO: 182

SEQ ID NO: 184 - protein, 170 aa, Porphyromonas gingivalis
GTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPEAIRGRIQGTWRQKTVDLPAGTKYVAFRHFQSTDMFYIDLDEVEI

SEQ ID NO: 185 - protein, 170 aa, Porphyromonas gingivalis
GTTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEI

SEQ ID NO: 186 - protein, 170 aa, Porphyromonas gingivalis; identical to SEQ ID NO: 184

SEQ ID NO: 187 - protein, 172 aa, Porphyromonas gingivalis
FTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQF

SEQ ID NO: 188 - protein, 172 aa, Porphyromonas gingivalis; identical to SEQ ID NO: 187

SEQ ID NO: 189 - protein, 172 aa, Porphyromonas gingivalis
FTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQF

SEQ ID NO: 190 - protein, 172 aa, Porphyromonas gingivalis; identical to SEQ ID NO: 189

SEQ ID NO: 191 - protein, 9 aa, synthetic construct
GTTTLSESF

SEQ ID NO: 192 - protein, 9 aa, Porphyromonas gingivalis
GTTTLSESF

SEQ ID NO: 193 - protein, 17 aa, Influenza virus
MKAKLLVLLCTFTATYA

SEQ ID NO: 194 - protein, 16 aa, Influenza virus
MKTIIALSYILCLVFA

SEQ ID NO: 195 - protein, 15 aa, Influenza virus
MKAIIVLLMVVTSNA

SEQ ID NO: 196 - protein, 12 aa, SARS CoV-2
MFVFLVLLPLVS

SEQ ID NO: 197 - protein, 21 aa, SARS CoV-2
MFLLTTKRTMFVFLVLLPLVS

SEQ ID NO: 198 - protein, 71 aa, Varicella zoster virus (VZV)
MSPCGYYSKWRNRDRPEYRRNLRFRRFFSSIHPNAAAGSGFNGPGVFITSVTGVWLCFLCIFSMFVTAVVS

SEQ ID NO: 199 - protein, 30 aa, Varicella zoster virus (VZV)
MGTVNKPVVGVLMGFGIITGTLRITNPVRA

SEQ ID NO: 200 - protein, 20 aa, Varicella zoster virus (VZV)
MFLIQCLISAVIFYIQVTNA

SEQ ID NO: 201 - protein, 21 aa, Varicella zoster virus (VZV)
MQALGIKTEHFIIMCLLSGHA

SEQ ID NO: 202 - protein, 23 aa, Measles morbillivirus
MGLKVNVSAIFMAVLLTLQTPTG

SEQ ID NO: 203 - protein, 21 aa, Rubella virus
MGAAAALTAVVLQGYNPPAYG

SEQ ID NO: 204 - protein, 23 aa, Rubella virus
MGAPQAFLAGLLLAAVAVGTARA

SEQ ID NO: 205 - protein, 19 aa, Mumps virus
MKVFLVTCLGFAVFSSSVC

SEQ ID NO: 206 - protein, 32 aa, Ebola virus
MGVTGILQLPRDRFKRTSFFLWVIILFQRTFS

SEQ ID NO: 207 - protein, 16 aa, Smallpox virus
MRSLIIFLLFPSIIYS

SEQ ID NO: 208 - protein, 19 aa, Rabies virus
MVPQALLFVPLLVFPLCFG

SEQ ID NO: 209 - protein, 17 aa, Influenza virus
MKAILVVLLYTFTTANA

SEQ ID NO: 210 - protein, 17 aa, Influenza virus
MKVKLLVLLCTFTATYA

SEQ ID NO: 211 - protein, 17 aa, Influenza virus
MKAILVVMLYTFTTANA

SEQ ID NO: 212 - protein, 16 aa, Influenza virus
MKTIIALSYILCLVFT

SEQ ID NO: 213 - protein, 16 aa, Influenza virus
MKTIIALSNILCLVFA

SEQ ID NO: 214 - protein, 16 aa, Influenza virus
MKAIIALSNILCLVFA

SEQ ID NO: 215 - protein, 16 aa, Influenza virus
MKTIIALSCILCLVFA

SEQ ID NO: 216 - protein, 16 aa, Influenza virus
MKTIIALSHILCLVFA

SEQ ID NO: 217 - protein, 15 aa, Influenza virus
MKAIIVLLMVVTSSA

SEQ ID NO: 218 - protein, 15 aa, Influenza virus
MEAIIVLLMVVTSNA

SEQ ID NO: 219 - protein, 17 aa, Influenza virus
  VARIANT 3: note = X=A/V
  VARIANT 4: note = X=I/K
  VARIANT 6: note = X=V/L
  VARIANT 8: note = X=L/M
  VARIANT 10: note = X=Y/C
  VARIANT 13: note = X=T/A
  VARIANT 14: note = X=T/A
  VARIANT 15: note = X=A/T
  VARIANT 16: note = X=N/Y
MKXXLXVXLXTFXXXXA

SEQ ID NO: 220 - protein, 16 aa, Influenza virus
  VARIANT 3: note = X=T/A
  VARIANT 9: note = X=Y/N/C/H
  VARIANT 15: note = X=T/A
MKXIIALSXILCLVFX

SEQ ID NO: 221 - protein, 15 aa, Influenza virus
  VARIANT 14: note = X=S/N
MKAIIVLLMVVTSXA

SEQ ID NO: 222 - protein, 15 aa, Influenza virus
  VARIANT 2: note = X=S/N
MXAIIVLLMVVTSNA

SEQ ID NO: 223 - protein, 23 aa, Influenza virus
ILAIYSTVASSLVLLVSLGAISF

SEQ ID NO: 224 - protein, 23 aa, Influenza virus
ILAIYSTVASSLVLVVSLGAISF

SEQ ID NO: 225 - protein, 21 aa, Influenza virus
ILWISFAISCFLLCVVLLGFI

SEQ ID NO: 226 - protein, 21 aa, Influenza virus
STAASSLAVTLMLAIFIVYMV

SEQ ID NO: 227 - protein, 21 aa, SARS CoV-2
WYIWLGFIAGLIAIVMVTIML

SEQ ID NO: 228 - protein, 21 aa, Varicella zoster virus (VZV)
FGALAVGLLVLAGLVAAFFAY

SEQ ID NO: 229 - protein, 21 aa, Varicella zoster virus (VZV)
AAWTGGLAAVVLLCLVIFLIC

SEQ ID NO: 230 - protein, 21 aa, Varicella zoster virus (VZV)
IIIPIVASVMILTAMVIVIVI

SEQ ID NO: 231 - protein, 21 aa, Varicella zoster virus (VZV)
YFWCVQLKMIFFAWFVYGMYL

SEQ ID NO: 232 - protein, 21 aa, Measles morbillivirus
IVYILIAVCLGGLIGIPALIC

SEQ ID NO: 233 - protein, 21 aa, Rubella virus
LDHAFAAFVLLVPWVLIFMVC

SEQ ID NO: 234 - protein, 21 aa, Rubella virus
WWQLTLGAICALLLAGLLACC

SEQ ID NO: 235 - protein, 21 aa, Mumps virus
IVAALVLSILSIIISLLFCCW

SEQ ID NO: 236 - protein, 21 aa, Ebola virus
WIPAGIGVTGVIIAVIALFCI

SEQ ID NO: 237 - protein, 21 aa, Rabies virus
VLLSAGALTALMLIIFLMTCW

SEQ ID NO: 238 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 239 - RNA, 100 nt, synthetic construct
  source 1..100: mol_type = other RNA
cgggtggcatccctgtgacccctccccagtgcctctcctggccctggaagttgccactccagtgcccaccagccttgtcctaataaaattaagttgcatc

SEQ ID NO: 240 - RNA, 110 nt, synthetic construct
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 241 - protein, 5 aa, synthetic construct
DGGGS

SEQ ID NO: 242 - protein, 5 aa, synthetic construct
TGEKP

SEQ ID NO: 243 - protein, 4 aa, synthetic construct
GGRR

SEQ ID NO: 244 - protein, 5 aa, synthetic construct
  REPEAT 1..5: note = The sequence can be repeated one or more times.
GGGGS

SEQ ID NO: 245 - protein, 14 aa, synthetic construct
EGKSSGSGSESKVD

SEQ ID NO: 246 - protein, 18 aa, synthetic construct
KESGSVSSEQLAQFRSLD

SEQ ID NO: 247 - protein, 8 aa, synthetic construct
GGRRGGGS

SEQ ID NO: 248 - protein, 9 aa, synthetic construct
LRQRDGERP

SEQ ID NO: 249 - protein, 12 aa, synthetic construct
LRQKDGGGSERP

SEQ ID NO: 250 - protein, 18 aa, synthetic construct
GSTSGSGKPGSGEGSTKG

SEQ ID NO: 251 - protein, 437 aa, Porphyromonas gingivalis
EATRYTPVEEKENGRMIVIVAKKYEGDIKDFVDWKNQRGLRTEVKVAEDIASPVTANAIQQFVKQEYEKEGNDLTYVLLVGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLVNYTGHGSETAWGTSHFGTTHVKQLTNSNQLPFIFDVACVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTEMQVTAPANISASAQTFEVACDYNGAIATLSDDGDMVGTAIVKDGKAIIKLNESIADETNLTLTVVGYNKVTVIKDVKVEG

SEQ ID NO: 252 - protein, 47 aa, synthetic construct
GVTPKNIIDAFNGGISLVNYTGHGSETAWGTSHFGTTHVKQLTNSNQ

SEQ ID NO: 253 - protein, 36 aa, synthetic construct
NGGISLVNYTGHGSETAWGTSHFGTTHVKQLTNSNQ

SEQ ID NO: 254 - protein, 988 aa, synthetic construct
DVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 255 - protein, 1004 aa, synthetic construct
KAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 256 - protein, 1004 aa, synthetic construct
KAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 257 - protein, 1032 aa, synthetic construct
KAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 258 - protein, 1032 aa, synthetic construct
KAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 259 - protein, 818 aa, synthetic construct
DVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 260 - protein, 834 aa, synthetic construct
KAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 261 - protein, 834 aa, synthetic construct
KAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 262 - protein, 862 aa, synthetic construct
KAILVVLLYTFATANADVYTDHGDLYNTPVRMLVVAGAKFKEALKPWLTWKAQKGFYLDVHYTDEAEVGTTNASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYTAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNITHIGAHYYWEAYHVLGDGSVMPYRAMPKTNTYTLPASLPQNQASYSIQASAGSYVAISKDGVLYGTGVANASGVATVSMTKQITENGNYDVVITRSNYLPVIKQIQVGEPSPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 263 - protein, 862 aa, synthetic construct
KAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 264 - protein, 568 aa, synthetic construct
NTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 265 - protein, 584 aa, synthetic construct
KAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 266 - protein, 584 aa, synthetic construct
KAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 267 - protein, 612 aa, synthetic construct
KAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 268 - protein, 612 aa, synthetic construct
KAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 269 - protein, 398 aa, synthetic construct
NTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 270 - protein, 414 aa, synthetic construct
KAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVN

SEQ ID NO: 271 - protein, 414 aa, synthetic construct
KAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 272 - protein, 442 aa, synthetic construct
KAILVVLLYTFATANANTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFPTDYTYTVYRDGTKIKEGLTETTFEEDGVATGNHEYCVEVKYTAGVSPKKCVNVTVNGGSILAIYSTVASSLVLVVSLGAISFGG

SEQ ID NO: 273 - protein, 442 aa, synthetic construct
KAILVVLLYTFATANANTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVNGGSILAIYSTVASSLVLVVSLGAISFGG

SEQ ID NO: 274 - protein, 1005 aa, synthetic construct
KAILVVLLYTFATANAEPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNIATEDKWLGQALCIASAEGGPSADNGESDIQHENVIANLLTQYGYTKIIKCYDPGVTPKNIIDAFNGGISLANYAGHGSETAWGTSHFGTTHVKQLTNSNQLPFIFDVASVNGDFLFSMPCFAEALMRAQKDGKPTGTVAIIASTIQQSWASPMRGQDEMNEILCEKHPNNIKRTFGGVTMNGMFAMVEKYKKDGEKMLDTWTVFGDPSLLVRTLVPTKMQVTAPAQINLADASVQVSCDYNGAIATISANGKMFGSAVVEQGTATIQLTGLTQESTLTLTVVGYNKETVIKTINTNGEPNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVN

SEQ ID NO: 275 - protein, 614 aa, synthetic construct
KAILVVLLYTFATANANPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVNGVTPKNIIDAFNGGISLAQYTGHGSETAWGTSHFGTTHVKQLTNSNQ

SEQ ID NO: 276 - protein, 642 aa, synthetic construct
KAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 277 - protein, 820 aa, synthetic construct
KAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 278 - protein, 1080 aa, synthetic construct
KAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 279 - protein, 1005 aa, synthetic construct
MKAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 280 - protein, 585 aa, synthetic construct
MKAILVVLLYTFATANANTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 281 - protein, 1004 aa, synthetic construct
KAILVVLLYTFATANADVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 282 - protein, 584 aa, synthetic construct
KAILVVLLYTFATANANTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 283 - protein, 643 aa, synthetic construct
MKAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 284 - protein, 821 aa, synthetic construct
MKAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 285 - protein, 1081 aa, synthetic construct
MKAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 286 - protein, 642 aa, synthetic construct
KAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 287 - protein, 820 aa, synthetic construct
KAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 288 - protein, 1080 aa, synthetic construct
KAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 289 - RNA, 3021 nt, synthetic construct
  source 1..3021: mol_type = other RNA
atgaaagccatcctggtggtgctgctgtacactttcgccaccgctaacgccgacgtgtacaccgaccacggcgacctgtacaacacacctgtgcgcatgttggtggtcgccggcgccaaattcaaagaggccctcaagccttggctgacatggaaagcccagaaggggttctatctggacgtgcactacacagacgaggctgaagtgggcacaacccaggccagcatcaaggcattcatccataagaagtataatgacggactggccgcctctgcagcacctgtcttcctcgcactggtgggcgacactgacgtgatcagcggcgagaaggggaagaagtccaagaaggtgacagacctgtactactccgccgtggacggcgactactttcccgagatgtataccttccggatgtcggccagctccccagaggagctcacaaacattatcgataaggtcctgatgtacgagaaggccaccatgcctgacaagagctacctggagaaggtgctgctgattgctggcgccgattatagctggaactcccaggtggggcagccaacaatcaagtacggaatgcagtactactataaccaggagcacggatacaccgacgtgtataattacctgaaggcaccttacacagggtgttattcccacctgaataccggagtgtccttcgccaactacgtggctcatggatctgaaactgcctgggctgatcccctgctgacaacaagccagctgaaggccctgaccaataaggacaagtacttcctggccatcggaaactgtagcatcaccgctcagtttgattacgtgcagccttgctttggcgaagtgattacaagggtcaaagaaaagggggcctatgcctacatcgggtcctctcccaactcttactggggcgaggactactactggtctgtgggcgccaatgccgtgttcggggtgcagcctaccttcgagggcacctctatgggatcctacgacgctactttcctggaagacagctacaacaccgtgaatagcattatgtgggctgggaacctggccgccactcatgccgggaacatcgggaacattgtgcatatcggggcccactactattgggaggcctatcacgtcctgggggacggatccgtgatgccctaccgggccatgcctaaaactaacacctataccctccctgctagcctgcctcagaaccaggcctcctactctattcaggctagcgccggcagctacgtcgccatctctaaggacggggtgctgtatggcaccggcgtggctaatgctgccggggtcgccaccgtctctatgacaaagcagattaccgaaaatggcaactatgacgtcgtgatcacccgcagcaattacctgccagtgattaagcagattcaggtgggagagcctagcccttaccagccagtcagcaacctggcagctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctggcaggcagcagcgtgggccagaaagtgactctgaaatgggatgccccacagggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcgccaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaattgataa

SEQ ID NO: 290 - RNA, 1761 nt, synthetic construct
  source 1..1761: mol_type = other RNA
atgaaagccatcctggtggtgctgctgtacactttcgccaccgctaacgccaataccggagtgtccttcgccaactacgtggctcatggatctgaaactgcctgggctgatcccctgctgacaacaagccagctgaaggccctgaccaataaggacaagccttaccagccagtcagcaacctggcagctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctggcaggcagcagcgtgggccagaaagtgactctgaaatgggatgccccacagggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcgccaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaattgataa

SEQ ID NO: 291 - RNA, 1935 nt, synthetic construct
  source 1..1935: mol_type = other RNA
atgaaggccatcctggtggtgctgctgtacaccttcgccaccgccaatgctaaggccccttacaccggatgctactctcacctgaacacaggcgtgagctttgccaattacgtggcccacggcagcgagacagcttgggccgatcccctgctgaccacctcccagctgaaggccctgaccaataaggacaagccttaccagcctgtgagcaacctggcagccaccacacagggccagaaggtgaccctgaagtgggaagctccttctgccaagaagtccgagggctccagagaggtgaagagaatcggcgacggcctgttcgtgaccatcgagccagccaatgatgtgcgggccaatgaggccaaggtggtgctggccgctgacaatgtgtggggcgacaatacaggctaccagttcctgctggatgccgaccacaatacctttggctccgtgatccctgccacaggccccctgtttaccggcaccgccagcagcaacctgtattccgccaatttcgagtacctgatccctgccaatgcagaccctgtggtgacaacccagaacatcatcgtgaccggccagggcgaggtggtgatccctggcggcgtgtacgactactgtatcaccaatcctgaacctgccagcggcaagatgtggatcgccggcgatggcggaaatcagcctgccagatacgacgacttcaccttcgaggccggaaagaagtacacattcaccatgagacgcgccggcatgggcgacggcacagacatggaggccgaggacgacagccctgccagctatacctatacagtgtaccgggacggcaccaagatcaaggagggcctgacagccaccacctttgaggaggacggcgtggccgccggcaatcacgagtactgcgtggaggtgaagtacaccgccggcgtcagccccaaggtgtgcaaggacgtgaccgtggagggcagcaatgagttcgctcctgtgcagaacctggcaggctccagcgtgggccagaaagtgaccctgaagtgggatgcccctcagggcacacctaatcctaaccctaatcccaatcccaaccccggcaccaccctgagcgagtccttcgaaaacggcatccctgccagctggaagaccatcgacgctgacggcgatggccacggctggaagcccggcaatgcacccggcatcgccggctacaatagcaatggctgcgtctatagcgagagcttcggcctgggcggcatcggcgtgctgacccccgacaactacctgatcacacctgccctggacctgcccaatggcggaaagctgaccttctgggtgtgtgcccaggacgccaattacgccagcgagcactacgccgtgtacgccagcagcaccggcaatgacgccagcaatttcgccaatgccctgctggaggaaaccatcaccgccaagggcgtgagaagccccaaggccatcagaggcagaatccagggaacatggcggcagaagaccgtggacctgcctgccggcacaaaatacgtggcctttagacacttccagtccaccgacatgttctacatcgatctggacgaggtggagatcaaggccaatggcggcggcagccctacacccaccgactatacatacaccgtgtacagagacggaaccaagatcaaggaaggactgaccgagacaacattcgaggaggatggcgtggctacaggcaaccacgagtactgtgtggaagtgaagtataccgccggcgtgtctccaaagaagtgcgtggatgtgaccgtgaacggcgtgacccccaagaatatcatcgacgccttcaatggcggcatcagcctggcccagtacaccggccacggctccgagacagcctggggcacctcccacttcggcaccacccatgtgaagcagctgacaaattccaaccagtaatag

SEQ ID NO: 292 - RNA, 1935 nt, synthetic construct
  source 1..1935: mol_type = other RNA
atgaaggccatcctggtggtgctgctgtacacatttgccaccgccaatgccaaggccccctacaccggctgctactctcacctgaataccggcgtgagcttcgccaattacgtggcccacggcagcgaaaccgcctgggccgatccactgctgaccacatctcagctgaaagccctgaccaacaaggacaagccctaccagcccgtgtccaacctggcagccaccacacagggccagaaagtgaccctgaagtgggaggcccccagcgccaaaaagagcgagggcagcagagaagtgaaacgcatcggggacgggctgttcgtgacaatcgagcctgccaatgacgtgcgggccaatgaagccaaggtggtcctggccgccgataatgtgtggggcgataataccggataccagttcctgctggatgcagaccataataccttcggctccgtgatccccgccaccggccccctgtttaccggaaccgcctcctccaacctgtattccgctaacttcgaatacctgattcctgccaatgctgaccctgtggtgaccacccagaatatcattgtgacaggccagggcgaagtggtgatccctggcggcgtgtacgactactgcatcaccaatcctgagcccgctagcggcaagatgtggatcgccggcgatggcggaaatcagcccgccagatacgacgacttcaccttcgaggccgggaagaagtacaccttcaccatgagacgggccggcatgggcgacggcaccgacatggaggccgaggacgacagccccgctagctacacctacacagtgtaccgggacggcaccaagatcaaggaaggcctgaccgccacaaccttcgaggaggacggagtggccgcaggcaatcacgagtactgcgtggaggtgaagtacaccgccggcgtgagccccaaggtgtgtaaggacgtgaccgtggagggcagcaatgagtttgcccctgtgcagaacctggcaggcagcagcgtgggacagaaggtgaccctgaaatgggacgccccccagggcacccctaaccctaatcccaatcctaatcccaaccccggcaccacactgagcgagagcttcgagaatggcatccccgccagctggaagaccattgatgccgatggcgacggccacggatggaagcccggcaatgcccctggaatcgccgggtacaattccaatggctgcgtgtacagcgagtccttcggcctgggcggcatcggcgtgctgacacccgacaattacctgatcacccctgccctggacctgcccaacggcggaaagctgaccttctgggtgtgcgcccaggatgccaactacgccagcgagcactacgccgtgtacgcatccagcaccggcaatgacgcctccaatttcgccaatgccctgctggaggaaaccatcaccgccaagggcgtgcggagccctaaggctatcagaggcagaattcagggcacatggcgccagaagaccgtggatctgcctgccggcacaaagtacgtggccttccggcacttccagagcaccgacatgttctacatcgacctggacgaagtggagatcaaggccaatggcggcggcagccctacccctacagactacacatacaccgtgtaccgggacggcacaaagatcaaggagggcctgaccgaaacaacattcgaggaagacggcgtggccaccggcaatcacgagtactgtgtggaagtgaagtacacagccggcgtgtcccctaagaagtgcgtggatgtgaccgtgaacggcgtgacacctaagaatatcatcgacgccttcaacggcgggatcagcctggcccagtacaccggccacggatccgagacagcctggggcaccagccacttcggcaccacacacgtgaagcagctgacaaactccaatcagtaatga

SEQ ID NO: 293 - RNA, 2469 nt, synthetic construct
  source 1..2469: mol_type = other RNA
atgaaggccatcctggtggtcctgctgtacaccttcgccaccgccaatgccaaggccccttacaccggctgctacagccacctgaacaccggcgtgagcttcgccaattatgtggcccatggctctgaaaccgcttgggctgaccccctgctgaccaccagccagctgaaggccctgacaaataaggacaaaccctaccagcctgtgagcaacctggcagccaccacccagggccagaaggtgaccctgaagtgggaggcccccagcgccaagaagtccgagggaagcagagaggtgaagagaatcggagatggcctgttcgtgacaatcgagccagccaatgacgtgagagccaatgaggccaaggtggtgctggccgccgacaatgtgtggggcgataataccggctaccagttcctgctggatgccgatcacaataccttcggctctgtgatccccgctaccggcccactgtttaccggcacagccagcagcaacctgtactccgccaattttgagtacctgattcctgccaatgcagaccccgtggtgacaacacagaatatcatcgtgaccggacagggcgaagtggtgatccctggcggcgtgtacgattattgcatcaccaatcccgagcctgcctctggcaagatgtggatcgccggcgatggcggcaatcagcccgccagatacgacgatttcaccttcgaggccggcaaaaagtacaccttcaccatgcgcagagccggcatgggcgacggaaccgacatggaggccgaagatgacagccccgccagctacacctacaccgtgtaccgggacgggaccaagatcaaggaaggtctgaccgccaccaccttcgaggaggatggcgtggccgccggcaaccacgaatactgcgtggaggtgaaatacacagccggcgtgagccctaaggtgtgcaaggacgtgaccgtggagggcagcaatgagtttgcccccgtgcagaacctggcaggcagctctgtgggccagaaggtgacactgaagtgggatgccccccagggcacccctaatcccaatcctaaccctaaccccaatcccggcaccacactgagcgagagcttcgagaacggcatccccgcctcctggaagaccatcgatgccgatggcgacggccacggctggaagccagggaacgcccccggaattgccggctacaattctaatggatgtgtgtactccgagagcttcggcctgggcggcattggcgtgctgacccctgacaactacctgatcacccccgccctggacctgcccaacggcggaaagctgaccttctgggtgtgcgcccaggatgccaattacgcctccgagcactacgccgtgtatgcttccagcaccggcaacgacgccagcaatttcgccaacgccctgctggaggaaaccatcaccgcaaagggagtgcggagccctaaggccatccggggcagaatccagggcacatggagacagaagaccgtggatctgcccgccggcaccaagtacgtggccttccgccatttccagagcacagacatgttctacatcgacctggacgaagtggagatcaaggccaatggcaagagagccgacttcaccgagacattcgagagcagcacacacggcgaggccccagccgagtggaccaccatcgacgccgacggcgacggacagggctggctgtgcctgagctccggccagctggattggctgacagcccatggcggtagcaatgtggtgagctctttcagctggaatggcatggccctgaatccagataattacctgatctccaaggacgtgaccggagccaccaaggtgaagtactattacgccgtgaacgatggcttccctggcgaccactacgccgtgatgatcagcaagaccggcacaaatgccggcgacttcacagtggtgttcgaggaaacacctaatggcatcaacaagggcggcgccagattcggcctgagcaccgaggccaatggcgccaagcctcagtctgtgtggattgagagaaccgttgacctgcccgccggcacaaagtacgtggccttccggcactaccagtgtagcgacctgaattacatcctgctggacgacatccagttcaccatgggcggcagccccacccctaccgactacacctacacagtgtacagagacggcacaaagatcaaggaaggcctgacagagacaaccttcgaggaagatggcgtggccaccggcaaccacgagtactgcgtggaggtgaagtacaccgccggcgtgagccccaagaagtgcgtggacgtcaccgtgaatggcgtgacccctaagaatatcatcgatgccttcaatggcggcatcagcctggcacagtacacaggccacggcagcgagacagcctgggggaccagccactttggcaccacccacgtgaagcagctgaccaatagcaatcagtaataa

SEQ ID NO: 294 - RNA, 2469 nt, synthetic construct
  source 1..2469: mol_type = other RNA
atgaaggccatcctggtggtgctgctgtacacattcgccaccgccaacgctaaggccccctacaccggctgctacagccacctgaatacaggcgtgagcttcgccaattacgtggctcacggaagcgaaaccgcctgggccgaccctctgctgaccaccagccagctgaaggccctgaccaataaagataagccctaccagcccgtgagcaacctggcagccaccacacagggccagaaggtgaccctgaaatgggaagcccccagcgctaagaaatccgaaggaagcagagaggtgaagcggatcggcgacggactgtttgtgaccatcgagcctgctaatgatgtgagagccaatgaggccaaggtggtgctggccgccgacaatgtgtggggcgacaatacagggtaccagttcctgctggacgccgaccacaacacattcggcagcgtgatccccgccacaggccccctgttcaccggcaccgctagcagcaacctgtactccgccaatttcgaatacctgattcccgccaacgccgaccccgtggtgaccacacagaatatcatcgtgaccggacagggcgaggtggtgatccctggcggcgtgtacgactactgcatcacaaaccccgagcccgccagcggaaagatgtggatcgctggcgatggcggcaatcagcctgcccggtacgacgattttaccttcgaggccggcaagaagtacacattcaccatgcggagagccggcatgggcgatggcacagatatggaggccgaggacgacagccctgccagctacacctacaccgtgtacagagacggaaccaagatcaaggagggcctgaccgccacaacattcgaggaggacggcgtggccgccggcaaccacgaatactgtgtggaagtgaagtacaccgccggcgtgtcccctaaggtgtgcaaggacgtgaccgtggagggaagcaatgaattcgctcctgtgcagaacctggcaggcagcagcgtgggccagaaggtgacactgaagtgggacgccccccagggcacacctaatcctaaccctaatccaaatcccaatcccggcaccaccctgtccgagagcttcgaaaacggcatccccgccagctggaagaccatcgacgccgacggcgatggccacggctggaagcctggcaatgcccctggcattgccggctacaatagcaatggctgcgtgtacagcgagagctttggcctgggcggcatcggcgtgctgaccccagacaattacctgatcacacctgccctggacctgcccaatggcggcaagctgacattctgggtgtgcgcccaggatgccaattacgccagcgagcactacgccgtgtacgcctccagcacaggcaacgacgccagcaatttcgctaatgccctgctggaggaaaccatcacagccaagggcgtgagatcccccaaagccatcagaggcagaatccagggcacctggcggcagaagaccgtggatctgcctgccggcacaaagtacgtggccttccggcacttccagagcacagacatgttctacatcgacctggacgaggtggagatcaaggccaatggcaagcgggccgatttcacagaaaccttcgaaagcagcacacatggcgaggcccccgccgagtggaccaccatcgatgccgacggcgatggccagggctggctgtgcctgagctccggccagctggactggctgacagcccacggcggctccaatgtggtgagcagcttcagctggaacggaatggccctgaatcctgataactacctgattagcaaagacgtgacaggcgccaccaaggtgaagtactactacgccgtgaatgacggcttccctggcgaccactacgccgtgatgatctctaagaccggcaccaatgccggcgacttcaccgtggtgtttgaggagacacctaatggcatcaacaagggcggggccagattcggcctgagcaccgaggccaatggcgccaagccccagtccgtgtggatcgagcggaccgtggacctgccagccggcacaaagtacgtcgcctttaggcactaccagtgttccgacctgaattacatcctgctggatgatattcagttcaccatgggcggcagccccacacccaccgattacacatacacagtgtaccgggatggcacaaagatcaaggagggactgacagaaacaacattcgaggaagacggcgtggccacaggcaaccacgagtactgtgtggaggtgaaatacaccgccggcgtgagccccaagaaatgcgtggatgtgacagtgaacggcgtgacccctaagaatatcatcgacgccttcaatggcggcatcagcctggcccagtacaccggccacggcagcgagacagcctggggaaccagccacttcggcaccacccacgtgaagcagctgaccaatagcaatcagtgataa

SEQ ID NO: 295 - RNA, 3249 nt, synthetic construct
  source 1..3249: mol_type = other RNA
atgaaggccatcctggtggtgctgctgtacacattcgccaccgccaatgccaaggccccctacaccggctgctacagccacctgaacaccggcgtgtccttcgccaattacgtggcccacggcagcgagacagcctgggccgaccctctgctgaccaccagccagctgaaagccctgaccaacaaggacaaaccctaccagcccgtgtccaacctggcagccaccacccagggccagaaggtgacactgaagtgggaggcccctagcgccaagaagtccgagggcagccgggaggtgaagagaatcggggacggcctgttcgtgaccatcgagcctgccaacgacgtgcgggccaacgaagccaaggtggtgctggccgccgataatgtgtggggcgataataccggctaccagtttctgctggacgccgatcacaacaccttcggcagcgtgattcctgccacaggccccctgttcaccggcaccgccagcagcaacctgtactccgccaatttcgagtacctgatccctgccaacgccgaccccgtggtgaccacccagaatatcatcgtgacaggccagggcgaggtggtgatccctggcggcgtgtacgactactgcatcacaaaccctgagccagcctctggcaagatgtggatcgccggcgacggcggcaatcagcctgctaggtacgatgacttcaccttcgaggccggcaagaagtacaccttcaccatgcggagagccggcatgggcgatggcaccgacatggaggctgaagacgatagccctgccagctacacctacaccgtgtacagagacggcaccaagatcaaggagggcctgaccgccaccacattcgaagaggacggcgtggccgccggcaatcacgagtactgcgtggaggtgaagtacaccgccggagtgagccctaaggtgtgtaaggacgtgacagtggagggctctaatgagttcgcccctgtgcagaacctggcaggcagctctgtgggccagaaggtgaccctgaagtgggacgcccctcagggcacccccaatcccaatcctaatcctaatcctaatcccggcaccaccctgtccgagtccttcgagaatggcatccccgccagctggaagacaatcgatgccgacggcgatggccacggatggaagcctggcaatgcccccggcatcgccggctataattccaatggctgcgtgtacagcgagtctttcggcctgggcggcatcggagtgctgacccctgacaattacctgatcacacctgccctggacctgcccaatggcggcaagctgacattctgggtgtgcgcccaggatgccaattacgccagcgagcactatgccgtgtacgcttcttctaccggcaacgacgccagcaatttcgccaatgccctgctggaggaaaccattaccgccaagggcgtgagatcccctaaggccatcagaggccggatccagggcacatggagacagaagacagtggatctgcccgccggaacaaagtacgtggccttcagacacttccagagcaccgacatgttctacattgatctggatgaggtggagatcaaggccaacggcaagagagccgatttcaccgaaacattcgagagctccacacacggcgaggcccccgccgagtggaccaccatcgacgccgatggagacggccagggctggctgtgcctgtctagcggccagctggactggctgacagcccacggaggcagcaatgtggtgtcctccttcagctggaatggcatggccctgaaccccgataattacctgattagcaaggacgtgaccggcgccaccaaagtgaagtactactacgccgtgaacgacggcttccctggcgaccactacgccgtgatgatctccaaaacaggcaccaatgccggcgacttcaccgtggtgttcgaggagacacctaatggcatcaacaagggcggcgccagattcggcctgagcacagaggccaatggcgccaagcctcagagcgtgtggatcgagagaacagtggacctgcccgccggcacaaagtacgtggcctttcggcactaccagtgtagcgacctgaactacatcctgctggacgatatccagttcacaatgggaggcagccccaccccaacagactacacctataccgtgtacagggacggcacaaagatcaaggaaggcctgactgaaactacttttgaagaggatggagtggccactggaaaccacgagtactgtgtcgaggtgaaatacactgcaggcgtgtccccaaaaaagtgcgtggacgtgacagtgaataatccttaccagcccgtgtccaacctcgctgcaaccacccagggacagaaggtcaccctgaagtgggatgccccttccaccaaaactcaggccactactaataccgccaggagcgttgatggcatcagagaactcgtgctgctgtcagtctctgacgccccagagctgctgcgctctggccaggcagagattgttctggaggcacacgacgtgtggaatgacggaagcggctatcagatcctcctggacgccgaccacgatcagtatgggcaggtcatccctagcgatacccacaccctgtggcctaactgtgctgtgcctgccaacctctttgccccatttgagtacacagttccagagaatgccgatccatcttgctccccaaccaatatgatcatggatgggactgccagcgtgaacatccccgcaggcacctacgactttgccatcgccgctccacaggccaacgccaagatctggatcgccgggcaggggcctactaaagaggacgattatgtcttcgaagctggaaagaagtatcattttctgatgaaaaagatggggagcggcgatggcaccgagctgactattagcgagggcggaggatccgattacacatacaccgtgtatagagacgggactaagattaaagaggggctgacagccacaacctttgaggaggatggagtggctgcaggaaatcacgaatattgtgtggaagtcaagtacacagctggcgtgagccccaaggtgtgcaaggatgtgacagtggaagggagcggagtcaccccaaaaaatatcattgatgcttttaatggcggaatctctctggctcagtacaccgggcacgggtctgaaaccgcctggggaacctctcactttggaacaacccacgtgaagcagctgaccaactctaaccagtgatga

SEQ ID NO: 296 - RNA, 3249 nt, synthetic construct
  source 1..3249: mol_type = other RNA
atgaaagcaatcctggtggtcctgctgtacacattcgctaccgccaatgccaaggcaccttacaccgggtgttactcccatctgaatacaggcgtgagcttcgccaattacgtggcccacggcagcgaaaccgcttgggccgaccctctcctgaccacctcccagctgaaggccctgacaaataaggacaaaccttaccagcctgtgtccaacctggcagccacaacccagggacaaaaggtgacactgaagtgggaagcccccagcgccaaaaagagcgagggcagcagagaagtgaagagaatcggcgacggcctgttcgtgacaatcgaacctgcaaatgacgtgcgggccaatgaggccaaggtggtgctggccgccgataatgtgtggggcgataataccggctaccagtttctgctggacgccgatcacaacaccttcggcagcgtgattcctgccacaggccccctgttcaccggcaccgccagcagcaacctgtactccgccaatttcgagtacctgatccctgccaacgccgaccccgtggtgaccacccagaatatcatcgtgacaggccagggcgaggtggtgatccctggcggcgtgtacgactactgcatcacaaaccctgagccagcctctggcaagatgtggatcgccggcgacggcggcaatcagcctgctaggtacgatgacttcaccttcgaggccggcaagaagtacaccttcaccatgcggagagccggcatgggcgatggcaccgacatggaggctgaagacgatagccctgccagctacacctacaccgtgtacagagacggcaccaagatcaaggagggcctgaccgccaccacattcgaagaggacggcgtggccgccggcaatcacgagtactgcgtggaggtgaagtacaccgccggagtgagccctaaggtgtgtaaggacgtgacagtggagggctctaatgagttcgcccctgtgcagaacctggcaggcagctctgtgggccagaaggtgaccctgaagtgggacgcccctcagggcacccccaatcccaatcctaatcctaatcctaatcccggcaccaccctgtccgagtccttcgagaatggcatccccgccagctggaagacaatcgatgccgacggcgatggccacggatggaagcctggcaatgcccccggcatcgccggctataattccaatggctgcgtgtacagcgagtctttcggcctgggcggcatcggagtgctgacccctgacaattacctgatcacacctgccctggacctgcccaatggcggcaagctgacattctgggtgtgcgcccaggatgccaattacgccagcgagcactatgccgtgtacgcttcttctaccggcaacgacgccagcaatttcgccaatgccctgctggaggaaaccattaccgccaagggcgtgagatcccctaaggccatcagaggccggatccagggcacatggagacagaagacagtggatctgcccgccggaacaaagtacgtggccttcagacacttccagagcaccgacatgttctacattgatctggatgaggtggagatcaaggccaacggcaagagagccgatttcaccgaaacattcgagagctccacacacggcgaggcccccgccgagtggaccaccatcgacgccgatggagacggccagggctggctgtgcctgtctagcggccagctggactggctgacagcccacggaggcagcaatgtggtgtcctccttcagctggaatggcatggccctgaaccccgataattacctgattagcaaggacgtgaccggcgccaccaaagtgaagtactactacgccgtgaacgacggcttccctggcgaccactacgccgtgatgatctccaaaacaggcaccaatgccggcgacttcaccgtggtgttcgaggagacacctaatggcatcaacaagggcggcgccagattcggcctgagcacagaggccaatggcgccaagcctcagagcgtgtggatcgagagaacagtggacctgcccgccggcacaaagtacgtggcctttcggcactaccagtgtagcgacctgaactacatcctgctggacgatatccagttcacaatgggaggcagccccaccccaacagactacacctataccgtgtacagggacggcacaaagatcaaggaaggcctgactgaaactacttttgaagaggatggagtggccactggaaaccacgagtactgtgtcgaggtgaaatacactgcaggcgtgtccccaaaaaagtgcgtggacgtgacagtgaataatccttaccagcccgtgtccaacctcgctgcaaccacccagggacagaaggtcaccctgaagtgggatgccccttccaccaaaactcaggccactactaataccgccaggagcgttgatggcatcagagaactcgtgctgctgtcagtctctgacgccccagagctgctgcgctctggccaggcagagattgttctggaggcacacgacgtgtggaatgacggaagcggctatcagatcctcctggacgccgaccacgatcagtatgggcaggtcatccctagcgatacccacaccctgtggcctaactgtgctgtgcctgccaacctctttgccccatttgagtacacagttccagagaatgccgatccatcttgctccccaaccaatatgatcatggatgggactgccagcgtgaacatccccgcaggcacctacgactttgccatcgccgctccacaggccaacgccaagatctggatcgccgggcaggggcctactaaagaggacgattatgtcttcgaagctggaaagaagtatcattttctgatgaaaaagatggggagcggcgatggcaccgagctgactattagcgagggcggaggatccgattacacatacaccgtgtatagagacgggactaagattaaagaggggctgacagccacaacctttgaggaggatggagtggctgcaggaaatcacgaatattgtgtggaagtcaagtacacagctggcgtgagccccaaggtgtgcaaggatgtgacagtggaagggagcggagtcaccccaaaaaatatcattgatgcttttaatggcggaatctctctggctcagtacaccgggcacgggtctgaaaccgcctggggaacctctcactttggaacaacccacgtgaagcagctgaccaactctaaccagtgatga

SEQ ID NO: 297 - protein, 596 aa, synthetic construct
MKAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 298 - RNA, 1794 nt, Synthetic construct
  source 1..1794: mol_type = other RNA
atgaaagccatcctggtggtgctgctgtacactttcgccaccgctaacgccaaggcaccctacaccggctgttacagccatctgaataccggagtgtccttcgccaactacgtggctcatggatctgaaactgcctgggctgatcccctgctgacaacaagccagctgaaggccctgaccaataaggacaagccttaccagccagtcagcaacctggcagctacaactcagggccagaaagtgaccctgaagtgggaagcaccttctgcaaagaagtccgaggggtctagggaagtcaaacggatcggagacggcctgttcgtcactatcgagcccgccaacgacgtgcgggctaacgaggccaaggtggtgctggctgccgataacgtgtggggcgataacaccgggtaccaattcctgctggacgcagatcacaatacatttgggtccgtgattcccgctacagggccactgtttacagggactgcatccagtaacctgtattccgcaaacttcgaatacctgatccctgccaatgcagaccctgtggttacaacccagaatattattgtgactggacagggcgaggtcgttattccagggggcgtgtacgactactgtatcaccaaccccgagccagcatctggcaagatgtggatcgcaggagatgggggaaaccagcccgccaggtacgacgactttactttcgaggccggcaagaagtatacattcacaatgagacgcgcaggaatgggagacggaaccgatatggaggctgaggatgactctcctgcaagctacacctatactgtgtatagagacggcaccaagatcaaggagggcctgactgccaccaccttcgaggaggatggtgtggccgccgggaaccatgagtattgcgtggaagtgaagtacactgcaggagtgtcccccaaggtttgcaaggatgtcactgtggagggatccaatgagtttgcacccgtgcagaacctggcaggcagcagcgtgggccagaaagtgactctgaaatgggatgccccacagggaactcccaatcctaaccctaatccaaaccctaaccctggcaccacactgtctgagtctttcgaaaacgggatcccagcttcctggaagactatcgacgccgacggagatgggcacggctggaagcctgggaatgcacccggcattgctgggtacaactccaatggatgtgtgtactccgagagcttcggactgggcggcattggcgtgctgactcctgacaactacctgatcactcctgctctggacctgcctaacggaggcaaactgactttctgggtctgcgcccaggacgctaactatgcatcagaacactacgcagtgtatgccagctctaccggaaacgacgcatctaatttcgccaatgccctgctggaagagactattacggccaagggagtgaggagcccaaaggcaatccggggccgcattcagggcacttggcgccagaagactgtggacctgccagccgggaccaaatacgtggctttccggcatttccagtccactgacatgttctacattgacctcgacgaggtggaaatcaaagccaacggcggagggtcccctacccctaccgattacacctacaccgtgtatagggatggcacaaagatcaaagagggactgaccgaaaccacattcgaggaggacggtgtggccaccggcaaccacgaatactgcgtggaggtcaagtacactgccggcgtgagcccaaagaagtgtgtggacgtgaccgtgaattgataa

SEQ ID NO: 299 - protein, 595 aa, synthetic construct
KAILVVLLYTFATANAKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 300-358 - skipped (59 entries)

SEQ ID NO: 359 - protein, 989 aa, synthetic construct
MDVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 360 - protein, 988 aa, synthetic construct
DVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 361 - protein, 819 aa, synthetic construct
MDVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 362 - protein, 818 aa, synthetic construct
DVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 363 - protein, 569 aa, synthetic construct
MNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 364 - protein, 568 aa, synthetic construct
NTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 365 - protein, 399 aa, synthetic construct
MNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 366 - protein, 398 aa, synthetic construct
NTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFPTDYTYTVYRDGTKIKEGLTETTFEEDGVATGNHEYCVEVKYTAGVSPKKCVDVTVN

SEQ ID NO: 367 - protein, 990 aa, synthetic construct
MEPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLANYTGHGSETAWGTSHFGTTHVKQLTNSNQLPFIFDVASVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEPNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVN

SEQ ID NO: 368 - protein, 989 aa, synthetic construct
EPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNITTEDKWLGQALCIASAEGGPSADNGESDIQHENVIANLLTQYGYTKIIKCYDPGVTPKNIIDAFNGGISLANYTGHGSETAWGTSHFGTTHVKQLTNSNQLPFIFDVASVNGDFLFSMPCFAEALMRAQKDGKPTGTVAIIASTINQSWASPMRGQDEMNEILCEKHPNNIKRTFGGVTMNGMFAMVEKYKKDGEKMLDTWTVFGDPSLLVRTLVPTKMQVTAPAQINLTDASVNVSCDYNGAIATISANGKMFGSAVVENGTATINLTGLTNESTLTLTVVGYNKETVIKTINTNGEPNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVN

SEQ ID NO: 369 - protein, 990 aa, synthetic construct
MEPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNIATEDKWLGQALCIASAEGGPSADNGESDIQHENVIANLLTQYGYTKIIKCYDPGVTPKNIIDAFNGGISLANYAGHGSETAWGTSHFGTTHVKQLTNSNQLPFIFDVASVNGDFLFSMPCFAEALMRAQKDGKPTGTVAIIASTIQQSWASPMRGQDEMNEILCEKHPNNIKRTFGGVTMNGMFAMVEKYKKDGEKMLDTWTVFGDPSLLVRTLVPTKMQVTAPAQINLADASVQVSCDYNGAIATISANGKMFGSAVVEQGTATIQLTGLTQESTLTLTVVGYNKETVIKTINTNGEPNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVN

SEQ ID NO: 370 - protein, 989 aa, synthetic construct
EPGRYTPVEEKQNGRMIVIVAKKYEGDIKDFVDWKNQRGLRTEVKVAEDIASPVTANAIQQFVKQEYEKEGNDLTYVLLIGDHKDIPAKITPGIKSDQVYGQIVGNDHYNEVFIGRFSCESKEDLKTQIDRTIHYERNIATEDKWLGQALCIASAEGGPSADNGESDIQHENVIANLLTQYGYTKIIKCYDPGVTPKNIIDAFNGGISLANYAGHGSETAWGTSHFGTTHVKQLTNSNQLPFIFDVASVNGDFLFSMPCFAEALMRAQKDGKPTGTVAIIASTIQQSWASPMRGQDEMNEILCEKHPNNIKRTFGGVTMNGMFAMVEKYKKDGEKMLDTWTVFGDPSLLVRTLVPTKMQVTAPAQINLADASVQVSCDYNGAIATISANGKMFGSAVVEQGTATIQLTGLTQESTLTLTVVGYNKETVIKTINTNGEPNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVN

SEQ ID NO: 371 - protein, 599 aa, synthetic construct
MNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 372 - protein, 598 aa, synthetic construct
NPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLTGSAVGQKVTLKWDAPNGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 373 - protein, 599 aa, synthetic construct
MNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVNGVTPKNIIDAFNGGISLAQYTGHGSETAWGTSHFGTTHVKQLTNSNQ

SEQ ID NO: 374 - protein, 598 aa, synthetic construct
NPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSNEFAPVQNLAGSAVGQKVTLKWDAPQGTPNPNPNPNPNPNPGTTTLSESFGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVQVTVNGVTPKNIIDAFNGGISLAQYTGHGSETAWGTSHFGTTHVKQLTNSNQ

SEQ ID NO: 375 - protein, 627 aa, synthetic construct
MKAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 376 - protein, 626 aa, synthetic construct
KAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 377 - protein, 627 aa, synthetic construct
MKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 378 - protein, 626 aa, synthetic construct
KAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 379 - protein, 805 aa, synthetic construct
MKAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 380 - protein, 804 aa, synthetic construct
KAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 381 - protein, 805 aa, synthetic construct
MKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 382 - protein, 804 aa, synthetic construct
KAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 383 - protein, 1065 aa, synthetic construct
MKAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLANYTGHGSETAWGTSHFGTTHVKQLTNSNQ

SEQ ID NO: 384 - protein, 1064 aa, synthetic construct
KAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLANYTGHGSETAWGTSHFGTTHVKQLTNSNQ

SEQ ID NO: 385 - protein, 1065 aa, synthetic construct
MKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 386 - protein, 1064 aa, synthetic construct
KAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 387 - protein, 989 aa, synthetic construct
MDVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 388 - protein, 988 aa, synthetic construct
DVYTDHGDLYNTPVRMLVVAGAKFKEALKPWLTWKAQKGFYLDVHYTDEAEVGTTQASIKAFIHKKYNDGLAASAAPVFLALVGDTDVISGEKGKKSKKVTDLYYSAVDGDYFPEMYTFRMSASSPEELTNIIDKVLMYEKATMPDKSYLEKVLLIAGADYSWNSQVGQPTIKYGMQYYYNQEHGYTDVYNYLKAPYTGCYSHLNTGVSFANYVAHGSETAWADPLLTTSQLKALTNKDKYFLAIGNCSITAQFDYVQPCFGEVITRVKEKGAYAYIGSSPNSYWGEDYYWSVGANAVFGVQPTFEGTSMGSYDATFLEDSYNTVNSIMWAGNLAATHAGNIGNIVHIGAHYYWEAYHVLGDGSVMPYRAMPKTNTYTLPASLPQNQASYSIQASAGSYVAISKDGVLYGTGVANAAGVATVSMTKQITENGNYDVVITRSNYLPVIKQIQVGEPSPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 389 - protein, 569 aa, synthetic construct
MNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 390 - protein, 568 aa, synthetic construct
NTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 391 - protein, 627 aa, synthetic construct
MKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 392 - protein, 626 aa, synthetic construct
KAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 393 - protein, 805 aa, synthetic construct
MKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 394 - protein, 804 aa, synthetic construct
KAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 395 - protein, 1065 aa, synthetic construct
MKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 396 - protein, 1064 aa, synthetic construct
KAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGSNVVSSFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 397 - protein, 580 aa, synthetic construct
MKAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 398 - protein, 579 aa, synthetic construct
KAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVNVTVN

SEQ ID NO: 399 - protein, 580 aa, synthetic construct
MKAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 400 - protein, 579 aa, synthetic construct
KAPYTGCYSHLNTGVSFANYVAHGSETAWADPLLTTSQLKALTNKDKPYQPVSNLAATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLAGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGGGSPTPTDYTYTVYRDGTKIKEGLTETTFEEDGVATGNHEYCVEVKYTAGVSPKKCVDVTVN

SEQ ID NO: 401 - protein, 821 aa, synthetic construct
MKAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 402 - protein, 820 aa, synthetic construct
KAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 403 - protein, 805 aa, synthetic construct
MKAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 404 - protein, 804 aa, synthetic construct
KAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 405 - protein, 805 aa, synthetic construct
MKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 406 - protein, 804 aa, synthetic construct
KAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 407 - protein, 1081 aa, synthetic construct
MKAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 408 - protein, 1080 aa, synthetic construct
KAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 409 - protein, 1065 aa, synthetic construct
MKAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLANYTGHGSETAWGTSHFGTTHVKQLTNSNQ

SEQ ID NO: 410 - protein, 1064 aa, synthetic construct
KAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPTDYTYTVYRDGTKIKEGLTETTFEEDGVATGNHEYCVEVKYTAGVSPKKCVNVTVNNPYQPVSNLTATTQGQKVTLKWDAPSTKTNATTNTARSVDGIRELVLLSVSDAPELLRSGQAEIVLEAHDVWNDGSGYQILLDADHDQYGQVIPSDTHTLWPNCSVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLANYTGHGSETAWGTSHFGTTHVKQLTNSNQ

SEQ ID NO: 411 - protein, 1065 aa, synthetic construct
MKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 412 - protein, 1064 aa, synthetic construct
KAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPTDYTYTVYRDGTKIKEGLTETTFEEDGVATGNHEYCVEVKYTAGVSPKKCVDVTVNNPYQPVSNLAATTQGQKVTLKWDAPSTKTQATTNTARSVDGIRELVLLSVSDAPELLRSGQAEIVLEAHDVWNDGSGYQILLDADHDQYGQVIPSDTHTLWPNCAVPANLFAPFEYTVPENADPSCSPTNMIMDGTASVNIPAGTYDFAIAAPQANAKIWIAGQGPTKEDDYVFEAGKKYHFLMKKMGSGDGTELTISEGGGSDYTYTVYRDGTKIKEGLTATTFEEDGVAAGNHEYCVEVKYTAGVSPKVCKDVTVEGSGVTPKNIIDAFNGGISLAQYTGHGSETAWGTSHFGTTHVKQLTNSNQ

SEQ ID NO: 413 - protein, 827 aa, synthetic construct
MKAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 414 - protein, 826 aa, synthetic construct
KAILVVLLYTFATANAKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 415 - protein, 811 aa, synthetic construct
MKAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 416 - protein, 810 aa, synthetic construct
KAPYTGCYSHLNTGVSFANYTAHGSETAWADPLLTTSQLKALTNKDKPYQPVSNLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYNCSDLNYILLDDIQFTMGGSPTPGGSPTPTDYTYTVYRDGTKIKEGLTETTFEEDGVATGNHEYCVEVKYTAGVSPKKCVNVTVNGVTPKNIIDAFNGGISLANYTGHGSETAWGTSHFGTTHVKQLTNSNQ

SEQ ID NO: 417 - protein, 811 aa, synthetic construct
MKAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ

SEQ ID NO: 418 - protein, 810 aa, synthetic construct
KAPYTGCYSHLNTGVSFANYVAHGSETAWADPLLTTSQLKALTNKDKPYQPVSQLTATTQGQKVTLKWEAPSAKKSEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEAEDDSPASYTYTVYRDGTKIKEGLTATTFEEDGVAAGNHEYCVEVKYTAGVSPKVCKDVTVEGSNEFAPVQQLTGSSVGQKVTLKWDAPQGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFANALLEETITAKGVRSPKAIRGRIQGTWRQKTVDLPAGTKYVAFRHFQSTDMFYIDLDEVEIKANGKRADFTETFESSTHGEAPAEWTTIDADGDGQGWLCLSSGQLDWLTAHGGTNVVASFSWNGMALNPDNYLISKDVTGATKVKYYYAVNDGFPGDHYAVMISKTGTNAGDFTVVFEETPNGINKGGARFGLSTEANGAKPQSVWIERTVDLPAGTKYVAFRHYQCSDLNYILLDDIQFTMGGSPTPGGSPTPTDYTYTVYRDGTKIKEGLTETTFEEDGVATGNHEYCVEVKYTAGVSPKKCVDVTVNGVTPKNIIDAFNGGISLAQYTGHGSETAWGTSHFGTTHVKQLTNSNQ